SEQUENCE LISTING (2 sequences)

SEQ ID NO: 1 - DNA, 250541 bp, Homo sapiens
  variation (3)..(3): t/c
tatgacatttttagggtgtatttctttcaaagaatttagaatgcccggagcttcgtctggagtagttacttgtgcacagttttttgtttttgtttttgtttttgttttgcaatctcctcttctcatttttttttcaatgaaaaacagctgaagaatagttttattagttctggacatatgagtggtagtttctggagcagctgttttcacaacctattccaagcaagatgtcttttttaaagactcttgcttctttcctgcttactaattacaatattagtctttctctttgttagttctctgttttcttttagtctcattatttgatcattttaatcttatgatgggaaaaaaaaactaaacatttgcaaatttatacctgtgaagcctactatgtgcagagaggtaggccagtcagtctccacatgcaaagaatgaagagccagacattgcacaatgagtgacaaaggcattagattcaatagaaattcaggaaacagaagaaactttataatgtctgatctattaagcagataacattgaagttgagggaacaggttcatccagggatcaaacttgccctgagaagcctaacaaggaacaaagactttgaaatagtagaaactttgagggttgtacttatgtcccacaatagtacggattaaccaaaacttttgatctcatccaattctgcttcattcatcaaaatgaatgtatattttctcacaatacgttttctctttcaaagagcaagggcttttgaatgtcaagttttctgcaatctagctcacaaacacttatgaggataggcataagaaggtaatgttaacctgaagagtcttgcctggaattaaaagtgcacaatacacattaagacactcattatactatatgagtatatatacattatactttaaagcacatgatactcatgtgttttaaagttgatactgggaggaagagagacgatttttaaattttctcaaatccgtgagagtccttaggtatggtatgctgaagatagctccctgaggattggagtaaaagaaaatggaatgtggtcagaacatgtaacttgagattgaggggctgcccataggaaaatagttgtaggccaaaaagcaggagatggaaagctgagcatgacacctgaggcagcctaccgagttgcaaaggaaccaccatttgcaagtgagcactcagtcatggttggctatttttaaagcatcattacacttagagaagtacttatcacatttgcttggcttatcacccattttgcactctgggtaattgagttgcaaacaaagacatctttatttctcaaaggatataaaatacaagcttttgtatcagaaagtataaagcaaagtaaaaaagatagattccctaaagcagtgattttccaagggtggttccacggttggcagcagctgctgggaacttgttagaaatgcaaattctagcagaatcataatcagaaactcagggagtgtgactcagtgatatctgttataacaaatctttacgataaagtttgagaaacactgtttaatgaataggtaggaaacaccatactttaaagttcagcttcttaagagaccaaagactttatttatttttaaattttttatttccataggtttttggggaatgggtggtatttggttccatgagtaagtggtgatttgtgagaacttggtgcacctatcacccaagaagtatacactgaacccaatttgtagtcttttatccctcacccccttccgactctttcccactgagtccccaaagtccattgtgcccttcttatgcctttgcaacctcatagcttagctcccacttatgagtgagaacatactatgtttcattttcattcctgagttatttcacttaggataatagtctccaatcccatgcaggttgctgtgaatacaattcattcctttttatggctgagtaatattccatcatatatatatgatatatatatcatatatatgtgttatatatatatatatatcatatatatgtgttatatatatcatatatatgtgttatatatatatcatatatatatatatcctagtaggtatatgaactttattttgtaggaattaaaagggtaattgtataatttagggggacagcagacacttattggtagcctatgtaatatatatatatgatatatatatcatatatatacaccaaagtttctttatccactcattgattgatgggcattcaggttggttctacatttttgcaattgtgaattacaaccactatgaaaaacattgtggagacgtcttaaagaactaaaagtagaactaacatttgatccaacaatcccactactgggtatctacccagagaaaaatatgtcattatgcaaaaaagatacttgcacacgcatgtttatagagaccaagactttaaatcaatttgtaaagaatgtgctgtgtgtttactctgtttgcctgaaggagaagtttgagcactgcacagcaattaaacaatgagtcaagacaggccatcatcaggaaaccatgtcaaatatctgaggagagctatcaggaggtagggaagagataagggcagtataagctgaaggcatccagaaagagtaaagacaagatgaaatggggttcttataagaaacttgcctaacacaccaacatttctcattcataagggtatgcttactctgtttctgggaagtaggagaaaaagactctgcttcccattgctttcataaggggtaaacaatggcctcctttagacacaaattgatgaccctcttcagcctccactttctgtgctgaatgtaccagagtttacagccagccaagaagtaagaggtggacatgctccttgatgagtggtttcaggcaacagcctgcagcaccaagtcttctgaaaaacctcccaggtctgaaaacgactatgagaagagtgaaaaaaaaaaagtgtgttggggctgcttctagccagatcctattcagagttccacagctgaacttccctttggactgtgtttagagttcctaaacaaaatagatgggagatgtatcccactgaggagcaaaacagacaaagccattacccctctaagtaagcacagccctgagccaggaacaaaaattggcccagtgtgtgggaacgtgggcctgccaggcctatctagaatccactcccaggcagtggcagtaaggaacatgctgggaaatgttatgctacccctccccaaccaagctttctactcaaaggttaaggaaccaccctcatttccttgaattagccactgtgaattgttaaatgtgagtctaaccaaatctttttcaggttacttatatttttacagaggcctgggatttggtattctataggcactacttcctggattgtaatgggattttagactgacaaccctataccacaaatatacatcagttcttgagagcagacaaagggaaccatgccttactttaatctttcctaagtatggattgttggccatctataaaagaatcccccagattcttgttaaaaaatactggttcacaggtcacatatcagaccagtaaaatcagaaatgtgttagggtggacattgggattctgcatttttaacaaaactgccaagtaatttgtattttcattaatattgaaaacatggtcagttggcttaacagtcaaaatcaactttgcaaaaaagtccaatcatacccacatgcattaattattgttacgtactttgtcttggcaatgatacaacaagacagagggagagagagagagacagataaatggacaaatgcagaaaaggatagaaaaactgaaatgaggcagaaaagaaagtgaaaaggagctatactactatcctttgcaacccattacacaaactgggaagcctaaaacaacagaaatgcattctctctcagttctggaggcagaagcccaccatcaagctgtcagcaaagctatgcttcttccaaaagctctagggcagaatcctctccttgcttcttcaagtttctgattgatcttgacaatccttgatttctggcctcatcactgtcctctctgcctctgcagtcccattgcctcctcctcttccatgtatgtctcctatgcacctgtaccctaaaagatacatgtgattgaatagcccaggataagctccctctcccaggatccttaatttaatgacatctgcaaagaccattttgccaaataaggtcacattcacaggttccaggaattaagacataaaacacagacacagctttctgtggctaccattaagcccactatagaatcaaagaggggaaggaagtgagaaacacggaagagaagaaaacagcagcttctttgccttgggcacatttagccctcttagccaggaaggctgaagtgttttggattgaaggtgagacctcgtgtcaaaagagattgaaatttaaataaatagaaaagatatctctatgccctgtgttgttttaggcatgcttcagtgttctacacaacactgaggaagtctgcctcttgttgatgtcaataataatttagcgaaaggaaaattacaagctgaaaagggtaagtacagctaaccagatgcaatgctattagcctgagtaagaagagttctggaaatgtgacgttaacaaaacatctgttagtgtatcaatcatctccctcatctttggagacacctgggggagggggtacctaaaatgaaaacttcaatctcttatacatgccaaatcctctaggcttgggaaacttacttcacactaaagcacgctaaagtcatgcacgccaagcaataaaatctcattgtgcaaatcgtatgttctgtttgcaataagagacagtaacacttggcttaaattattttttgggtagggtaaatggcttcagaagtataggaaagcacaatcaattcatgtgtaagacataaagaactttagaaagtgcaactcaaataatctaattccaaccttgctaatgcacaggaagtcactttagcacacaaatggaaggtcatcactgtcagcacattgtcacatgcgactcactcatactgaagtgtgcatagtctaagatataattcttcagaattctgccccttcacattttaaaaactcacatgatgggatttcctatgtatgtgatattagaccacaatttggacttcactaacgcatgtaaagggagagagaatcctgtgcccaagggatcatgttgagattgtgaaatatctcccagaactttctggaaacttagagatcctgggcccttacgtggaattctccatgcctgaacgttagacactggcaaatcctgctcatggctcacttcttcctctgtcaagagggcagtgagagggaactgaagctagcaaagccaatcttgaggtgttagtctcacactaaccagtattattgggaagaactttttcattttttaaatgtaacgtgtccacctaaggacagggtcttcctgctaatgatatgagtaacaactctttgaaaacaagtgatatctcgttcttctcaagttatttcatttcacttcccaactttaaggagtacatagcaacccctatcttacagagaacaaaactgaaacttacagaagtgaagccccttgcctaaatgcacagttaagaggggagagcgaggtttcatttccaatctatctgataccaggcccaggccccttgcctgtacagcctctcctctttgacccccactgaagtgatttcttcctgggcccaagtatcactttgaatgttcctcattttctggtgttgctggcagaggtgtggtatgatatgctcatctgccttgactaattttttcttttcagcttctttgatgtagattcaaatttcaaatctgtgaaacctacagaaggtagtatgaaatcataagcatcttttgcttccttcctctcttaggtttatagaacagatctctaaattttcatcagctttttttctacaaattctctctcttccgagaagtagcataaatcctggggaaactgatcgttgtacctttgtagtgagtcatctaagccacagcaactttgtctggaagttccatttccttgcagcatcacactactttttgaaaaaacataaatataggtttggccttggggacttccatctctcagccttctactcatttctcaacacccagcaccttgaggctcaggttattatagagaaatcatcccctgccaatgtactttatgagctcaatttttttccaagcttcacctacacgttgctgcattctgtagctagcttcctgataaaaattattcctctgctaatactgccataatttattgtccctccccaacataagttttacaaaatcacctctgaatgtggaattttaaatacagaggcaagctaacaaaacagaaaattttgacaaaattgtcctaaaatcatgaaattttagagctcagaactttagaaaccatccaagtgaaatttgcctaaaatttttgaaaatttaaaattaaattaaattttaaaaaaagaactttagaaaccagcgcttttacctctccctatagcctttgttttatggttaggaaattaaagcccacaatgggaaaatttatctggaaccagccagaatgcaatctgggtctcctagtcaatgactttcaatgaacaggtaatcttgcaaattcatcctacattatgccaaatctatagctgttgattctcaccttcaatctgccacctttggtaagtattcaggaatatagatcaagtcatttaaattggcttagctcaggctctgctcatggaccaggctcagccagacagcacattgttttatttggtctgtgttaatgccccacagctagtgagatggttttcatctgtcatctctctgttgacagacacactctccccttgctgggcttcttgctacctgaacctcaaatgttcccccaaagtcttttacaagacttccccagaaagtgatgaatgctgcaaaaagggaaaacccaagtcagtgaggaggggaaattggcaagaatttagagttagaatgagtgactggaaaagagaactctggaagtaaacaggggtgaagcataggcaggtaaaagagaaaaccaaagagatggtgcaaacgtacctagattctgctattccagtgagcaatggagaggcctaatgatgttaacagggaaggagctatgcctatccaggctgagggaggaaggaggcatgctggacacctgcagcactgctggtttgggttagaagaacctagcagagcactttctccacagttgttatccctgcctctctctcttatccacttcccatccataattgacagttaaataaagaaacgctccatttagataacactttctccctttttacaggactcaacagggagcagtcggctatttaaacatactttccatagttggtcatttgaacacactgtgtggaaaggaaatgactgtcatgccaagggaaggagaagggatcatcacttttaataatttccccgactttcttcccctgagagtttgctcaattcctgaactaatggacactcgtaaattgggaaagaagaagttattaagaaaacccgatgaacatccaaccacactaaattctccactctagttgccttacacaacccaactttttcctcctttacataagaacaggagagcaaaagttgtatttaatgaagaaaactaaatttgaaagatgacttaggttaaaaatgtattccaatctcttttatatttctcacataatttattacccacattaatgtaattatgtactgttgaaatcaagccaaaaaaaacaaaagttagcataatatgtcaaaaggttactgcaggtccataaaagatgctagtagtttcaatacaaaaattggttaaataatctagtgtttaatttgaagctgcctttaacacagtttgaagaatttttgttttatgaaagctacacaaagtcctaacaaccatctgaaattttttcaatgtctcctattgcaactaaattgctacttctggcagggtataatggctcacacctgtaatctcagcattttgggaggccaaggcactaaaatcacttgaacccaggaggcagaggttgcagcgagccaagatggtgccactgtactccagcctgggcaacacagcaagactctgtctcaaataaataaataaattgctacttttaattttatttaaatattctgtatgtgctctgaagcattatgagaaaatgaaataattggcaggactctgcagtcagtgtttcgtttggttaaaaaataatttgaagtatatagtggataatacatatcagaagacaaaaaacgaaaaatacatgtctgctttataaatctctataaaatctaattggagtcagttgttgtataagcagagaatgaacagaaaagctaaatataaaattgaaaatttaaaaatactctaaaaactcttatgaaaaaaattggtcagcctctcctggaatgactcagacaagctcttctttttggtaatgggcctggaatcatatcaaaaaatataagttaagaaaacactatcttttttatattatgaaactaaaattcatcaaaagataatttggagatctatgggaactattttaaaataacaaatgccaaaaaaattatagtccttaatttgttatgttaatgatttgaaacatatgtggtaatatcaaaatatttaactcaaatgtggcaactccacatgtgttttaccaagaataactttgtaaaaacagactctctaccccacgctcctagaaaaaggagagaaacaggtacaaaattttctgcttaagtcaaatctatgtgggtatcaaaaattaacaacccttactattgtggtagtctctgaacccctttaagaatcagaatcttcttctagagaaaaaaaaatagacctacacatgcacagaattctggaagcttaagaacttgtgaagccaatttataaaatccatgctaagaaccctggatcctaaggtaaactcctgagagaaagagaaacaaacgtcaaacaagcctttagataatattctgtatcagtcgtggcaaagagtgatctccatccacccagaaccactcacgtgatttatgactgtttttcagtgttgggagcctgagagctgtaggacatgatttagtggctcctacgacactgggactgtggcctgggttgctggtgtcaaagaaatgggacttttttcaggttcccagctaacttggtgacttgaaggttgtaggctggtactgaaaattgctcactaagcagtgatcacccaccctccacccaccactccgtacccccaaattcacccagttctctcagtagctgaaaaataggatagcttgctgttggtttctttagacctgaatagatgtgggcttagcattttcccatgatgtctttgaagaagaaagcgggagtaaaatcacaatagtgggaaaggaaaattctttgacgttggcatcttcattctaagtacccctcatccgctggtacacttggggtgaattttgtcatctgcaataggtgagaaaaaatggtagaaccgagttgtaaatctccagcagatgagaggcaagaccagagtttcacatgaggaggaagcaacggccattcacagtaatgtctagactggctctggccatagcacatagctgatcatggctgcagtgtagcctcccacagtgcagccatcaggaatgtgcaggattgctctgatcccagggcctaggatagggcatgcacagagtaaggtgctttcccaacatctctggaaggaagggagagaggagaaatggaggaaaaggtggtaatggagagtagtaaaatatacatggaaagtgatcagaggatatattagtttgccagagctgacataagaaaatgccacaaactgggtggctaaacaacagaaatttttttttcccacagttctggaggctggaagtccaagatcaagatgtcagcagaattggtttctcctgaagcccctctctttggcttgcagatgatcaccttcttgtttttttttctcacatgatctttcttctgtactcacacactcctagtgtctttctgtgtgtccaaatttcccgtgtttataaagacacagattaggctggattagggcccatcctaataacctcatgttaatgtaatcacctctttaaaggccctgtctccaaatatactcacattctgaggtaatggggattagggcttcaacatgtgaatttggtagagatacacatcagccaaaaacagagggaaatactggttttgttgaaccctagccccatctcttctccctttcacatcccaaaatcttaggtgggagattagatctgagagatgaggcatggtcaagggtcccagaccctcaagtgaaatgagcaatggcactaagaagtctctccagctcttcctctaggacacccgactggctacatagacagagtcataagtaccaggagggccacagacagccacatagacaaaagccactcccagtggtacagaggttcccaggtaccagcaaaccattagactcttccatagaaagcctcttgcttttagaaaaatcaaatcttaggtactttctgaggttgttatctgccttttgtctgtattgagaacatataaaggactccttcaaattaataagaaaaagcctatttttaaaaatggacaagatttgaatgggcccttcaaaaaagaggaaattcaaataggcagtaaacatattaaaatatattcaaagaaatgcaaattataaccactttcagacaccagttgatacccttctgaatggccaaaagcaaaaggagtatctatatcaaagtttggcaatcatacggagcaagaggaatgtgtttataagggtaaatttgtaataatcattttgaaaaaatatatataatgttaaggtattattaaaacaataataagacaaaacaatactatgaaatatcacatattcaatatgttaggggaagaaatatgttatttgattttggtattcaacatagagcaaaatatgagaatctttgaattaatgcaaattaagtgtgttaaattttaatagtatttcctgtacttgaccaaaacgctatgtttttagaacactcggagtaaatctaatgtctccttcacatggcagctcctcagaaattaaaagacagtttgttttcacatccttttgagacttctctgcccttggtagtgccttcatctgtttttctgcaccaccctgatcactttcctatgaacaaattcttttttggtttatatctctttaactgttggggcccagaataaaacacatcgctgtctgtatattttgacaaagcaaactagagtaagactgacatttctttcttatttgatacaaaattttcagtaatgctaaagtcccatcaactttttgatagtcacttaacactttacccgtttggatacacattacctcctctgaactaaaacctttaagtcttttctacacatattgttacaaagcaccctctgttttgtgtatttttaattcacttgcacacatttatatataccaatttttaaatgcatttttttaaattcaacacaatgctccaaactccctttgggattttgattttgtcatctaatatgtacattaattgaaaaaacagtacagcatgaccttcatgtgtgtatgtgtgtcagtgtgtgtgcatgtgtgtgaagatggttatagataaacataggtatagatataaatatacagacagacatggatatataaatatatattactataaatctatatggcaagttataactacagttattactaagtgacaggattgtgtgtgattcctattttcttgcacttgatttttatatttcctttgaaatgtgtatctagtgaggcagaatgctcaaaattagtgccatcatcttctatgtttagaggtggagaaggcaagtcatattggaacagtttagtccagataatacttgggttacagatgcatgagaagaatcttaaaaaggaatataagtttgcttcagattaatgggtaccatcagcaaagaatatagatttgtttagagatcaaacagaaaatgtactatggcttgatcagcaatgactttgaatgtgtaccaaagagtttattatataaataatttattctagagacattagaaaaaattaaatagctttcaggaaaaggaaaacataatttgacctatttattggaaagagaaaaggtattaccagcgaaaacaccagatttagagacaagaggttagaaataaggataactgttaagtgtccactccagaaacctatagcaaaatgactaggttcaagtagaaaattgtcaagaaaataaagagtggattcagcagtatttcccaaactatattcaacagaacactattatttaccaataaacatctttaaatatttattagtgtcaacattattattaatatttatattatggcaagttggagggattatgtttgatcaaactttagaaatgtcatctattacattatcctgttaaacatttgaaatgcatacaaacatattgaagacctgagaaatcttgccataaaaagcatatttaacattctttaatgcaccattttccaaattcatttgatcacatggaaagcctgttaacgtctcactggatactagtattccatgcacctgaatttgggaaacacagcattatagaaatattgagaaggcagaaacaacaaaacgtagtatttgattgcctgagaaagaagagaattcaaagatgatgttaagttttagccactctgactaagaaaacacgctataccataattatcagttttatgagaacagatttttgggtgggaattagtgtaggcttgggttagttaagtgtcaggtgtcagggtatcaacatttgaagatattcatataataaacataaatatctgcaaaaattatttcaaaaacagtaatggaaattcaggtaaactacttgcaattatttcataaactgttgctaccaaccaaaatgaatgcctagcagagcgctccagcacatggcacagctagaccatgccatgccccatgatgtgtcacacatcggggataagaacaatagcaaagatgctgctaaagaagccacagtaaatggttctcataattgtgaaagctgaataattatgtccacaggatactcctttgctagtcaccgtaactaaagaacttggagcctcttgcccattggcacatgaaattccgggaaacacctttaatctgtatttcaatctcctaaagtttcttgttatttaagagcttaattgttatgactgtgctaagaaaaactcaagaaggtaaaaagagatgttaactacttggacctgaaattacagagcatgaagtaaattctcagaaagaacattaatattgagtagctgcagatatacgtcatacctgggaaaaagtgtgcttaaccaccctctgtcatgcccaatccaatcaagctctgaacatgtgtgagaaattcattccccttcagaaaggagaaagggagaatgcaaggacagtctgtcctgattccagttgttttgctgaactgctctgtgagctgaagcaattccaggaatttatcttcctcaattccacatctgcagaagggtaaaaacagcaatagcaaggtaccttggccctcagctcttcgggcttgctacaaattaccagaacaattcacacttgtaagtatggtggcaggaaaaagctttggggtcagaagaacaaagactacattccacaactccttcaccaattaaaaaatatgatcttgggtagatgttagcttttgggccctctagttttgtcatctgtaaaatgaaaataataattctaaaattgtgagaagtcatagaattaaataatatgtatacacattttaaaagttatttaaaatatgtgctaaatattgtagttatattatctgtcctgtataagtaagattaatggaacatacctgatttctatgttagagagacaatatatacatatgtaattcccaacactaaataataaaccatttagttccaaagttgaatgcctttcctcacactcggctcaatcattttcaaaaagtctacttcatatttaaagcacttgtctctttttctctgccactagttgatatttcactacttttatatccaacttttccttaagtcattcatccaaagaagaaaaaattttgtccctaagaaggagatttgcttagtactttctgttaaatttagaagtggctcccaaggagtgagctctttcaaagacccctttacctctctagttaatgaaagtttaagaccctttagggagcaggcttttgtttgttcattgaatcatctagttaacaagagaatacatcagaatttttttgattcatctattcatgtttctaataagctgatctctcttgtaaacatagttaaatcagtctttactgtatgaaactcttatctgactcagggctgtcattttacatagagatttttacattttagaagttggggaacaactgtaaactcagttcattttacagcattgaccatgtggaagcaaattcttcaaagtcatcacactcgacaccttatagactggttctaagataaagtgagaaattggtgcttttaactgactacatttcacagctaattttgccactattttccgtttcgtttgaaaagtgtcacacctgaaaactatcatgattagttgaagctttgggttaaattccaataccttcagggatccaaaatagttcatagaataattttagttcatcatttattctgtacccacagtgagcaaggtacttaatgcggggtggggggggctaatttcaaatgaaacgacttcccaaagtgtccatatcctgtgtcccttggggacttctctctcctccacacctttagcctttcctaaacacctagcactttatgccaacaaaagtagccaggataaatatgcccattgattaggaacattaaatgggcaagagaactggaaatttagaaagtgtaaattttaccccttaatttagaaccactgatcaaagcattcaagtggcaataccaggtagaccatgaccacagaactctttccatacttgtactccctgcagccatctgactttccctatagcaagctggaacaaattgcagaaatcactgccattcatacctatttagagtccgttgaccctccagggagcctgtaaccagctgcaggcaggccaagatgctcccctaaccagaacttatgtggtggggatacctggcattaaaaaacatgcagaaccatggcgcctgttttctctgtccttcctgcttctggtctcttctgttgctcagtctatttctcccctgagctttgcctctgttttgtggtagtttctttatactgaagttttctttattcctattttattgcaaatatggggtttatccagaattttatctaccaacaaacagtaaattgagtctacatctgttcactctgttcttgagatagtttgaatagtcctcattttcatacctggcaatttatgtttgaacacggggccaattagtctttctgaatattcctgaaatacctacacaattgagggtgatatttttatgagtactgaggttcatttatagctattaaatgaatttaagaaatatatttttagccatcacagcagatgcagtcgataactctcttccgtatgcactgacctaacttctaactgccaacacccgcccctctttacctgaggactttctccaaaacccagaatggcctttcttcccaagcagagcagttataaaatctgaaatttatcactcctcaggaacagcctgcaccagatagccacaggaattgaggcataaatatcccacctcccttggccctcagatggggtagctttgaggtacctgtgcttcaagtttcccagggtatttttgtgggattaatctccaacagcccagcatatccatagccacattttcttgataacacaccccttgtcgtcctgcctcccttcccttttcaccttccctcttccacatcagtgtttcctgaacttccaaactacttttctcaagtctttgtctcagagtctggatgatctcaagttaagaaaactgtctacaatgtgccagacatagataagcactaaaatgacagagatgaataaaaaattatccctgcttcaaggtgttcaccatttgatcagagacaatagcaagaggataagtcaaaaaacctattacccaccttcctttcaagcccacccaacaccatagcacattcacttgggtcctgactcctccccagggccgcgtatctgatgattgtgtctgaccagttgtctcagcaggattgcctagacatgactcagtcgaaactgcttaagagtacataggtgaggtctgaggttggcccacatttcacaaaaaaagaatattctaatcattattgaagaaaatttggtgtcatattgatgatgcgcactgctttccactctgctgaaaaccaggttgactttttaatgcatttttcaatgcctagcattgcccaaccgtcaggtaaataattatattggaccccaggtaataaatcaaactttggtctgtatatcagaccctgacataaagtttcacataaaagatgtttcataaggagtgttcttggaattaatatttggggggaggagtggaaggaagcagaaatgggaaaaaataaaagccaaactgcagtgattgtcccgaaagaacattggccaatgccatgaggacccttggaggtaaaattgaacattagcattgtcctaagttaaaccaagatagttagacttttatttgccatctgtcattggacatgggtcatctaggaaaggatttgaccttgggcaagatggctatccacaacagaggcaagccctggagtgttgacaacagaaggatatctgctgacggcattaccaacaagtccttcattaaggggtatctggactgtgcatcacctaagttcacatcatgtaaatttggaacataataaccccttgcaagtacatcaggtaatgatattgtaggttcactgccaactctggtcgtggtatttacagacacaagaatgctgtttacagaaacaagcatgctaaagctcctttaaaaaaaaagtcttgtggactttttcatgaagtctgtcaagctggaagcaactattaggcatctgaaagcacactttaatgcatgctccagggttcctgctggtgcctccttgaggaagactgtgggagtacatggaattgagctggatgtatcagacagttctccataatcctattattaatgtgccactgataggacagggcaccgccattcctaagtgtggtcataaaaaaaaagtggactcaggtcagaaatctaaactatgaaataatgttactaatcttggtcagaatatgtaattagtcaaaaatacctctcataaaccaaataaagttaatgctatttcagtagtttgctttacttcataaactttcaagagccagggtgatactgcgcagctgttgcatagcgtttataaagaacagggtcttcttagctactgtgaactatgttcacatcccaattatgaaaagacaaagcacatggtcacagaaagtattaatcacattatttatgtgaacccctacttaaccttatagcttgaagcatttcataataaaaacatttgagttgttttaagtcagaggctggttgtgctcccgttaatatttaagaaaggataatcccagcccagtgtggcaagggggaaatatcactgagctgagaattggtgcaagtttctgatatgcctccagttgaatggtgtggaaaattagcctcattttcttctctataaaagcatggggtttgaatattagataatctctaaaggtcctcccaatctaaaagtctatgatcctctttgcctttcagattagttagcagcctaaattgagagacaccctgtgacttaatatcataattttgcatgatgaccagtcctaggttttctttagattggagatttttttcttgcaaattgaaattttccagttttttctttttcctatgacaatgacctcatttcaaagtgttaatattgctatgatatatctctcttgcaaatgttttcccaaacaaaagaatcatccaaatcaatgttatccacgagcttcatatttaagaacatctttaaaatgttggatttgttttactacctcctaagtttcatactaagatctagccaggaaaataaggttgattttcaaacagtagattttagtaaaggggctcactgactgatgcactagaagccaatactgcaacactgggttttttagaaaagaaatgctttttattgcaagtcagctaacaaggagacaggagtccagctcaaatctgtctccctgtgctggctttaaggcagcaattttatctgaaaaggtctaaggtgtggattctaggattagtaagtgattggtggaaggaaaggggaggtgtgggaagtctttggccatgcacagttattccatcatgctacctcttagatcatgtgtgcaaatttgggggaattagtgtaaaacgtgcaatggaaactcaggctgtggcatcagcaagttcgttctatgcgaactccagttggccgtgttgattccaaccaatttctgccaattttattatcttacaagtggaggacatttcagaatttcagcaagttctttcttttcttatctgctgtcctgtaaattcaagaatttatgttagttattgttattaactcttattaatagttattaactctttggggaacaatttcactaggatctgtaacctatcactcttacacactcatagctggtatattagggttatccaaagagatagaaccaatagaatatatatagagaaatatacataaaaagagatcatttatttatttctgtatttctgtatttatttatttattttggggcttttttttgtttgttttttgttttgtttttttttttgtttgttttttgagacagagtctcactctgtcacccaggctggagtgtgcagtggtgcagtcttggctcactgcaaccttcacctcccagattcaagcaattctcgtgcctcagcctccctagtagctgggactacaggtgtgtgccaccatgcccagctaatatttgtatttttagtagagatggggtttcgccatgttggccaaactggtcctgaactcctggcctcaactgatccatctaccttgtcctcccaaagtgctgggattacagatgtgaaccaccacacccagcctaggaattggttcatggactgggaagtcccatggtctgccatttgcaagctacagaactgattgtgtaagtcccaacctgagtgcaaagccccaagaaccaggagtgctgatgtccaagagcaggaagtgatgaatgttcaagctcaagtagagagcacattttcccttcctatgccttttctgttctattcaggcctttaaatggataggatgatgcccatcagaattcttcgctcagcctaccaattcaaatgctaacctcttccagaaacactctcagacacacacgcagaaataatgttttatcagctttctggacatttcatagcccacttaagttgatgttaaattaatcatcacagttgatgtatgtgctcattggccttttgtggcttttttaaggaaacagtatcaacatgatttggaaaccactctgacatgaaaaaattcgggctctattttcatcacattcctacttatttggaccaatctttacaaagagggaaatcctgtgctatgactcatcaggtatgagttctatatctacccttgtgccagagtgtaggcagaactctgggaatggcactaccattatagctacaagggataatggaggggtggttcccaattagaaaaggccttgctgaaaagacaacacaatcaataccatctacatggtgacctgtctatctttcttgaaagtctttccttctgtggcttctttgacgctatgctctcctgatcttctcttacctctctggcaacttttcctcaatatccttccagggctctcgtattccatgcctctttcaaaagttggtcttccagtgggctccatgctcagccctctcttcaatttctacagtctcttgggtgaatgcatataaactcatgacttcaacagccacttctgtggctaattcttatcttttgcctagacttttcaacaagctccaaacacaaattttcaattgtctgccatatgtattttataaacacttcagatgcaacatattgtaaagctgaatttatcaagtttctcagaaatctgattacccctctcttagtcactatcatttccatcacaagctaaagtacatcttaggccaatgccaaaaccctctcttcccttcgtggtttatatttgagtattgtagacaaaaaaaaatcaaagtaagggtaattctctacagatctttagcgttaactgtgaagttaatgtcacagagaaccagagggttgcatggatgttctagtagcaggtgcgggttttggggtgtgggtagggcttgggggtgaaaagtggctataatgacattaagaaagagaagggaagtgcaggaagaaggatggtcagcctcaaaaacaaacatcactgctttactactttggcaggggcttgcccttactgatcccagagcaagcaaggccatgtgacacatctcagtaaaaatttatgcttggaatatacatccctagagactcagagaggacagaatcacagacatttatgaattcaaatgaggcacttaagatcattatgatactcatgaaatttttgctaaaatgggaaatgtaatagtaattttagccaaaactcacacagaagctgtgtcaaaagccaaaagtaagtattctttgagcatccactagaagcccagttttatgccatatttattaatagtaaataaaagtcagaagacttggttatttctcacatggaatgtaaatctacatagggagatgaaatgaacaacaaatacctcacctactgtgtggtccatacattctgtctcttttccacattttcggttcggctttcctgaatttgttgctgagttctttacatactctgctgcttgttatttctgagtgttttccaaagttatgtcttctctcattctcgtttctgaagttcaccttcctcattcctgtttcatctgactaattcctaattagttctttgattagaaagccttccagacctcaagatgaatttggatacgtgagattagagcattaaaaaaagtaatagaaaagcattcactctgttttggttgctaattatttatttatgtttcccacaatgccatgaattctgggacactgtcttgttcattgtatacgtgtgcacttgataactacttgttgaaaaagaatggatcattgattttttattaaaacaatgtaggaattcaataaagaaagtggcacttgagtttaggcttataggacacctaggaaaccaagtgatagaggagacagcagaaggagccaaaatcagttgacagaaaggaccatggacttgtatatgaggattaaaaggagatttgattcatgttaaagcaaatcattcccaacttgagacctgcaaatcccaatgtaataataatgagaacattatttatttttaacatttcaaaaaaccatatacaatgttagtgtatactacatagttccatctatgccatatgtacttatgttgttatgaaacattctcaaacaataatcatcaaaaaagaaaaatgaaaagaagtaggagttgagtgaatgaaagaaggcttcactgaacactttctctgtgttattttacttaatctttaaaacaaatctatttttctcattccttttttttttttttttttcttttgagatgaagtcttgctcttgtcacccaggctggagtgcaatggcgcgatctcggctcactgcaacctctgcctcccggtttcaggcgattttcctgccttagcctcccgagtagctgggtttacagacgcctgccactatgcctggctaatttttgtatttttagtagagacagggtttcaccatgttggccaggctagtcttgaactcctgacctcaggtgttccgcccgcctcagcctcccaaagtgctgggattataggcatgagccaccgtgcccggcctatttttctcattctaatagacgagaaaacaaaggcactaagaagctactaattagaacttctgctaagttagtttttattataaaatcatttagaaacaaattatttcatatggtttcagcatgtatgaaagatactagagtatacccaggattaaaaagagacaaaaggtcatatccatagtaaatgttcaggaaactaacatacgaagaagaaaaagataaatccttaattgtcaggctatgaagattagttgctgtgttcatagaacatgtctatttctgattaaacatttttgaaaaattgtttttctcaccacaacaaacaatatatttggcttaatgtcaataaattagttatatttttattggaacacctgatgaccaagacattttcagtctatctaggcacaatacaagatttagcaagaaaccttttgaaaaattgaaatgggctacttatgaaaatcttcatgtgggtgggaaaaaaagaagttagaacttctcttgaagtacgagcttattttcctaattgaacacagttttccggagaagtgtcaagagaagcacgtgactgctgaactgttctaggaacatgcagaaatctagcaaaataaagattatagatattcaaatctgcaaagcaaaaatgtagtttacagtattcttttttgaaggaaattatttttatatttatacagatatacaatttttattaaatatgtaaatgtgatttttaacatatatattttcaacattattttccccccttttttacttgttttcttttcctcctacctttatttttcacatgcatgtcagggcaccctgccctgtacctgtctgacaagccatattaacatcctagtattgtcctttaatatttttcaacttcagcctcatgtaacctaaatcagatatgggttcacacacatgtaggatgtttaaatattatttattatataaaaagtggaattgtataagcacatgattttctgtatcttactcttctcattcaacactatctcaggtcaaccaagagaaataattcattctttgtaatagctgcataatattccatgttgtgaatagccatttttttcctactgtttcttctattcgtggacattatctttagtttgaaaaattggcttttagtttgttttatcattaagaataaggctgttgtggccttaaataaataccttatgtagtggtgctttttttctaatgaatgtattctgaaatctgtgatcgctggtttaaaagatgtatgtgttttaaattttagcagatactaccaaattgtttcacaaaacaattgcagtgatacacacgtttaccaacaatgtatgagaaggcacttttaatgttcctaacagtgttaaggattccattatttgaataaattttagtatataaagttataactatttaacttgaatttccctgatcattggaaaataataacatttcttcttatgtttctggatctttctaatttgttctttcgttttctctttatccacattctttgcttaattttatattggattgcttagttttatcttgccaatttgaaaaaaactcttttataaaatagatattaacatttttttcatctgtggtactaatatttttgcccagtttcttttcagtcaattcactttacttagagtagctttgaatacaggtttttttaaaaaatcaaatatttatgttattttattccctacaaactctgagtttctagtacttgttactagaaacctaaaattaatttaaaatgaaacttcccagatttttacttatagctttataattttaagttatttatactcaaatctctaagtcatctgtatatgggatattcatggagttcaacttaataaaatctttcaatttgaagacatatttcttttcatttggtatatatcattttatgaatgctaacaagttttataatttcccatgccatcccaatgaaatttgtttctaagacttttatgagattttgttgctattgtggatagaccatttttcccattccagttctaatggcttattgcttaaatagaaaataaagactattgatattgcatggttttcttttcttcatctatcttaccaaatgctctaaccaattccatttgttttcatatgaaaacctcttgttttgtaggtaaactgtctcaattacaaagaatattaaaaacatatgcgaagcaaagcatgcacagaatgtattgataagaaaaccaaagttctgtgtgtagttgactgtatgtattttcatgtactactagagttgttttgtatattattcctgggaaaataaaactgactctgtgcttggtcttctttgaactaaatataatagagaaatagaaatagttattgtccattttcccagttatctgtttctgaacatttccaggaaggattaataggctcatattgcaataccctgaaaagggtcttagagataattacaccaaatgacacaaacagagtcaacggagcttcagttttgccttcctaatgcaattcaaggtgacattgatcaagttgcttccatctcctgtttcatgaaaaaactatttgtgttcagtggagtcactgttaatagcttccctctagtagtgatactctttcagagacctagcagagaggtccatgtaccttagacatgggttcattaaaaaaatgcacatgaaacctcatcatcttctagggaatttttttattattattatactttaagttctagggtacatgtacactacatacaggtttgttacataggtatacatgtgccaagttggtttgctgcacccattaactcatcatttacattgggcatttctcctaatgttatccctcccccagccccggacaccacaacaggccccggtgtgtgatgttccccaccctgtgttcatgtgttctcattgttcaactcccacctatgagtgagaacatgcggtgtttggttttctgcccttgtgatagtttgcttagaatgatggtttccagcttcatccatgcccctgcaaaggacatgaactcatcctttttatggctgcatagtattccatgatgtatatgtgccatattttcttaatccagtctatcactgatgcacatttgggttggtttcaagtctttactattgtgaatagtgctgcaataaacatacgtgtgcatgtgtctttatagtagcatgatttataattctttgggtatatacccagtaatgggatcactgggtcaaatggtatttctagttttagatccttgaggaattgccacaccgtcttccacaatggttgaactagtttacaatcccaccaacagtgtaaaagcattcctatttctccacatcctctccagcatctgttgtttcctggctttttaataatcgtcattcttactggtgtgagatggtatctcattgtggtttggatttgcatttctctgatgaccagtgatgatgagcatttttgcacatgtctgttggctgcataaatgtcttcttttgagaagtgtctgttcatatccttcgtccactttttgatgggattgtttgttttcttcttgtaaatttgtttaagttctttgtagattcttgatattaccctttgtcagataggtagattgcaaaaattttctcccattctgtaggttgcctgttcactctgataatagttttttgtttgtttgtttgtttgttttcttgctgtgcataagctctttagtttaattagatcccatgtgtctattttggcttttgttgccattgtttttagtgttttagtcatgaagtcttcgcccatgcctatgtcctggatagtatggccttggtttccttctagggtttttatggtcttacatttaagtctttaatacatcttcgttaatttttgtataaggtgtaaggaagggatccagtttcagatttctacatatggctagtcagttttcccagcagcatttattaaatagggaatcatttccccattgcttgcttttgtcagatttttccaagatcagatggctgtagatgtgtggtgtaatttctgaggcttatgttctgttccattggtctatatatctgttttggtaccagtaccatgctgttttggttactgtagccttgtagtatagtttgaagtcaggtagcatgatgtccccagctttgttctttttgcttcagattgtcttggctatgccagctcttttttgcttccatatgaactttaaagtagttttttccaattctgtgaagaaagtcagtggtagcttgatagggatagcattgaatctgtaaattactttgggaagtatggccattttcacaatattgattcttcctatccatgagcatggaatgttcttccatttgtttttgtcctcttttatttcattgagcagtggtttgtagttctccttgaacatgtccttcacatcccttgtagctggattcctaggtattttattctctttaataattgtgaatgggagttcactcatgatttggctctctgtcggttattggtgtataggaatgcttgtgatttttgcacattgattttgtatcctgagactttgctgaagttgcttatcaacttaaggagttttggggctgagatgatggagttttctaaatatacaatcatgtctctgcaaacagagataatttgaattcctcctttcctaactgaataccctttatttctttctcttccctgattgcgctagccagaactttcaacactatgttgaataggagtggtgagagagggcatccttgtcttgtgccagttttcaaagggaatgcttccaggtttgccctttcagtatgatattggttgtgggtttgtcataaatagctcctattattttgagatacatttcttcaatacctagtttattgagagtttttagcatgaaggactgttgaattttgtcgaaggccttttctgcatcttttgagacaatcatgtggtttttgtcattgtttctgtttatgtgatggattacgtttattgatttgtatatgttgaaccagccttgcatcccagggatgaagccaacttgattgtggtggataagctttttcatgtgctgctggatttggtttgcccgcgttttattgaggattttcgcatcgatgttcatcagggatattggcctaaaattctctgtttttgttttgtctctaccaggctttggttatcaggatgatgctagcctcataaaatgagttagggagaattccctctttttctattgattgaaatagtttcagaaggaatgatgccatctctcctttgtacctctggtagaattcagctttgaatccatctggtcctggactttttttggttggtaggctattaattattccttaatttcaaagcctgttgttggtctattcagagattcaacttctcctggtttagtcttgggagggtgtatgtgcccaggaatttatccatttcttctagattttctagtttatttgcatagagttgtttatagtattctctgatagcttgtatttctgtgggatcggtgatgataccccctttatcattttttattgtgtctatttgatttttctgtcttttcttctttattagtcttactagtggtctatctatttgttgatcttttcaaaaaaccagctcttggattcatcgatttttttgaagggttttgtgtgtgtctatgtccttcagttctgctctgattttagttatttcttgccttctgcttgcttttgaatgtgtttgctcttgcttctcaagttcttttaattgtgatgttagggtgtcgattttagatctttcctgctctctcttgtgggcatttagtgctataaatttccctctgcatactgcgttaaatgtgtcccagagattctggtgtattgtgtctttgttctcattattttcaaaaaacacctttatttcttccttcattttgtcatttacccagtagttattcaggagcaggttgttcagtttccatgtagatgtgtggttttgattgagtttcttattcctgagttctaatttcattacactgtggtctgagagacagtttgttgtgatttcttttatttcacatttgttgaggagtgttttactaccaattttgtggtcagttttataataagtgcgatgtggttctgaaaagaaagtatattctattgatttggggtggagagttccgtagatgtctattaggtttgcttggtccacagctgagttcaagtcctggatatccttgttaactttctgtctcattgatctgtctgatattgacagtgggcaggtaaagtctcccactattattgtgtgggagtctaagtcacttcgtaggtctctaaggacttgctttatgaatctgggtgctcctgtattgtgtgcatatatatttaggatatttagctcttcttgttgaattgatccctttaccattatggccttctttgtctcttttgatctttgttgacttaaattctgttttatcagagactaggactgcaactcctgcttttttttttctttccatttgcttggtagatcttactccatgcctttattttgagcctatgtgcatctctgcacatgagatgtgtctcctgaatacagcacactgatgggtcttgactctttatccaatttgccagtctgtgacttttaattggggcatttagcccatttacatttaaggttaatattgttatgtgtgaatttgatcctatcattatgatgttatctggttattctgcccattaattgatgcagtttattcatagcatcgatggtctttaccatttggcatgtttttgcagtggctggtactggttgttcctttccatgtttagtgcttccttcaggagctcttttacagcaggcctggtggtgagaaaatctctcagcatttgcttctctgtaaagtattttatttctccttcacttataaagcttagtttggctggatgtgagattctgggttgaaaattcttttctttaagaatgttgaatataggcccccactctcttctgacttgtagggtttctaccaagagatccgctgttggtctgatgggcttccctttgtgggtaacccgacctctttctctggctgcccttaatattttttccttcatttgaaccttggtaaatctgacaattatatgtcttggggttgttcttctcaaggagtatctttgtgttggtctctttagtttctgaatttgaatgttggcctgccttgctaggttggagaagtactcctggataatattctgaagagtgttttctaacttggttccattctccccatcactttcgggtacaccaatcaaacgtagatttggtcttttcacatagtcccatatttcttggaggctttgttcatttcttttcactcttttttctctaatcttgtcttctcacttttttttattaatttgatcttcagtcactgattccctttcttccacttgattgaattggctactgaagcttgtgcatgtgtcaagaagttcttgtgctttggttttcagctccatcaggacatttaaggtcttctctacactgtttattctctttatccattcatctaaccttttatcaaggattttagcttccttgcagtgggttagaacacgctcctttagctcagagaagtttgtttttactgaccttctgaatcctacttctgtcaacttgtaaaactcattctccgtccagttttgttcccttgctggggaggagctgcaaccctttggagaagaagaggtgctttggtttttggaattttcagcttttctgctcgttttctacccatctttgtggttttatctagctttggtctttgatgttggtaacctacagatggggttttagtgtggatgtcctgtttgttgatgttgatgctatttctttctgtttattagttttccttctaacagtcagatccctcagctgcaggtctattggagtttgctggaggttcactccagaccctgtttgcctgggtatctccagcagaggctacagaacagaaaatattgcttcctgatcctgcctctggaagcttcatcccagaagggcacccgcctgtttaaggtgtctgtcagcccctactgggaggtgtttctcctagggaatttttaatgagtaaccaagcacaagacaaggagagagcaggggatttaaatatctggagaagcttcaaaaaccattgatgcacatgtacatacacacacatcacacccatggacacacttacatatgatgagacatatttgagcagaaagttattgttgccctcactattcaaggcaatagtaggggcatgcagaagctgatcttttcattttgtctcctgtgatggttatttcccacaagatgactttctgaaattgtaggctcagaaattctgcaaggtattgatgaccaacccaaatgtgcacagaaatctgaaattaaaatacttattggaaacagtagccaggaaaaaagggagtttactcccagttgggtttatctccttagttaaaacagctctgtatcaggaacatgaactggttcatcactttggattctccagatgaaattgttgaccaggaacccctggtccttaccaatgaaacaagtttctgtattaattccatcatctttgggtgccttattacttcaatctgttatttataagctgaaaaaagtgggtactgactcatgattgtcctctaacaaataaagttgagaagcatcttattggcaataatattactaacatgaaatcaagtacgttttccaaatgattctcgatcttcactgaagagttgccatttttcagttttctatccttagagctcaaggaatagatcttggaagctaatgtaccaagagtgctataaaagacacacacatactggaattttggcgagatattgtacccttttgccagtatgctatgtgttcttcagagtgcagcttcttataaaagctcttttgtgcctttttctttctagtttatgttagagttgatcattttttcctagcactacaagtcccaagtttgtgatcattcaatgctaaaacatttttacttttcttatcaaaagttttttgcaattttttttggtagtgcctctttttttatgcattgaaaatagtgagccaacatatatactgcttgttaattattctcctgtgatcattaattgctgaatgattaatcagaacccatttattattgctgttgtttaagtataattcaacagcaccttttttattaagtcattacctacttcatcataatctttagtcacccttacatctaccagagtcaacatggatgtcctagcctgtgttcccaccgaaagcagagcttatgacaagggcttatacgcaattggtttattctgaaaactccagggaagaggagagaaggagagatggccatttgaagaatctattgaattgacaactggagcttgatcccactgggaccctttgagaagctgtgtagaatgtatggaagctgtacagaattgttcttccaaaagatgggaaaggagatatatagccaccaattccaattctcactgggcacaggtttcccattagcattaatttctacttccagtttgcacagaattactgcagaggtttccgaaagcatttcacctgtgccatcagagacacctcatggcagaaaaaatatatatacatgtgcacatgggtaagcttttgctgcagcaagagcttgcagagattgaggtgaatgaagaggtcatgaggctggacacaagaggaatctaatatagttaaaagtttagtttaaacataagtgagcaaataagttgttcatataaaatcattggtacttctacctctcgcattatagacagccattccacctaatgggtaacaattatattataatgatttgtataatgatttgtataatgaaaatgctttatatttatttatttcatataaatatgctagtatttcaatatggctaacataggattcttatgttctttaaagtttagactttatcctataaccatggtgtggttataaacagaaaagaaatattattgtatttatattttaaaataaacagtttggcaatagcagagaatgaattaaacagactattgcattagtacaatttataaactaatttagaactcaggtagtagccataaaaatgaagaaaggggaaaagacacaagacacgttgaggtaaaatcatcaggagaataattagataaaatgattaggaaaaaggatgaagctgagataactccaagacttctgatttgaattgaaaggtatatttgaatgtctttcatctccatctccaagactacaatccttctttaaacagaaataatgagagataagaccataaactctcacttagaacactgaaatatcctcctcactggtttccctgaatctgctcttccccctacaaatcagcctcccaacagcagccagaatggcaccttcaaaacataaattttattcctttactacaataattaaatgctcctaagggcttttcactggtcttagaataagaggaaaatacttaacacatcttagaagggcctacatggtcttgtccttgctacccttgtaactttgatgactgtcccagatataccaattttttttcagtctctctaatatgctaagctccttctcatctcagggcctttgcaggtgttggaatcactacttggaagtcatcccttgcctagttaactgctactgaactttttgatcccaacttaattagcaatttataggggaagcccttcttgcattccccactccgtatcttcccctcaaagaaagcaggtcttctattagatgctcccacagcatattttattttcccgtcttagcacttattccagcttggaataattatctatttgcaaaataatgtaattaatgagagcctcgctctctgaaaagtaaactctatacacgcaggaaccactattattttgttcaaaattttatccttagtgcataggctttcatgtgactctccatgatgcttgattaattcttattggttgaatacatgaattaatgccaaaagggaagcaggaataattggaaatgtgttttatgtagattggctttaagaggcctataggagattcaagggaagatgcttagcaggtggttgaaaacaaaagcactcactttagagtcaaactgtcagggtttaaaacccagcttagttgtttattatctgcatgactctaggagaattgtgtgatatcactcaactttagttttttctcctgcaaaatgaagatgatgatggcatctacctcacaggtggttgtaagaattaaatgagattatgcgtgttcacttgttaacccagtgcccagcacatagtaagtatacaatgaatgttgccattattgttgttatcactttgtgggtttttttttttttttttgagttggagtcttactctgttgcccaggctggagcacagtggcatgatctcagctcactgcgacctctgccttccaggttcaagcaatcctcctgcttcagcctcccaagtaggtgggactacaggtgcaagccatcacacccagctaatttttgtatttttagtagaaacggggtttcatcatgttggccaggctggtctcaaactcctgacctcaggtgatccgcccacctcggcctcccaaagtgctgggattacaggcatgagccaccatgcccagcttgttgttgttatcactttggaccacaagaaaatgttggaacccaatacaggtttccaaatcattcaagatggttgaagtgatgagagtggttgagatggcctagagagagaacacagagcaacaaaaggccaaaagagagaccttgagtaaatgccaaaagaatgaaaagggataggaaaaaaatacaaagtattgataagatgattagaaaaaggctagggaagtttgacattaacaaacaagattttattcttttctattcatttattaatttaaaataaacccattctttaggttttaagtcttaaaatgtctgggaggcaatatcactacacagagccaaagagcatttattgtattctgtagaaattatctgttttggcttgaaaccactcttaccaggttcaagattcttttctcttcattatatcagagaatagcttccttggtacttagttgatctaaaaatgacacatttatggaggaaagaaatgggtgagtttttcactaaatggggtaatgccctacataaaccccaggatcttttttttatcaaggaattttctgcagaaaatcaaatgattttgagtggatgacatttttttatgaattgaataacagcaacaagaagcagcctgctaagtttgtttcttatctgattcaatggattgtattttcattcctaattgctctttattagctctgacatttctaggtttcgggaaaacaaaaaaaatgcgtatttacttatggtattgtctacttttctcctaaggaaaataaatgatggttcaaaagcaatccctgagaaattcatctccaagggtagatatcctaccaagtgaccatgccctctgtaaccacggcatattggggatcatctggaaattaagaacacacacaaaaaagattccttacattgcttgcttccagctggtaaacaaacaattaagcctcagaatcagaacacatattctttaattagataggaaaataacagttggattatgttatgacatttgctattaataaataacagttctcataattttacaaagagtggatatgacaattctaattattttaccacattgaaagcgctgtatcagagtcccatgggtagccagtcatctatttatcatgaataattcagagaactatggctataattctgattcaatgctgattgaaaatttagagttgttagacaagatttctacagactgttctcttgagtgctatttctggaatttgtatctttgcatatgcaaattctagaaagaatgtaagcaatgagatggttttgcattcattagtaaagaactttcagttttatcagagtccacatcaacaaatacaacctgtgtggaacaggacatgaagatcatgttcagttaatatttgctgtgctaattttaaaaaattatcattatgaacatctatacaataccatctgtctaataatattatacacagtgggggaaaattcccttttggaccttgccagtattttatagagcaacggcaagagtcacagttgttctggaaaatatttccaagtcatagtgaccagaattacaagctttggtttggaagttgcaactactatttctctaagttttcatttacttctcttttttttttttttttttttgagacatggtcttcctctgtcacccaggctggagtgcagtaggatgattatggctcacttcagcctcaacatctggagctcaagtgatcctcccacctcagcctcctgtgtagctgagactacaggtgcgcatcaccacacttggttaattttttactttttgtagagacagggtctagctatgttgccacgctagtctcaaactcctggacccaagcgatcctactgcctcagccttccaaagtgctgggagtacaaggcattagccacagcacccagctctcatttacttcaattacagatcactttattatctaaatcacttttatctattatagtctcatatataatctggaataatttacaagtgtcaaacaccacatttcagtattcagcatgcaagtctatttcatgaccaattacttaaataaataaacaaagaaagaaagaaagaaatggatataaataataatgaagtgcatggaatgatagacaagagtttacctccaaagttacaataacctgtttggacatatcctgtgcaaatccctttccactcagacacattctgagaggcactaagtgaattcagagaggatagagaatgaaaaaaattactcatttcagaatttgcttatctttgacacaatcaacatatctgatagcctgttacagacaaggtactaaaggcaaggctgagagcatggtctctgccttccagaggcccagagtctagaccagttaaattcatggaactatactctccaaataaatctacagtttacatggtcttttatttcaccagcaggcaaatttttgtccctgggcagcttcctagaactatttatagcctgagaaccaatttatttcttcttagtagatttctgttctcctttgaaatctcagcagtacaaacctgattagcagggaggccaggtccaagtattaatgagataattatgattgttttcttataccaggcaaccagcgacttctggcgaggaccagtaggatgaaaagcaggaggtcaagggtaaaatgctggtttccagaattacttgggctcttttatatcttccttgaagaagcaaaatccaatcatgaagatcacaaagacaatagcacacagcagtgggatgaagagcaatgagtacagagccctgagaaggaaaaaggacttgttagaagtttgtctgtggcctggcatccaaggtttatgaaacttttttatgcatacaccctggcaaatatcttctataactacagtgtgtttaaaaagaatttttttcctcactgggcatgttgagaggtcacaaaattcatcatgctaccactcagaaatacatagaaaatttaatttcttaattttctttaaatatctttcacttaagcccaagacattggttagtggaagaaagagacaaaatgttccatttagtttaatgtttattgaatccattaatttatatacctttccctaaaattgtgagtggatggcacaagaagtgaatagcttacctttccctgtggtgtgagtcctcaggaatgttctggtgcctataagtacccagcaccatgccctttgttcccctaggcagtaggcagtgctagagttagcataagaaaaaggcaaaattgaatggctatggagaagtgttccagataaaggcaggccaaatgaaagggcaagaaggcaaatggcaacatgacatattcagggcaactgcagatgattcaatagaaatggatggtagaagcttatatgagaatgtcagaataaatctggaagtgcaagcaaggcctggagccaacgttttatcctgtacatacatgacaggtatgtattttgaacatatcactcaatgtctacccccaggcccctgtaaacttgtctttacataattatcatttaaagtttttgactgtcaccaagggctaaggtaaaaaaatcaaaataaaaacactgaacgtgtaatctgtaagaatttttagtaaaaagaaaaaaggctttgagtagataattttgtgtgcttgactacatttatagatatggttaccaacctgcaggttgaggttcataatattattcaatgctgtcataatacataaagacatcataatatattcacctctaatcaaaggaccaggaaaaggtttgggttttgtttaatgagctaaaataggtacatcttgtagttagacaataagaactacatcatcatctagtttccctcttgcctgggctactgagaatctgagtattaagtttaagtttaatgatgatactattcttagtctgagatttgttttatttttgcttctcttctagatgtctgtccttcccaacattattactttaacttttattctatgttacttgtctcatccatgtgctctctatgagccattttagacttcctcaaaaactagcgattcctgtgctactagagatcatagaatcctgcccacttactaaggggcttcttgagttctcaagctatataaatattttggcgggatcattttttttcctcaaaagcaacatcaaatctccagctgctgggaaatggtgctttttccctattattggaatcaatgagtcaaggcatatgtctttaatggaagttttggcaagcatggtattagagttaatattagattcaactgaagtaaactgaatggtttgttttctgctctgcaagcacaaatcaatggttttaatcaaacactgcagtttatatcatggtagaaatatttacacaaagagaagcactgatagatgcacagcacttaagacaaatgtaagtttttagaagtctcgggagcatagattacaggaaaggtaagacacacacacgaaaaatctccccagccaaaaacagtatataaacctatgttaggaaattgtgcttcccctcaaagagtctagttcagttagcatcttcttgttgcatgcaaaaaaagatttactgtagttatccccagaaatatgggggagggcaagatggtatacagtgaatacaaatgccatggaatacaggaaaagtcagacaactcgagtgggatccacagcagagccatgctacagaatttctacctacaggctgaatttcaaatctgtaaatgtagccaaacaaataataaatacttcactgatattgcattacaatgtacaataaagtattatctatgtttccggactttatggctgccctgtagtgatatgccattaatatttctgcttggcattttctgtcccatcactattttcttatttttttttctttttaccttcatatattttcttctttcttctttaatattttctacttttgtccaactccagcttggccttgacttatgaccccaagcatatattgtgaatgctccctatatgtcctttgacgtacctccaatttctctctgcatttcctaatttggtttatatgaaaaaagtatgattggcctgtacatttttttgtaccaggacacattgtagataattggctcatctctggatttgggtagggaagactatggggtcacacagtatacctgccctgtaagctgtgaacaaagaattttcacttaggagggacacagggcctggtaccaacactgctgggactaacatacagtcctctacttcactcatgtaataagaaagggtccagttctcctaacatgctccctcttaagaaaaagaaaaagtaaaatttcatcttctgagttttaggtggattttcctaatactcaacattggcttctggagtgactacataaagcctaatcaaaatataattaatattcaataacgtaaagcacatgtctctaatgagaacaagttaccacccttaaaaaagtttcatatgcattccaaaaataaatagaaatgaatttctgattattgggtggtattctctatcctcatcactcttcctttgagtggcatactctcctgctgtaattgatacctccaaaggcattattactcaactcctaatgcttcacttgcatatcttgcatcttgcaggtttcaagaatttgtggaacaacacatctttgcttcaaagtactgaagaggttaaagttgcatgaacttaataaatgtgaattaatataatacaatattataaatagacttagagctcattgaaagcagggactatgtctctcttattcactgctctgccccagacactagtgcgatatgtgatacatagtatgcgttcaatatatttttcaaaataaataatcaatcaattaattcatgactaccatatctacttgcccacaacttctaaacaatgggaagcatggagatgagagtctggtgagggccatttttgtaaatatcaaagagtacctctaggttctacagaataacttagtagaatattaaacgagaagtggaataggtttaaatgcaggtcttctaattaaaaatgagttcctagataaaagcaagagtaaaacaaaaacttgcagaggtacttaattattcttagcctctctgtctctctctgtctctctctctgaaggtaacttctaccagccaaagcttaatgtttactgtttggtcttcttagaagaaaagattctttaaaacagccatgaaacccctcatcaggccagggcaagatctatcctccagcaggagttagagtctcattgcccatcttggagctgagtttgaaggaaaagtccctgctctggggtacctctctcatctcacttggtcaacacagagttattagattactaagctcatattctctctccaaatagaagtaacaaaaggcagaaaaccaacagaatgttatacgttactcattttcaacttagtgtgaataatttggcagaacttgataagggcaaaagcatgattttgctactgatgagaaatatttacgtatgctttcatcacacagctgagcacccaaatgagaggcattgttctgaacaatgccactgcctttctcctgaaagcctgttgtcattaactgaaatcagagaaaggggctgatttcaccaacctgaatcactcacttgatggccaaagttctagaagtttgtgctctggggccctgagactggattctttctgcctctgatttggcaactgatgcccactcatttgttaaggattagaggaactcttgagtctcttcttttaaaataaaagccaactttgccattttgttctttcacaaagaacaatctctttgctgaatgaaaaaaaaattgtaattcttctagctgttgagccatgatctctgctaaggggtaagaagatcacaatattccctttcatgaagtgtgttaatggtgtcctccctctgtaaaggcctgaatgtatagtttctgcctggtttaatgtatcatttttgaacaatatgtacctgagccactaaggaccaatgagtaggggctggcaggtggccctcaacccttcctcttgatacctttaatcctcctccttcagccagagaagctctgctttacttctgtttcatatgttgaatttctgcaaaagaagtcatttgacaaaatgatttggaggtttggaaaagatggaaaaccagcatgatggggaatgaacactgaacgtagaaaatctggattctagtcctgctactagccccagttagctagtagacattgagaaataacttcaactttgtagtgtctgactcctcctctgttaaaatgaggcatcaaatgggggaactcaagccaggccatacagtgttttagaggttcctgagaggtccttagggtttactctgaaaaggaaggttggaggctccatttcctcaaccactgcaaacagcacaactctattatatagatacatagagagaaattttatataaaatttagtttgagaaaagctttaaaaaattgaaaagcactcaattctatatgaaaaacctctttcattctaatagtcaattgctccataattcctactttccactggttaaaaaaacaaaacaaaatactttttagtgactttcttattcatagtattatgctatttgttggtgctgatgacctagatgacacaataatataacacacacacatatatgtatgtatgtatgtatgtatgtatatacagatgttccttgacttacaatgggttacattttgataaacccatcataaattaaaaatattgtaagtggaaaatgcatttaatacacccggtctatcaaacatcgtagcttagcctagcctacctcaaatgtgctcagaatgcttaattagcctacggttgagcaaaatcatgtaatgcaatgccaattttgtaataaagtgttgaaaatctcatgtaatttattgaatactctacagaaagtgaaaaatagaagggttgtttgggtacctgaagtactgtttctactaagtgtgtatcacttttgcactaccgcaaagtcaaaaaatcacaagtcaaactattcacaagtcaaggacagtccgtataaaacagtatttttctaagtactcagaatatatatatataattacatatatgtgcaaatttgtgtatatatacacaatttgtaatttcatacatatttaagtatatattggttagtattaacattctctatattgtcttccttgaaatacttttctaggagatattagtgagtatgcatgaaaaaaatgccatattcaaatgagtttaacagattatgcatatttaatctccctgtaggtaattcataatatacatgtgcatgtttaaggcatctcgttgagaagaacttcaatggaaaaaatctatttgatctggtttaacttagagttcccaaacttacctaagtgaagcattctttatcaacattctattgaaattgcacgaaccatgattatgaagcaaactggaaaacaataacgtagtatttgtccagtgtgatggactgcatatttgtgttaccctaaaattcatgtattgactgaagccctaactctcagtatgatggtatttggggtggagcctttgggaagtaattaggtttgaatgacatcatgagggtggtgccctgatgatgggattaatgtccttataagaggaagaagaggcattaaagagttttttctctctctcactctctttctctctctcttcactggcacaaagaggaggtcatgtgaaccacagtgccttctgcaagtggccatctgcaagccaggaagagggccctcaccaagagccaaatccagcacctgaaacttggacttcccagtgcccagaactgttagaaataaacatctgtgcttcaagccacccagtttatagtatattgttatggcagtccaagcaggctataatatccaggtaagaaaattatgactagtgaattgatttggccacggttacaaaaactactgacacaaatagcattctaaatttctacctgaaaatcgatttgaaaagtccagtcaagatggtgaatgaacttcatactctgacatctaatttttatccaaaataatattattgagaactctaaaaaataaaattcaacaatatacgtagggttaaaaacaaaaacatcattatggactataattggaacagaaacacaaagcagggaacatggctaaatcccacggcctgcaggatctatgattaggaaacaggcaggagtgatggcaagtaagggagattgaaaattctatgctagacaagggacaggatccagtctcactgcttgaagccagatgctgggatggaggtagtttcccctccccattgctcttaggaaaaggctaataaaactgctttcatcctagacccaagaccttatacactgtggaatcagcaaagctggtttacaaagcatcaccccacactcaggaactggctgtcagactgttattatttccagcatcacaacaaaggaggggccacaacacatcaaaacatctgtttccagactagagatccagagggctagatgaaggcaacaaagaataccactggacgaagaggagatgagcacaaaggagaaaagacagaaaataaaatcatcccactctggagggagactgcataccatgggaggaaaatgagtgctaagtacggcggccaaaaaaatcacaatggggataataatcccctttgagtgagtaatattcaaataatattctgcctaaaaatttataaaaataatttggaatatccagagagattaaaaaagagaaaatttctttaaagggaataataaattatgatagaaaaataggaaaataagaatgagtacataaaaaacaattgaaacttcttaaaaataatgaaacacacacaaacaaaatttagatgaacccaggatttgacaaagaaggaattaatgaataaaaagatagcactcagaaatttcttaagatgcagcccagagtgacaaagatgaaaattttgaaagagcatctaggacacagggtgtgaaataagagaccccaatgtctgtctgaagggatgcggaaagaagaatgtcaaaagaatgacaaaggaaaagtagtgaaaatcattttacttctgctcaaaatccttcacaactttctgtatcactcagagtgacatctattgtcctgaaaatgctgtgcaaagcctgtgagctcccacacccactctcaccttccagctttctgacctcaccttctcctgatttttgctttgctctctgtttccagtcacactgtcctcatttcttttctccgaactttccaggcatgccccagtaccagaaatttcacattggctgttccctctgcctggaactctcctcctccagaatgccatgtgccttactcactcacctgtttcaattctttgcccacatgtcatcttcctcagaggaccctccccaaccacctgtttaaacttgcatctccagcccctccccaccgtcttttttccctgcattactcttccccttagaactcagtgctatctcacatactatatgcttcatacatttacttatgtctgtctccccacactgggctttaaacccctgagaacagaagtgttttgtttgtatttacttttgtacttccactgcctgacacattgtaggtaccctgtacctaattattgatagaatccataaatgacccctagcatatgagaaacactcaataaaggttagtgctgtgataaagctctacacattaataaaggttaggtattgcaacttagccacaccctttcaattatcaataattccctacttagactgttctgctaatcactctactccagaagagtcagtcacattattttaataatggcaaccatctgcgacatttcttattttactttagcgtcaattccaaagtggggaaattatctccatcttattgtgattatgagactgagatatggcaaaattaatttccccagattcatatacccagagaatagtagagaacatttggaacctcagactgttcacctccagagactctgctgccctctgcactagtgaattgatctgcttgcttctctggcattagaatcctccataaccaccatctctgctccatgctgcctcttgttttgataatcatcttccacagtgactctagcgaacactgttgccattcctcctcactaaaccttgcttatctggatatctctctcttctaaagataaatctgggcatcccaaggtccaagggtaaatctggatttatttaagcaaatcagaaagctaaggtgacatcatcaaaattggcagaatagggaattcttaagactttgtcatgtcgagaaaccaagtaatacactggcaaagactgtcggaatctgagccaatcatggcaatccattctccttgccagtgattggcttaggaatagacatttgatacaattccagccaatcagacatgagttacctggagtacttctgatgatagttctccactcctgagtgggaaaaacaggaggaatggactctccttcctccagacatgttcatatttaaataagatgcctgaactactaccaccatcttttaagcaacccaaatatgaaattaacacatggaagagaacagaaccaataaaatctcagagaagttagagacattgtataccaagcttaatgcctgcattacccatgaaagttatgtgaaacttattaaaagtgtatggatgatataagaaaagttgagttgattttctgtagctctaactgaaaacatcctaataatacatatcacccccttttcacctgagaagtagcccaacctccaaaaatcacttaaatggatgtatctttatctctactatttgaacattgacaaaaatttgaagctcatagtttctgagattggattggttacagacctgccactaaaataacttaagggggaactttagatttttctcacagagcagttgaaagtaggggtggaagagttcaggtcaaaaatcaagctctagttttgtcatggagggtgacatataaagtggtccattgaggcataaagaagttgagtgatttggccaagctcaccatgttagttgataggcaaagtgagaccacaagacaagttcctcaccttctgttacaagacattgcccatgcccaggttaaacctagctgtattcagtgacatataaagcaatgcattgaggcacaaagaagttaagtgatttggccaagctcaccatgttagttgatggcaaactgagactataagacaaattcctcaccttccgttgcaaggcattgtccatgcccatgtcaaacctagctgtattcatactcccagatgcattatgtaactgaaagaagggcagaaaaaagttcaaagatttaacggaattcaattatacctggaagataaatattttgcctcccaatgattattctagcaatgcagaaacaaactaccatttagaaatacgattctacagatctattgaccccttggttgcatctctctgtcaggagaacaattaaactacaacatccaccctctttgtcaagaacattttggaaaacctaagcctgtttcacttcttgcatcttatttggcaaatgatttcttttgacctcattacaggaaaacaattgtcctttgacatgcaagtatagttctcataagcctggctcctttctgacctctgaagtcattcttgggaggatgtttatttatgcttatatcagaattgtttcagagccttaggtctgctttaaatgaatacataaatatatagatgttttttaaatcattggaggtagaaaatgccttaagttgaaactgtctaatattacagctaaaaatgcacactcagggactagatctactggcttaaaatctcagctttaccattttctagctgtatgatcttggacaagttatttaatttttctgcaccttaatctgctcatttttaaaaccaaaaccttttacaatagtacctaactacttatgatgtggactaaattacaccaaactgtctgttaaaaatgtgttaaataatactcatttgtcatgtggattaaatgagttaatatatgtgaagtttctagaactcggcctggcacatggtagtcactatacaagtattatctattattattattatttagcacattagtggcagatacaaccagcaaagcaaactgagtgcttgctatattgcaggcacagagttagatgtctcaagtgcaattgttcatagagtggaaatgcatagttcttgccctcttgcttcactctggggataagtaccataaacagacaattgcaaggcaaagtaatcagtgctgggatggaggtagtacagaatgccatgctaaatgtgggaaaggaaaagtgagaaaagggaggaaacattacctaagctgcaagaagcctttttgaagggagagactcctcagctaggacatgaagtaagggcagaagttaactaagcaaacaattgaggatgaaagtgccacagggagagagaagagtgtggtttttttagcccaataataaaataaaacagctctcctgctggttccaccctcatcaatgaacacactgaaaatgtcttagttgtgtgctgttgcccaggtataggaggaagatgtggtttattcccttctttttgccatgttaacacatacccaaataagattcagtcaactattgacagtggtttacagtgtacatttcaactatagagagcttgggaaaggggtgaaatcaagaaggacttttcataacagtctaattcctcccactggttggagacccactgcccagcccttctgtggcaccccatgtgaatgctttctagttagttgagaatattccacacacacacccatatttcatcctactaactcctttccccaatgccccacttcttttagcatcctcttctagatcaaaatccttataccttcccttttttaaacacagttggattatttacctagtgggtagcaagctctaagttttcctgttccttagaaatttttccagcttcctgagctgtgggtcacagctattctttctaactctcattctgctataggatagagaaacagaaagtttgagtggaaagggaagaaaagggcattggggtggtcttgtgccagtttcctgatagtcttttctttggctgactgaaaaatataataataataacaggcctggcgcagtggctcatgcctgtaatcccagcactttgggagactgaggcaggcagatcacatgaggtcaggagtttgagaccagcctgggcaacacggcaaaatgctgtctctactaaaaatacaaatattagctggttgtggtggcacatgcctgtagtcccagctgctcaggaggccaaggtgggaggatggcttcagcctgggaggtggaggttgcagtgagccgagatgacaccacggcacaccagcctgggcaatagagccagaccttgtctcaataataaaaacagcaacaaacataataataatcacagacactacacattgagaaagtcaattaccttctaacagctatctccagtgctatgcctcatgttcccagctctaagagcataagtttcagatatcaattggctaaagagaatcttatttcctttcaaattacattacattgtcctcctccatgaatttaaccttaagtgtgtttttctcagctcatggatatctcctgcttccatcccatgagttgaacatacaagaaataagaaagtctcattcaataaaaagatatttatcaaaaacactaatagcgatatttaacagtgtgatttagtttttatgggtttcatttatttttttagacctaacaacgtttccataagatttttacaatataataagaaatactatttttttaagtggggggtaactggcttcaaaagacactcggagaaaaagtaaggacttctacctaaagagggtaggcacacatgtatcatttccctcaggcctgatcatcttggatttctaaaaatgacatcaaattatcttttcaaaccataactacattcaaaatcaagaaaaggaacctccatggaccagaaaccattaggaatccctggatgagtgataagcagaaagaggtcatcaaatctaaggaataaaatcaggctagtgggggaaaaaaataccatgagaaaatagtattgaaatcaaaattaaaacatttcaataatatgaatagcaggggaaaaaacacagctgaaggtggattggtgagctaggagtctgaattgatgaatttcccagcaaatattgtaaaggaattttccagaatatatcataaagagataaagaattagaaagtatgaaagaaaagtcaagaaacatgaaggattcagaacgtccagtagtcacctcctcagaaaatcaaaaaggagaaactaagatttttagagaggaaaattgacagaaaaaaaaagtaattctggtcattcctagactcctttttaataatgaaggaatcatttttagaattacttagtcatacttcagtcaactcatatttattcagatttaacaatgtgtcaagcactgtgcaaggagctgacaatctaataagagtaaaaaaatcctctccctggaaaatgcatacattcacagatcacaattttctacattattttaggggttaagagttcatggattcataatcttttatatttttatttggaacagttcacctgttttgacttggcaaaaacaagactattttttccctattgaattactggattgtttgacaaagggtatcttagcaactgattgtttgaagacatgaaatcatacagagactcagtttagaataaaactggaattggtaaactcacactctcagtgctatcctacaatagttatagaatggtcatactcacattctgagtgttatcccacaggggttatggaatggtcatctttcatcaattgaagtaaattgaagtctctgaacaattgttaattacattagtaacaaaaatcactagttttctactaatggttagtagacatctcacagagaatctcagctgcaaagaaagtcagcaacctgaaaagaagacaatcaatctacaggattttgagtgaggaaaaattttgttggcaccacctagggagactaggggggaaaagcacttgtctgagataatatctgtaaataaagagttaactattgaattaacttccagaaaattaagtgtatttaagaatatcaccaatttatattggaacattcattgagttttagatcagatacttattggcacagagaaaagctagagaaaattaggaaagaatcatttaaaaacagcatcaaatagaaaagactgatttgataagaactaaagatggaagaatggctttatagataggcagacaaccaacttaaaaaagacaaaaagacaggttggtcaaaatactggatttactaataatgtaaggaaataacattacacaatggaacataaattacataatctaaaacagaataaaaactgactccacttgtgcaagatgtgaatatataagagaagaactacagtagtctttcaagggaccaatacaatatctatctacatgcctatgccctggcttctataccccagtggtcataaacataatattttaagatctgtatttttacagagctttctgattatgacatactgagtgaaaggtatggcattttattatcagcttcagagtacaccctggaggcaacaattataatctgttaaataattgtggggtttttttgaacagaatctcactctattgcccaggctggagtacagtggtatggccatagttcactgcagcctcaaactcctgagctcaagtgatcctcctgccttagcctcccaaatagttgggaccactgaggtgccccaccacacctggctaactttctttattttttgtggagatgaggtcttactgtgttgcccaattcaatttcaagctcctgacctccaggcaccctcctaccttgccttcaaatgcactgggattacacgcataagttaaataattttagatatcagcctggcttctctgaagttatatgaagacagtgaagttgtcaaaataatactatctgatctatatttgttgttattaatttatacctctgtgcactctaggaattacttttacttattgtttagttttcttctttctcagagttcttaaacttttgtggttctttctgggctcctaacaaattattaaaacttgttacatattgtattagtccattttcacactgctataaagaaatacccaagactgggtaatttacaaagaaaagaagtttaattgactcacagttctgcatggccagggaggcctcaggagacttacaatcatggctgaaggagaaggggaagcaaggcaagtcttacttacatggcagcaggagaaagagagcaaagggggaagtgcaacacttttaaaccttcagatcttctaagaactcactatcatgagaacaatatgggggaaatccacccctgtgatccaatcacctcccaccaagttcctcccctgacgcatgggaattacagttcgacatgagatttgggtgggacacagagccaaaccataccttatatggtctgacaaagacattgttaagaagatactaaattacattaaatctgtagtggaaagaggtaactcacaaagctggccatcaaaggtcagggtcctttaagtaagtactcgagacatgctgaggtcttcagagtcaaatgcctgtagtaattgcaactgggttttttttttttttgcaaccactgaccaatcacacttttctgcccattctttgcctgacttctttgttttatgcaacatttaataaccatttatcttctttcgcattgccaccaaagtatttagccaaaataagcagttgggtgattttaataaaactcaagaaacatatcaactataagctgactgtcttgtagaggttaaaccaaattttgaaatctgataacgttcatcacagttatatcttgctttgaaatataagttaattgctaagacttggatttttagaaacaacaaaataaatcagttttattagcaacttatgatgaaggagattcctattaggaaaaggctaatggattatgagacccagtaacaactttgcagggctcaaatgctattagaagcctctccttcctagtatttattaattttgcaatgaaagaaggttggggccttttctaagtaccaagaacactaattaaaactaatcaatcaaattccttacaaaccatgaaacggcagaataaacaggaagagcacagcgtggctaagaatgcaaagatgaacaggaaataaaaccaaatgatcccctgctgctgtctgggcacatctcttgcacaaacgttgtatttaattctgaggtgattatctttgatatgcctgccataggtgagggcagataacacatgaaggagtgagcagtctctgtgtagctagctaaaggaagaaacaggtcctcagagagaaaaccggcactaagaaagccatattgtaagaaagaggtacagtgtggtcaatgagctctgtgcttgcaatcagaagatttgtgttcaagtcttggctttgcatgttcctatttggatgactttgggtaaagcactgaattgcagtttctcaactgtgtagcatgagataacaatgctcactttgacctctccatatgaatgttataatgatcaattaatctatatatttaaattaagatttttcacgtataaatttttcattttaataataaaatagtagcatactgttatcaatactggtagcagtaactaggaaagagcagtatatcataggccaaaagttaagagcaagaccaaaggagctgacatactacagaagcaccgtaatatagaagaagcaaatggtacagcaattgagagcctagactttgaaaccagcctgtctagatttgaatcccggctctgcaatgtagtagatatatgactgtggtcaagttacttatgctatctgtgcttcagtttccaaatctataaaattcagataatagcgttaacttcatgagtttgagtatatatacatattagtgttcaacatggtacctggcacatagtatatattatacaagtgttttgctggtattacaatgctggcaacactaagttgtttaactccctcctcattggaaactggactgatcacaaactctaggattgggtttgtgactccagatatgaagaattagaggcaccccatgcaggcatctaagcaggttattgcatcagaaagctactccacattgaccacagctcggattttaattcatgtgagaaatggacagaaaaagttaacttttccaccgcattactacattctaatcatatatttgcacataagaaagcaaaataatttgtttaggaaactagataaaccctggtcattgtctctattgaccaagtgaaaaaactatgtacaatcagttttatcatctccactaagctggttttacctattttcacatttggtatcaaagtaaaggtatacatttttcttatacattgtataccagttgtacaaggcccttggttttcttgttttgttttatgtttagtggtttccatttatgtaacatatttctattgataaaagaaaaacttcagctgaattaatctaaagaagtttaattgagcaatgagagattcttgaatcgggcagcctccaaaatcacagcagattcacagagactccaggggtgcctcgtggtcctcatggtcagaacaaatttatagacaaaaaggtgaagtgacttacaggaattggaagtgaggtacagaaacagtgagattggttaagcttgacgtttgccttatttgaatgcagtttgaacattcagcagtctgtgagtggttgaagtatggccactgggattggccaacactcagccattgttacaggtgcatagtattaagttaggttttcaattttgtctgactattaagctaggttacagttcattcacaaggactcaaatagagaagtatggagtccttctcaggccatatttagtttgctttaacaattccccacttttggtcattttctcaattttgagaaaatgaccaaaatcttagtcattcatgttactattactatcgtaaatgtacttatatggttttgaaactcactgggaaacaatagaatagtgtgttttgcaaggagagaataaggactgagtagagggtacctccttatgctggaacaacctgtccacaggagaaaaacaaaaccctatctgttctaggatctatgtgtttcttaaagccttagtttggttatgtcacatttagcacacgtgactccattttagtttggtttggtttgttggggcctagtgcataagctcagtccaaaataatggcctcccataatttcgtttaaaaaaaaaaaaactcccccttttcagccaggttctcacttcggtgagagtgtgaccaaaacttagggcctcagcactactctcagttaccatcattttgggtttctggtctcagtatgtcattcatagtttatggtgtcctcatggtcacacatttctttcagcttttgtcattctagttgaagagagaccatttgatgttctagagatggctgcatgcaaatacttaaaacctttaagagaatacagcgcaccagggagactattactgtgaccatcaggaggataataccaagagtttggagtatgtgccttatccagagtccccataaaccaaaccacataaaattaaatagattaaagaatgagctagatgaagaatctactcatttgactaagtgtcctttttattgaaaccctacaaatgaatttttataatctacatttgatgtatttctccataggccactagtgtcagcatctgcacaggtacttttctatttagccaattgtattatttagcataactttcacaagaggatttaaagtctgttgtgtaatgatagcctttaaggtagaatttgctgtagagcatattgtgagggtgacatttctaattattgtctcttttattctaaatcatggaaaaaggacctaacaaatgatgtccttctagaagagtgaaggcctcctggcactgttctctttaatccttgatgtggattaagaggagttttgacccccgaagtttctcacctacattgggctttcatcttttatctattaaagtataaggttattcatgtataagactggctgcaaattccttcacaaataaaatatgccctataagttcaaataatagacaccctttttatagaggcataaacaaagaaaaaatattcaaagataagagtctgatgataatagaagtcttgatctgtgatctcgggaaaagttgttgacatcaaggatgccatctttttctggggagaaacattcctggttagttttaccttaaggtttccaatgggtgcacagttccaggagtgtggtgggacccttctcagttgtgagattatgaacccaaagttcaagtctccaaagatttgctgtagtgtggatggcaaggacagtctttctccgatgttttcagaaaatccaatcttccagttctagattgtgaagggattgtcctcaatgaattttacctggcaaaaaaacactgtagcataataatctactgttataacatcagctttcttgcatgggaaagcttttatacaaccagaaagcatgcgttgaaaatgacaattgaatgaaatccctttataaatgtttaaatggcccatcgggtagcgaaatgtacccgaagctttgattgtcttcccaggaatatggaactgaacattggttttaaactatttctgcaatttataagtcaccacatcaatatattcagtttggattattttatcttttccatgatgagtcatggaatgcagaacctttaataacaaaagctttaaggactcaaaaaggacaagcgtggctgtcctggttgttccagagtctgtgcttaacattgaacttatgtcctcttgaatagcagttgtttctccaatttaggtgcatagcactgataactaatgggttatcacaggtaatttgacttagaccatggagttcattcaaattgtatatttaaacaattttaatattggctgattaagcatgataatctcaagcttgattttgaaaggtttgttaaataccaaagtcttaaaaacattggatattacaaaatagaatcttaggaccataagtcattcattaggcaaaatgataactcaaaattttttttaagtaaaaaacattattctgagagacaggagtctcagctttccaaacaagacccaatgaagacagcataaagccaactgactctcttttctttccctgctcccctttttcttttatagtttacttaagaggtaaacaaaaaccttttgttatcttttaatattacataaaaatcctttttaaaaggccaaattttatgtttccattagcgtatttttaatgttaaagctagttttttaataactttttataaatctattcagttttaattagtttaaccataaggtaagatttttataaatcttttatagccctttacaattttttctcagagcagtacaatgttctatgaaaactctgttgtgcttctctattccaatgtccaatttatgaaaaaaaaatcctgaataatgccattttaactttagccaatatgttcacacatagaatctcttataattagctgttataaaccttccacaaatttttcaaacttcatttaacttaaaacagtcctttaaccctctaacccaggcaaaaatttacattcccatactttcttataatctcttacaaaaaaacacacttcattctcattatacaccttgcatgtaaacctgttttttccgtaatctcaattacatattacaatgttgactcttagaaacttttacttttggtgaaaaccttggtaagtaagggattttaattatgtactaggtatggagtctaggacgcagacagaaatgcagataaggtctgactctttccagcatctaacgccatgtgtcccatgccttgcctagctgtaaaacaggcatctgtacagttaagagtcatagtggcattttatgaagcatttgggaggcctaatcacctttaaattatacaacatttctggcatgaacttcttttcataaattctttcacaacttacatagaccatgtacaacatgtttagatattctgacttgccttaaacatccccctttttaaacaactagtcattttactttaggacaagaatttaccgtataacatcctttcttatataaaatctcttttctttataacattctttgcatagctagggggcatggctaattccatatatctcctggtcttatttagaacttaatgcctccaaaataaattgaacaattttcaaaagtcaaagcagtttatgacgacctaaaagcagttagcaaacctaatatttgacctgcataatttagacaaaaatgtattcattttatcaataatctttaaagctgtttttatttcctaaagattactaaagttacataaactaaaaggaattacagtttttattttgttttcaaaatatttgatttaagcgcttatttttatttaagccaattaattaattagtgctcttttatataaacattacacacaacacatatataattacacagacagaagaagattacttcagtagttgtaaaatgtttcatttgctggtttttaagtttcttaattggttattggctttagggtggagcccttggaagaacagggccaggaaaggggtttcagttgcctcctgtttttcccaaggcgtccaggctgttagagcttgaatatccacttttaattaagctgacttttaaccatagcactcttcaataaagtccttttaaaatttcttaccacccgattttagccaggccagtgaattttcaaaagaaaatgtcaaaggtaacctcccaggtgatcagagaaaagacaatttaagatagtttatagtggagaagagacaagacaaggttatgcagatattaaaccaaaaaaggacttacttcctaagcaggaaatcaaacccagaccaccagtgtgaaaggccaaaatcttagatactgagctacagcacagggcagtctcgactgtccttcccaggagtctatagagtagttccttttgaaattgcaaaggcttttaactactcgagataatttttagagctaactatgacataaaccccaaaattcctgttccctggaaggtggaatccaagagaaaatactgtcatgtggtcataaggtcaagttcccaaggacctaaaacaagacagagaccctatctagtttttttgtttgtttcagggacttgcaggaaagtttgttattgaccagcttgctgggccgtctggagcagcaagcttatgagggcctaagcctatgttttatcctaaggtacccctcaacccagaaaaacaaattcatagtacaaatacatcagtttaagactagcctcagaattcttttcaacattaatcaaaaccttacagaagagataaacagtgatttttaccattcattcaactgtacagagagagagaaaccaaaaatctgactggtaagaaattcttacccttttgccagcatgccaggcttctgggttccctttccctgagcggccctagtgactcagcttgctgtatcattgcaatggggccaagctgcatgataaaggaaaattatatttttttgttctggccagagtaaaatatgtgtgataaaacatagacattagccactctgcttagcacttaatatcaaactggcaaggcttaagtttgcccctggttgggccccatcatcattaatccaacctctgaccatgagtttcaacatgtggtctctgggcaagatggttgccctgagcaataggcaagataagaaagggaaaggagagagagaaaagcattgcctgtggcagggtggggaaggcaaaaaggccagagaagatcaccccccattccagcgacactgaaaagttcaggtggctgcttttttatagcaaagggatcttttccagcagtgtcatcatctctcatgtttccccttttagggaggaaaaagctccctatgtcccacaatcctgtacattcctaatcctgtcacctgcagccgtcaggaaagagcgcaaggcagattattccaaggagaatagcagttgacatgctgtagtgccaaacccattcttagccaaaaggaactttactgagagccctcattttcaaatgtacttcaatgcattgttgttcattctgatgttccactgtgagttatctttagtaatattttggtgtttctgtaagacttcgctgcctcccagacctaatgtataagccagaaggaactcagttttccagaaattaaggatcccatttttacctaaaatattgactttactctcaggttctcttgattaacttagccaatgattgtttttttcctacctaagcgcacaagaaaaatgaaaaaaaggggtagaacagaaaaattcctgtgaattttcaaaagacaaattttataaaccctgcaaaatactgcttactaccagttcctttctgacctagtcagatataagaggcctctaactggattcaaaccagttaattcccagatcaaatccattcttggacccagtccagtttctgtcacaactctaaacccagtttggatcagaaatctgctcaaagaaactcggagagctcaaaacgcaaatccgtggagctccaaaatctgagagggagcttaaccgtgatccccagctgctctgagagatcaatggacacaagtgagtcctgcaggtatcttgagtgttcactcagcacttctgggggtcactggaagctccacttcggatcctactcctgacaccatctgagaaaagaaaaacttcagccaaattaatttaaaggagtttaattaagcaatgaaagatttgcaaatcgggcagcccccagaatcacagcagattcacagagactccaggggtgcctcgtggtccttgtggtcagaacaaatttatagacaaaaaagttaaagtgacaaacaggaatcggaaattaggtacagaaacagtgagattggttacagcttggtgtttgccttatttgaatgcagtttgaacattcagcagtctatgagtggtatagccactgggattggccaacactcagccattgttacaggtgcctactattaagttaggttttcaattgtgtctgactattaagataggttacagttcatccacaaggactcaaatatagaagtatggagtctttctcaggccatatttatcttgctttaactctatcttctattttataaccattggttcctcaataacatctagaatgccacaaatcaccacaaaagtaaaaactgggtgatggattaaatgagagggagggagggagggagggatggatggatggatggatggatgggaagagagagagaaacatatgtccttgactctttattttggtgatttacaccaagaaaagaaagctaacaaactgtttaatcagaactatatcaacttttaggtttaaaaaattattttgtgaaaaatccattagaatgtttcagagatatgatgattggtatgcagataaatttcatttttaaatatttggttagaaaaagttagaatttttcaaaatataaaattatgaattacataaacactcaatcatattttgtcccttttgatagattatgtgagtaaaagaaacgcaaaaagacaaaagaatatagatggattcaatgacatataaaatctttataatctaagccaaataaaattatatagcactggcaactttgtcatgactaaatttatgagtgtaacactaaataatgaactgttttgtgtgtgacttataaagtagactcattgttgaatgggagaaatatattatccaatgcattttgagcaagatgaaatgtaaggtaccatttttatagaaatattttagaggaagctgttcaacattggaggataatagccgttacttataaaatacagcatttcccctctgattcataggaaagtaacaagatctttatcagaatgttctcatttccacataattcaaataccatgtatatcaaaaaataacaagtaaagacaaaatttattaaattttagaatatgaatggcattgatcatagtgaatacatttgacaaaatcaaataaccaatggagcatattcattttggtgagtattcttagtaaagtctttaggtctcagtttcttttgaaaaataaactggttgtgaatattacatgagataaaataaacggaaaggcacattgtaaaagagtgataaagcaatacataaatataaattctaaatgttataacattgtgcatagtcattgtgcatgttaaatgttcaaaaaataactgagttaatgattgaggcattgcaggaagtctggggtgaggcacttggcaggaactgccacacaggactattttcttcactatgacaatgctctagctgagagtgaatgtttttaatataaatggttttactcagcttaacaacctactggattcactattacagtcatgagtcatttaagcacagggacatattctgagaaatgtgttgttaggctattttgtcatgtgaacatcgcagagtgtacctacataaacctagattgtgtagcctactatgctcctagggtattgctcataggctgcaaatctgtacagcatgcgattgaactgaattttgtaggtagttgtaacaaaatggtatttgtgtgtatctacacatagctcaatacagaaaaagtacagtaaatataggttataaaatattaaaaatgatatacctgaatagagcacttaccatgaatggaggttacagaactagtattggcttgggtgaatttgtgagtgagtaatgaatgaaggcttaggacaatactgtacactacagtagtgtcagtaatgtttaaactgtgcacacttaggctatgctaaatttattctttttagttttcttcagtagtaaattaaccttagcttactgtaattttttcttcatagacttcgtaattttttaactttttaactctctcataactacacttagcttaaaacacgaacacttttttcctttatatacttattctataaggtttttctattattaggcatttttttttactttttaaacttttttgctaaaaactaagacacaaacacacacattagtctgggcttacaggggatcaggatcattaatatcactgtcttccacttccaaatcttgtccaactggaaggtcttcacaggtagtaacatgcatggagctgtcatctcctatgataacaatgccttcttctggaatacctcccgaaggagctgcctgaggctgttttacagttagcttttttgattttaataagtagaagaaatgcactctaaaattacaataaaaagtataatataataaaacataaaccagtaacatagttgtttattatcattaccaagtattatgtactatgcataactgtatgtgctacacttatatattcctggcagcacagtaggtttgtttacaccagcatcactataaacatgtgagtagtgccttgccctactacatttggatggctacagtgtcactaggtgatagcaattattcagctctgttataatcttattcaaccactttagtgtaagcagttcattgttgaccaaaatgctgtaatgcaatacatgactgtatttaatactgtttccactattcaagcacaacagatcattcatcatcacaatattattataatcaatactattcaaaggagaaaacacaagctctggaaacaatttcaagataggagtttaactcactaatgcaaaaaagacacatcatcaaaaaagcagtattcaaaatgtgaggttgatgacacttatgatatcattattcataggatgaagaattctggcatttccattcatctttctaccacctatatgtctaaggcagacattgcagagcaactacctcaacaaccttacagcccttttccttatgcctttctataatgcagaaacaggaaactaaaacaaaatgacatttctcagactgcttgcagctgaattttaaaatatgatttagatccagatgggattaagacttgggcaacagaatgagaaagagggcacctttctgctgcttcagataattctgctggcaaacccaatggtggtggtagttggatttccaaagcagcagtactagaacttgtagacacaaacagggcaggatttgaataggtttggacatgtaatttattacaccttatgtttgcaaaaatcttaaatattaaaaaagaatgaaatgaaaattttagaactaaaaaaaatacaacaatcaaaatagcaactcactggatggactcaatagaggatggtcatcacagaggaaagaatcagtaaacttgaaattatctgaacaacaaaattttattctagaaaaatacaataaacagaatcttagagacttgtggcaaaatattaaaagacctactattcacatcatccgaatcccaaaaggaaagaaagtttgatgcagaaaaatatttaaagtgataatgcactttaaaaacttccaaaattgagttgggtgtggtagtgaatgactatagtcccagctattcaggaggctgaggcaggaagattgtctgaacccagaggtaaaaattcagctggggcaaaataataagaccccatctctaaaaaaaaaaaaaaagaacaacagggaggcttccaagatggccaaataggaccagctctggtctgctactcccagagagatcgacacagatgggtgatttctgcatttccaactgaggtacatggttcatctcactgcggctagttggacagtgggtgcagacaatggagggcaaactgaagcagggcagtgtgtctcctcatagaggaagcacaaggagttgggggatttccctttcctagccaagggaagacatgacagactgcacctggaggaatgatacactcctgcccaaatactgcacttttcccacagtcttagcaaccaacagaccaggagatcccctcctgtgcctggatcggcaggtcacacgctcacaaagcctttctcaccgctagcacagcagtctgagattgacctgcaatgctgcagattggcaagggaaggggtgtccaccattgctgaggcttgagttggcggttttatgctcacaaagtggccaggaagctcaaactgggcagagtccactgcagctcagcaaggcctactgcctatctagattcgacctctgtggcagagcatagctaaacaaaagaaagacagtctctgcaggcttaaacttccctctctgacagctctgaagagagcagtggttctcccagcatggtgttcgagctctgagaacggacagactgcctcctcaagtgggtccctgacccccgtgtagcctgactgggagacaactcccagtaggggccaacagacacctcatacaggtgggtgcccctctgggatgaagcttccaagggaaggatcaggcagcaatatttggtgtactgcagcctccactggtgatacccagggaaacagggtctggaggggacctccagcaaactccaacagaccttcaactgaagagcctgactattagaaggaaaagtagcaaacagaaaggaatagcatcaacatcaacaaaaaggacattcacaccaaaaccccatctgtaggtcaccaacatcaaagaccaaaggtagataaaaccacaaagatgggtagaaaacagagcagaaaagctgaaaattccaaaaaccagagcgcctcttctccttcaaaggatcgcagctgctccccaacaagggaacaaaactggacagaggaatgactttgacgagttgacagaagtaggcttcagaaggtcggtaataacaaacttctctgagctaaaggagcatgttctagccaatcgcaaggaagctaaaatccttgataaaaggttagacgaatggctaacaagaataaacagtgtagagaacaccttaaatgtactgatggagctgaaaaccaaagcacaagaacttcgtgatgcatgcacaagcttcagtagccaattcaatcaagtggaagaaagggtatcagtgattgaagatcaaattaatgaaataaaccaagaagacaagattagagaaaaaagagtgaaaaaaatgaacaaagtctccaagaaatatgggactatgtgaaaagaccaaatacacgtttcattggtgtacctgaaagtgacagggagttagaaaacactcttcaggatattatccaggagaacttccccaacctagcaaggcaaaccaacattcaaattcaggaaatacagagaacaccacaaagatactccacaagaagagcaaccccaagacacataattttcagattcacccaggttgaaataaatgaaaaaaaaatgttaagggcagccagagagaaaggtcagattacccacaaagggaagcccatcagactaacagcagatctctcggtggaaaccctacaagccagaagagagagggggccaatattcaacattcttaaagaaaagaatgttcaatccagaatctgatatccagccaaactaagcttcataagtgaagtggaaataacatcctttacagacaggcaaatgtctagagattttgtcaccactaggcctgccttacaagagctcctgaaagaagcactaaacatggaaaggaacaactggtaccagccactgcaaaaacatgccaaatggtaaagaccatcgatgctatgaagaaactgcaccaatcaacgggcaaaataaccagctaacatcataatgataggatcaaattcacacataacaatattaaccttaaatgtaaatgggctaaatgccccaattaaaagacacagactagcaaattagataaagagtcaagatccatcagtgtgctgtattcagcagacccatctcatgtgcagagacacacataggcttaaaataaagggatggaggaagatctaccaagaaaatggaaagcaacaaaaaagcagtggttgcaatcctagtctctgataaaacagactttaagccaacaaagatcaaaagagacaaagaaggctagtacataatggtaaagggatccattcaacaagaagagctaactatcccaaatatatatgcacccaatacaggagcacccagattcataaagcaagtccttacagacctacaaggagacttagactcccacacaatgatagtcagagactttaactccccactgtcaatattacacagatcaatgagacagaaggttaacaaggatattcagtacatgaacgcagctctgcaccaagcagacctaacagacatctacagaactctccaccccaaatcaacagaatatacattcttctcagaaacacatcgcacttattctaaaattgaccacataattggtactaaaactctcctcagcaaatgtaacggaaatcacaacaaactatctctcaaaccacagtgcaatcaaattagaactcaggattaagacactcacttaaaaccacacaactacatggaaactgaacaaactgctcctgaatgactactgggtacataatgaaatgaaggcagaaataaaggtgttctttgaaaacaatgagaacaaagacacagcataccagaatctctgggacacatttaaagcagtgtgtagagggaaatttacagcactaaatgcccacaagagaaaggaagaaagatctaaaatcaacaccctaacatcacaattaaaagaactagagaagcaagagcaaacacattcaaaagctagcagaaggcaagaaataactaagatcagagcagaactgaaggagatagacacagaaaaaacccttcaaaaaatcaatgaatccaagagctggttttttgaaaagatcaacaaaatagacagaccaatagcaagactaataaagaaaaaaatagagaagaaacaaatagatgcaataaaaactgattaaggggatatcaccaccaatcccacagaaatacaagctaccatcagagaatagtacaaacacctatacacaaataaactagaaaatctagaagaaatgaataaattcctggacacattggattagtcccgagactaatccaggaagaagtcaaatctctgaatagaccaacaacaagttctgaaattgaggcactaattaatagcctaccaaccaaaaaaagtccaggaccagacagattgacagtgaattctaccagaggtataaagaggagctggtaccattccttctgaaactattccaatcaatagaaaaagagggaatcctccctaactcattttatgaggccagcatcatcctgataccaaagcctggcagagacacaacaaaagaagaattttaggccaatatccctgatgaatattcatgcaaaaatcctcaataaaatactggcaaactgaatccagcagcacatcaaaaagcttatccaccatgatcaagctggtttcatccctgggatgcaaggctggttcaacatacgcaaatcaataaacattttccagcatataaacagaaccaatgacaaaaatcacatgattatctcaatagatgcagaaagggccttagacaaaattcaacagccctttatgctaaaaactctcaataaactagatattgatggattgtatctcaaaataataagagctatttgtgacaaacccacagccaatatcatactgaatgggcaaaaactagaatcattccctttgaaaaccagaacaagacaaggatgccctctctcaccactctgattcaacatagtgttggaagttctggtcagggctatcaggaaagagaaagaaataaagggtattcaattaggaaaagaggaaatcaagttgtctctgtttgcaaatgacatgattgtagatttagaaaatcccatcatctcagcccaaaatctccttaagctgataagcaacttcagcaaagtctcaggatacaaaatcaacatgcaaaaatcacaagcattcctatacaccaataacagacaaacagagagccaaatcatgagtgaactcccattcacaattactacaaagagaataaaatacctaggaatccaacttacaagggatgtgaaggacctcttcaaggagaactacaaaccactgctcagcaaaataaaagaggacacaaacaaatggaagaacattccatgctcatggataggaagaatcaatatcatgaaaatggccattttgcccaagataatttatagattcaatgccatccccatcaagctaccactgactttcttcacagaattggaaaaaaactaaagttcatgtggaatcaaaaaagagctcacctagccaagacaatcctaaataaaaagagcaaagctggaggcatcacactacctgtcttcaaactatactacaaggctacagtaaccaaaacagcaaggtactggtaccaaaacaaatatgtagaccaatggaacagaacagaggcctcagaaataacaccacacatctacaaccatctgatctttgacaaacctgacaaaaacaagaaatggggaaaggattccctatttaataaatggtgctgggaaaactggctaaccatatgtaggaaagctgaaactggattccttccttacaccatatacaaaaattaactcaagatggattaaagacttaaatgtaagacctaacaccataaaaaccctagaagaaaatctaggcaataccattcagcatataggcatgggcaaagacttcatgactaaaacaccaaaagcaatggcaacaaaagccaaaatacaaacatgggatctaattaaactaaagagcttctgcacagcaaaagaaactatcatcagagggaacaggctacctacagaatgggagaaaatttttgcaatctacccatctgacaaagggctaatatccggaatctacaaagaacttaaacaaatttacaaggaaaaaacaaacaaccccatcaaaaagtgggtgaaggatatgaacagacacttctcaaaagaagacatttatgcagcccacagacatatgaaaaaatgctcatcatcactggtcatcagagaaatgcaaatcaaaatcacaacgagataccatctcacgccagttagaatgacgatcattaacatgtcaggaaacaacagatgctggagaggatgtggagaaataggaaagcttttacactgttggtgggagtgtaaattagttcaatcattgtggaagacagtgtggcgattcctcaaggatctagaacaagaaatacaatttgacccagccatcccattactgggtgttacccaaaggattataaatcatgctactataaagacacatgcacacgtatgtttattgtggcactattcacaatagcaaagacttggaaccaacccaaatgtccatcaatgatagactggattaagaaaatatggcatatatacaccatggaatactatgcagccataaaaaaagatgagttcatgtcctttgcagggacatggatgaagctggaaaccatcattctaagcaaactatcacaaggacagaaaaccaaacatcacatgttctcacttatagatgggagctgaacaatgagaacatattgacacagggtagggaacatcacacactggggcctgtcagtgggtggggggttaggggagggatagcattaggaaaaatacctaatgtaaatgacgagttgatggttgcatcaaaccaacatggcacatgtatacatgtgtaacaaacctgcacattgtgcacatgtaccatagaacttaaagtataataaaaaaaaaaaacagggaaaacatcaacaacaacaaaacaaacaaaccttcatgaacttaattaaactaaaaagcttctgcacagcaaaagaaataatcagcagagtagacagacaacccacagagtgggctaaaatctttgcaaactatgcatacaacaaaggactagtatatggaacctacaacaatctcaaacaaatcatcaagaataaaacaaacaatcccatcaaaatgtggcctaaagacataagtagacaattatcaaaagaagatatataaatgggcaacaagtatatggaaaaatgctcaacatcactaatgatcaggaaaaagcaaatcaaaaccacagtacaataccaccttactcctcaaagaatggcgataatttaaaaattaaaaaaaaataatagatgttggtgtggattgggtgaaaagggatcacttttacactgttgatgagaatgtaaactaatacaaccgctatgaaaaacagtgtggagacacatgtttatagcagcaccattcacaattgcaaaaatatggaaccagcccaaatgcccattaatcaatgagcgggtaaagaaaatgtgatctatctatcttatctatctatccatcacattaattactactgagtcttaaaaaggaatgaaataatggcattcacagcaacctggatggaattgaagaccattattcaaagtgaagtaactcaggaacagaaaaccaaacatcgtatgttctcagttataagtgggagctaagctataaggacacaaaagcataagaatgataaaatgcactttggggacttgaggggagggaatgcagaagggagtaagggacaaaagactacacatcgggtacattgtacacagctcgggtgatgggtgcaccaaactctcagaaatcaccgctaaagaacttatccatgtaaccaaacaccacctgttccccaagaacctatcgaaatataaaaaagaaaaagaataacagaattggaagactcatactacctgattttaacaagagctacaataattaaaaaggtgtggtagtattaattaaaggatagacacatacgtcaatgggacagaacagagagtccagaaaaagacccacacacatatgatcaactgggttttgacaaaagtaaaaaggtaactcaatagaaaaaggataatcttttcaacaaaataggttggaaaatttagacatacatatgtaaggaaaaaaaaagatcctcaactaagatgccttaggtatgacataaaaagcgtgattcataaagaaaatgtaataaattttatttcaccaaaattaaaaactttttgtgtgtaaggccttattagacaataaaaatgacagttacagactggggaaaaattattgcatatcgcacatgcagtaaaagactagtgtccataacctctcaccttaagatcctagcaaaagaaaagcaaactaaacccaaagctaaagaaaggaaacaataaaaattaaaatggagtttttaaaaatcgagagtagagaaataagagaaagccaataaaaccaaaagttctttctttgaaaatatcaacaaaattgacaaacctttatttacaattaccaagaaaaaaatagaaataattcaaattattaaaatcaagaatgaaaggtggaacatcaccaccaatcttacagaagtaaaaagaacgtataagataatgtcataaataaatgtatgccaacaaattagataactaatatgcaatggaaatattactacaaaaacacaaattaccaaaactgactcaataagaaacaaaaaatctgagcagacctataacagttaaagggattgaatcagtaatcaaaaaacttccaacaaagaaaagcccagggccatatggcttcttagtgaattctactaaatatttaaagaggaattaacaccaatccttctcaaactctttttaaaaatggcatagatataaacacttcttaatttattttatagggctagtattatcctgatagcaaaatcagattgacattaaaaaaactacagatcaatattctttatcaatacagatgcaaaaatccttaacaaaatactagcaaatcaaatccagtagcatatttaagaaattataaaccacacaccatgaccaagaagaaattttatcccagaagcataaggcttagtttaacattctaaaattagttaatgtaatacactatattaatgaatgaagagaaaaatatctcgatagatgaagaaaaggcatttaaaaaaatccagttgtcatgacaaacacctcaaataaaggaaaacttcaacttgaaaaagaaaatttatgaaaaatccacagctaatatcatacttaatgtagaaaaactaaataattttctgccaagaagaagaaaaagataaagatgtccactctcactacttctaatggaaattgtgctaaaatatctatccagagaaaatagggaaaaacagtgcatataaaaacctgcacataaattttcatagaagcactattcataatagctcaaaattgggagcaaccaattatctatcaacttataaatggataaacaaaaaaacaaaaaatacacaataaatatatatatccatacaattaatatatatatatccatacaatgaaaaaatatatatatacatctccatataataaatatatccataccttgaaatattgtttggcaataaatataaatgaagtactgatatgtgctacaatatgggtgaaacttgaaaatattctgttaagtgataaaagccaggcacaaaaaaacacataccgtatgatcccatttgtgtaaaatgtttttcatttctcttgttttgcttttctgttttccatagaatagcttactattgcagggggggggtagggtgggaattagtattatgtcctgaatttttatgtgtatttttctataatgtacaaagagttacatcctcaagtgaaaagttatgccccttttctgtcatcttttttcattacagctaacaggttttgtttttctaatttctcaataattttaatattagagatgccataattcttacaacagggaaggaatctcaggaggtcaggctgtcagagaagaacacacaatttgttacatagaacgtaactattaatgggaaaaagggttctattagtaaaatgcaaggaagataacacttaacagctagaaattttctccaaacgatctatgtttctattttaaattgtatcatggatattcagtatgtcttctagctgattctatttgttaactgaatttgcactagaaatttgttcatattacattatgtgatatgataaataattttctgtggccatgccaagttcatgaactctacaatattccctgatatagcacatatctactagctcatatgaaatatgtgggcattatttcttttctaagaaatatgccaaaatcccatcccctgattcaccagtggttattttagtggtgccctaaatatttttcttttcttagtgtcacacagaatctgataatataaaaattaaatgctaaccatggttaagtgaatgaaacatatgtaatttagaggcatttatatagaaatgattcaccaaactgaatattgaccttaaaaatattaatggatgccattagaaaaaatagataggtacaattaaatcaaagtatactattggaaattttttaaatctaactttaaacactaaattagtgtaattactactaactctcttcaattatttcacaaatatttattgagtgggtacgatatggaaggtagtaggtcaagaactgtgtaagatatgaagatgaataagataagatcctgccctctagagtttataatatagaaccagagataggacatgaacaaaaactatcactgtacttgaaagtacaatgtgccaaacagcaaaacgtaagtacagataagctgctatagatatttctcatctcagggactcattctactgccttaaggtaactggtagaaaggctgtctttttcttgcctgcatttattactaatgttatactcctttattcaaatcataatctaagaaattaatttaagaaagtaatcaacagaagctgggcattatatctataccaggaaaaagtagaaaaaaacacctgtacactaaataaatgggaaataaattattacacaatgatttgatgaagtaggatgaaatcattatataatgttcaactaagtatagacatatgcagcataatttttaaatgtaaacaggaaattagttttattaggatagtgagactatgggtattatttgggaggcaatctttaatgttgtgatatttttatgatggaaaaaataaaagaacatacattggcatttatttactgaggtatcacatcaaagtattctgttttccacattatgcagaaaaattaaaagattaattaccgaggaagcattacttctatactcaactgcacatctgtattgatagtgtggttgacccagtagcacctagattatgattaagacagacgcacatagcaaccaggcattatgaggttatgacatgatattccatggagttctccaaacagctccaagagcatttacactttactggcaaaccatgtccttaagatggatagtcatgaagctaaccaaccccagattattgctggttttttttttacaaaaattaaaacatctgctttctttttatctatgtccctccctctactttcattgaaaaaaaagaaaaacaatatgtttatatcttgcctaaacagtttagtagatggataagtgttgatcattatactaataactagaaatgttaatacagaaaattattaaaatttcatattatatcccttttcactaattgtactcccaaacaggcttatgtaataaaagttgccttcaagccaagtcctaatgtctacagtcacatggaatggttatccacatttcttcctcttaaagttttattttctgatttggaaagtattacttattgagccaacttttcattaaatactaaattttttcttagccagctcccagaggggaggtagttttattgcaatatatgaacacatggaatttgcagatatttgtgggcaagcccctgcaactatctcatagttcactgtaccaaattttttatatcacgagccaagtttattcttcctcatccaggatagagttcccttctagaggctgacacacacaaaaaaggaggagatattgtgtatcacacctaaatctaaaaacccaaaccatgttaaaatagattttcacaacaagaagtagacagcagtttactatgagtagagtttttaaaactagtgcagtgcctccaattttctaaattcaatcatagaagtatcagggaaaattgacgtttcatctttgtacctcacattcgtgtttaattatttactaattcattcattcattcaatcattatttgttgagtgctgactatgggttaaaacactctgttaggcactaggaacatattgctaaaccgaaagaagatcgtttctatcttcacagaggtgaatccagacattgaagaaccaattacagaattgcttatttaaatacacgctatgaagaagaactacaaatttttgatagtatgtaactgaggtggttttgcaagattcctctgggggaaaaaatagcacatattctgaaatatgaaggacaaacaaacctaggcaaggtcaagctgcgaataaggagacaggaaaaaagcaacaaaggacatttcagatgagaaaaaagcatgtgcataggctctgaggcagaaagaaaaaaaaggaaagaaagcaaagaaagaaagaaaggaaaggaaaggaaggaaggaaggaaggaaagaaaaggaaggaaggagcaaactagagacaaggagggcgggggttgaagatggctgattagaagcattttaagcctgctgctaaacttagaagaactagagtagtgtagtgcatagatagttacactttgagtttattttccaagagagaacactgaattcaacagggaagtgacaggaggcaccaaaagtgggagtcggtggggcggatgggagaagaggcaacctccctggccgggattggctgcaagcctggagtgacttctcaacacagggaagggatgagtaaaagacttccagcagcccacatccccaccatgcaatcacgcaattctggcctacggagatgccctcatttctctcaacccctgaaataacgtaggaagctgctgggagactgtgggatggaactcctccagggagggagctcatacggtatctcacaccctttctaagacctatgcagctacagcaagttgtcattttcaaaactagctctggtactgcacactgttctgggacccaggggcactgggaatgaggcattactgaaacccaagctgtcattgctgggacagcagagcaagcctggagcactgccaccgccagggctagaaagcaagcaaggagtaggcagcagtttccagtcatgggaagcaagtgcttagctgctgagatctaagatggggatgcgagcatagtgtgaattgccactgggacttggtcataaactgggcgtagtctcctgtagccagagcaagagacaagccctgaagggactggggtgtgaatgagatccacgtcgcccacatatcagctgagaccactgagggaggccccaatctctgccatggcagtgcctcagcttttctgctattgccctttattccaagcactgctcctgggacctgaagatcaccccaccctggccctccacatctggtgcccactttcaccatggtggggggttgggggcacacctaagcacaagctcatgcagcccagctttggccccctcaggacagtgcacatagcctgaggttctggggatcactaatccaccatcttgggtacctgagcactcctcctgggggcctaaggtcagacctaacctcccagctgctaccacctcagctgccacccacctgcaagttccacttgtgggcctgtagactcgctcacccaacccctcacagccatagccaacagcaacacataccacttgagacccagagaattttcccaccattgccactattatcacccaatccacaccagctgtcagggacgtgagtacttaccagcctgcccagtccactgcgtcattatcagcatttgagcatgccacctagagacccaagaatagacccaccagtaactgccaacacaggtgccactggggcacaaaggtaggcacactcagcctatggctgccactactggggcctgaggaccagcccacctggtatcctagtctccagaacaactttaccatagcctccgtaatagccacaccctaacccaccaagtgaatcaaagacaccactaatatcaaagaaatcatacagagagactacactaccgaacatgtccagaatcagagccagagtaacttatccaaccaacaccatagatacatctttggaaaaagatcctccctcaatgaaagtatattcaaaaatagtaagaaccgactgttaacaacaaatgcgtaaatgtcaatgtatgtaagaacaaatgaagaatgaaaaagcaaggaaatataacacttgcaaaggaacacaataattctccaggaatagatcttaatcaaaatgaaattttcaaaaccccagataaagaattaaaaacattgattttaaagaaactcagtgagatccaagggaattctgaaaaacagtacaaaaaatcagaaatataaatcagaaatatagttcaggttatgagtgagaattatactgaaaagatggatatttttaaaagaatcaaatagaaattcctgaactgaagaatttattgaaggaaatccaaaatacatttgaaagcttcaacaatagactaggttacacagaagacagagcctcataacttgaagacatcttttaaaataatccaaactgaaaaaagaagaaaaaaggaaaaacaagaataagcaaagccattgtgacatttgagacaacataaagtgactgaatatacaaattattgcctccctgggaatgaagaaacaaggaaaggatcagaaaacatatttaacaaaataatggatgaaaacatcccaagtctagcaagaggtttagacatttatattcaggaggctcaatccccagatagataaatacaaaaaatcttccccagggcacattaaaatcagaatgtctaaagtcaaagttaaagagaaaatcctaaaaacaccaagagaaagctatttaataacgtacagagaaaaccctggcagacaggggttttaaatcccatggatttaaacagggctttagaccaaatggacctaacagacatttacagagcattctatccaacagcttcaaaatatacattcttttcgtcaacactttctccaggatggaccatatgttaggccacaaaacaagacataattacttttaaaagtcaaaatcatgaagtatcctctcacactacaatgtaataaaaccagaaatcaatacaaagggaagcttgaaactatacaaatacaggaaattaaacaacatgtttctgaatgaccactgggtcaacaaaaatatttcaagatggaaatcaaaaaatttcttgaaacaaataaaaatggaaacacagtataccaaaacctgtgggacacagcaaaagcagtgctaaaagggaagtttacagcaataaacaccaatttcaaaaaaagtagaaagatgagaaactaacgatctaagagtgcatttcaagtgactaaaaagcaagaacaaaccaaatgcaaaattagcagaagaaaagaaataataaagaccagaactgagctaaattaaatagaaattaaaaataaaaatacgaagaatcaatgaaattaacagttggttctttgaaaagaaacaaaattaataaaccagtagttagattaatcaagaaaagaagagagaagacccaaacaaacaaaatcagaaatgaaaaaaaagagacattacaactgataccacagaaatacgaaagatcatcaaagactactatgaacaactacacactgacagactggaaaacctagaggaaataaattcctggaaacatacaacctactaagatgaatcaagaagaaagagaaaaccagagcataccaataacgaaaagtgaaattgaatcagtaataaaaaatgtctcccagcaaagaaaagtccaggaccgatggattcataaccaaattttaccaaatgtacaaagaagaactaatgtcaatcctcctgaaactattccaaaacagcaaagagataattcctcccaactcaatttacttgcccagtattaccgtgatactaaaaccaaactaggacatgccaaaacaaaaacatgcaggtcaatatccctgaagaacacaaacacaaaaatcctcaacaaaatactagcaaactgaatccaacagcacataaataagataatacactataataaggtagaattttcaataaatgtaatacatcacatcaacaaaatgaaggacaaaaccatatgatcatctcaatagactcataaaaaattttaataaaaatcattataataaaagccatatatgataagcccacagcttacatcctactgaatggggaaaagttgaaagcattttctctaagactggaacaagacaaggatgcccactttcaccactcctaataaacgtattactgaaagttcaggccagactaatcaggcaaaagaaaaagaaaagaaaaggcaccaaattggaaaagaggaaggcaaactcactctctttgttaatgaaaggatcttatatctagaaaaacctaaagaatccactaaaatactcttagatctgataaataaattcagtaaagttgcggaccacaaaatcaatatacaaaaattaacatttccatatatcaacattgattgagctgagaaaaaaaatcaagaaaacaatccttgggctgagtgcagtggcccacatccgtaacctcaacaatttgggagaaggagatgggaggattacttgaagccagttcaagtccagcctgggcagcatatcaagaccctgtctttacaaaaaatatacactaaaatttaaaaaaataagctgggtagagtgatgtgtgtctgtagtcccagctactcaggagactgaggcaggaggatcacttgaatccaggaattcaaggctgcagcgagctataatcaccactttactccagcctgggtgacagactgagactctgtcagtctgtcagagaaaaatcaataaataaaagcgatctcatttgcaatagatacaaaacaaatacctaggaataaatttaaccaatgagataaaagatctctacaaggaaaactacaaaaaggtgatgaaaataattgaaagtgacacaaacaaatggaaaaacatcccatgtttatggattggaagagtttatattgttaatatgactatgttgcccaaagcaatctacatattcaatacaattcctatgaaaataccaatgtcattcacagacttagtaaaaacaattctaaaatgtattaatatatgaaacaaaaaagcccaaataaataatgtaatcctgagcaaaaagaacaaagctagaggtatcacattaaaagtctatagtaactgaaacaatatgttattagtataaaaatagacatatagatcaatggaagagaatagagcacgcaaaaataaagccacatatttacagccaaccgatatttgataaaactgacaagaacttacactggacaaaagacactctcttcaataaatgctgttgggaaaattaaatagccacatgcagaagaatgaaactggatatttatctttcaccatacacaaaaatcaaccaagatagattaaagacttaaatgtaagacctgaaactataaaagtactagaagaaaatctaaagaaaactctcctggacattggtctaggcaaagaatttatgactaatatctccaaagcacaggcaacaacaacaaaacaatagaacaatggaaattaattaaactgaaaagcttttgtacaaagacataatcagtaatgaagagagaaccttttgaatgggagaaaatatttacaaagtatccaagaatggactaatattcagaatatacaagaaattcaaacaagtcaacaggaaaaaataaatcccattaaaaggatgcaatggacatgaagagacatttctcaaaagtacaaataactaatagatacattaaaaaaactctactaatcattggagaaatgcaaatcaaaaccacaattagataatattttaccccagttagaaagcctattattaaaaatacaaaaataacgaatattggccaggatacagggttccctttgggtatttacccaaaggaaaataaatcaacatatggagtgacacctgcactcctatgtctattgaagcactattcacaatagcaaagatatggaattaacctaagtgttaattgacataatattgacaatgacgtaatattcagtcattaaaaaaatgtcatttgcaataacatggatggaactggaggtcactatcttaagtgaaataaaccaggcacaaaaagacaaatatctcatgttctcacttggatagctaggatacacagagggtaaggacagaatgttgtgaagtgagactaatggacgagtagaggcaggtgaagtcaagcataataggtcatagtaggatttggcttttgttctaaaagcagtgtagctccactaaagtgacaggtcatgtctcattatcaaataactatggcatcatcagtgacctctggcttctcagcgcttccacggtttttattgtctgcatcaggaatcagtcatgtaatcaatttctaccttgtattattcacgagtttatatcattttgatggcagcagcagcccatctggagtggccactgccatgatgtcagctgcagtggggaagttgcagccagggctgctcactccatggagccagtgggagctaggaacaggtggaatccccgccccattccaagttggagtggtgggagccccaccttcctgggcgcagctgcaactgcccagccccagctgcaaacccagccatctctgcactcctggggactcaggaagcccccctccccaggaaggcttgaaagtgcctgctcctgctacctgtcttctccccactcccagtgcccactccgattttgaagcaaagttgtggctgagcccaggcactatcatgagtcggccgggtgtgtgtacactcaggacaaactgacatgtcaaccccctgccaccttggcccactccggactttgggtgccaacaagcacaagagggaggccaagggggtgctaagggtgacttaacatgggccttaaggcaccccttggcatgaacagcctgggtgccatgggcatcatggatggcaggtaaatggcagcaggaggcagacaggttcctgggcagaaagagtcaggtcctcagtaaaaccccacttttaggccagggatggcctgaggcctgggggctggtttgccagctccacagacccgagtgagaacttaagagtgctttttcggagcctgcccatggccacccatggaccaatcagcacgcatttcctaccttctgaagccaataaaaacaccagattcaggcagacttgggcagatgacaggacaacctgcctgtggagaggagctactgactgtgggtctcctctctgctgagaggtggacactcgtcaagatgatctgcctgcggacaggagctacctactgcaggtctcctctgagctgttctgtcactcaataaagcaccttttcaccttgctcaccctccacttgtccatatgcttcattcttcctggacacaatacaagaactcaggacccaccaaatggtagggctaaaagagctgtaacacaaacggctgaaacacgtcccttacttgccatgttgggggtgacaagaaggaaaggagggagaaggagagaagacttatggcccttcagagagcccagaccaatgggctccctgaaggctgtgacaccctcttcgggatctgtggttcctggcatctccaagcttagggacaacactgcattccccggtgccagccatggaagctgcttgcagtacgcttggtcaagccaaagcctctcagggagttggcacccatgccagtgcctggagctgcccaccccaccacagctggtgtgcctggctgtgcactgtggctggaccccatgctcactcactcacacaccccacacacccctcaccacttcgtgtctggcttgcccttggcaggccaggggatcctggctggtagcacaagccaagtgcagcctgccaggccaagtgggtggaacaagcccagcatgcgtgagcaaaacatgggcaaaggtgtcaccacccacagaggtttctggccggaaaagcaatgccccaaggctcctgtgaggttttcatttaaatttccaacattgacatggactaagagtcaaggggaaagggaacctcatttattgagtacttattcaattgaaagcattgtgtaggaactttgaatatatcatcttatttaatcttcataaaagcccatgagatcaatatctccatattgttaccagaaagtggtcccaatttagaccccaagagagggttcttggatcttgtgcaagaaagaattcggggagagtccatagagtaaagtgaaaccaagtttattaagaaaataaaggaataaaaactggttactccacaggcagagcagccccaagggctgctggttgcccattttatggttatttcttggctatatgctaaacaaggggtggattattcatgagttttcagggaaagggatgggcgattctgagaactgagggttccttccctttttagaccgtatgggataacttacttcctgacgttcccatggcatttgtgaactgtcatggcactggtgggagtgtcttctaccatgctaatgcattataattagcatataacgagcagtgaggacaactacaggttactcttgtcaccatcttggttttggtgggttttggccagcttctttactgtaacctgttttatcagcaacatcttgtgctgacctcctatgttatcctgtgacttagagtgatctgtagcttgtgcctaacctcctgggaatacaacccaataggtctcagcctaattttacccagcccctattcaagatggagtttttctggtttaaatgcttctgacagtatgacaaaagaggtcgactcaagaacaatccccaatcaacaacagtccaagcttactgacctctagtaaactgtgagtctgggtgcttttgcaataagatcacaaattgcagaagtgttcaaacactgcatatatcactggtatgtcttctcccaattcttagaagactaggtctatttcctatttagtatagcctgatccaaaaggcagtttgcccttgttaatcagttttttttttttttcaaatctgcggggcttaagaaaagtagagaaatgttaatacttggttttttaggaaaattctattataaggcacatttttgtgatcatgccactgcactccagcctgaggaacaaagtgagactccttcttccccctcatcacccaaaaaaagattctaggaatcatttttcacaataaagtcatcaacaaatgtccatttgcaaggaaaaatggctagtggctgattttcaagtgttctttggaaatatgattttaaacacttcataagcttaacaatagaacagcactggcctgtctgttgttaacctttacataataaaattaattaggaataataactcaacccatgcagaatttgtccatgatttaaatgaagagaatgttgttagcccagtttatatcagaaaattagcaattttacttggtaatgcgaatcttctggttatatttgtttgtgtgtttgttggcttgcttgccttaaatttatgatttacttaatgatgtcaaactaagtatgtgctttagcttaggtttacaacaaaataataggctaaattcacagagcgccatggtgtctcgaattagaaaataatttgcctttgcttttcttgaaatagaggaaacagttttgctttaaagtaccagtgcagtgttttctaaaagaggtcaagctgaaactggtgtttaacctggagaggcttccgtgaatttctaatggagttttagaaagactgtatttcctgatttaataatacatatgactgcagtgaaggagcattttaatatgactctgaatctgagtgcaacacacagtcttatgagagagaaacaagagggagggaaggaaggaaaaaaaggagggagggagagaaaggaaaggaaaataggatatattattcaaatttgttgttacaatactatgtgaatgtgaagttaaggatttaaagtagctagcagaaatatatattctgtaggacattatttttagtctgatgccaatgatgcaataataattatagtcactaccatttactgagttctgactaggaggtgggcgctgtggtaggcatcattcattatgccagctggtcctcaccataccccaagaagtggatatgaattatacacacattttaaagacaaaaagcctgcagcttaagagagctatggagcttgaggaaggtgccagaactagaaatggaaagcaaaagccctctcaggtccaactccggagtttaacactcattacatgttcatccatttatcttatttgttttatgcttttcagttttcatttgttattctttttggggagaaggtacacatttaaaagtgcttactgaagaaaataatcttttgttttaaaccctagggctcgccagacattttttttttttttttttgagaaggagtcttgctctgtcacccaggctgtggaatgcagtggcacaatctcggctcatggcaacctccacctcctgggttcaagtgattctcctgcctcagcctcctaagtagctgggattacaggcatgtgccacaacgcctggctaatttttgtatttttagtagagatgaggtttcaccatgttggtcaggctggtctcaaactcccgacctcctgatccacctgactcggcctcccaaagtgctgggattacaggcgtgagccactgcgcccagccccaagacaaacttctaagggaccatataatttattcaaatatatctgtgaatatagcaagcattcacatacacacacacacacgcacacacacacacacacacgcagcatgtctcttcctgcacatccttaaaaatcacatcaaaccctgcaaaagaaaagtcatacataataataagaatctggatttcatgtctgtgttattttacttatctatctcacctgcatctctcagattactattacattttagcaagtaaaataatagataataggagagaatgtaaatctgctatatcctaagatatatacttgttactcttatgaaggtatactatactctttagagcaaactgtacttggtaaatcatctttttgagttcctcaaaatgttgctaataaaataaaatgtaaagtttgcaagtccattttcgtaaaaattcccagtctgcaattttataaaactagcacctaatttaaaatcagccttcaactatgtgaacctggagagaatacatgcattacctgaaggccaaatccctgtattctacaattctttgtaggactttcttagcatgtaattttagagtatccaaactatttctttcctccacctacatcatcattttacacacagcgaaatgtggtcattttttctgcttaagtggtacatactacagatcttatgacagaatgtgctaaagtggacacacttttcatgttcataaaagtggggaagatgccactcttgttgtaactttttagtttaaaaaagaaagacaatgccaaaattcttccaaagaaggactgtaaagagaaaaggggagaaggggacatattcccccatgccagcagtggtagtgagggagcagagttggaacatagcttcagacagtggtgataggaggcaacaggaaatgccattgctctacccacatttctttggattctttaccaactcttgcactcatcctccctgtgtactatcccctacaacagtcagcacctgtatctcttaatttgcgaggtggcctcagactgctagatcccctttgagaactgatgtaaagttggaatctcctggatatttatatcactcctggggaagcctttaactacagattaatctgtagctacccctactcccttccctctatggaggagggatctaaggtgtgacctttgaatctgcacttcaaaactgagccaaagttttcttagcagaactttgatactatgcccttgcttggccaaattttctttccactcccatttcttcacaccccaatttttccagcgaacactaactatgttcttttcatataaattttcatgtcagtgtctgcttctgaagaaataacatggtgttgagtctctgccaagtaggggctgcactggctactctgcctaaagtaagagactgaaataatggtcttttcagtggaagaagccccactagcctagggaaacagaaagaatttctctgaatggaaataagaaaaagaaaatgctaccatgagaaagtgagacccatgcctccaaaacatgcacatatgaaatcagaatttacatgcttgtcgagaacaagaatcctcaagctaagaaattaacattaaaaaacttgtcttgagtgggtaagatgcagatggaaattcaaaactaccctgcaaaatttctgccatgcacaagacaagcaactcccataggagggagggtagggagaggatgtggtggataatgacagaagctcacagtccaaaaaattacagaccacacaaagaaatggtttcacatcgatgaagtctcatttcaacaactggatgattctatgatctgaaaaaagaaaaaacctatataatcagtatgtttaaaatgattgaagaaagtaatatagaatgcaaatcataatgaaagaaaattcatcataaaataacagacacttaaagaagactgtagtataacatctataaagattatgatggcagattaaacacacgcccagaatttattcactccagaaacgctgccaaaattacaagccccaaaaaaccttttaatccacaaagatgaagagagcagcaggggagacaacagtgacaggtctttgaaacctgaaaagcagataaccagtgtcagtgacttgtgaaacctgtgaaagatgaataaaaattggctgaaaaactaataacaaaatccattccaccacagagtcttccaaagattctggaactaccaacacccaattaactctagacttgtaatttagggaaaagtgactccatgaaaaagagtgagaaaagctgtttctgaagcagctagatttctactagattccttctcctttccctttcccagtttgttcttcagaactattaactctgcccaagaaatttgaaggactttttcctggaaaggataaaaacaaaaagttttagggactgctaattgccagtcagagttgaaggtctgtatgggaaataataccaagaaaaggaaagtgaagtgatcatatgaatactaaatgctgaatggtttggctcccagacaaaacacatcagacctttgctatccagtagactcatagagtagacttcggagtatgtaatccacccataaggaaaacgcacagatactgataatcccattgagatattcaaatttgaccagtctcacttatgcactcagatctttcaatcagtttttaaggccccatcccttaatcatgaataaccagtcaatagaggacaaaagatccacacaaaaatattaccaaagaaaactttaaattaagaagtattaataagtaaaaccattattaatatccccaaagaaagaagactatatttggattcataaaaagaatagattctatagaaacaacatgaacattcagataacacgggaaaaaaagtttgtggaaatctgttttttttttaattattgtaaaaatgaaactctcaataaaagagttggtagataaagttaaggaaatctttctgaaagtagagcaaaaagagatggaaaaaaaagaaaattagaagactatcccagaaagtctaagatcaaggatgaggaaatacaggaagagagaaaacataacaattgaaagaattaaagatgaaatattgcaagcaatattaccccaaaatgagaattataaatttccagattgaaagggactccatgagtgctcagcataatgaaagaaattagattctcacccacatcaacacatcaacatcgcagcaacaagtcagaacacaggaaataaaaagaagatgccgcaaacacctaaagagaaaaaacaaatgaacaacttttgatatgagtaaagtaaccacataatctattatcctaaccagaacactttaaaattaatgtttttaaattttttgattgacaaattggtttatgtcattggcagacttatgaaatagttctatttgaaaacttgttttaaaaatcaaattgcttcaaaaatatagcacttatctgttgcattgaagcaaaaaaactttatataaattttctgaacattaaataataacaaacagaataactgttttggcaatattcatttacagtaatcatttttagctgtttctatatactttttagctatctattaatactaagatgtcatttgtatttttctcaagaatgcttctttttattgtggtcatatggttttcatcatttatatgaaattttcatcaatcctctacacttaatagttaataaattagaaaatgtttacgctcttgcttggctcttctctgtagaccatatttttcattgaaagactactttccccatagatggtgaagaagttgataatatcagataaattttaatttttcctaatggaattatatgattatttcagctcaaatattttcattaagtatcttaatttgtaattattttctcagtaaacattttattgaacatttactatgtgctgagcactatctctgatctgttgtaattgttgttgttcatttagaaaattccactttttgtaatcccagattttgggaagctgaggcgggcagatcacttgaggtcaggagttcgagaccagcctggccaacatggtgaaaccccgtctctactcaaaatacaaaaactagccgggcatggtggtgcacacctgtagtcccacctactcgggaggctgaggcaggagaattgcttgaatctgggaagcagaggttgcagtgagccgagatcatgccactgcactacagcctgggcgatagagtgagcctccatctcaaaaaaaaaaaaaaaaaaagaaaagaaaattccactttttattttaggttcagagatacatgtgcaagcttgttataaaggtatattacgtgtaataggggtttgatgtgcagattatttcatcactcaggtaataagcattaatagttaataggtactttttcaatccttaccctcttcccaccctccaccctccaccctccagtaggcccttgatgtctgctgtttccttctttgtgttcatgtgtactcaaagtttagctccacttatgagtgagaacctgtggcatttggttttatgttcctgccttagtttgcttaggataatgacttccagctccatccatgctgctgcaaagatacaatcttgttcttttttatggctgtgtagtattccacaatgtatgcataccatattttatttatccagtctactatttatgggaatttaggttgacttcatgtttttgctattgtgaatagtgctgacctatggagtggtactatgatgatgggaaaggccaagcagaagccacgataactgcctctacctaaaaaaagtaagccaaaaatgataccacattcctggaaggattgaagagattagtgccatcatcaagaatttgaaatatgttggagtgatgaaacctattcaactctcctatttatcctgtactaaagacaggttgatcttggagaatgacagtgcattatcataagcctaacaaggtggtggctcctattgcagctgctacatcagatgtggttttattgcttgaccaaattaatacatcccctcatactggacatacagttaatgatcaggcaaatgttgtattctccatcccctttaataaataaaataaagcccatcagaagcagttcacttttagctagtaaggccaccaatacacctccacagtcctacattaggggtatatcaactctctagccctatgttcataatttagtttgcagagatcttgatcacctttccctttcataagatatcacgctggtctatttcttgatgaaattatgctgattgaacctagtgagtaagaattagcaactactctagacttattggtaagacatttgcctatcagaaggtggaaaataaatttgataaatattgaggggtcttctatcagtgaaatttcctgaagaccactggtgtcggccatgtcaaggtatgccttctaagatgaagaacaaattgttgccttgacccctttacaactgatatggtttggatgtcttagccaaatctcatgttgaaatgtaatccccagtgttagaggtggagcctgataggaggtgattggatcatgaatgcagtttctcataccatccctgtcctcacaatagtgagtgagttcttgtgaccatttaaaagtgtgtagcaactcttgcttgtctttcttactcctgtgctggccatgtgatatgcctgctccccctttaccttctgccatgattgtaagtttcctgaggcctccccataagcagagcagatgccagtatcatgcttcctgtgtagcctgcagaacgatgagccaattaaacctcttttctttataaattacccagtctcaggtatttctttatagcaatacaagaatgatctaatacaacaaccaaaaaaaaagcagcaccaagcctagtgggcttcttgagattttggaggcaacacattcctcatttcggtgcactactccattcccatttactgaataactcaaaaacttctagttttgagtgaggcccagaacaagagaagactctgcagcagatccatgtaagctgctctgccacttgggccatatgatccagcatatccaatagtgcttgaagtgtaagtggcaaacagggaagccgtttgtggcctttggtataccgctacagatgaatcaaagagcaggcccttgggattttggagcagtcttgccatcccctgcagataactactctccttttgagaaacaattcttagcctgctattgtgtcttagcagaaactgaacatttaaccacagctcactaactcagcatgcaacctgagcttcccattgtgaaatggatgttaactgacaaaccaacccataaagttgggcatgcacagcagcactccattatcaaatggaagtattatagataggatcaggcctgagcaggcccggaaggcataagtaagttacatgaagaagtggctcaaatgcccatgatcccttattactctacctcgactctcccagcttgcacctatcgcctcatagaaagtttcctaccatcagttgacagaaagagaaaactcgggcatgattttattcttactggaatagacacttactctgcatataaattttcaattcctgcacacaatgtttcttccaaaactgtcatccatggagttacagaataccttatctactgtcccagtattttacacagtattgcttctgatcaaggaactcacttaccagtaaataaagtgcatcaattggcccatgcttatggaattccatggacttagccatgtttcccactatgctaaaacagctagcctggtgggacaatggaatggccttttgaagactcagctgcattgctagctaggtggcaacaccttgcaaggctgaggcaaggttctctaggaggttctgagtcagcacctagtacatggtgctgtttctcctgtagtcaggattcatggatccaggaatcaagaaatagaaataggagcagcaccactcactataattcctggtgacccactagcataatgtttgcctcctgttctcatgaccttgtgctctgcttgcctataagtcttagttctagagtgttctagagggaagaaacacaacaatgatttcattgaactggaagttaatactaacacaactactttagtgtcctcatgtctctaaatcaaagggcaaagaagagagttactgtgctggctggggtgattcatccaggctaccaagatgaaaatggactgctattttacaaaggatatagagaagagcatgtctgaaaaaaagagatcccttagatgcctcttaccataccctgtgattaaggtcaataaaaaactacatctcagttcagacacggctactaatgacccagattcttcaagaatgaaagtttgggttaccccaccaggtaaaaaaccatgccagcttaggtgcctgctgaaagcaaaggcaatacaaaaggagtagtgaaagaagtttgttataaataccagctatgctcagaggatcagttatgaaagaaggactctaactgtcacgagtatttcttccttattttgttatgaatatgtttgtgtgtgtgtatacatagcaaatgtctttgttttcttcactttcttagcccattacaatgtaacataagaagtattgactttgtatttaagataagtttatttaagtacttaagtattggtaactttatatcatagtatttaccttatggaatgccaaggaaaagggtaaacaccacccaagaactttgtatcctcttctgaggagagggttagtgtgtttttgcttgtactcaagacaggtgtatcatattaggttgaattatgacatttttattgtctttatttggagattaaggatagtttaaagagatgcatatgggtgccagattgataaggagtagaattgtgagagctaattttatgtgtcaatttggctgggccatagtgcccagatgtctggtcaaacattattctggatgttttggtgagggtcttccagatgaaattaacatttaaatcagtggattttgagtaaagtatatcgccctccgtaatgtgggtgggcctcatccgataagttgaaggcttacatagaacaaaggactatctatctatctatctatctatctatctatctatctatctgtatctctatctagctacccctacacatatcctattggttatgtttctctggagaatgctggctaatgaaaatggttaacaaaaagtcaggacaatggttaccagtggaggaaagtatgattgagagagacacacaggaggagtctaaggtattgataatattctatttttttacctaagtagaggtgcatgcattttatattaccactatatatgtttaacacatttttctgtatgaatactgaatatcacacatgtagacacacacaaatttaactcgaaaagtataaaatagaagactatcagctgaagatgaaaatagcaaactaaaacatacatgtaaaaaattatacaaaatatatgagaagataaagaaatgtaaaatatgagagattaagaaacagaaacaagagaatgagaaagtcaagctcaaataagaattatgagaggataacatataattagtggccaaggggaaatatttaagaagatcatggctgatagatggtcatataaaataagcagaagcagcaggattaataaaataaatacaaataaacatatgtgtagaataacatcttattccatttggtgctgcaatgacagaatccttaagactgaataatttataaaaaacagaaatatatgtctcacagttctggagactgagaaggtgatgattatggcactagcatctggtgggccttcttgcttgttaccacgtggcagaaggcagaaaggcaaaagagaattgtatcagtcctttcttatgctgctaataaagacatacctaacactaaataatttataaaggaaagaggtttaattgactcacagtttaacatagctgaagaggcctcaagaaacttacaatcatggtggaaggggaagcaaatatgtccttcctcacatggtggcagcaaagagaaatgctgagcagaagggtgaaaaccccttataaaaccatcagatctcataataattcactcacaatcatgacaaaagcataaaggtaactgcccccattattcaattacctcccactgggtccctcctatgacacgtgtggattacgggatgagatttgggtcaggacacagccaaaccatataaaaaatgaactcacttccataagccctttttaaagcagcattaatccttttatgagggcagatccctcaggacttaaacacttccctcaaggccctacctctcaataccattgcacggggatcaagttgccaacacataaatttgggggacatattcagatcataccattccaccctgggccctcaaatttcatttccttctcacatgcaaaatacgtttattccaacccagtagccctaagtcttaactcactccagcaccaactttaaagtttaagtccagagtctcatctacatcaaatatgtgtgtgaagtacttcatactgaggcaaaatgctctccagctgtgagcctataaaatcaaacaaattatgtacttttaaaatataatggtgtctcagacgtatgatagacattctcatttcaagaaagagaaatacaaaagaaaagaataacaagtttcaagtaagtaaaaatcccaatagggcaaatgacattgaatctcaaggcttgagaatagtctctttggactccatgttctgccttctggcgcactagaaggctggagttgggtttcaaggcctcagacaacccgtgtgtcagtcctcacacatttaagtcaaaagcttatggttttctcaggcttgtgctgcatactggtagctcggcagttctggggtcttgggaggagccttgctcccatggctgcactaggcattatcctagtgaggactctctgtggcagcccccaccccgcatttccgctggatattgctctaatggaggctctctgtggtcactccaattctgcagcaggtctctgcctgggctccgagactgcctgagacatccattgaaatctagctggaggtagctatgcctccatagctcttgctctctgggtcagtgatgagaggagcaggcttgaagatctccaaaatgactttggggctactctcccattgtcttgatgaatagcgcctgacttccttctattcacactaatttccttatcaaacagttacttggccatacccttggctgcttttcctaaagatgcttttttgttcttgacatggccaggctaagaattttccaaatctttacattctgcttcccttttgattataaattctatctttaaatactttctctcttttcaccttttactataagcaattaaaagacgccacacagcaccctacattttttgctacctggagatttcttctgccagatatcctggttcattgttaagttctgtctttcacaaagtcttaggacaaaaacacaattcagtcaagttctttgcctcttcatagcaaggatcaccttttctctagtttgtactaccttgttcctcctttcaatctgagacctcattgaaatggcctttactgtccatatttctacattctcttcataatcacttaagtaatctctaagaagatacaggttttggcttacagctcttctcttcttttaagacttcaccagaattgcccttaatgctccatttatggaaatacaggcttcttcagcactcagttcaaaactgtctcagcctctactcactacatagtttaaatgctgcttccacatttttagatattggttgtaacaaagcctcactattctgattccaatttttgtcttagtttgtttggtgttgctataacagaatacttgagactgggtaatctataaagaacaaaaattcatttcttacagttcttgaggctgggaaggtgaggatcatggcaccagcatctggtgagggccttcttgtggtgccctcacatgaaagaaggtggaaggccaaaagagaatgaacccattcccacaagccctttctatagcagcattaatccattcatcagggtgaagccctcatcacctaaacacctcccaaaaggccctgcctctcgacactgctgggctggggatcaagtttccaatgcatcatctggaagacacaatcagactaaatatcaaatactgaaagaaactatagtaaatgaagtcttaaaggaaacaaaaagaaaacaattagatgtacaagagattttacatcagaaagagattctatacctagaaaatcctaaagactgccaaaaagctcctagaatttattaacaactttagtaaagtttcaaggtacaaaatctatgtaaaaaaattagtagcatttctatacacaaacaacatccaggctgagagtgaaatcaagaatacaattccacttacaatagccacaaagaaaatgaaatacctagaaatacagcctggtatttaccaaggaggtaaaatatctctacaaggagaactgcaaaacattgctgaaagaaatcagagactacacaaataaatggaaaaatattctatgctcatatattagaagaatcaatattgtaaaaatggccatactgcccaaagcaatttaccgactcagtgctattcctatcaaactaccaatgtcattcatcacagaattagaaaaaaaatatctaaaattcatatgcaaccaaaaaagaccctgaatagccaaagcaatcctaaacaaagagaacaaagacatcatactacctgacttcaaactatggtttaaagccacagtaaacaaaacaggttagtactggtacaaaaacagacaaatagactgatggaacagaatgcaaaactcagaaataaagtcacacacctacaactatctaatcttcaacaaggccaacaaaaacaaactatggggaaaggactcctttttcaataaatgttgctgggacaactggctaaccatatgcagacgactgaagctggacaatttcctttcaccatatacaaaaattaactcaaaatggattaaagctttaaatgtaggacttcaaactataaaaataatagaagaaaacctaggaaatactcttctcaacattggccttggcaaggaatttttggctgagtccccaaaagcaattgcaacgaaaacaaaaatagacaagaaatagacaagtgggacttaatttaactaaagagcttctatacagtgaaagaaactatcaacagaataaacagacaacctacagaatggaagaagattttcacaaactatgcatccaacagaatcctcatatccagaatctacagagaacttaaacaaatcaatgggcaaaaaccaaataataccattaaaaaatgggcaaagtacatgaacagacacttctcaaaagaaggcatacaaatgaccaacaaacaaagaaatgcccagcatcactaattatcagagaaatgcaaataaaagtctcaatgagataacatctcacaccagtcagaatggccattattaaaaagtcaaaaacaacagatgttggtaaggctacggagaaaaggggaatgcttatacactgctggtgggaatgtaaatgctattcacaatagcacagacatggaattagccctggtacccatcaatagtagactagataaagaaaatgtggtacatagaatactacacagtcatgaaaagaatgaaatcatgtcatctttagcaatatggatggaattggaaggcataatcctaagcaaattaacacaggaaaagaaaaccaaatactgcatgttctcacttataagttggagctaagcattgagcacacatggacacaaatattagaacagtagacactgcagactaccagaggatgaaggtagggtgggttaaaaaactacctatctggaactatgctcactaccagggtgacaggatccatacggcgaacctcagcatgacacaatattcccatgtattaataacaaatctgcacatgtactcctgtatctaaagtaaaacttaaaattttcaaaaaaagagacaaaggagatatctactaaagtgtgctaggagaaaataactgtcaatctagagctctattcccagatgaacaatcatttaaaagttgagaaacaaaacatgttgatggaaggaaagattgagcttattctttgagactcttactaaaagaatcagaggaagatatattctgtttaaaaataaattaaacctctaataaggtaagctaccaaaaaatagcagtgggaaaataaagtaatacagattaggtaagtctaaattaaaactaatggtacaaacaattaatgtaaaagatttaaaaaaaacaagttggaagtaaagtactagaaaaatattatatggattgtaggagaaaaaatagaaataattactctaacatttttgtatcatttgggaggagggtaaatatacttattaacttgaaactttgattaagtgcatatgccaaatatttcagagaatcattgaggtagtagactgtgcaatttcccaaagtgtagaggatgagaaaagaaaactttattcaacatgaaacaagaaaggaggaaggtataaagcaacaaaaaggtgatcataagaagaaaacgtgatagtaggaatatatccaagcatatcagtaagcatatttcataataaacacaaacagttttaactgacaaacccacaaacctattagtacattttaacattagaaactctaactcttagaaattagagagaatggcactcaaaaaattaatatagacactacaatttagaaatttgatctaatggttaatatggttcattcttttgaaaacttgcatccaacaattagacattaagctttttttttccaagcacacaaagaatacttataaaacctgatcattcacatagattattttggaaaagtatggcaagcactgcctcttgcctgcccaatgcagtgtctgtctcaattccttattagatgagcctggtttttctgttcaggagataaaaagcgtttccttggtctgtagcaagggatgcctctccccaactccagagagtgactaatgatcagctccaactattttatacctacacattgttggcgtataggtatgcacattattttttttcttttctttttctttatttcaaacaccgtcttgctctgtcacccagcctggaatgcagtggcacaatcgtagctcgctgtagcctcaaagtcctgggctgaagtgatcctcctgcctcagcctccccagtcactgggactacaggtatacaccaccatgcctggttaattattattattattattattattattattatttgtaaaaatgaagtctcactatgctgcccagggtggtctcaaactcctaagctcaagcgatcctctgcctcagcctcccaaagtgctgggattacagttgtgagcgaccacactcagttctgatattcttatattaatccaacaatgatattaaaaaaaaaaaacaaaaaacctctctagttttctatgtggagaatcatgtggtatgcaaattagtgttttgtttcttttcagttcttatgtatcttctttgtttctgatgcacaactatccctataatacctactaccatatgaatctatatagatgtacctacagatgaactcatacactgatagcaaacattattactttattcctaattttcaagggaagatttttaatacgtcaccaagaagaacaatgtttataataggtttttagtggattgcctttaatagaataaggaagtttcttgctattcttggtctgctatgagttttgtctattgtcatgagtgatgattgaatgctaatacttcttctcctgaatctattgaagcaatcataagattttttcctttaatctactattgtgctaattaacataaatcattttagtatttcaacttagaaatctacacaaataatttggaaaaatgggataaatccgacttggtcaagatacatttgtttatgcattcatctattaatttcacatttcgatatgtatgtatatgaacatatatgcatatgtacatagaataaacactcctgcatttaatttattattttttcaggatttaaaaatctatgttcatgagtgagattgatttataaattttcctgacccatattttgtctgttttttataccaaaatcttattgaactcataaaaatttaatgttctaatcttcatatgctctctaatttttgcacatcgtttctcagatctgagagacatttatgaaaatcctgtcaacttttcctttcaattttgccaacttttactttatatattttaatgctatgctattagatgtctagaattcagaattgttagatctttctgttgaaatacatatttcagtattatgaagtaatccctttaatctctaaacacactttgccttaaagtttattttgtctgaaaatactattgatttataactgacaccagctttttttatttaacatttttccatcatatattttgcatcctaaaatccagcctgccaatcattgttaactgtcgaaattagaacatttgcatttattacaatcactgatgtgattgaaattatttctacgattttattctgtattttaattcttttagctttttcccatttaccccctgcctttctcttcaaatattacaattcctgcaattccctcttatagtttcctgatatatgtatgttagatcttttagttttctccatgttcctgaacttccctttcagaatagattggagaaggcacaatgttgttaaattctttgtcttctagtggtgtgtttttcataattcctccagatcctatcttcttgcaagctcagccatgcatttacaagactgtttgttgtattttatccaatatttctaggcgtttgaagtggggaggtttccaggaatagtttgccgtgttcccaggctgcaagttgcattaacaagagatccacactccagtctcagcttcttccctatagagctatggaattatagtcaagtcacttagcctctcttagcttgttcatcttcaaggttatcaagctgaggaaggcaacttggtgcagctcggagcttttaaaacatatgatcagatgacttatcttctttttttttttttttttttttttccgagatggagtctcgctctgtctccaggctggagagcagtggcgtgatctccgctcaccgcaacgttcggactctctggttcaagtgattctcctacctcagcctcccgaatagctgggattacatgcatgcgccatcacacccagctaatttttgtatttttagtagaaatgggatttcactatgttggccaggatggtctcaatctcctgacctcgtgatcctcctgccgcggcctcccaaagtgctgggattacaggcgtgagtcaacagatcactctgcagtataacattgaatgtcttctttgttttgttttccaaggttttattaaatgctaaaaataatcactcactctcttttagggtctagaggtgactgcacatttgggagtcacgaaacagtctgttcctaaagctgtgccagttatgtgaatttgaccaagtcacttaaagcctgtttacctttacttcctttcctctgttgtagaagttattgctttaaaaaatgcacataaatagcacgggactaaatcttggcaatctaatccacttgtgaaaaggaagtgaggactttaaagggaaagaaattaggaagaaaacaggagttcctatcttcctattattactggcagccccttctttcaatttctcttgttgaattttagataaaagtaaagggaaagagagaaagcatagcattttagctctatccttgaccgtgagttatttatgaacaggaaatatcaagattctaatgatgacaggagcacctttagcaattctacgggcagtgccagaacattgttgacaaggtgggccccagtagaatcaacctgaaagttggttcatcacattttaatttaaaaggttgaggtagaagaatggccagggagtatccctttcactcttgctcagaagagctttgtcttaattattgtggccccatcctattctaaatgatgtccttaataactgccctcctactctcccgttgtttccccgtcacatataaacagtgacgtggtctcagcttcagattgatcccatagggaccactggagcctgagttgttcacagagttattccaccttgaggtgtgggggttggctttaagaaaacctgtgtcagtcacttgttggctgaaaactgctcctaggggcaggggctataacctcccatgatgtggccgctggttgaggcacttcccgagagaagagggtagctgcaagcttttatgaagtactcaccatacctagagtgaaagtagggaaatgtaatagctagtaatgaagacctagatggggacatacagaattcactacagtttccagatttcatattcctgttgcaaactctgcacattaaccctgcctacatggtgatcgtggccctgtgaatgatattaaaaggaacaattaggctttccaactctcacatttgccctagagtttactgtcaacttttcaatagtcaataccctcttcatagtatggaaacatgtatttagtttagagggagtggatagagaaaagaggaagatgtttctaaagactcctccgaaaactgcagttccaccacagtattttgctgtcttattttaaagatcatattttgaaactcatagaaaaagccagagctaatttcagactttttatgccttgcaaagggcagaatagactttataaggaaaatatatttgtatcaggatacacacatacccaataaattccattaagtacagcaagttctttatcatctgggtttaaccatattgaactgccatataaatttaatatgcaacaaggtaatgtttggcacaaatactagagccataaaatatgtctcacagtactaagactagatggagggcaaattttagctacggtcttaggaaactaacaacagacatatatgtatcaaagtggcagaacctcaggtcctggtgaaaaataataattcttcttagcagtatagatgggagagattcagatttaaaaacacttttaagtgcaatttgggggaaatgtctttttgcttgttgctggtgggtgcactaattttctctaagatatttgaggaactggtaactttagttacctgtgagaggcaaactttgtagctgagaaaagtgagcgatacaaagactttcaggttttacttcctttttttgctttttgaattttattatttatttatttattttagcagagatggtgtttcaccatgttgcccatgctgatcttgaactcataagctcaaactgatccacctgctttggcctctcaaagtgctgggattacaggtctgagccactgcacccagcctgctttttgaattttaaagcatgagaatatattatatattcaatagttattaagagtgttttgtccttttgaaattccccgaaatatctgaattttggtttgggggagcatgcttaaaatggtttatattgatataggtagaggttagaataaacttttattgaggaataagtataatgatatcataattcatcagttgcttattacgaggtagctattttgctaagagatttaggtactttcttatttaatcctcaccacaaaatatggagtatgcgattatattttccccattgtacaaataagggaattaatgtttattatggataagcagcttatctgagatttcatagctagtaagtaactggtataaaatgttgctgtcacccagacagtctaactataggtccttgaagctataaccaagttagtagacatctcccaatctgagccctccttttctgtaacattcattggtaggtgcaggttaatggtttggtaatcattcattgcccttcaaattagcgttaggaaaatttacctcggtcactgtcatcctgcttggatgggtctaaatcagacctgaagaaagcaggcctccacgggatgctcgccaggttaacccttaggccaaaggatggactaatgttgagggacagtctctttccccatgttttgtgtttcatgttttccctaaaattatttgatctacagacctcaaagtcacctaaccaataacatttaagtttctaacaggccattgggattggtaagacagatggtataaaacaatcatcacttgacctagttcatctcattattgtatattttgcataaactcatctatgttatgacttattttcataaatatatgaatatatagccagtgttaagccatggtaattacaagcgaacaagggacttcccagacagaattgacctgtgtgttattctgttctaattctgaagtcaggaattactctaccagacccatccattgctaaaaacattctggtcttgtgtcttgcccattccaggtgagaagaacctgcttagtcctacctgtggattcaaccacagaccctggccacaaacatagtgtcctggaagaaaatttattgaacctgtttatctcatttcctcctgaaatatgttagacatagtaataagaagacctttggagtattctaggcaaagtatggtagaatcgaatataggacaatggcaataggaaggaagaaggaagaggatgaattgtggatgtttcaagggtgaaatctattggacctgaataatgataggatgagaagcctgagtaaggaaaaaaaaaaaagtcaaaaaaaaaaaaaaaaaaaagactaccagatttcccagtaaggtaatgaaggagataatttgtgccatctatggaggcactgagaaagaagctgaggtttttggctgttgattttggctttcatttggtttcacttatgtttggttgctttttattttggtgagttattatgttggtgcaaaagtgagtgcattaaaggtaatggcaaaaaccacaattacatttgtaccaacctaatagacggtggttagagtgagatgactgacttcatttcaactacaacaagcactaactaaccaacttctcatttccttctctttggtttctagtcaacactttgtggttctctaaggaacacaaagagagaccccaaagcactcacaagacagaagtccggaagttcctacatccaaaaagtttcctaaggccagatgttaccaactggcaaccatcagactgactctagccttcacagcattaatacctttcatatacctttgaaattatttgccaaggtttaaaagtagagacgttttattttaaaatatagatttttattttctatgtttgagaaataagcacagattcccaccaggcattatcagtaggagctgacccttcacacagggaatctgcccttcgttttaccactgcctcctccattcagtttcgtttttgaatagtgcctgcctgccttccctaggcatgctattttaaatatccggccttaggccaaaagcctcaataagcttcataaccccttccccactctttaaatgttatccctcagctccccctagcccaggtttgtgattacccctaaggaccatccaaagtaaaggaggagaaatattttcttcttaattacagggtggacattattttttctctaaccactggattctgggtaggtgccaatactgtttcacatttccatggtggccccaaattctctctccacctgtagccattattaggcctttagttaacactgtttaccccaaaggagacttaatagagagagaaaatataaaatgggtacagctcaggtgttacatgcctggggtctctggatttgggctaaacctcatctgatgagtcctcttataacctgctgtttcctgatgttgaattcctagacacctaggcctaatctgccaggttagacacctaaatccaaccatccataacaaacacttgattccaaacacagtatttgctcttggtaactgttacctctcagtgccctcctgccccattcaaaacaccggacattaagccaacagcatatctcagtgagtctagcagaaaatatgggtatgagttgtagcagaattatatatggttgagggagagcttccagtgaaaaactctgttatagttagttcttcttactgctggaaatctgcctaatctcttgggtggcttgaggcagaaaacatattcagaatcactggtgtatatgattcttgttttagtccattcttgctgctataacaaaataccacagactgagtaatttatgaacagtagaaatttatttctcgcagttctggagggtgggaagtccaagatcacagcaccagcagattttgtgtctggtgagggccttttccataaatggcattttctcatggtgttctcaaatggtagagaaaaccaaaggtacaattgctgtgttctcacctggcagatgggatggaagggcagactgctctttgaagcctcttttataaggatagtaatctcattcatgagagtggagtgctcatggcctaatcacatcccaaagattctacctcttaataccattaccttgagatttaaggtctaacatgaatcttggagggacacaaaccctccatagcagtccccttaagtaactctaccttctcccaatatccacttacttgtggactaaagccgtccctcatgagtttcctatcatgccattacttcctggttcctaggtccaatacctacttctttctcactggtagccctggaaagctggccactaccctgtcttctaatcacattttgacttcaagtgtgtgaggatgcatagccagttataaatctgagaaaactacaattggatctgaaaaactaattttatcatgtgaaagttttcttattcattcattcaacatgaattgagcatctaatatatgccatgtgatagaaatgcagaaatgagtaagacataataattgacctagtagaactggcaaaatgtatgaattctgtaattcaacatggtaaatgctctaatagaggcagatcacagctttatggtatattagaaggcataaacaaggggcatctgacttatagaacagtgttcctcaaactttatcatgcttctgaatcacaaaaaatcttgttaaaatgaagacgctggtcttgagcagggctgagaacctgcatttctaacaagctcccaggttatgtcaatggtgctgatccgtggaccagtcttgtaacaaattctagtgacttgaaaattagcatgaagaatgttagaattgggctccctctctggtgaaattaacagtgtttgagacatttggaataactaaatattttagaaacagtctccacctttgtcttcataagaaaccactgagacctataccctgagttccttagggaaagcactgactttagtcactaatgagttggtgtccagccaatagtttaggacggcacaagagccaacctctgaatgtcaccatgtgtgcttcgggtgaatatttcttctgtgttgttaaagaacctccaatgatagttcacttgaacttcgtcagacttacagactcatttggtgcctgctcctctaataaatgtccatatgtataatagaccattatctgaaatatgctgtcattacaacctgggcttgcagttatggggagtggctaatcctcaacgactcactctttgattgctcctgttttcaactaaaacctgaagagttgttggctaagtttggtcagttaacaatatcatatctgtaaaatcaaggtaaaaatgctcaccctgcctactttacacaacaaattgagatgagatgtgctttgaaaacttgaaatataatgaaagtactagctgtaattaaaagaacgtagatgcttttcctacagtatctagttaagataaaagtttctgaatttgtgattataagaatagtctaaggttcccattaaaaaatacatactcttgggtcttatcattggaaattttggtttagtagtctgggggaactatatatgtacgtacaatatatgtaataagcacccctaaatgatccttgtaattgtgtcacctgcattttacagatatgatgttgttaaccgatctaactccaccaacaactcctcgggttttgaccaaaaacaggcgcactcaaagagtgaatgaaatagtgagaagttaagaaattctgcattgaacaatggaatttagtgcacacaagagaactctacctcccatcaccagtttaattatgccagatagtacgtctataccattaccattactacaaatgactcatggcaaatccttcccactcacttagctcctttcctcctgagagtttgtataaaacattagagactacgttgacagtatgcataatacaacctacaaaaatatgtgagaagataaaatttgagacttccaatataataactttttttcttaatcattttataatcaagaaattgcccttgcctggaagttatatcatttgaacttttatccccaagatcttcaaaaaacaatgagtcacttaaagatgtcatctggtaactatttcattctaaagctgtgctatattatgttccttaaaggtgtggccaccagaagaacattactttatgatgaataaatatcctctgttacacttatgtttatgtaaagtatctatgactaaaacaaacttacaaggtcatctatgtaccaaattataactgccacctaaagactcacaggtggagagtacacatggattacttaagtaaatttcttacctgagagtcacaactggctggatgaatcctcttttacctccttcagagaaacagacctatgactctaactagacaaaatttcagactgaatctcttagagaatgcaatcaagtctattgattcaattataaagtgctactttggttacagggtgacaaacttccagatattattatccctaaaggttatataggctgaaaatctcagtcgatttaagataaatctgggctgaaagatcataaatggggaaatgtaggacattccagatacagcctttacttgaaggttgacagaaggaggataaacagatgcctgcaggatgatccatggtgacactgtcacaggtaaactgaagagactgtgtccaagtcaatcagtatgtgtggaggatttgttgctatttgttcttctcatttttccaatgtgatcgaagttccttaggtttagcccaataaacatctccctcactactagtgaccacagcaaaaaaaaaaaaaaaatgtgtgtaacaaaatatgcactatagaggatcaaactgttttacaaaatcttcaaaaacctaaatgttttcttagcaaatgatggcatgtccttcaaacactagaacactatattgtttattttgctttcaggctgattataaggtaaattcaactcagagagacttgatcttcttcctaatttacctgcttcaacaagaaaccatgagtgttcattatcaaccatattttgaaaattgaataataaataatggtaactactttattaataaaacaaatatgtttttaattaagtgactgtggctgccaggctccagaaagttaaaatgccatcaatagataggcagtgaaattctgaaagcagttaaattgcagagtacttgagaactggtaattttagatgaaagacagctaagacacaaatttaaaagcctatccattgttccttttttaaaatgatatgaagagatattgtacatcacaactgctatggactgaatatttgtgtcctcccaaaattcatattaaagcctatatccccaatgtgatggtattgggaggttggacttttgggaacttactaggtcatgatggtggaaccatcccctaatctcattcatcctccaccatgaatgagattagtgcctctataataagaggcaaacaagagatcatctctctctgccatgttaagatataacaaaaaaacagctgtctgcaaactaggaagagtgcactcaccagataccagatctaccagtgctctgatcttagacttcccagccttcaaattgcatagtattttgtttagtctatggtattttgttgtagcagcctgagctgattaagacaattttcttcatagatcattttcttgggaatggtggtaagatgctcataaatgcttaaaactacacataacaatcacacagctagaagagatcccagaaaatctttcaaagaaaattcagaaccaaagttctggctctcactttatctccagccttaaatgagctggagagtcagttatgtaactaaatgtctcatgcctcagattcctcatgcattcattgggaagaatgcctgtgcaatgtgtctttagggttaccatagagatgaatgaggtaatgtatagaagaagcacaatcccaaataatatttcccttaccataacttttctgctaaataacatctaaaattttagcccttgtttaatttagcctcatatttctcgtctcttgccctttgtaactactgtgccaaactttccactttatgatatacagtaagtccacttaatgctgtcgataggttcttggaaactgtgacttaaagtgaaatgacatacaacaggtctttgaataatatcatttctttcaacatccttgataagaataaaacattggtttcattctatgttgttttgtttcaagtcacagtttccaagaacctatcaacaacattaagtgaggacttactgtatagggtttcctttattttcattttctattttgccaacataaccaccagcctttgctttagtacagtacctactcttgaagctaccccatcagtgttcactgattaaatatgaggagaagggagaggtaggctgctgctcagggacttttcacactaagacaagaaaccagagcaaaggtgattcccccttgcgtggctaaactatggttgctgaagcaaaaggcattggagcagaaatatttcccttctaatcactagtaatgcctcaggcaataatggatgcttcataagcaaaggaattcccactaaaccatcaggaagtctagtgtaatagcctatttccaacctgccagattcattgagtgaattggagcaaatctcatctatacttctcactttctgatataattttatagagactaaatagtaatgttcaccataatgttggatgagcctcacttgtgaagcattcactcagtatatacctggcataatctcctcagattggtactttttcagatttaagagggaaatagcttgaaattgagaactaatagcaaattacatgcccttaagatgatacacccttcaatgacaaaacactgtagcacacctccaggtctgaagttaacaaagagggctgtcacttcattcagctgaatccctcctgccctccccctgcatacaccccaggcaacctttgccctactacaatctagctcattaatttggtcaatgcaattgtttctcatttaaatcttcctgtaaaacatagaatagtcacttcactattacattgtccccaattttttcaaaagcttgccgtgaagggaaggaagctaagtaagaaaaaggtcaatgaagccagttgcaaagaagattatgaaatgttgctcatttctgcttttttttttcttttgaacctctgaattacttcatctttccatctgtctacaaagttgtgggtgcacattaagatatacagacagaattcacaaaccctttggtaaattcactcttagcactaaatatcaacggaggagaaaaaaaaaacgtactatgtttcccaaaagaagtatggggagactattttctatgattaaaagcataagaaaaaaaataaaactgatccaagagcccagagactatttttatatgtgaaaaatatatactttagtcaggatttgtaacttcagtaactccctttggaaaccaaatttccaccctctcacagtgtttggttatttaggactgagtcaatttgagcatgatgagtttggaatgccagctggctgcagaagtgtgaagaaacctgtgtggtcaccaacactttaaacatcactcacagttctgaagtagggctatcccaggagcacttcctggatgctggtgatccacaaacaccagactatttccactcctcaaaaggaaaaagaatggggccctaccttgccaggaagaggaatctggcaagcaggctgagaacaagcaccacacaaggcaatttttgccaagtgagtcacccataccccctgggtacaggtgaagctcaaggaaaggaccattcaaacttccagcctgcaggagctgcaaaatcagctcctgaactccagtattcaactcatctagaaacacatttttattcaaatctttaaagccaaagaaggtatctaaccattgtcccagccttctcttcttttatagtatcactaattccatagttatgtaagtgccttttatgtgaatgcactgtactagacccttgatagagaatggttagcagaaaaattaaaaaagcaaccaagattctacattcttgattctcatgggaaaaatagagaagaactgagaaaagtcctaagaagaaaacactaagatagacactaacagggaaagttttactcaaggtggtcttatgttctaattttaatgtgatcacttggggatcctgttttaaaaaaaaaaaagaaacatagattccagtactaaatgcagatttgatatgtcttggggagagtctgggactatgcattttttacaggctcccagctgctgctgctgttgctgctccacaaaccacattttttaataacaagcctctagggtttctctacatctgtggttgtagttctctaagtgtggtcaccagaccagcagctacagtgttactgggaaacttcttagaaatgcaaattctcaagcctcaccccagacctaccgaatcaaaagctggagagggggacagggcggcaaggatgccaacactctatattttaacaaactctcaaggtgattatgacgcctgctaaaccccattcatccaatactagtcatgagccacatgtggcaattgagcactcaaaatgtggctagtttgaattgaaatatgctgttaggataaattatatgctagatttcaaggacttgaaaaaagaatataaaaatcccttattaatattttttatattaagtgcatgtggaaatactatcttggatataggaattaaattatattattaagattagtttcacctgtttcattttgcttattttaatgtggctactaaaaattttttaattacatatgcttgtaatggttagcattatgtttctactgggcaatgttgcactaacctttgagaattattgctatagagcaatggttttcaagtgaggcaattttgccccctagcagacatttggcaatattcgaagacatttttgatttttacagatggggtcaggggttctgtaagtaaaggccaagatgctgctaaacatcctataatgcacaggactgcttgctacaacaaaagactctccagtccaaagtgtcaatagtgtggaagttaagaaatctgttctattacactgattctgctcattagaaacactttaggagagttaaaaatactgatgttcttggtttatccccagattaattaaattggaatctctggtacagaagccgtggcatcggttgttcattttgttttgttttaagttccctgtggattataaggtgctgccagggtcaagaacctctttttctagtggaatggacagaggcccctcattcattcgtctctgtactgccaacactcatcacctccagacctgggaactggcatttccaacaagtgtccagtgatgctgaagctgctgctctggtttcctctggagaattttacgtcccgttccacataatgctacacacttcaactctccagatggcccagggagcctcatcagacctgggcagttgttccccactcagaagacagtcccaccaccaacgattccccttccagactcaatcaaaatgacttcccattcagctgaggtagaagcagctcttttatcaagggcatgggggcatggttaaaggagacataaactaagtcctttctgtttgcactccaagcaggaaacaaatttttctgttactgggtcatcttagggaaacctgtttcattcaactatttaatttttaccaatatataacttaaatcactttaaaataaatttgtacagattacaaaatgacatagattaaaacaacttctcagccaggtgcggtggttcacacctgtaatcccagcactttgggaggctgaggcaggcagatcacgagatcaggagttcgagaccagcctggccaacatgatgaaaccccgtctctactaaaaatacaaaaattagctgggcatggtggtgggcgcctgtaagcccagctactcaggaggctgaggcaggagaatcatttgaacctgtgaggcagagtttgcagtgagatggcgccattgcactccagcctgggcaacagggctagactccatctcaaaaaaaaaaaaaaaaaaaaaaagttctccctaaaactaatcccctaagaaagtaggaactagagaaatttgaactttaaatattttatggtccttcctccttcataattaacttttgaaaaaatatatatatacataaatctaaaatcgttggccctacatgatctctctctcacacacatgcgtgtgcgcacacacgcacacacacactctattatcttcatgaacacatcacctactttgctccagtccagctgacagccttaggcccttcgcactaagtgtctttccccaacatcatctgaatgcataacttccccactcttgtctcccctggtctttgcccagatgttatctgctccctcccatctgggttcctctttctcaaccttcttctctctgcctgattggttttttcatagtacttattatatttttaaagtaccacctaatttacttattctgtttattgtttactgtctttctcccatgctggaatcctcgcaatctttttgttcatttacatttctcaaatgtctaacaggacacctggcactcagtaggtctgtaataaaaaatttctgattgaataggtgtttctaaaaatctaaaaatctttcctttttctcacaactttattgcatttgttaaataaattctttgcaaaattgtttttgaagttcttttaaagtatctttgctatacactaagcaagtgattagaatgcctatttgacagccatagccaaatacactgagaattcaagcagcttctagaactcaaatgatgtcacagtagctgcattgtggtgagtaaggggaaattactatagtgaaggtcaaagaaatacacagaggccaggctgctgagggtcttgtgagccagggcaaggagtctgaattatgttttcaggtaatcaggaagctactggaggttttaggcaggaggtagacgaagacactgagcatcatagaatatggttgataaggcccactggattgctctgagggtggcattgtccattttttttttattcaatgtccctcatcttctgatgatgcataatttagcaagcttggttgtgctggatagtagagcactagctagaagtgtagggtcaaaccaactttggggtgctgaatttcacccctggatcctcactaactaaagtgtggtctgtgggttcataaaacatgcagaatcccaggccccatgctggacctaccaaactggaatttgcattttaacaagatctccaggtgatttggaggcacactaaagatgaagcggcactgtcttaaggcatagaatattgattgagcccctcacttctagctatgtaaggacaatactggggagggttggatgagagaattctttctttccagagtctgggctacatgttgacccgctccctatggcagtgacttctatccttggctaagagcataaaagtgtccacggatcagaaagcttgagaactactaaattagatcaaagtaaaataaagcctcttgcgggaaacaagaaactttttgagtaggcttttgggtaagcaggatggaatgtactgacgagaagaaagaaatgacaagaagaaagaagagtcaacctgcttctaaaacaaataaacacaccatgagaaggaatgtcagggtctaaatttggatctcaatgtcctcttcattgtgacgttttctttgtcatacataactgacttgctaaaaccaggcactctcccccacttagggccctgctgttctctctgccccaaatgctcttctaccaggtagccatataattaattccctttcctccttcaagatttttatcaagatttaccatcccaatgatgcccacctgaacactcaactgaaaattataatccacctcactgcattcaccacattcccaactcccttcacctagctttgcttttctttttccatattactcacaacatgctaaacttccatataacttatttatttattatgtctactgatactgtactctctcctggctaaaatagaagctccatgaggaaaggctttttatttgttctctgatctatcccaagcatctactccagagcaatgcctgatacattgtacatccacaataaatattttttaattgaagaagagagttttgcagatagaggcagtggagcttaagagatcaaagtcggctgttagactcaggccgaacctgaatttgaattctggttcaggcaattatcaggctcatgatctttggcaagttattaagctctctagacctcaatttcctcacatgtaaaaatctgcttcacagtgttgtggtaagaataaataagataatatctgaacaccgcatatcacctggcacttagtaattggtcatacactatggttgtcacatcattattagctactttatcttttatgctcccaggagtttgtgtccaactgcttcctgattggattaaaggcaacaggcagccccaggttctctttggctacataaagactagctcaattttccactgctcaagaagataatattgttccaaactcaaggatatgaataaaacacatttacctcacagcatgtctgtgccaaaaacattgtgagaggtcatttagttgaaactttatattactttaacaggtgaggaaggcctgaggccgagagattttctaaaagtcacatcgcttgttagtgtagagtagaacctgaaatccaggcttttagattcccaggccagctcaattttccattactcaagaagaaaatattgtcccaaactcagggatatgaataaaacacatttacttcacagcacgtctgtgctaaaagcattgtgagaggtcatgtagttgaaactttatattactttaacaggtaaggaaggactgaggcaaagagattaaaagtcccaccacttgttagtgtagagtagaacctgaaatccaggcttttagattcccaggccaacattctttccacttcattcagcatggaaactgttgtaaaaagttccaaagttttcaccctatacaaggttagcatagagaagtagaagataattgagatcatgtctgggaatgacccagggtgcctctcttgcactcagtagcccctgccaaaacatgcccatcatcgttgtaattcataattaatgatctgcacatgaataaacattagacaaaccctgcctataacctgcaagaacactaaagtcctttatacggtcatatagaagatcctaatcaagcacagataagtccatcaaggaattgctgcagcaatttttcaaccctaggatagtaattttaaataccttaggctatacctataattgcatatttggattgagatttattaacccatgtagtaaactattttggctgactttttttcccagaatattgtttaaaagtgtcagaaaagtaaaatctgtgggatttagatatatgtaagtaattataaagttggttcttccacttgtacagccaaatgaccaaataacatacagtaaaagagaagctgtataaaaattcaaatatgcaagatataacagaccagtctgtgattaagcaaacctaaattactaaaaataaaacaaaacaaggaagcaaacaaaacagttgttatggaaatatgtttgaatcattgaaagcaaattaaagatccctcctatttgccaaacattacactcagactccactgcacagtacaagcctgtgcaaatcaacatcacaaaacaatacatggaatgcagtaccatagtagacctccaagacatagcaaaacagtggataatatgaaagtccccatgggtagttgattatgaagttctttacctgaagaatctaatcttctaatccattttaagctcattctttttttcataaatatttattaagcatctcccacgtagggtttgaaacatcaggaattcaaagatagataatgtttttatatattccaaacccagtttagaacttagggaagaaagtctagacagagtttgttctcaaagcgatgggtagattaggaaatgaaaataaaaacatccatgtttagctggggcagatgtcccacagccctcactgtcaggcaaaggctaaatatataattatacggggctgatttggaaggcaaataaacaaacaggaaatgtaagcattgttttaaaagactaaatctaagtttcagggattgaataggaaatacaagcagtaatttccttgtggctaggcaggaaatatataatcaaggaaagtcaagcttctgcctagaacaaatgacaagtaggtagaatgtgaggaaatagataaatgccagcctgagtctgaagtgtattagagcttccactgaggaagccagccatggcattatcacagatgagcactcttctgcatagggatcaggacatctctgatataagacaggtttattcccatcacatttcttaccccagtagacagtacccagagagacccaacacagaaatatgcaacagatcaggcaggacatggcaaaatgcctaataatagaataacagaaagttattggggtcattctagccatctgagaatcaaatgagtaaacattacctatgtgtttagctctggaatagtgaaggaatagggagtgtttgacaatgcaacttaccttctctagctgcaaatgaaagattctagaagttttggggaggattttaaggtcctgaactcccaggtgccagttcctaaataatggtagtcaactatttcttaatcccacaaacacaattagaccagctcactcccatttatctgcttgtaagtttctgcagatggtctccctaagacaacatcaagcattgcagtaggagcacaaactgaaattggacagcctgggtcagaatcaggcccaaccctactggttgtgtgatcttagaagagttaacatagctaagcctcaatttcttcgtgttttaaagggggaagttagtagaaaactaacagagattaccgtgaggcttgaaaaaagttatcacatgtttaaagttcagttaccttcatcatcattagtgtacagtgtcctcggcaatgttgagtgtggtgccagtggccatcaggatgcagtcagtgtggcatttttaatattttgttgtcattcttactggtatccttgctaaatgcattcaaatattgactaatatgtactatttagctcatccacaccagacggccccgagtggtccttcagggtctctccatatatccttatcccccctgaactaatacctagaaggattgtgcctctgtattctccatgagttaggctctcagtaaattagttcccatttcagacttcatttgatatccaaggaaggcagattccatgactgaatcctcaatttcaacatcattcagtagggattggacagtgggtgtcctcagggcattaccctgttcagccaggctaggggcaaagttccaggagcagtgatggcttctagacccaagggatcctgggggtcatcgttgtatggtttccttctatacatatccgtacctagacaacatactattctccttaacattcctttatgtgtgctcacaatgtgcacattatgttcaatgtacaataattaattggatcttcaaattatcaagacaaagatagcttgataatttgaaaatttactttgaaaaagtaaaagcaaccaaaaaggtacttcaagtaagagcaaaacattgaggagcagaaacatctttcctgtccttgggtcctttttttaacagaatgtttcatctgttcagcccttaatctcaatacaacatgatcttgaaatcttataccaagtttagatacaaattccagagccattactgaaacttgaattgccgatgcttagaatgtagaattagaaaatgtcaaagtccgggtccctctaaatcaagactgtgtagcattataaaaacatggtgaaattgtcccaactgtctctctatcctgaggcactctcttcagttcgtcttatgcagagagctgaaggacaatttgatggtttatgaatctatcctatttcttccacacacaccaagcaactgtactttatacttctaggcctaatgatgatggaaattgctgtaaggacatcatgcctaaaagaggattgacttgaagcaaacactttacaaatgtgtatcttttaagagttgccacggtaagaattaaaataaagcacatatgtgtctcctaaaacataataaagcatacatttgtattactagtgtatatttgctattataataagacaaatactcttatgaggcaaagactgattgaagtatatcagttccaaatggagacagatgtgttttccttcagccatttcttttttttaaaaaaaaaacgcatatttcaaacatttatgtgctctttcattaacagcagtatgaattcaattttccagacattctcattatgtgttggtaagattaccattttcattttgtctgtgaataactgaagaccaaggagaacagatagctgtttgggtaagacaaccagataacgtggaaagcaaagctactctccaaagcagctggaaaagctcattaaaataatctgagagaaggccaactcactatgctggtggttgtcaaaacctgcagcgtataaaaatcacctaggagtcttgtaaaaactacagattcccaggaccccactttcttgagattccgatttaggagcctgaggaataaggcgcagaaatgtgcacttatgacaagcattgcaggaatatgattgatatatgattaaagggttggaaataaagccttcagggaaaagccaaagaatgtgagttatttagacaaaagaaagattggatttcattttcttaaagtactgtatatatagattacagatgaagatatatagataataaagaaacaattctatatttccaccagacatgacaacttagaattgcaacatttacataaggcactatttacataataacactacgttatttggctaattataatagggaatgggagttacagattttcatctggaaagatgacgagaacgaagtaccaagagttctttccacgttttttaagagcagggctctctagaaaacgggagacaaggagtttacctggacagtggtatcatctgactctgctattattattttatgaaagaaaaaggcatctttccaaggactcctgatgttattgtaaaactgctctcacatgttggacacatgactgcaacactgcttgcagctgttcgtcctgctcttgccaatcattatttatgcctgtgaagagttgaataagatgcaaaagagtcagtgtcaaattagtcctgaagaaactgtatttcttttgccttgatgctatgtagtggggaaaagctggcttttaagtatattcttgatgttcttaaacccaggataaggaagatacacaatcttatacctctgcttccaggaaacacatgcagcccaagcaagttccaaaaaaaaaaaaaaaaaaaaaaaaccgtaacaatatcttcaatgtgagtaaaccccatttcacatacaatagcatgactgttgtgaaagaataacaataactttggaaggaggaataaaaatatgctcattttcctctaattgccatatagaaatttttgtaaaaatgacattaatttccaacgtgtcttattagtagagggtttctcaaccttggcaccactgacattctaggccagataattgctttgggggctgttctgtgcattttcagatgtttagcagcatctttagccgctacaaactgtatgacaaaagcatccccatcctccttgtgatgatcaaaaatgcctccagacattgtccatgagggcaaaatgaccactggttagaataatagtccatggtggtaatcacataacctgcttttacctgactgaagtagcccagtatagactggaatttacccattcctggaatgccctaattagtaccatattgctttccctatactgttgtttaaacatagcaatactcagacaaagaaaggcaagctgttagtagttgtcataaagagggtacacacaagcaggagaagaccactacaatcacaatctaccacagaaacctcagagtgaaaacacactgggattgattcaaagtgcttcctagtatccataacttatccataaccaagggtgaccaaagaaggggactggagccatggctctaccactacctatcccttatttggcatgatcctcttatgtacccaggtctgaatgtacataaagatatggtgtaaacagtgtttgttttgattgtccccttggtgacttgatgttctcaagtcatgggctttggagtcacactaacagaagttcatattctagttatgccactaactggttctgcgatctttaataagttatttaacctcttcaagtctcagttcctttatctagaaggtgagaataataaatcaagcccataaggtatggtgacaataaagaaagtatgtaaaacacaggcataacataagcacccaatgaaaacaaatttttcattgtttttgctgttgcttttgctgctatggtaatcattggcattattatcattataccaattgatgttaagtttcttaaaccccagtgagttcaggaatttcaaggcaaggactactgccccagcccattcctctttttctctttcatatttaggaaatccctacatccttctaacttagatgagtgatttcatagaaaaatgttatcatcttggtcactaaccaagccaccaccaccaattttatgaatatctttaaaagaatgccctccatgacttaatttgaagccctaatcctcatgatggaaattttggcatttcagcagatttgcggtcctagaatttggaggttaggaggatctttcagtggtcctccattcacattagcttgtaagatgtcacctaagagtaagaagaatcagctcttctagcatcttagtgctgggtgaagagaggataaagacaattgagacagagaatttggctagtgacaacacgtgattacaaatttccactttcaatccccctttcatccttccacccttgacacttgccagagaaattgcatacttcccacagcctgaaaagccatgttcctcttccctccctagggaaatcttacccattcttcaaagatgaactcaaaagtagttttctgtgaagccttccctgctaaacccaagttgagggtaatcgctcacagaactttagaactggacaaaatctctgtatttttatagttagggtccatgttttggaggtatgattaaagagattttagtaggaaaaaggactgactgattccagctgttggtattctatttggaattatgattgttatttttaatgcttttaaattgcactagattctaagttgttggggtggaatccctgttgtactcaatattgtctgtccatagcatctaaaacagtgcatgaaccgtagttcatacttaataaataccagttgaatgaattaaccctatattattaaccaagcaggtttaatagacactatttgctgcttcttatcaatgtcagcttaattgttcctcctgagtatccatggatatctaccagccacattgatcatgttaaaggtcatgaggagcagttttacaaaaatcatgtattacttaagtttttttggttttcttactattattcactccaacacaagaattttaaaaaattgaaatagaataataataaaaatcaacacgagtagtgctatctaaaccaagtgatttggtgtacattttctcatttaatcttcagagcaacttgtagttaacataattattcccagtctacagaaggttaagctgaggatgctttgctcataatcacatggccaagcagagaccatgtttctcagcgccaaccacatgtctttcttacttcattgttagattcactgtgactagggagtacataatctctcaaacccatccacactcattgaagtctatggaaaacaaactggagacaaaaacacccataggcaacaatgagttgactctttctgtggttagtttttctctgtattttttcaaatctattcattcttcctgactacagcatcatttgccatagtcttctcctaagtcaatgtcatacttcctctcaaccacttgagagattgcagatctttgtcattcaacatcatttaatttcctgaataaagaaacaaaaaaaatggaagaggtttaggtatacatctagagttgattttaatttcctgtttgcccttgtaattatggtttttcttctttgcttttgaaaactataatgagtgtttgaagtattacaattactttcgattgcaaaaactgcaattacttttgcaccggcctaatatatgttctatgtggtctttctcatctttaaaagagcaagcaattgattataacacaatggtgtcaacagccattggatatctaccaattatccaataggacaatttatctaatgaattaccttttaggttacctatatgcttaaatttggttacaacacacttttacactggtatatgtaagatagaagaacatacagctcaaatctcgagttctcttgctgccaatattgatggagtttatgaaaaattaaaatgatgaagcatacttcaggtgttcatttccaaatgcaatgcattttctattttttccattttaaaaataatatgcacccattgtaaaaagaaacttcatcattatcatcgttaaataaacagaggcaatttccttcataacctcttctgttccaccttcacctccaagtcaactcccactccagtggtattatgttaataatttgtgtgtttgcgtgtttgtgtgtgtgtgtgtgtgtgtatacacgtgtttccaggaagagacagaaaaaatgcagatatatagaaagcatgatacacatttttaatttgattatctttgcttactaatatatcataaacgtctctcttaccagtataatacaaattgacctcatgctttttaatagcttcatagtgttttatggaatgcttatactacaaatttgctaaatcattcccctactagtgatattttgacatacattgacagatatttttaattttttttattgtgtgacaatccacagaggacatcttcatgtttacatctttgtgctcatttgggcattactactaaagaaagtcttagacaaagaattcctgattcaaagtgtatgcatttttaaaatgcaagtcatgagctttagagtcacactgacagttcatattactttcttacttcataagcctgttcaattaccctccaaagggctatgaaaatttctgctttccacacaatgtatgagtcgccttatttctttacaccctcacagtgtggatatcatcaactggcttcacttgaattcccctgaaagcaggtcctgagacaaaaatgtgaaggcaatacagatggtcactgacttccaatgattccacttatactttttcaactttacaatggtgcacaaactatagacattcagtagaaaacagtgtgattctctcacatgatgctgggccatgcagtaagctgcagctctcactcagccacacaaaggtaaaaataccagtactgtacagtacattgtgttgccagatgattttgcccaactataggccaatgtaagtgttccaagcatgtttaaggtgggctaagctaaagtatgatgtttggtaggttaggtatattaaatgcatttttgacttagtcatattttcaacttacaatggtttatcaggacataaccccattgtaaatgggggagcatctgtagtttattttgaagttaattccaggaatcactatgaggttgcaaggagtaagacaagaagtggggaacaaatagacagaacattgactagccagttacctctattcgtagtgtggtcaactatcacagtttgcatggaatcaatggggtgtcccaggacacagaatttccaatgctaaaattgggacagttctagaaaagcagagataagttggttaccctactgtatgggcaattgggctcaattattctggagacccatggaaaggccatatggaacacaacttaggattgttccaacaaaaggcaaagatgtgaagtatttattcaccagcccttagtagtttggaggtactccaaggcattctggctcagttacttcccatctgacctgtgcagggacaaagcatgctcctgtgtccaaagatagcccactggaggaaaggtgcaggaatttgttgtcatgtctgagaaatgtctgcagacaaactctgggatcggccaaaaggatgtgggggtgggagtgccaataccatctatgcatgattatttgtccctagtctgatcagcaaaatgatgctatctcaatattgttctcatttacattctctaatgagtgggaattttggacataacttcatatttgcatccctttttgagactcaagttttcaagcatttaaatacagttttagggtaggttattttcttggcctgggacaaccaacttccataatagaaaaaaaatttcaatcagtttacctaaaatatgtgtgtattggaatggaatgggatgggataggggagcagggtaaactcttccaggtgaattaaaggtgagaattaatgatttaattatagtattttcaaggccacaatccttgttagactttcctttaccttatttttaaaaaactgtacatatgtatggggtaagtatatacatatgtgtcattatacatatttatcatgtacatattatcatatattgtatacatatacatatcattatatattgtacagttttatgtacagttttttaaaaaggtacgtatgtatggggtacaatgtatagtgatcaagtcagggtagttagtatatccatcacctcaaatacttatcatttctttgtgttgggaatatttcaagtcttctcctccaggtattttgaaatatacaataaattattgttaactatagtcaccctacagtgctgaagcggaattgagctccttaagttttgtacatcatgtttttttaggttcccactgagctgatttttggccatgattcacacatatctctcctccaaggctcctctcacaaagcatttcctcccagtcacgttttcaaatagcttctcattccctgtatgcctgtgtgtgcatggcctcatctcactttcgctgtgaccattgctgctcatttctatttccttcaagcctcatttactgctgcctaatttgccttctagctcagaggaaagcagagatgtttaatggagctctcagaatcatcattactgaaacttgacacagtcaaatcccttgtgtcccttaatacgcataatattgcttagcctaaaagccacagtctgggagactcactgtatcataatgagaacattcagaagataactaaatcataaatttaaacttcagattctatcacttcattgattaagctctcagacatttccccaactacctaagacctgtcattattaatcttacctgttttattaattccatttcactgtgtttcttaacacatatgttacagcctcccaccaagtctatcctccaatcatgattacaaactggcagtaccttagaatgatgatcttttgatagttttattaccgagcagaaagcaatctgtatagtttctgtctcacaaattaattgcttagacttttcctgctaagcttccacctcagaacttgtccccatttcaaagagtaaaccaaccctcattttacaaagatagcaactaaaattccaagacattaaatgacttgcctgaggctacacaactcatggccacagacagaactcttcctcctaggctacttgtgttatgtcagggtgattcatcttctctatgcttctctgaaaaaaacagaatctgtcaagcagtcatctccccatccccccacctgcctcagaccctgtgcttcagttctgttctctaccacgactctaccctacccttcaatcacttcacttcagagaagctaagaccccttcagagcattagcatgtccacaggcatggcatgaactccagtcttctccagatacttgggcactcacagtgtcctatctggatagtcattcacctctttgtgaataatgcatctgcctgaacaacttaaattcaaggcccctgagtgcatatatatttatagaattccttgcactcaccattgtgcctaaatgcctatcgcaattcatatggtagatatttcacaagtatttaatgaggatgataattataacaatgtcatgggaagaatcaatttttaaaattgtgttgtggaaaaaatatacacacatatataatatacatatataatacacatatacatatagctgtatatatatacatatatgtatatgtacatatacatatataatacacatatacatatagctgtagttttctattcttaagaataaaattaatcatattgggcttgaaagaatggtaagagataatggcttaccaacatagtgctgcctgtacagagaccagaccggatttcacaagggtgtgtatttgtaagaaggtaaggaacttgtttctttttttgtcttgcttttgtttttgtgttgctgtttattagagcaactgaattacaaacctgaagagatgaatttaggtttagaaagctgacaattttagtagacccctgatgtaaaatgcctttttcagtatacttccccagagacggcaaagatcctggacctaaagtcaaattctgcaacattaggcaactgatgaatcccaaaggtgattgcctggttgatgggtgatggtcaaaggagtatcagacacaggagaccacttctgtgggtcaatgaacaataaggccaggaaaaggcaactcacatgtgtaatcttgccacagttttggtaagtaagatgttaggagtttgaaattcagcctacatctctgccttaaggtatatgtggagatctttaaagtgagtggtgaagaaagggttaagaaataggatttaatggggaatagattaaaaaaaatgagaaggagtcttagctaaacaaaagaagaaagcgggttttatttttgtttttgtttttgttttttaattttctgggtgtttaaaggtaagcaatggtttttcatttctaccagactaagacagattgcaattgccttagagcagttgggaattttgtaggcaaaggagaaaccatattaggaaaaaagaatatatcgctaagctgacaagatactgtaaatacccaaaactaggacttatacaagccgagggggttaagctgccccttgatagactggtacaatgagaaaaaacaaagttcgcatgtctgattatgcacgaatttcctgagtgaaaatcttagcttggatttgtaacccaagtcttttaatgcattgaggaaagattaacagtaagagcctaatatggtttggatctgtgtcctcacccacatctcatgtcgaattctccagtgctggaagaggggcctggtgggatgtgattggatcatgggaacagacttccccttgctgttctcatgacagtgagttcgttcttacaagatctgcttcttgttgttgttgttgttgttgttgttgttgttgttgtttttgagatggagtctcgcattttcccctgggctggagtgcaatggtgtgatcttggctcactgcaacctctgcctcctgggttcaggtgattctcctgcctcagtctcctgagtagctgggattacaggtgcctgccgccatgcccggctaattttttgtatttttagtaaagactgggtttctctatgttggccaggctggtcttgaactcctgacctcgtgatccacccacctccgcctcccaaagtgctgggattacaggcatgagccaccgtgcccagccacaacatctgcttgtttaatagtgtgtcgcacctcccacttcgctctcttcctgctactttggccatataagacatgcctccttcctcttcaccttccaccatgaccataattttactgaggcctccccagccatgcttcaattaaacctcttttctttataaatgacccagtctcaggtagttatttatattgcaatctttgccattattttcaattacttttgcaccaacctaaatagcagtgtgagaatagactaatacacagcccagtgtttcaaaggaccctgaataagtggcctctgaagataactgcttttaacaatacattgagggtaactcattcagcatttctgaagccatcagagtgtctttcaatcagaggcaaggcatttaggcctttcaggcttaaaattaccagtttgaaattagccaacaaaaattaataaatgcaatgaactatgcccaactacagaatataaacagaaagatactggcacaagacattaaaccactcatactcattctatcttttcagatgcaaagagctagaaagaatcagaggactaggaaatgccacagaaacctccattgatcacatggttctgtcatatgaaagagaaagcaaaatagaaaacttcacatttatgaagattaggaaacacatgtatacatacatatagttcatgaggcagtgtaaaatgaggaaatctattttttctacttggatagaactggtagagtttggcacaattccactttaaaaaataaagtattcagtattccttgtgtaaggtgttgccagaagaagaaatttgctgagaaaaaaattcatcctttctttgcaaggaagatcttaggacagtggcatcaacatgaggcactgcattgacaatgcttttgcattctgctttgatttctttttggaagttggtagaggtaaatcacaatttatttttaataatatgaaactgtgaaagttaagtatggataatgacaaggaaaagtatttaattctgtctttatacctggacttgaaaaagaacagtaaccattctagccatttaatggaatttctaaaccatattatccttccccagaaatggacatcaagttctatggatcaaaaagctgtagctgaccttagactccaaaaagaacccagtaggctggacttaagcatgctggaggatgttcacattcttaagtcagggttatcgtaagttatttcctgcatttcatcataactggtcacatgtggccacttgtcagatctgggacaagcatttccaaaagcaaataaaagtgtgtatttttttcatctttctaaacagctctgagttcagatcctgtcaaggttaccttcctatgattcgtgaagatgtcatcctaaaatataaaaggaaagtacattggtaaaaagtcaacctttgaattttgtgcttggagtctttaggagtcactgattagactacaaacacccttgtcagctctattcttttaggtctcatcaggactttcttggccaccagcacacagcaaagctcggagggaccgggacccatgtttagtaccaaggtggtaggttgcgatgacggcaagtagacaccacaagaagctgaaagggaattcagggtaatataacctctttggaatttcaagagaatgaagaacaaaatttgtcttttcagagggctgagatttaaaaaccaccctgtcaatgactcctgaaaaaccaagagaagtttggataaaactcctagtgattagttccccaactgcttataccaggtaagaaatgatagctcttcagagctccctttccatatctaaagaacttgaattgttctttgaggcaatttgaggccactgtcccagtggaaagcttattttcaaattgtgtagcgtgctactatatcagactctgaaacactgcatgtgctaaagtaagtcagagatgcctagtcagattaggaaaaaaatattgtaggcaggcaaccagacaaatttggcaagaaaggatgtcagggggatttccaaagtctcctaactgagggacaatcgtagtgactatgaataattcagagtgagttaacgaagaggatccaccaatttcagaaactatataatatttttgaagaaaaagagagtttaaggactatgaaaacacatgtcaagaaatattattcttcttttaattgttttcaacaagaaggtcccccttaaatatcaattctgccatgaagaaaaataaaggaaaagctgtttcatagaaaagctctcctcctggcaagatcttaccacagatgtttttcagatctttaaagatggattattaatggtttagctctccactacattataaagcatttttcaaaggtttgttctatctctgtttacactatctgataataataaagcagagttcgtttttagccaatgaaaatgactcttcacaaggctgtacatttgtttgaacccagacaagccacaaaaagtacaaactcttctgcatgttcaaagttctcgaaacacacatgcctaacaccaccaacaacaaagacaggaaccgcggaccagttctccagtcccagcctcaattttttgagtaactgcttcaccatgtagagaccttgctctgatccatctctgcatcagagagaaaagaaagaaaagaaaaacgtacactcctattttcatcactaagacaggccctttcctttctctgtgtgaaaacattaagagtttatggttgcctggagaaaatttacaatctggccccacaacctcatgtgactttttatagaacaaggaagtacgggagcctatatgcaacagtgacaatgtgctgtggccgctcagtgttggccacaacagcttcagttactgaaaactctgaccaacaggggaaatgtacaaggaggctatcaggcattagctgtgggagaagaaaacaagttcccctcggggcacaaagagaaagcaactgttatggcaggtcaaggcctagagctgaaagaaaaggaaatacaattacaaaaccagatgcaaggcagcagaagggtctggatttctctttcagctggtgaatctacatatggctataaccttgctgacctctctggtcatcaaaccacagcagaaggagtgccccagatgatgaagaaatattatagtgtttggaacaccttacctgtccataaaatgttgatgtatttaaatatctatatcctgtgtgcttatcatttgtgttcttcttgtgtgttacaagtgactagattttctccaaaaaattctataaaccaggccctaaactgacattatcttagcttgtgttttctttagctatttctacacagtgaaagacagggctgattcttctttccaatgaatgaaaaaaataaattaggccaggtgtggtggctcacacctgtaatcccagcagtttgggaggctaagaagggcagactgtttgagcccaggagttcaaaaccagcctgggcaacagggtggaaccccactctacaaaaaatagaaaaattagttgagcatggtggcaggtacctgtagtcccagctactcgggagactgaggtgggaggatcacttgagcccaggaggccaaggctgcagtgagccatgattgtaccactgtactctggcctaggtgacagagtgagaccttgcctcaaagaaaaaaaaaagtatatcttccaaatgatattatgtaagatgtccttaagtatagtatataggaatgtagtttagaacttctctcccggagagctggggttcagggtgggcctgcaggcctcctagcttctcttttaagtattttaggaggtgaagtagactgttttggggaagaatcactttctgaaaaaggtacccagtgtcctttggctattccctcataaccctgcccagtctgagctggtcctctctttgtctagacacctgacaattccagggctcagaaatgagaacataaaagacagtttaagaaagataatatgttagaaggacaaaggaggctgtcctacataacggaaataagtgaagttaaaaatcaaactagaagcgccctatggcagattaaagatggccacaaattctttaacactcttccttttgagagatggggtctatttcccctccttttgaatctgagcttgcttgtaactgctttgatgcatgcagtgtggccgaaatgacactctgccctctgggcgttaactctaagagaactggcagtctccaccttggtctctgggagctttgagccgccacagctctgactgccttgctcaagagatcatacggagaggccctgagactatatggagaaggggccagctgagccccctttcccaccatccccatcaaggcagcaggcacagaaccctccagagcagcctgggcaccggctgaatacttctccagtaatgccagtgaaaccgtgtggagcagaagaatccccagctaagccctgcctgaattcctaatccacaaaagtatgtcatataaataaataatttattaaaatggttattattttaaatcactgggtttggggtaacttgcatgcagtaatagataaccagtacacagcctctgagtagagtacataatttttagaataaaatctccaattgtggaaagtgtgataaattccaaatttttgtggtcagttaaaaagaagattacattctaggtgtgtatgttccacaacatgtatacagagaagagaaaagctaaggagaaaagacatgaatctcttgcctgctggcaaagcttccccaaacagatattcttccaaaggctgcatagaagctggatcattgtaaatctctccaacaaatatctactgagtaactgctacaagtacatcactgtgttaaccactaaggttttaataatgataaaaatataatttctgccctgggaaatttttctctcttgtgtaccattagcaaattaatatgtaaattttaaaattggtttaaatcagaggttgacaaactttttctgtaaagagccagagagtgaatgtttcagctttgtaggccaactctgccagtgtagtgcaaaaacaacattttagacaatacataaacaaatgaaagcaatggaatcccaataaaactttatttgtggatgccaaaattggaatttcacatgattttcatgtgtcttgaaatattattcttcttttaattgttttcaacaatttgaaaatataaaattggtctttaatacacaggctgtacaagaggcagtgggctgaatttgatcctcaggtatagtttgccaacctctttttaaaattcttatcagatgggcctaaagcaggtagaacttgcaaaaagttaaagagcattatcagacgaatttaatgttctggattgtcatggatacaaataacctgccttctatttccagtattctccatgcataaggaagggcatgaacttatagcctgcaagagcagcttagagttttaccagggaggagctctgggaagcatcaagaccccagaggaggggaggactcagcccaaggaaggagagaaggaaccatagagatgagtgggagggcacgtctgcaaaggcagccggatgttgtgcaaacattcagcaagaagcactcaaagagcgaaagtgggacttcatggagaaagaacagtgtgacaatattttttaaaaattttccccctttgtaaattcctagtgccttcgagtaaagctgttcatggagcatatttaagacatctagagccaagtaaattggaagcacagaaatgaatacagaagaaaatttaggacatcctttttattgaaaggagttcatcaggttgccacctacccttcctttaaattttgctattccactttatgctgaaataagctcattttggcaaaagacttgctaaagacttgatgctttgtatattaatgttttgtatttgctttgtttttatataaagagccttcaaaaactattgtaaaaattaaaaatatatgcaatagatctccacagtaacagtgcacaaacatgcaccctgccccacaccatccgccaagtacgattatatgattgcttaaatgagctaactcagaagaaaaaggaagtctgagacacacacatttctacgctttgtattcagagatgatgtgtctcctggtgtatattccccctgcatccttctcatgctttcaacgtcatgaaggtataaaagtgtactccctatcaccagggtagcagccgtgacctacattttcttcatgttctgatgctgtttgtggcttcctcttcccttcaggcagcgcatgatgtctttgttcaaagcactctttccaatagctgatgacttagtgtcaggcaagtccacagctgtttcaatcattctgtccacacagacaccaaacaacaggaattaggtatgcatggaaaccaacctaagataagagtagtctgctctggggaaaaaatagaaagtcatttcacgatatacaggcaacattttgtgtgatttatttgacatcaactggatgactaatcgctgtggatttcttttcactcagatcacatgattcttctctagggtctcaatcaactatttccattttgcttaacgtaaaaaaatcagaagcctccaacagtcaacagtaccatttttaaagtaaattttgtacacctcccccactccccaatcatgccctttttaattactgctttcgctctcattaaaagaatataagtttaaaatttcgaatgtggatgtcaagttttgctgttatctttagtgttgttttctatatgaatgagcctagctttcacactatggacacagcctgtttagacaagagcagtgcttatgtaatcatagcttcccaaaccacagcaacagcacaaatttggcagctagaaggaaggaagtgagggtggggaagggaaaaggaaagaaagggagtttctgtttcaatcaaagatatatttggctcatatttccacctcttttaactgagattccaactctacaggaggtcctctccactcatccctccatgcctgacctgaggagcagcttggcttttccctcgctaagggaaaaacctttagtaccttccttctctacaaacattagaaacagcttaatcaagttgtcctcaggcaacaaggatacttcaggctttccaggtatgacacagcctcttcccctcctatctgctagccagctaacagctcttccactactagtgctctgccagtgtgcgtgactgccaaaggcatctcactggccaagtggaaggaagctttccttccaagtcaaacatccagtcttcaatgaataagccctatgtgctgaaacatgcctcttttccccagtcaccttactctttaggaaactgtggtaggcaagtaaagactccccaaaatacttatattcatatcctactccctggaacctatgaatatgttgggttacatggaaaaagggaaccaagatggaattgaggtttctaataagctgactttagaatagagagatcatcctccattatctgagtgggcacaacataatcacaagggttcttaaaagtggaagagaaagacagaagagaagatcaaagtgataccatgtgaacagaactcaaccagccattgctggctttgaagatggagaaaggggaccatgaggaaaggaatgtgggcagcctctaaaagttggaaaaagcaaagaagaggattctcccctagagcttccagaaatgtacacagccctgctgatgctttgagtttagcccagtaagacctgtatcagacttcctacctgcagaactgtaagactataaatttgtgttgctttagctactaagtttgtggtaattgttatagtagcaattaaaaactaatacaaaaaccattggttcaaggcaagtccatactgcttgggaagccaccttccatttatcaaagactctccattgaggcccaggtgcacagctcattccatctgggcatgtggttccaagtctctccattgtaccctggagattgacaccagaccacatctgcactgccattgcttacatttttctttcatgttggtcttccctagaaatttaaagaattacagatcaagaaaagctggatgcagtgttgcacacctgcagtcccagctacttgggacttgggaggctgaggtgggaggattgtttgagcccgagaatttgagtccagcctaggcaacatagcaagacctgatctctaaaacataattaaaacaaaaacaaaaacaaacaaacaaaaaaaacaagctctaagtggcatgaagttcatggctttgcctccataagaacttgtcttaatatgaaaatctctttgtttagcaaaagcataaatgagttttccagaaaaccagccctttactagtgatatagtgcagggtgaaggtctagaccccagggccttctgctctgagaagatcagactgctcaggtccagacaatcctggctatttctcacaagctataggaggagacaagttatttaacaccttacttctctattttcccatgtgtaaaatgggaaaggtaataatatctactacatagggtagttgtaagatgaaataagttaattcatataagaagcttagagcagtacctggcacagagcaagtctctttgatcaccgtcatactttgtcatgtctttggttctcccatcatcatgattttgcatatcctgatagcatctgttcttgccaaaggtctgactttccaacacacactttccaaaaggaaataaaatgatgtacttgtttccagtggccatccaaatgtaaaatccatgtttggaaaactttgcaatgcttcttttatacttctaagtgccccttgattaaggttaaagtaagttcctccagtggttttaaagcataggtcactcaaaattaactcccataagctattttacaaacttccttttgagaagtaattcctcaaaatgtaaaaggcagaatattagcttcaaaagtacgtggagtattcattaaaatgtaaactattgggctttactgaagatttaacggaggcccacaagtctgaatattaacaaacatattacatatagcatgtgattgttatgcatacaaaggttaagaatcattgccttaacatatctttagtttaaagcctaccctaattttattatactagatagtttggcaggtaaaaacaactaaaaaaaaaggccaggtttagcagatttgtacatcattctctcataagatgtttaaagcaaatcagccatttactttctcaagaaagagaaataggaaggcctggcatagtaaaaaaaagtgattcactaaaagagaagtgcatttattatcttctcatagcacagacacaactgtatgtgtgtctgtgtatatacatgtaaaatatatacacatatggatatagaacaagtgagttttataatgcagtctttgcaacacaattatatgtggttttcacaatggaccttccattatcaaaatctagattctaatattggagtatttgaaaagaacccacgtatgataataatattgaaaatgctacttgtaactgaatgcaatccttattaactttcctttaacttgtgatataattgacatacaattatcagctcataaaacgaaaagaacattgcctcagcatgtatattgtgaaataaagggattacgtgtcaaaaatgtgccaggaatagaagggttgacatcatctacagcttggtgatgagttttccttacagatttccaaaaaaggaaaagcaaaactatgttttgggaggcaacatcaaatattttttagtactttcttcatcttattggttgttttattttcgtagtgataaattagtgttcaactccaaatatttaatgaaaatgttttatggcacatatgtgttcatgtggctatatttttcccccaaagtgtgaatagtagagtatctacatcagaatcttcttttctttctttcaaacaagtatctccaaccttaccctaagcctctcatattagggcctagaaacccacatttttagcaagcttgtcaggtgatccttgcgcacatttaattttgagaaccacttctttgagaatatataatggaatttaaatttaatgatgtattttttcttatttttgaagtattttttaattatcttaatgttttatctaaactcaataaaaatttctctttacctaaactcaataaacagaaatctagcaaactcattcatgggatatcccacttcacaaaaaatcatgactaaaactttataagaatcaattaatatttgccaaataaatgaataaactaattaatcaacacatgaggaaataaaattcagacgatgagctatgaattctttgacttgacagaaaaataaatcattaagtctacatcatctctattaaacttgtactcataggcctaagcaacacagatttatcttaaaacctatatatcccactgatacctgcccttcattttctcttacttgtcaaggtgggtgggggaagggggcgggaagcatattatttattctccaaaaacagaaatgaatggaatgtaaaataagtcaacaaagtttgaaatttcttgttgttgttgttgtggtggtggtggtggtggtggtggtggtggtattatctttgttttgattttatggatttttcctgacacaggtagtatcactcaaaaattagttaccatctgcatgagttcttcttcatggaggccacggtctgtgtatgctcaggggtttgtttttggccccataactcaagagtctagttcactggcaatgcaggtaggaatcatctgacttgctactcctcccacgatatgtgtttgttcaagtctgtttgagaaatgatgtttgcttccactcaatgccaacagcagtgctgcaagcatctcagcctctatccaccgccaaacaggtgagcaggactatctatagcacttatcattcgcattaagatgcctgagtctgtgaattatctcttttacattatgagtttcctatttaaaactataaagaaatatttttaagtggtagaaaattcataacatggaaatgtttttaaacattgtgacattacttcatgtgctttttacaattacaaatacatttttagttattattctcctttttcacttaacaatatatatagctatttttcatagctacattttaaaaataacagtataatgttccttcaaataagcaaagcatagtttacctaaatttcccctatttttaggcatttatcttgcttccaatgttttactattataaattatgttttgataattaccaattatattttgatgagtatttgtggtgttttctatatttggggttattatcttcgcacagattcccagttctgggattaataaggcaaaagcagaatacttttgttgatttttttggaaaagcttacaccagtttatagaaccctaacagaggtaataacattcacttttttttcatcttccttgtcaaaattggaaatactttgtcgtttttgctaattaagtgaagtccctttgctgctacagtaatcttcagtactttgttattttgttcccaaagcacatttattgtaaagcttcttgaaagctggaaccagggaagaactgatgcacaaaacaattgcaaccagccagggaagaacgcctgagactgggacataatgcagaaaaaggaaaaggccaactgagtgaaaacagaatgggttcagggtagggaagagctctacaggggaggtcacagggctgggactggagaataagaatgagaactcataattgttgaacgcctactgtgtgccagcaactatgctagataattcacattatattatttaatccttgcaatagtcctgtatgctagtatactgggttaaatagtatcctcttaagattacatgtctatccagaacctcagaatgtgaacctatttggaaatagagtctttgtggatgtaattagttaagatgtgaccacattagattagagtggaccataaatccaatgactaatgtccttataaaaggccatgtgaagacacacacaaagaagatcatgtaataatagagttagagagtggactggtgcagctataaacctaggaacctcaagaactgctggcaactaccagaaactagaaagaggcaggaaaggatctttctctagagccttcaaaaggagcagggtcctgctgactgtttgattttagacttctagcccccagaactatgaaagaatacatttctgtcattttaagccacccagttggtgataatgtgtgatggcatccttaggaaactaatacaactaagtatttagtcatggtcttcaaatgaaaatacacttcagagagactcaataacctctaaggatatgtaagtggcagaaccaggattcaaactaaagcagcctgactccaactgtgttctttctttcacattttgctgtttcataggaacctttgtaaccataaagcatttactgccaatgttcacagcaagtcaatacactgagacaccaggtagagcagagaaagaggtttaatcatagggctaccaaacaaggaaagaggaggaaacttcaaacatgtcaccccagggagtttagggccaaggattttaaaggttttgggttgaaccaaagggtggcaatcattgatgaatcagaatgcagggtgaagtcaagggacagagagatgaagaaattgcattctccagatgatggagaccaccaggatcagttcctctgtgggggtcttcactggttagcatttactgttctgctggaatttgtcaccagaaaaacatcttaattcttaaacaaatgccttataattttaacctcagaaatccatcctgtaggaacaatggggatgcaaatggtcaatatgtgacttttagttacaaggaagtggaccaaagtgcagcctgattcatctttaatttatttttaattatactttacgttctagggtgcacgtgcacaacatgcaggtttgttacataggtacacgtgtgccaagttggtttgctgcacccattaactcgtcatttacattgtgcatttctcctaatgctatccctcccccagcccctcatcccccaaatggccccagtgtgtgatgttcaccgctctgtgtccagctgttctcattgttcaattcccacctatgagtgagaacaagcggtgtttggttttctgtcttgtgatagtttgctgagaatgatagtttccagcttcatctatgtccctgcaaagaacatgaactcatccttttttatggctgcatagtattccatggtgtatatgtgccacattttcttaatccagtctaccattgatggacatttgggttggttccaaccacaataaacatacgtgtgcatgtgtctttatagtagcatgatttataatcctttgggtatatacccagtaatgggatcaccgggtcaatttggtatttctagttctagatccttgaggaatcaccacactgtcttccacaatggttgaactagtttacactcccatcaacagtgtaagaacattcctatttctccacatcctctccagcatctgttgtttcctgactttttaatgattgccattctaactggtgtgagatggtatctcactgtggttttgatttacatttctctgatggccagtgatgatgagcattttttcatgtgtcttttggctgcataaatgtcttctttggagaagtgtctgttcatatgctttgcccactttttgatggggttgtttgtttttttcttgtaaatttgtttaagttatttgtagattctgaatattagccctttgtcagatgggtagattgcagaaatttactcccattctgtaggtttcctgttcactctgacggtagcttctttttctggcagaagctctttagtataattaggttaccattttggcttttgttgccattgcttttggtgttttagtcatgaagtccttcccatgcctatgacctgactggtattacctaggttttcttctagggtttttatggttttaggtctaacatttaagtctttaatccatcttgaattaatttttgtgtaaggtataaagaagggatctagtttcagctctctacatatgcctagcaagtttcccagaaccatttattaaatagaaaatcctttccccatttcttgtttttgtcaggtttgtcaaagatcagattgttgtagatgcatggtgttatttctgaggcctctgttctcttccattgtctatatatctgctttggtaccagtaccatgctgttttggttactgtagccttgtagtatagtttgaagtcaggtagtgtgaagtctccagctttgttctttttgcttaggattgtcttggcaatccaggctcttttttggttccatatgaactttaaagtagctttttccaattctgtgaagaaagtcatcagtagcttgatggggacggcattgaatctataaattaccttgggaggtatggctattttcatgatactgattcttcctatccattagcatggaatgatcttccatttgtttgtgtcctcctttatttctatgagcagtggcttgtagtcctcctcaaagaggtccttcacattccttgtaagttggattcctaggtattttactctctttgtagcaattgtgaatgggagttcactcattatttggctctctgtttgtctgttatttgtatatatgaatgcttgtgtttttttggacatgattttgtatcctgagactttgctgaagttgcttataagcttaaggagattttgggctgagatgatgggattttctaaatatacaattatgtcatctgcaaacaaggacaatttgacttcctcttttcctaattgaatactctttatttctttctcctgcctgattgccccagccagaacttccaacactatgttgaataggagtggtgagagaagacatccctgtcttgtggcagttttcaaaggaaatgcttccagtttttgcccattcattatgatattggctgtgggtttgtcataaatagctctcattattttgagatacgttccatcaatacctagtttattgagatattttagcataaagggctgttgaattttgttgaaggccttttctgcatctattgagataatcatgtggtttttgtcattgattctgtttatatgatggattacatttattgatttgcttatgttgaaccagctttgcatcccagggatgaagccaagttgatcatggtggataagctttttcatgtgttgctggattcggtttgtcagtcttttgttgaggatttctcatcgatgttcatcagggatattggtctaaaattctctttttttgtggtgtctctgccaggctttggtatcaggatgatgctggcctcataaaatgagttagggaggattccctctttttctattcattggaatagtatcagaaggaatgataccagctccactttgtacctctggtagaattcggctgtgaatccatcttgtcctggactttttttggttcttaggctattgattattgcctcaatttcagagcctgttattggtctattcagagattcaacttcttcctggtttagccttgggagggtgtacatatccaggaatttatccatttcttctagattttctagtttatttgcatagaggtgtttatagtattctctgatggtagtttgtgtttctgtgggttcggtggtgataacccctttatcattgtttatagcatctgtttgatttctctctcttttcttctttattagtcttgctagcagtctatcagttggcttgatcttttcaaaaaaacaggtcctggattcattgatcttttgaagggtttttgtgtctctatctccttcagttctgctctgatcttagttatttcttgccttctgctagcttttgaatgtgtttgctcttgcttctctagttcttttaattgtgatgttagggtgtcaactttagatctttcctactttctcttgtgggcatttagtgctataaatttccctctacacactgctttaaatgtgtcccagagattctggtatgttgtgtctttgttctcactgatttcaaagaacgtctttatttctaccttcattttgttatttacccagtagtcattcaggagcaggttgttcagttttcatgtagttgtgcggttttgagtgagtttcttaatcttgagttctaatttgattgcactgtggtctgatacactgtttgttgtgatttctgttgttgttttacatttgctgaggaatgctttacttccaactctgaggtcaattttggaataagtgtgatgtgtttctgagaagaatgtatattctgttgatttggggcagagagttctttagatgtctattaggtccacttggtcctggatatcctcgttaaccttttgcctcattgatctgtccaatattgacagttggggtattaaagtctcccattattattgtgtgggagtctaagtctctttgtaggtctctagggacttgctttatcaatctgggttctcctgtattgggtgcatatatatttaggatagttagctcttcttgttgaattgatccctttaccattatgtaatggccttctttgtctcttttgatctttgttgttttaaagtctattttatcagagactagaattgcaaccccagcttgtttttgctttccatttgcttggtagatcttcctccatccctttattttgagcctatgtgtgtctctggatgtgagatgggtctcctgtatacagcacactgatggttcttgactctttatccaatttgccagtctgtgtcttttaattggggcatttagcccatttacatttaaggttaatattgttatgtgtgaatttgatactgacattatgatgttacctgattattttgcccattagttaatgcagcttcttcctagcatctatggtctttacaatttggaatatttttgcagtggctggtaccagttgttcctttctatgtttagtacttccttcaggaactcttgtaaggcaggcctgatggtgacaaaatctctcagcatttgcttgtctataaaggattttatttctccttcacttatgaagcttagattggttggatatgaaattctgggttgaaaattcttttctttaagaatgttgaatattggcccccactctctaacggcttgtagagtttctgctgagagatccgctgttagtctgatgggcttccctttgtgggttacccgacttttctctctggctgcccttaacattttttcctcttttcaaccttggtgaatctgacaattatgcgtctttgggttgctcttctcaaggagtatctttggggtgttctctgtatttcctgaatttgaatgttggcctgtcttgctaggttggggaagttctcctggataatatcctgaagagtgttttccaacttaatttcattgtcctgtcactcaggtacaccaatcaaatgtagattttgtcctttcacatagtcccgtattgcttggaggctttgtttgtttctttttactcttttttctctaatcttcttttctcactttatttcattaatttgatcttcaatcactggtaccctttcttccacttgatcaaatcagctactgaagcttctgcatgtgtcacgtagttctcatgccatggtttttagctctatcaggtcatttaaggtcttctctacactgtttattctagttagccttttatctaatcttttttcaaggttttcagcttccttgcgatgggtttgaacatcttcctttagctcggagaagtttgttattaccgaccttctgaagcctacttctgtcaacttgtcaaagtcattctccatccagttttgttcccttgctggcaaggagcagcagtcctttggaggagaagagctgctctgcatttttggaatgctcagcttttctgctctggtttctccccatctttgtggttttatctacctttggtctttgatgttggtgacctacagatggggttttggtgtggatatcctttttgttgatgttgatgctattcctttcagtttgttagttttccttctaacagtcaggtccctcagctgcaggtcttttggagtttgctggaggtccattccagaccctgtttgcctggatatcaccagcggaggctgcagaacaacaaatattgcagaacagcaaacattgctgcctgatccttcctctggaagcttcatccccgaggggcacccacctgtatgaggtgtcagttggcccctactgggaggtgtctcccagttaggctacacaggggtcagggacccacttgaggaggaagtctgtccattctcaaaactcaaatgccatcctgggagaaccactgctctcttcagagctgtcagacagggacttttaagtctgcagaagtttctactgccttttgttcagctatgccctgcatccagaggtggagtcaacagaggaagaaggccttgctgagctgcagtgggctccacccagttcgagcttcccaggctgctttgtttacctactcaagcctcagcaatggcagatgcccctccccctgccaggctgctgccttgcaggtcgatctcagactgctgcgctagtagtgagcaaggctctgtggctgtgggacccactgagccaggtgcaggatataatctcctggtgtgccatttgctaaaaccattggaaaagtgcagtatttaggcaggaatgtcccatttttccaggtacagtctgtcatggcttcccttggctaggaaagggaaatcccccaaccttttgagcttcccagctgaggcgatgccccgccctgcttcggctcatcctccatgggctgcacccactgtcaaaccagtcccaatgagatgaactaggtacctcagttggtaatgcagaaatcacccatcttctgcgttgatcacactgggagctgcagaccagagctgttcctatatggccatcttggaacagacccctcatccttaattgtaaaattcttaactctgtgaagatggattcagctttagttctaagcctgattttgccaccaattctctgtgtgaccttgagaatttcatgtaatctctttttgaccttagttccctgtgtgtaaaacaaaattacttgataaggtgatttctgaggtccttcttcctctacaacattctgcaggcaatcctgggagcactcctaaggggggatgttcaagaaccctggagaatctttgctggcaactccattcaaggcattttcaatgccttacctgcacctacataattcaggtccacaataggtatgtggaaaaagacatagaagaaagaaataaggcttgagatccttaatatcatggtgtctaaatctgctgttatcaaggctaggtcatatcatattaaagaaataacttaagataaaatcaccatgctcagagtggaataccctgcaaccaaatttttcccctatacctgacttcagtaactggttttgccaatataatgccattcctacaccaagattcacagtatgtcttcatgctgtggtggttataaacacctcatactggacttactatgcctttataaattgtaagtaaaggccagcatgaataattttacttcctctgaataaaccattaataacatctctgtattttcaatgagaaataaaataatggcaataatagcttccaattacacagtgtttgtcatgcaccaggtgataaataagcactatacatactaatctcattcaagcctcagaactctgggcactatagttgttgctattattctcatttttttctggaaactatttgagaaccaagaagttcaaagttacttattcatttaacatacatttattaaatatatctatgtaccaggtgctagggataaaaagaacaaacatgacatagtcactcaataataacaattttgattaaatatataactacagagagaagaatctggaacagtgtatcacaaaatgttacgagctatggatgacttttattttctctcttattcttccattttcctacacgaatattgattacctctgtaatcacaaaagaaaataattttttacaaggcaacagcttcaccttcaagggacttgcaatataatgagggagccaatttatcaacaaccataacttacttggtctcatagagctcattagcaatgaggccagatttgaacctagggctgtcagaattccaaattccattttatcttccccaccccatactgtctctggcaaaaatattcatttgcctggaggtgtgcttacatttgttttcctttattgaagttaagttatagacgtcagagggaagtgccaacaatgcaatctgaaggacttgccatttatgggctcactcaaggctgatggtggccctctctctttcccttctccacccacagatcttcatctcaaagaaaatgtggaggtggtgagcaatttatctgataccctggccccgtaaataggtgcttcagatgcaattaagagcttctaaaatattcatcacacaaattatattttacctgcccatcacttaaatccgaaatatgatacatattccttaatgataattatatcatcctaaccagcctaagactacagctggcagaagaacgttcaaaaggaaatgccaaactctttctccttctctaatttcttcacttgcaaaacatatatttattgagatcctaatatgtccaaatctgtctttcttaaatactcactaagtcacaggatgtgagttttgatatatgtccacctatgtttgatttgctttctggtcctactgcaatagactcatggggattttcagaattattttgatttttctaatttattaatgtttttagtctcctgtctgttggaagcagcccccccaaaatctggccataaactggccccaaaactggccataaacaaaatctctgcagcactgtaacatgttcataatggccataaagcccacgctggaaggttgtgggtttaccagaatgagggcaaggaacacctggcccgcccagggcagaaaaccgcttaaaggcattcttaagtcacaaacaatagcatgagtgatctgtgccttaaggacatgctcctgctgcagttaactagcccaaccaattcctttaattcagtccatcccttcgtttcccataagggatacttttagttaatttaatatctatagaaacaatgcgaatcactggcttgctgttaataaatatgtgggtaaatctctgttcagggctctcagctctgaaagctgtgagacccttgatttcccacttcacacctctatatttctgtgtgtgtgtaattcctctagcacccctgggttagggtctccctgaccgagctggtcttggcacctgtcttagaatcaggtcagagattatgatgaatatgtctatattcctgacagtccttgttatagtactaagcacagagtgagtaatggataaacgctgaccagcagagtaatttattgttgtaattaggcttaagggatcttttgctaaatctcaaaaatgatacagaatcatcatttgcccattataaactacgcaatgattaactcttaatcttacatttattcagtggaaaattctcccaaattccctgctttacctttctgatatctctgccatctccttcctctgcttgatgcagagtggaattcttaactgcttagtttttgtaaaaagcaactaaagcatggacaggaagctgagtccattttggactatactatttccctgtcctcctgtaacatttttcttttcgtattcagttcagtttctgtgtaaaatattgtttgacattgaaactctgtcaattagggtcatttgctgctcaaaaataagaagcaatgcaggagtctaagaagggcccatgtaatttagaaggtcacaaagtgacttgagctcacatcattacaccacaacatgactccctctgtacaatgagttcttccttctgcagtcacaaaaaaaaggaaggaaggaaagaagaaaggaaggaaggaaggaaggaaggaaggaaggaaggaaggaaggaaggaaggaggggcaaaaagtaaagattattaaaaaggagagtaaaaggaaagagaagtataagaaaacaaaatgggatttttttgaattctgtctccaaatggagcttttccctgaccaagggattaagaaattggcaaatggtaaacagatttgattttctggtatcttctgttatcaccatgacttcagagagacagcacatgctggggaaattgagcaggaacccacgaaaacggttttattactttccatgagggatgtactcagggttattttttctattcataaattggattatttcaaatgttaaataacttggatgaagaatagaaatgacagccactggacaaaatgatagtattgtgctactggctgagaccaatggtgaatgtaaactaaaatatattttaaagcagctatttaaatcacagtcattttcaaaggcaagcactacattgaaatacacatacacttgtccttcagtttccacaagggattggttttaggacaccctcactcccaccccatccaacaaataccaaaatctgagaatgctcaagtccctgatataaaatagtatagtatttgcatataacctatgtacatcctaccactttaaatcatctctagattacttataatatctaaaacaatgtaaatgctatgtcaatagttgttttactgtattgtttaggacatcgtgacaaaaaatagtctgcacatgtttaaaacaaatacaatttttatttctaatattttcaatcctcagttggttgaacctacagttgattgaacccatagattctgatacatatatatatagagagagagagcaaggtctcactctgtcacccaggctggagtgcagtggtgccatcatggctcactgtagcctcaaacttctgggctcaagcgaatctcccacctcagcctcccaagtagctaggactacaggtgtgtgccaccacacctggctattttttttagtttttaaaaatgttttgtaaggacaggatcccactatgttgctcagtctggtctcaaactcctaggctcaagtgatcctcttgtcttggcctcccaaagtactgggattataggcctgagccaccttgcctggccatatttttaaagctcacatgtagaatttcatatttaaaaatgtccttcagagaattcagctgaaaataatgctaaagagagtttgaggtgatagcatctaaaaaccaaagtccaaaacagaaacatacctactggcagccatggcttttccccagtccatctcaacctattcatttcatgagcaacttttctcaattcagctcatttttgcatgcctcacacccctaaccctcatctgttgctataaatccttattggcagcctcaaaccataaatctgccctaggcttgggccttttctcctttaacgatgccagccccatcaagcctattgtctagtgctaacataatatcaccctaatattgagtgttactcccaaaatactatattccagactcataattctgcccattcaggcttccaaattatgtaaaattaacttatcattgatttatcatgtgtgaaatttaaaaatcttctatggttggaatttccttagcaaaaatggtaatagtagctagctaactttcactgagtacttaagtggtgtcagaagcagttcctatgtataataatatctcattttatcctcacaaaactccataagggtgacactcatattatgccatactttgcaattattcaagggcctagagtagctaagtggctgttaggactggcaaagctgaatttcaaacgcagtcagctgtcctctggggacccacctctgactcaccatgttagactgccttgcataactgatagacagctgaataaccaacaaatttctgacaccaatgcaaggcctgaccacagaaagcagtacatagtaagcaaatcaactaaaaatgattattttcctggcattttctttaaattatacattttccccctttgagttataaactaatgaattaatatacaacttcaaagaatgccccaattatttcttttccaggtcccaatcaaaaagtgttacaaacttctagagctttatgaaaggtatcatgaggcctctcaataattgttttgagaatacaatgattttctactagcactgatacatagccagcaaagtacaaaagagttgatgcactagagtgagcttttaaagccataaatcagagagcctcagttccatgttggaaattcacaaggcttcccagtcagcagagattgaagccccccaccactatccagccccagacactgtcttatttcatttcctccacactctcctcccgccccaggtcttccttaggttacctctcgaacttaatgcaagtttctacttaatcactttttctcatatctctctctctacctgaaattgtattttatgttcacctgtttccctgtttttctctccctccagcatacttcatgaggtctaacccttgattcttttactacagtaaccctaggccctaataaatatctgttgacacaaaataaatacatatggaatgtttgactcattcaggacccagaaatgtatagaatacaccttagctgatgatctggagaaaaaattcactttacccttgtatccccttgcccatctcaatacgttagtggtcaattatattctaattccacaccaaggggctagaaggtaaaaatcaaaccgttcccttccactcttggttatttggctcctttctttttctgtagttgtcagcacttggaaaatgactttcttcctttaggcccagccaggttataataacctcaaatcttgacatttttttcacagcaaaagccctatggagttgaaatgctgaaagagcatttcacacacacatgcacacacacatacacatggttacacatgcagtgaaaacctgctagaaaacacctacccagggcaccagggttaagtagaaggagacatcagaaaatatatcacaatcccatatgctatcttacaactgaaaaataagaactgtgattccacaacagataaaatgaaaatgtcactgccattagagaaataatttttaccaagctgcacctatctgtcttcaaatctagtgcaaacccagaaaaagccacagatgcaatcaatagaaccactcatgacaaaactggcaagtaaagggaatgtggtagttccaagaacttacatacccaagccttgcctgaacatgttctaacagtcaatgataactgcatattccaaaccctgattttatcttcaaggaaacttgtattacccatataaattgagctggaaactgtaaaatatagataattgtaaagataaagttcttaaagtgtcttaatttccactttcccaggcaacatgcgctttgtctagaaaacttagtgcctctctctctcttgcaggtcatctggcacagactattaaatgactagcacttcccgttctcagggtgatgtatctgcttcctgatggtgttggctctgactatcttcctacttccacccagtggctccagctgtggctgccaagaaaaccccagctgatggaggatgagaggtgaaatcaggacacacaaagacctgttaccatcatctttcacctggattctcctaaagcaacagctgctcatctcatcactctgagttactaatccttctttctgagacaaaagcccaaaaggcaaatgggaaatgacttgtcagatgacatcaccaataattgaacataaagcagactcaaattaggacaaaatcttttgtatttactcagtttatatcagtaatatgattatctcaaagttctaacaaagcagtctgccacaaggggtaacaaaaaacatagtcactgatttgtcccagagcatctccagctgggacaacttattctaccatctctgctctgcccagacgaccacacacattgccttgcctagggaaaggggcagggaaatgatgcaggctttactgttccattgacccttgcagctaatccaaagggacataaaaatctctcatttcagacttttatgacatagcgtagccctccctggagataagtaggaacactggtctcccgtccccacttgccctttgggaaagatcatcacatcagccaggcagagaacagccccagctgacccaatgtactttctctttagcctctcaacccttcttcctttggttgttcatgacaacctgagttttcagggaatttcaaattgatgatgacaagctgtttgtcctgtctcaatctgctgaagatgtttaaaataaggagctatagtgaacagagagttgaaaaaaaaattctttggggtggatgacacattgacccaaaccattctgatcacacacaactctaagctcctatttggtggatgacattaaatgctgaaaaggtttggagtgcacatatattagctacttttcacagcctgtgtacaaaaagtcccatgccctactctgccatacatgttgactgactagcttcattagcaaggaagaacaaaatagtcaatgcaatctttttctgtgagttcagtgaagtcctagatctccttttggtgtcaaaccatcaccttccatgaaaggtttttgtgaggtggacacaattttcagaaggtagggtccaggttctctgacattagtgttaacctacttgaaatcttcctacttaatggctagacacagtattacttaggaattaagcttcaagcaacattcttaaagaacaggttatagaatttattatttgttagaatttctttatgcaaaagtgggaagtatctttaaaaatattttagccaaactacatgtcttgaataactttaatgttatcaatgtgagcctgtttctagtatccatcatgttacctagccacaaatgtcacaaattaaagaaatatatctgacattcattatgctaaccaactgtctggtgttgatcctcgtatggatagctgaaatgcttttggtagccaacttgggaagatggcttttctgatataatgcttgcgatccacctattctcccttagggcacttgacaattttaagagtcaaagtgctagctaactagaaattatataatatcacattgttccaggatgcaatatttaaaattaaataacctgcatcatagctagatctggaagaatagagtttcaatcatcaattgttagcaagaattatttttaaagtaactcaactcttctcctcattcagttgttagacatgaaagaaagtaaggtgaaagctcagagatttctgcctttggcccaaggagcagagcctacaaaaatacatatataaggtcttcttgctactagggagtgaggtcagagcaatgaaatagcatgggtgtgtgcatgcatgcatgctgtgtgtgtgtgtgtcctttgcccagaggtaaatattttcttttgatttttattgcgctttgttccctgtattggttgagtttaacttccaacctccactccaccccatagtagaagaattcttcttggaaaaggatgcctgaaataaggcaaaattattgaagttaaatataggtccaagagactctagcacttagaattgcagaagcttccatttaaagctgcttcatccttttgattcaaagccagaaaaaagacattcagtaggactaacaactgttcatgaatgtactgttaagctaattacttaatgctaaaattatttaaaaggcattacttggaaatgtttacagtatgtgatttgattaacaaaactattaagggtatgcttactatcatcaataatttttactatacatttatttgtattgggaatacattacttgttcccttagaattctgacaaatataatcttagcttcaattcggagaagttgttctgctggagataaatttgattgatttttattgctcctggaggcaaaacttaatgagtggaaacaacctgagaatctttctcaccatcagagctgtccaacagtggagtgtaattttttgaaacacataattctctctttttcctgtatttccagttgctttatgctcctgcattacttttcttcctggtccactgacatttctccacctccatttccaaacataaatccataaacacaaacccatgcaaccctctctatgtatcatcccgaatatatatacatcttgacttcttctgagttaagattctaaaggaaattttaataataaatgaagtgtgtagcagaagaagagggagagaataaagcacagggatacgataagagacccagttgtgaaacataattggtctgtaaactgaagaaagtcgtttttcagggtccagttggggcctcaaagcctccttaattctgtttcctaattgaaccaccaagaccatcatttctgactctgatccactttgccctctgcaaatcctgttcttacttttacccccagagattctctctctgaattgtgaataccatagaattaccccttttctctcccttgaagatcactattactggacccctggagtgccttgtcatgcttatttcctgagacaagtactagggcaagtgttgactgatatcttttacgaaagttaaactattttgtccactgacatttacccttgatagcagatgccacacttctcaaagtctgtctactcatcaagtctcttcctattttgaaggctaccttcccttttgagctgtctcttttttccactggtggttacttccaacacagctcactcaagaattggaggtaattggcatcacttaaaccaagtcaattaagcccagattgacccagggtggtaactgtcaacttctccaggttctctttaggtaactcaaattttggcaattttcaaggtggtagtttaccaaactcttatttctatagtgttgggatagtaaaatacttggtaatgtgttgagtgagagacacattctcttcccctaaaggaagaaggaaatttcattatttcacttaaaggtgcctttcaatgtgacataattaagtgattaaaaagcacagcctttgtctagataagaagctttaagcacaggtagcatgaaatcaatgcaaaaaatctaaaatatgtattcaaaaactgtgttctaagaaatatttagaatatgtgacccctgagcctgtgcaaaatgggttgtagggaaactggaaagtcaaggaaaaaggtcttcttggaggttctaagctatgacccttaattcccatgatgtatgtcagaaagtactcataggaaaatagacacaaaaagtgaagactaggacaaactcagcctaatgagaccttttcaaactaaactcattttctgaaaggaaagctagaacagctcttgctgtgtggggaatttctagtcattccccttactgctgaaagttatagagaaaagaaaggaaactgggacactctatagcaatggtccactaatagaattaagaaatttggagagtaaacaacatataaagcctggaagaagttggttcaacatacacatctaccacaatggcacccaaccagcagtctcaagcagcaaagatgtcacggaggaattcacgtcacccctcttcattctgccctccattcatcagggcttttatgcagcactttgtggtataagtgaaagttcagttccatcactgatgtactcctatacatcaaagtgtgttttctgattgattactgtttttggcattacagtacttcttctacttttcaaattgattccttttatctataagttttcttcctcctcccttttcattgatgatattcatgacataatgaattcaaataacgtttaaagaggtctctacagtggatttttttggtatttactgagagagaaacaaaaataaacttggcagttctttacactctaacatataaaataaaacattatactgtaaatttaagtaaaaactataatatacaataaatgcaaaatataattcatataaatgcaaccaactcaaagtagtaaatacaattataataaataatacaacacatatataataaagaaatgcaaaacaataagcataagtgaatacacaaatacaatatacaataaattcaagtaagaaatacaagatacaaaattttaaaatgcaaaatatattcaagaaacaaatgcaatatacatttaagtaattcaagtaatttacactctagcatacacaataaagcactaaatttaatacccattatgagtgatgtaagaattgagctttccattcctctgttcttcaagtggaaaattcaactaatatttctggtcttcagtactctcatctgtaaaatgagaaggcaatgttggtttgtgttgtctctaaggtttttataactataaatatcctacaggtctacaaatatttcatgtttgaggaggaagcgtggaagagtagcaaatgctcgtggtgaccaaggcaaagtctccttattccttggtaagagaagcccagttttatctgggacagcaatatgcatgtgatagaggttggcatgtaagatctaataatgagatataaacagacattgttgagtggagctttagggaaaatcgtttaggatagagatttaactggcattcctctttggccctttactctttatccattttcaaatgccccaataatgtacaggagtctgagggttccacagctagctgtgatcatgaggtggcaaacgagaggtccaattttacattttgaagacaggaacagaaagaatcagcctgagtccctaatggcatccctgaattacctacccccagattgtgtgtgtgtgtgtgtgtgtgtgtgtgtgtgtgtgtgtctgtgtttataacacaaataaattcctatttatgtaagccactgctagttgagttttatgtaattcctaaggtaaacctaattctaaataaaagagagtgaagatagaagcacacccatataattgtcatcattatataatatttaaaataaaacataattatctttgtctgaaatgacttagttttctttggaaagagatcacataagaaaacacgaattttaaaaaatcctaccatgatttataaagaaacatgcagctgcaagagtgaggcatttttttttttattacactttaagttctagggtacatgtgcacaacatgcaggtttgttacatatgtatacatgtgccattttggtgtgctgcacccattaacttgtcatttacattaggtatatctcctaatgctatccctctcccctccccccaccccacaacaggccccggtgtgtgatgttccccttcctgtgttcaagtgttctcattattcaattcccacctatgagtgagaacatgcggtgtttggttttttttttccttgcgatagtttgctgagagtgaggcatttttgaagcaccctaacttttgaaacctgtgaatacttttatattgtctggcaccacttcttatttcaatgacaactaatttgcttttgttcattccaattaggtccacatccataacatccagtaggtcattctctggagataagagtttgtgataccagcagaacctttgtcctccgtgactggtcagtggggtagtggctctgagtggagggcaagcttacgtggaggacaatgtgaatttgggttcaacattgagatagacaggacaggactcccctataaatatgtaggcatcaaattgcaaattgtactgttgttcccccaaaaatcagcagaccagttaaaatgaaaaactgactttttttgaaaaattacataagaaattattccttaggacatccccagagctttatccagttcttagatataacccaaaggaaaactttcattctttattccttttccctaataagaaagagtaatttgggggataaaaaccatgttttgttttccacccttacctgctaccccacaacaggcatgggcacgcgcacacacacacacacacacacacacacacacacgcaggctcctttcccaactggaaatttaagcactttgaaggcaatggatttttttctgagattataatagaagcaatacaaaaaccgaaaattaaacctcagactcactgtttgaaaaagtactagccatttcctgccatgaataacttctcgctggtttgttccccactcagccccataacatctcttccgcctctctggttcctttcctctggttcctagctgcagcctgtcacacgtcaatcctgttaatgtcttacgcataacattttagttctcaaccatctacctcaagcttaattccacactgaggtttctatatccaaaagtttaaaggccttctcctttccacttttttgagctcaaagaacaggcaggtatttggaaatgacagattatggagttagattccctacccactagtccctttaagaagaagctcctgtacatcacgagttctcttttcctctttctgcctcatacacctaatgaaggagacctactcaattcatcaaacctttattaaccactgtcgacatagcagatgctgctcagtatcagagaaactgaaggaaaaacatgcagtcccttaccttgagaagttcagagtcaacatctgtggagacacagatgttgacacacaacggtaaaaccttgtggtcatcaccacaagaaaatcatgtataaacgattatgaggacaaagctaaatatttttcaaatataatcttcaaaatcttaaatatacctacagcacaggtatggacaccttggtcaggtgacaacacacctgcacaaataccttttgacatgccagtgaatctatgtaaattcttctttcacccaattgtgctaggcttcagccttgtagaaattatcataagcttattgttagtatttctttaaattaaatgtttacatttgaaattactggtaaaagtaggcagtaacattttcacttctgcagtgggatttccttcatgtttcactaaacaggtaatatcagtggcttccccttatactctttcttgagtcaaaccagtgatgtaaccctacggtagctcatacacagatagcacaatgctgcaggctagaacccacccatttcactgcacccacctagcttttatcagtcaaattatcagcttgacaaacaccgattcagcaattttcagtatttcctggtgtgaaagggccatcacactcagtatttctcaagatgaaaatcatcaagtggttagagtgcaactgtgagtcacagagatgctaatgctagccctggccctgactcactctttgaccgctgtcctgtactctccacccgtaggttccccaacatatcagttacattttcctatttacttgtcataaggaaggagctatacttagagattatttgaaaattggctattgttgtggaaacctaaatagctgttcataataaatagttttccgaggcttaaatttatctattggagaaaggacccaagtttagaaattaagaaaacaaaatccaagtctacaaaaagcaggtccaggtcatcacctctatctcctgcctttgagacacatgcatactgaagcccagacagctaacaaatcttccccaaaaggcccagctctgcagtgcctaaatcagcaccatccaataaaaatataatgtgggccacaagtgcaaaccgtgtgtgtgtgtgtgtgtgtatatatatatatatatatatatacacacacatactatatatagtgttttttttgagacagagtcttgctaatgtgggccacaagtgcaaatcgtgtgtgtgtgtgtgtgtgtgtgtgtgtgtgtatattttgagacggagtctcgctttgttgcccagactggagtgcagtggctcaatcttggctgactgcaacctctgcctcccgggttcaagtgattctcctgcctcagcctcccaggtagctgggaatgcagacacatgccaccgagaggtgacagtgtgctggcagtcctcacagccctcgctggctctcggcgcctcctctgcctgggctcccactttggcggcacttgaggagcccttcagcccgccgctgcactgtgggagcccctttctgggctggccaaggctggagcccactccctcagcttgcagggaggtgtggagggagaggcacgagcgggaaccagggctgcctgcggcgcttgcaggccagctggagttccgggtgggcgtcggcttggcgggccccgcactcggagcagccagccagccctgctggcccagggcaatgagggacttagcacccgggccagtggctgcggagggtgtactgagtcccccagcagtgccagcccaccgacgctgcactcgatttctcaccgagccttagccgccttcccgcggggcagggctcgggacctgcagcccgccatgcctgagcctcccacccactccatgggctcctgtgcggcccaagcctccccaacgagcaccaccccctgctccacggcacccagtcccatcgactacccaagggctgaggaatgcgagcgcatggcgcgggactggcaggcagctccacctgcagccccggtgcgggatccactaggtgaagccagcagggctcctgagtctggtggggacgtggagagtctttatgtctagctctgggattgtaaatacaccaatcagcactctgtatctagctcaaggtttgtaaacacaccaatcagcaccctgtgtttagctcaaggtttgtgaatgcaccaatggacactctgtatctagctgctctggtggggccctctggtggggccttggagaacctgtgtgttgaaactctacatctaactaatctgatggggaggtggagaacctttgtatctagctcagggattgtaaacgcaccaatcagcgccctgacaaaacaggccactaggctctaccaatcagcaggatgtgggtgggggccagataagagaataaaagcaggctgccagagccagcagtggcaacccactccggtcctcttccacactgtggaagctttgttctttcgctctttgcaataaaatcttgctactgctcactttttgggtccacgctgcttttatgagctgtaacactcactacgaaggtctgcagcttcactcctgagcccagcaagaccacgagcccaccgggaggaaggaacaactccagacgcgctaccttaagggttgtaacactcatcgcaaaggtctgcagcttcactcctgagccagcgagaccacaaacccaccagaaggaagaaattccgaacacatctgaacatcagaagggacagactccagatgcgccaccttaagagctgtaacactcaccgcgagggtccgcagcttcattcttgaagtcagtgagaccaagaacccaccagttcagacacaccaccacgtctggctaattttttgtatttttagtagagacggggtttcactgtgttagccaggatgatcttcatctcctgaccttgtgatctgcccatctcagcctcccaaagtactgggattacaggcgtgagccaccacgcccggccaccatgtgtataatttttagttttctagtaaaaagggtaaaaaagaaataagtgacattaacttcaataatatattttacttaacctaatatgttcaaaatattattgctccaacatgtagtcaattttttagaattatcagtttgttgcactctttttatactaagtctttattcacactaaattttcatactaaaatacagtgactatttcacacttacagtatatctcaatttagactagccacatatcaagtgttccagtagccacatgtgactagtggctaccgtattggacaacataagcctaacctaactccagtgattcagtctcctagcagaggtcagcatcaactaacaggataaaatgaaaagcagcataacttctttaagcttatcagagccctactgctacctcttcttgtattctctatgacccttttaaaaaaagaatatcattcttctgtataacaacattctccctccctattccattctgggacttcactctgattaaatccttttgaaattcactttaaaaaaatcccttttctgtttacctgggctccctgagtatgcctagggaaattatcagtaggaattttcttattgcttgtaaattatgtttaattcctaaatttccctttttctgccttgtctgaaagcattccaaacagaaactagtcaaagaaactaatttttttgtttttccatcagttggtgacctcataggtagaaccgaaaaggtcattgtgattataaaccctctcaagaggttgttctaactttgtcaacataaggttcattctcaataataatgtgtcttaaaatgatgtttccttggatccttaaacagaataatgcatgacttcaagttaggaatttccagtgatgaatatttcaagtgagtaggattacaacaaacttagatgtccacagaaagttaaagggtgagtggacaagatctggggagcacactacttttttctgactttccaggagaaaccatattcctgggactgccagcttcactgcagacaggtgtgaggactacaatgaccccagtaagctattcaatgtgcaacagggctgtacatcacatttctcttggcattgactttttttgtggacaaacaacagaatgtaacctaagtagctcctaagcaaaaatacgaagtctacaaaattagattccatgccacatgaattcattgtcccacttacacaccactatctaatatgtgttacggataagaggttggattccagctgaaaggcagaaaggccatgacatcaagaaaaggagaatttgagggaagactttcaaaaaatcaagaggccagacgtgtctcagagaaaaaaacatttcggtggctaacgaaagggtgtgtgtgtttaaaataaaaaagaagaatgtctcagaacatatttttaatcatcatggaacctcctagccttacaaaacagagggtagatgccagaaaagagggatgaattggtctaccaggggctactggaaggacagtattggattgtggatacctgcgttctcctcgcagctctgtctttagggtattgtaggacttgatcatagagctaacttttgtttgcacagtttaactgatttttaagttccgttaatgctcaacaatggaaaatttattaatagaagcacagagatgtcttagaaaagcttatgagaaagaggcggggcaactggcaggaaagaaactagatgttgttaacacccatcccacatatttccaaccacaccacctcctttcagagaacaaatttcctctgcctctggcactcagctagatctcacaaaggttagagcaaaaaaaaaaaaaaaaaatcctgctgccccctcagttcacatccagccacgcttgctatcaccaaggacatcctggaaacataaaggtaatataacaaggacagactgggaggcagagccagacacggaagtctatgctagtacaatagcactgtacatgttactactgcctcctcccgcaccccctttcaggtacttcatgcccagccctgtctgtctgagggttgctatgattataaaagcaccataaattggacataaaatataaatgtgttttattgttgataaagtatcaactgtcataatctttaaatctatagtctacactgaatgccataatcactaacaggtcagtatgacatctattttttttatcaaagtattagttcccaaaataagttcacccagctttaatccttgggagagaaactgcctcaatctttggtatccccaaggaacaacataaagtaataagacatcaaatactagctgtagtctgagaagcccacgacatcatatgattaaaatgtactatttgtttgctatcacgacatgaattctccttccctgtggtgctgctcacttcctctctaacatctgaactgttttgaaatacattgtatgcatagcaatagctagtatgaaagggcttcaagaaagccttgttgtttcatgagatttggggccacaaaagttaaacatttaacctggcaggaactcaaagtccaggcagactcaagtgtttgagctaattgtgttttaaaagtatgtagattagtgttttgaacttggatacaaattgcaacagaaacattactgtgaatggtggttaggttcgagatccagcatgatgtccagtgtagctgccatactaaactttcataataaatatagttggaaagagtattgttttaaaaattttgtgtaatgattcagctctgtatatgcccctgcttgccattgctccttggaatattgaaatgaaccttcattcattcaggctcagtatattaattcagtataaactatgataaaggataactaacacgtctttaatataatgtgggcatgggacatgtattttgttttggaaatatccaaaattattcagactcaggtctaatgaatgaagtagaatgaacttgtcttccaaattaaaaattaaataccacagcaagaatagttggggatttccatattccactttcaatggatagaacaaataggcagaggatcaacaaggaaatagaagacttcaataacactacaaactaacaagacataacatacatctctgtataaaatactccaccaatcagggagaatatgtattcctgtaacgtacacatggaacattcctcaggaaagaacatacactaggccataaaatgaatcttgctacattttaaagggctgaaatcaaagcatattctctgaccacaatggaatgaaattaggaatcaataacacagaaatttgcttctcaacagataatgggttaaagaaaaaatcaaagaggaaattagaaaatattttgagatgaataaaatgaaaatacaacataccaaaacacatagaatttagctgaataagttcttacagaaacacctacagttgtaaatacctatgttagaaaagaataaagatctcgaataaataacctaatcttcaaacaaccttaagacattagaaaaagaagagaaatctaaacctaaagcaagaagaaggaagggaaaactaaagatcagcatggaaataaataacatagagaaaatagagaaaaataataaaacaaataatgaaaatcaacaaaattgacaaacttttaactagattgaccaacaaaaaattacataatactaacatttcaaattactagaatcagaaatgaaagaggagaaattaataataactttgcaggaatagaaagaataataaacaaatactatgaaccattgtataccaataaaagggacaagttcctagaaaagcacagactaccaaaactgactcaagaagaaatacacattctgaatacacctataacaagtaaagagattgaatttataataaaaagctattcacacaacaaaactccaggtccagatggctttaccgataaattctgccaaacatttaaccatgcattaataccaattcttcacgaactcttcaaaaaatagttgagaagaatacacttcataactcttttttttaggaaagtataatcctgatgccaaagccagataagaaaagaaaactatagaccaatatttcttataaatttgggtgcaaaaattctcaacaaaatcgtaaactaaatccggcaacatataatacaaattatatcatgacccagtgggatttatcccaggaatccaagattgaattaacatctgaaaatcaagtaatgcaatacaccatatcaataaaatataaatagaataaaaaattaaaaccacaagatttttttcaatagatgcataaaaagtatttgacaaaatctaataccctttaatgataaaaacacccaacaaactaggcaaataagggaacttcctcaaccaaataaaagttgtctagaaatacccacagctagcatcatacttaatggtgaaagactggatgcttttcccctaagatcaggaacaagatcaggacactatcaagaaagtaaaaagataacccacagtatgggagaaaattttcaaaaattacatataagagatttatatctacaatatataaagaatttttacaacttgagaataaaaggcaaattaaccaatttaaaatgggcataggctatgaataggtgttttagcaaagaagatatataaatggctcataagcacatgaaaaattatcaaaattattggttatcatggaaatataaatagaaatcacaatgagataccacttcacacctaccaggatggctagaattacaaagttcagttaaaaacaagtgttggcaaagatgtggagaaataagaaccttcatacacgtatgagaatatgggaatgtaaattggtgtgaccactttagaaaacagtttgaaagttcctcaagtagttaaacaggattaccacataacccagaatttcacttccagatatatacccaagagaaatgaaagcatgtgtccacacaaaaacttggacatggatgttcatgtaagcattattcttaagagccaaaaagtggaaacaatgcacatgttcatcaactaatgaaaggataaatgaatgtggtaagtccatacattggaatattattcagcaataagaaggtaaagcattaatacacagttagtactttactaatcatggttactcaaagctgagtgtatggaaggataggaggattgggaggtgatagcggaaagataaagggttttttttttttggaagtgatgaaatttttttaaattgtgttgatggttgcatatgtctttgaatacacaggaaaacattgacttgtacacattcaattggtaaattacatgataacttgaattacatctcaacaaatctttaaaaaaaaagcttaacactaaacaagctttagaaacctacccagttctctgctgtctgtgaaagcttctctttgcaactcctcttcctattctctcaattcagagtatgtgggcaattttggagtttgtattagcagattgcctaggttatagtgaacaatagattttgttgcattttctacaaacatgctaacttaattctttatcttatctcaattgccaatctttgagagtaatgcttgattctctgaaggaaatcctggtgctgaattgatttggtataaattaaaaaatagaatacaaagacaaaggaggacttaatctctaagattttcatggttatttgaaggagaaagcagatagattaatggaaaagaaagataatgggccaggcatggtggctcacacctataatcccagcactttcggaggccaagacaagcagatcagttgaggtcaggagttcaaaaccagcctggccaacatggtgaaaccccatctctactaaaatatatatatatatatacaaaaattagccaagcgtggtggtgcgtgcctgtagtcccacctacttgggagactgaggtgggagaattgcttgatcctgggaggcggaggttgtagtgagatgagatcacaccactgcattctggcctgggcaatagagtgagaatttatctcaaaaaaaataaaaattaaataagaacaagaaagatcatgactttgagataaactcttatgttagacctgcaaggagcttcagacatctgtattagtccattctcatgttgctatgaagaaatacccaagattgggtaatttacaaagaaaagaggtttagtccaggcgcggtggctcacgcctgtaatcccagcactttgggaggctgaggcgcgcagatcacgaggtcaggagatccagaccatcctggctaacacagtgaaaccccgtctctactaaaaatacaaaaacaaaattagccgggcgttggggcgggcgcctgtagccccagctacctgggaggctgaggcaggagaatgacgtgaacctgggaggcagagcttgcagtgagcagagatcatgccactgcactccagagcctgggtaacagagcaagactccatctcaaaaaaaaaaaaaaaaaaaaaaaaaagaaagaaagaaaagaaaagaggtttaattgactcacagttccccatgactggggaaacctcaggatacttacaatcatggcagaaggcacctcttcacagggcggcaggagagagaatgagcacaagcaggggaaatgccagaagttcataaaaccatcaaatcttatgaaattcatttatcacgagaacagcatgggggaaaccactcccatgattcaattacctccacctggtccctcccacaacatgcagggattatgggaactacaattcaagataagatttaggtggggacacagcaaaactatatcaacatctaatccatcctctttgctttacacatgaggaacccaaaactggtaaattgagtgtctaattcaagatcatgcacctaactgctgacccactacactggttgacccaggacttggaggtagtgggatggggaaaacgataaccaatgggagggaagaagacagttctcagtgcaaatactgggaaaatcctgggtgagccagcatgagtcatcctatgttgacagcaccagtcagagtcaacatacctcctgactcctcatccagtctggtgctctttcttcaagggcatgttttctcagacagacagctctctactctgagagatcttataagcccactttccaatgactgctctctggatgagaattttccaaaatgcaaattaccttgtgagttaattttattatatgaataaatacttatatttcaagaccttacattagttttttcctatgagtcaataattttaccatgatatttaacataaacctaacatcttatcacttttcttctcatttatcaaattaattccagtgagacttaagtttttaaatatactgaacagacgtttgctacagcatgctgctatggaataggtatgtggtcttcagagtctttgttatcactgaaataatccacactagaaacacaaaaacatgaaaactgttttatgtgactaatccaccacattccctagaggctgctgttcttatttcatcacaaacacaaagttattgatatggaattcatttcttcctgcttactttaaccaactacctacctagccacatggccattatagagttaaaatctaccatgagaaagaaatagagcccttcatggcctaaggccatgtggggtttctttgtgttttgttttgtttttaacattgatctattttttaattaacaaataaaattatatatattatgcacagcattttattttagaaatatgtatacattatggaatgacgcgagctaattagcgtatgtattacctgacatacttatcattttttaatgtacatgatcttattttattttattttttattttaagttctgggatacatgtgcagaacatgcaggtttgttatgtaggtctacatgtgccatggtagtttgctgtacttattgacccatcatctaggtttcctccccttgccctgcacccccccaacaggtcccagcatgtcttgttcccctccctgtgtccatatgttttcattgtttagctcccacttatgagggaggacatatgatgtttggctttttgttccagtgttactttgctgaggatgatggcttccagcttcatccatgtccctgcaaaggacatgatcttattcctttttatggctgcatagtattccatggtgtatatataccacattttctttatccagtctatcactgatgggcatttgggttagtttcatgactttgctattgtgaatagtgctgtactaaacatatgtgtgcatgtgtctttatagtagaatggtttatattcttttgggtatatactcagtaatgggattgctggctcaaatggtatttctggttctagatccttgaggaatcaccacattgtcttccacaatagtagaaccaatttacactcctaccaacagtgtaaaactgttcctatttctccacagcctcgccagcatctattgtttcctgactttctaataatcgccattccgactggtgtgagatggtatctcattgtggttttggtttggatttctttaatgatcggggatgttgagctttttttcatgtttgttggctgcacaatgtcttcttttgaaaagtgtcttttcatatcctttgctcactttttgatggccttgtttttttcttgtaaatttgtttaagttccttgcagattctggatattagtcctttgtcagatgggtagattgcaaaaattttctcccattctgtacattgcctgttcaatctgatgatagttttttttgctgtgcagaagctctttagtttaagtagattccgcctgtcaattttggcttttgttgcaattgtttttggcattttagtcatgaagtccttgcccatgcctatgtcctgaatggtattgcctaggttttcttctaggggttttatggttttgggttttccatttaagtctttaatccatcttgagttaatttttgtataaggtgtaagggaggggtccagtttctattttctgcatatggctagccagttttcccagaaccatttattgaataggaaatcctttcaacattgcttatttttgtcaggtttgttgaagatcacatggttttagatatgtcttgttatttctggtgtctctgttctgttccataggtctatatgtctgttttgttaccagaaccatgctgtttttgttactgtagccttgttgtttagtttgaagtcagctcacgtgatgcctctggctttgttctttttgcttagaattgtcttggctataggggctcttctttggttccgtaagaaatttaaagtagtttgttctaattctgtgaagaatgtcaatggtagtttgatgggaatagcattgaatctgtaaattactttgggcagtatggccattttcatgatattgtttcttcctatccataaggatgaaatgtttttcaatttgtttgtgtcctctcttattttcttgagcagaggtttgtagttctccttgaagaagtccttcacatcccttgttaggtgtctttctaggtattttattctctttgtaggaattgtgaatgggagttcatttatgatttggctctctgcttgtctattgtggctatataggaatgcttgtgatttttgcacaatgattttgtatcctgagactttgctaaagttgcttatcagttcaaggagtttttgggctgagatgatggggttttctaaataaagaatcatgttgtctgcaaacagagacaatttgacatcctttctttctatttgaatacactttatcactttatcttgcctgattgccccggccagaacttccaatactatgttgaataggagtagtgagagaaggcatccttgtcttgtatccgctttcaaaggggatgtttccagcttttgttcattcaatatgatattggctgtgcatttgtcataaatagctcttattattttgagatatgttccacaaataccaaactgtttattgagagttttcaacatgaagggatgttgaattttatcaaagggctttactgcatctattgagataaatatggttttcgtatttcgttctgtttatgtgatggattacatttattgatttgcgtatgttgacccagccttgcatcccagggatgaagctgacttgatcatggtggataagtttttggatgtgctgctggattcagtttgccagtattttattgaggattttcgcatcaatgttggcctgaagttttctatttttgttgtgtctctgccacgttttggtatcaggatgatgctggccttataaaatgatttgggtaggagtccttccttttcaattgtttggaatagtttcagaaggaatggtaccagctcctctttgtacctcttgtagaatttggctgtgaatccatctggtccagggctttttttgtttgttaggctgttaattactgcttcatttcagaacttgctcttggtctattcagggatttgacttcttcctggtttagtcttaggagggtgtatgtgtccaggcatttatccagttcttctagattttctagtttatttgcatagaggtgtttacagtattctctgatggtagtttttatttccgtggggtctgtggtgctatcccctttatcagttttttattgtctctttttgattgttctctctttcttctttattagtctagctagtggtctatctattttgttaattttttcaaaatccagctcctggattaatttgtttggccaggggtggtgaggtgttgtgtctctatcgccttcaattctgcactgatgttagttatttcttgtcttctgctagcttttggatttgtttgtgcttggttctctagttcttttagttgtgatgttagaatgtcaatttgagacctttctagcttttcaatgtggacatttagtagtataaatttccctcgtaacactgctttagtgatgtcccagagattcgggtacattgtctctttgttctcattggtttcaaagagcttcttgatttctgccttaatttcattatttacctaggagtcattcaggagcagattgttcaatttccatgtaattgtgtggtttcgagtgagtttcttcatcctgagttgtgatttgattgcactgtggtctgagagaatgttggttatgatttctgttcttttgcacttgctgaggagtgttttacttccaattatgtggtcaattttagaatatgtgccatgtggcactgagaagaatgtatatcctgttgatttggggtggaaagtttggtagatgtctattaagaccacttgatccgaggctgagttcaagtcctgaatatccttgttaattttctgtctggttgatccaatattgacagtgaggtgttaaagcctcccactattattgtgtgggagtctaagtctctttgtatgcctctaataatttgttttatgaatctgggtgctcctgtattgggtgtgtatatacttaggataattagctcttcttgttgaatttattcctttaccattatgtaatgtacttctttgtcttttttgatctttgttggtttaaagtattttgtcagagactaggattgcaacccctgcttttttttgctttccatttgcttggtaaattttcctccatctctttattttgagcctatgtatgtctttgcatgtgagatgggtcccctgaatacagcataccaatgggtcttgactcttaatctaatttgccaatctgtgtcttttaattggggcatttggcccatctacatttaagtttcatgttgttatgtgtgaatttgatcctgtcatcatgatgctatctggttattttgcacactagttgatgcaatatcttcatagtgtcattggtctttatattttggtgtgtttttgcagtggaaggtaccagttttttctttccacatttagtgctacctttaggagctcttgcaaagcaggcctcatggtgatgaaattcctctgcatttgcttgtctggaaattttatttctcctttacttatgaagttagtttggctggatatgaaattctggattgaaaattcttttctttaagaatgttgaaaattggctcccaccttcttctggcttgtaaggtttctgctgagagtccactgttagtcttggggcttccctttgtaggtgaccttgcctttctctctggctgcccttaacattttttccttcattttgacgttggagaatgtgatgattatacgtcttggggttgatcttctcatggagtatcttaggggtgttctctgtatttcctgaatttgaatgttgacctgtcttgctaggttggggaagttctcctagataatatcctgaagtgtgttttccagcttatttccattctcccaatctctttcaggtattccaatcaatggtatgtttggtctttttacatagtcccatatttctcagaggctttgtttgttccttttcattcttttttctctaatcttgtctgcatgccttatttcagcaaggtggttttcaaactctgatacccttttttctgcttggtcaattcagctattgatacttgtgtatgcttcacaaagttctcatgctgtgtttttcactccatcaggtcatttacattcctctctaaattggttattctaggtagcagctcctctaaccttttatcaaggtttttagcttctttgcattgggttagaacatactcctttaactcagtggagtttgttattacgcatcttctgaagcctacttctgtcaattcatgcatcagaccctccatccagttctgcgcccttgctagaaaggtgttgtgatcatttggagaagaggcattctggccttttgggttttcagtggcttttttggttgattctttctcatcttcatgagtttgtctaatttcaatctttgaggctgttggcccttggatggggtttttatggggactttttatgttattgatgctgttgttgttgctttctgtttttctttcaatggtcaggtcccttttctgtagggctgctgcagttcgctgggggttcacttcaggccctatttatttggttcactctcacaactggagatgtcactcaagggggctggagaacaacaaagacgggtacctgctccttcctctgggatctctggctgcaagggacttcaaactgatgccagtatgatcattcctgaatagggtgtctgacaacccttgttggagggtttcaccctgttgggtggcacagggaacaagacccagttaatgaagcactttgactgtgatttggtggaggaagtgtgcttcgctggggggaacccacttatctgggctgtctggattcctcagaactaccaggaggaaaggctaagtctgctggtctgcagagactgaggccacccttctccctagaggctcaggcccagggagatgagaaatctgtccctgagcccctggctggaattgttggagtttcttcagggaggcctcacccagtgaaaagggatgggtcaggatcaggcctgaagaggcattctggtcacagtctgccacagccagtgtattggcctgtgtgggacacctcatgggaccaagccatccagtctccttggctccagcaggggaaaagggtggcctggagctatagagatagctgccccccttcttccacccagggaggttagcatgttaggcagctatcagtcccagtgctgggtgctgcccctcccccaaggagctcaaagggcttagacaacaggcagccacagctgtggtgctcattgcctctccccctgggaacttggcaggcttaagcagattctagctgagatgctgttgagaatatgtgtgcctctggggttgggaccctaggccccagtgacatgggttcacgagtgggatcttccaatccatgggttgcacatttccatggaaaaagcacagtttccctggctgggtagaacactcactcaccacctcccttggctgggggttggaggctcccctcccttgtgtgtccctcaggtcagctgctgcaccacacctttcttccttcttttccatgggttacaccagccacctagtcagttctggtgagagaacctggatacctcagttgcttctgcaggattcacatgctattatggttctttttgatgggggcctccaatcgccactgcttctagtcagccatcttcgccctgccccaatgtcatacttattttttgtgatgagaaccttaaaatatactcttttagcaatttttaagaatataatacattgttattatagtcaccatgttgtacaatagatctcttgaacttacttctcctgtctaactgaaattttgtatcttgtgaccaacatctcactgacccctccccatccacccctgatagccatttttctctctagttccattagttcaagtattttagattccacatgtaaatgacatcatgcaatatttgcctttctgtggctggcttaattcacttaatataatgtttatatattacattctccagtttcatccttgttgttccaaatggcaggatttttttctgtttaaatgatgaatagtattccgttgtggatatataccatgttttctttatccattcatccactggacacttaggttgactccatgtcttgactgttgtgaatagcactgcaatgaatatgggagtgcacacacctctttaacacactgatttcattttctttggatacattgccagtagtgggatcatatcacttgggccttcctaatctggtgtctataacagacctgggaagtgtgaggagcctatgcagcatgtgaaatatatgtcaacatttttctgtacaccttggaattacagagaaactatagcaatttttatattctcaaagaagtccttcaacccaaaatagttaagaaatttagccttaagtcttccctagaacaatattattcatactctttgcacaagacacaaaaaaatgtaaagtagctaagtctgactcagtggagaagattgattaatccttgattagctctccaaagactaaagagacattagtacactaagtaagaaaataaatttggaataaaagtactgagaggagaaagggaacaaaacagaaacttaaaatcatttctgatgcataaggaagaaaatgaacatgaaggggaaagatgccattacagttaaggaatttatctaccttaagtgattgaggcagttgggaccatcctgaaatctaattagggaaaacataaatgtctcaaagtacagtgagaaaaatgtaaacactcgacaagttttctcaaggaagtcatgataagttattccacttcatcacttgcaccttttttacttctcactattgaaaaacacatttcaatattgagataaaacagcctcccttccctgaaaatactgaggagacatactggaaagcttgcaacatcataaaccactgaagcagacaataacctttttttccaaaaccaatctttgtatgactgtgcccagagacaaatgtagagatggaagaagatgaagttgtcttttatgctttaattttctgagggactgtattaaaaagaataactcaactgtattctcatcatcccttgtgttctcaaacatctaaatacagattgaatatttgaggtacaacccagaaaaggcaatgtgaatattaatcatatgcaagcaattgatattggaatgaagcaatattgttataaatttaacaccaaaacttgtcattcttgcagtaaaagaaagaaaaacactggttttcaaatcaatacatttgatcttgtaatgataaatttctgaatatattacaaaccagacttaatttttgttccaaggcctttcctctgttctatttcttcatttcatacttagtctcataaatcagtggtttcaattaagagttttccagcatgcatttcctaaaacatatgtatagacaatatatcacaaatgattgtgtgaatatgtttactatatttgcccagccagatcatttcatggctgtctacaaatgttatataggaaaataacataatttattataaatcctggcccttctaaaagtgggttatgctcattaggtgcgatgtttatgtaaatacaagtttctagagagcagaaattctgtcttttgaataatttgtgcctactctaatcccacgcatatctaggcacttaacattattcaatgagacataataatataataatgccatcttattgatattcatctattttttacaccaacgctttttcagctacttgttagacaatttcaatgagaacatttttgtaaattcaaaacctgaaagaaaagctcaattcaacttatctaatgttgggatatttctatatgttgaataatatgttaagtgctattgagcttacaaacgcaatatacgatattttatctgccctcaaggagcttaatagattgttgaaaatacagaacaaacacagagaccagttggaaaacaacacaaactagtataatcaaatgttaaatgtgagataaaaacaataaattcctgagaattcagaacaaagatacatctccatgagtaaaataaattgagataggtttcagggaagagatcagacaagagctggcatttaaaaaataataaaaagagaaatttagaaatcttagaacataatccaaagaaagaaatggaattagtaaaaccaggcaggcatggacctcatgcataagggagggtaacgaaaatgactggaagaaacaggctttattccagcaaaagaaagtaccaagttacagggtcaataggtaacaaaggactataatgggaatgactgtgaggattatgtgataaaaacaaataaatacgacttgaaatggactctaatcctcagcatgtaaaatgtcacaaaacacctttttgtttaatgccaagaaaactcgttgtggaaattccattttttaacacactgttagttttcaattaagactaattgaaggggtaaaaaaattactcacgagctttttcctataacttgtgcctgtctttggtgtctaaaacatccttctcagaccaagtgccagaatctttggaaaggaacataatatgcctaattggaaaaggctctgcacgcaccctgcttcagaagagaaccatagcagaagggctttggaagaccacataatgggtctttaatcaacaacacactgcaggaccgatatattttccttgcccaatgtgcctgacatttaaatgcacagagtaggtctcttcccccatatcctgctattctcacaaaacgtatgacttgagttttattctaacttgctgcaaagaaacaaaatatctcaaccagaagcaagcaactgaagttaattattgtgacacatggaaacataataaaaccaggtgtcaaaaaaaatacagaaacgtcgtaggtttatagcctatgttaaccttctgtggcatagattctggcattatcttctggtattactatattcctttaaagattttgtaagtggaaagaaaaattgatccttagattgaaaggtcatcagatagtaatagacagacaagctcatcttattatgtaatgaatggatattttattaattctacattttagtaaagcattcatagcttttgtttttatctttgtttattgttatgagctcagaacattctgggttgaagtatatttactaatgcagggaaataaaaatttgcaacatgacacatactgtggatacccagaaagtaccacatcttgatttggtgaccaccagcacagccaacaaatggtcctggtagatactagagaccaaagaaacaggactgctacagtttgaatgtcccctccaaaacttatgatgaaatttgtcattggaacagaattatgaggtggggcttttaagaagtaattaggtcttggagggaggtccactttcatgaatagattaatgccattatcatgggagtgggtgagttatcacgggagtggctttgttctaaagttgagctcccactcacctgcactttcttgcccttcaccgtatttcattgcagcaaaaacgtcctcatcatatgctgagcagatgccagtaccatgctcttggacttcccagctttcagaactgtgagccaaatatatcactttttaaataaattacctagcctatggtattctagtatagcagtagaaaataaactacaatggtttgaatgtttgtcccctctgaaacttatgttaaaacataacacccaatgtggcagtattgagaggtgaggcctttaagaggtgactaggatgctagggttctgtccttatgaatggattaacacacttatggattaatagactcatggattaatagactaatgtgttaatagattaatggattatcatgggactgagactggtgactttataagagaaagaaactctacagagagtacccaactataagaaagacctcaccagaagtgggcccctgaccttgaactttcgagcctccagaaatgttttttaaataaattattcaaataaaatattttttaataaattacccagtttcaagtattctgttataagcaaccaaaaatggtctaacacagaaaattgacactgggagtaggacattgttaataatgaacatgtgaaaacggggatgtcactttgcaactggataatgggtagaggctggaacaatttgaagcagctggttagtaaatgctcagattctggtgagggcttggaagacaagaccagggaaagttttaaacactttagagaatggttaagtggtcatgatcagaatgttgatagaaatgtggtcagcaggaaacattgtgatgaagttttaggagaaactgaggaaaaagatattggaaagtcaagtaaaggccattcttgttataaattgtcaaagcacttagctggactctgtctgtgactgaggtctttgtggaagtccaaacttgagaaagatgtactagaatatctggcacaaaaaaaaattaagtaaggtatagaaggcattcatagttacctttggccatttacactgagatacaagagcaaagtaataatttaaagacagaattaatagttaaaaaggaagcagagcagaaaaatttggaaaaccatttgaccacaaaaagaatgaaaaaaatgttttcaggagagaaacggagtgtagctgatggaccactttctaaggagattagcagagacagaaggcagccatgtgctatttgtcaagacaatggaagaaatacccccaaagatattgcccagatatttgaagatgccactcccatcacaggcttaggagagcagaatggtgtcaaggaacagccccagagtacacagattcacttgtcagggccattttgggactctgctacccacaccccagcacagcagttctgcagctgccttagccatggctcaagaagcctcaggtgcagctcatgctgcagctccagaaggtacaagttataaattttgacaacatccatgtggtactaattctgcaggcttgcagaaagcaagagctatggaagattgacaacttccacccaaatttcaaaagatgttgctgaaagcctagagacccaggcagagctactgtaaagatccctggtataataaagcagaggagaaatgtgaagttggagctgccaccaagagtccctgttagagtaatgcctaaggaactcaggactaccactgagatctcagaattatggaaccaccagcagcatgaaacatctaccaaaggaagccttaggcaaaggactttattctgcataagcagtcacgtgggctgcaccaagcaaagcccataggactgcctaaggccttgggggcccaaaccccatcccagtgtgtccagaaggtgacacatggagtcaaaagaaatcattcctcagcttaaagatttaatgtctgtcctactcagtttctaacatgcttgagcttgttactcatttctctttgcctattcctccctgttaaaatagaagtgtctgtcttatgcctgaaccactactgtatgttggaagttaataactagtcttaattagccgggtgcagtggcaggtgcttgtaatcccagctattcgggaggctgaggcaggagaatcgcttgaactcggagggcagagtttgcagtgagctgagatcgcgccactgcactccagcctggcgacagtgagattccgtctcaaaaaaaaaaaaaaaaaaaaactagtcttgatttcacaggctcacagatgggattctgaactttggacttttgagttggggctagtattagttaagactttggggttaactttggaaagtttggaaggggtgattgttccactcaatggaaggacataagaaacaggaaaaagcaaggaaatgtaactctactaaaggaaaactttttagtaacaaaccccaataaaaggaaaattaatggagaaccagaaaaggaattcaaaattatgatcttaaggaaactgaatgaggtataaaaaatacagttagacaattcaacaaaatcaggaaaaaacatatgcatgagaaattcaacaaaaagacagatataaaaaagaaaaaaaacaaatcatgcaactaaagaattcaatgaatgaaataaaaaaacaacacagagcttcaacagcagatttgatcaaacagaagaatctctgaacttgaaggcaggtcatttgaaattatttggttagaaaaaataagaatggaaaatagctcttaaaagattgtaaacttatgagacaccattaagtagacaaatattcattatagacattccagaatgaaaaaaaatggaaaaaatgcatagaaaacctatttaataaaataatagctgaaaatttcccaaatctggagacagatataaacatccagatccaggaagtccaaagatcccaaactatatttcaatcagaaaggtactgtccaaggtaaaaaatagtcaaaatatcaaaagtcaaagacaaaaagaattctaaaacaagagaaaagcaccaagtcacatataaaggaatattcattagactcacattaggtttctcttcagaaaccttacaggtcaggagagaataaaatgatatattcaaagtactgaaagaagaaattgacagtcaagaatactatactcaacaaagctatttcttagactgagggaggaataaagccattcccagacaagcaaaagtataaggaaatctatcactagactgactttacaaaaaatgctgaaaagagtcctgcatctggagacaaaaaggtgataatcacaatcatgaaaacatgcaaaaatatataactcattgatagggcagataaacaaagaagaaataaaaaaaagttcataccattcaaataatctatagattcaatgtagtgcctatcaaaatgccaataatattcttcacagaaatagaaaaaaaaatcctaaaatttgtagagaaccacagaagaccccaaataaatcaaaggattgtgagcaaaaagaacaaagctggaagcatcacactacccaacttcaaaatgtactacaaatctttagtaaccaaaactgcatggtaccagcataaaaacagacacatagaccaaaggaacaaaatagacaacctagaaatcaatccacatatttacagccaactcaattttgacacaggcacaaagaacattcagtgcagaaaagacagtctattcaataagtgatactggaaaaactggatatccttatgcagaaaaacgaaactagacctctatccttcaccatatacaaaactcaaatcaaaatggtttaatacttaaattcaagacctgaagctatgaaagtaccagaagaaaacatgggaaaacaattcagaacattaatctgcaaaaagatttcttgtataagagctaaaatgcacaggtaacaaaatcaaacatagacaaatgggattacatcaagctaaaaagtttctgtacagcaaagggtgcaatcaacagagtgaagagacaacatacaaaatggaagaaaacacttgcaaactatccttccaacaagaggttcatgactataatatattagcaattcaaataactcaatatcaaaagaacaaataatccaatttaaaaatgggtaaaagacttagagatttttcaaaagcagacataaaacgaccaacaaacatatggaaaaaatgctcaacatcactaatcatcagagaaatgcaaatcaaaaccacaatgagttatcacttcactccagttagaatgactattatcaaaaagccaaaaaaaaaaaaaaaaaagctagcaaggatttagagagaggggaactcttatgcactgctggtagaaatgtaaattagtacagccactatggaaaacaacatagaggttcctcaaaaaactaaaaatatatctactatatgatccagcaatcccactgctggaatatatccaaaataaagaacattggtatgttgaagagatatctgcatgtccatatttattgcagcactattcacaataaccaagacataaaataacctcagtgtctatcaatctatgaatatggaatatatacatgataaaatatttttaaatgataaaaagaatgaaattctgtcacagcaaaatgaatggaactggaaattattattttaagtgaaatgtgccaaggaaagaaagaaaaatatttcatgttctcattcatatatgggagctaaaaaagttgatctcatggaggtagagagtagaatgatagttaccagaggctgggaaggctcgtggaaggggtaaagagaggctggttaatgggttaaaaacacagttagatagaaaaaagaagttctagttatcaatagcacagcaaggtgactataggtaataataatttactgtatatttcaaagtagctagaacagaatatttgcaatgttcccaatacaaagaaatgataaatgcttgaggttataaatatcttaattatctttgattactacacattgcatgtatcaaaattatcacatgtaccccataaatatgtacaattattatgtgtcattttttaaaaccttggggctataagaatagaattaatgtatttgtgagaagaacatacgttttggagagccagggggagaattacatagatttaatagttgttccttctgaaacccatgttgaaatgtaatccccagtgtggcagtttagggaggtgggacctttaagaggtgattggattgacagggcttttctctgatgaatggattaatctactcatcgcttaactgattgatgggttaaaagattaatgggtaagcatgggagtggaactgggggctttataagaagaagaggagcctgagatagcaagctcagcccattgccctgtgatcccctgcagcacctcaggactgcagtgtcctcacggatgagaagactcacataagaatcaatctctcacccctggacttcccagcctccagaactgtaagaaatgtgttttgttgttattgttgttgttgttattgttttactaaattacccagtttcaggtatcctgctacgagcaacagatatctgactaagacagaaactaagacaagaactttcttcatacttattttttaggtggcctggagagtcagattttatttaaaaagagaaaataatggagttttgaaccacatttccttgattctaaaatacaatctgtcaattttaatgtttctaaaatcaggatacctctttcattgggcatgtacctttgatatagaaacttttcttctgaagatattattaaatagataatatatcttaaaatttattgtaccttataattgagtataacagtatgaaaaaatgatatgggaatatttgtggtgatttgtgaatacgcaggacagttcatggtaagattgatgagcatacactgtggcttcattcccacctccaccctttaaatacaagccttcaaataaggaattcctgacaatacctccacacagccatgtgtttatgatgagatccatctcacacgtgaaggatggtgcataaaaatataaaatgatttgagcaatgccaagtattgaaaagccagaagtataacgtagtgttctgacttcatcttacagtaagtaattaccttttccacccctacctcccatttgtgtaatcaattatcaacctaccttatggcaaacaatgtaaaacattttccctcttgtttcagtaaagatagaaattcatggaagcaaatttcaggtcaactgcttgtatcatattaggaaacagaatgctgttttaaaaaaggagcataattcagagagaaaaagcaagtatcacgatggaaggatttgggttttaaccctggcttcttcacctgggtttatataagtgatttaatttttcctcgaattcttttgactgtccccagctaataacatccgaaagagaataataaaatactgactcacttcccagagatattttaaccagagatgagtaatattttcaaaggtttttcactgctactcttaaagcgtatttcaaaatcaaaatagcataattttcttcaagctcttgcttctacaaaagcataatgctcttgtgtttaatagcttaatcagagacaaaactaaatttgagaagatggggaaagaagaatagagtatgtagtaaagaaaatagctgcagaagccagatttgtaagctcaaataaataatctattgtcaaccatgagctacttcaagcagtaagcttcccttcaaatataaaacttcttaactacccctcacagtgcacctggaatgtcagtgatggtagagttcacagattcaaagcttagtagctgcctagatctgaaaaacaaggctttcatctttaagaatgttccaataattgtttttgagaagctcagagtgaagggcaaatagctacaaacaacgggcagttaccttgtgatcacattgaagccattaacgtaaataaatattctttccctgaggaaacacgtggagaacaatcatgattgggttgtcttcataaagggaaaaagccctaattgtaaatcgagtagctatcagtagatcaatatcagattcaatcatggaaacacttctgaattcaagtggaagtaaccttgacaaaaatcaagcctatttgatgtgaagggaaaaatttgaaagctcacagcacacataaatgctctttcttcttaattaccagtgtttccagaagtacatttggtctagttagcataacttacctggaattgctaaactaccctactggatacatgtcagcttcccagaagagtccctacctgataatatgatatgatataaaattaaagtttattgtttgaggcagctttatggttttccagtagtacttccctcatcaaccagaggaaacgctccttgaagtaatattgctggagacttcctctgcataacaagaatcttggcttccaccaagtccccttatcttaacctaagcacttctttctgctgatttcaactctttaggcaaaacttaactctttcaaccaattgccagtcaggaaatctttgaatccatctgtgacctgaaaggccccacttcatcccacctttccagactgaaccaatgtacaccttacatgaattgatgatgtctgcctgtaacttctgccccctctaaaatgtataaaatcaagctgtgacccaaccaccttgggcgcttgttctcagggcctcctgaggctgtgttatgggcatgtatataaccttggtaaaataaacttcaaatttggggaaaaaaagtgtctacagaagtaaaaaagtctgcctgctctatctcaaatgtatctctctctctctccctctctctttctctctctctctctctcacacacacacattcatgccttctcacacttgatgattaacaaagattgatagactcaatcaatagtactatttccctaataaatataggagatttttttctctattttatcacaaaataactcaaaaaatagtgaaaaaaaattatgaaaggaatgaagatgttacactagaaaccacttaatgcaaaagaaagtggtaaagcagaaaaacaggaacataaaagacatgagacaaagaaaatgaagcataaaatagcaaatgtaaacccagttatatgaacaattgaatgaaatgtgaatgaattaaagaactcaaaatcagagattattagactatataaaaacaagatccaactacatgctatctacagaagaaccttgtctaggttcaaagacaaaatgttagaagtaaaaggatgtaaaatatatatattgtgcaaacagcaaacacaagaaagctggagtagttacactaatattattccaaacagactttagaacaaaaattaattactcaaaataaagatagacattttataataataaaatggtccatccaggagaaagttatgaaattgataaacatacagacatttaacaacagagcttcaaattatataaagcaaaagctgataaaatcaaggagaaattaggtgattcaacaataatagctatttctttctagaaaagtagaaaaactagagagaagattttaaaataaatggaaaatttgaacattataagaaactagacctaacaaacatctatagaatgctgtaaagaacagcagcagaatacacattttttctaagcacacacagaaatttcttcagaattcaccgtatgttagatgataaagcaagtcttgataaacttaaatgactgaaattatgcaaaatgcacacaccaaccataatagcatgtgattagaaaccaatagcaaaaagaaatttggaaaacttgtgaatatgaggaaattaacatctttctgaatagtcagtggctcaaagttaaaaatcacaatagaaattttaaaatctttgagatgaatgaaaataaaagcatgacatcaaaatttatgggacactgctaacgcaggacttagagaaaaatttatagatggaaatgcctatatttaaaaagtgggacaatctcaaaatcagtaaccaaaccttcaccttaaagcactgaaaacagaacaagtcgagcccatttaagcagaaggaagaaaataataaacttttgactggaaattaaggggaacagaaaagcaaaagagaaaataaataaaaccaaaaatttgtcctctgaaaggattagaaaatattgacaaacttttagctatactgactaagaaaaaataagagaagacccaaactactaaaataaaaaatgaaagaggaaacatcattgtcaacctcacaaaatttaaaagaattataaagaaatactctcaactaatgacaacaaattaaataatttaagtcaaatggacacaattttagaagtaaaactgtctcaggaaaaaaagaattctgaatggacctataaaaagtaaggaaattacattaataattttaaaacttccataatgaaaacctaaacacatatggattcacagttgaattctactatttaaagaagaattaatatcaattttttacaaattgttacaaaaagggaaaggaaatatttcccaaataatactatatgtccagtgttatcctaataacaaaaccagacaaaaacattacaagaaaagaaaaacacagatcaatatatcctatgtatataaactcagaaatcttcaacaaaatgctgtcatatcaaatccagcaacatataaaaaggattatacaccatgatcaagagaatttatccccagaatgaaagattagtttaacatacttcttatcaataaaataaagaacaaacttacatgaccatcttagtaaatgcagaaaaagcatttgacaaaatttaacctactttcccttttaaaaaactcaacacactgaatagaagaaacctcatcaacctcctaaaaggcatctatgaagaaaacacacacacacacacagtactggaggctttaactagggaaaataggcaagcaaaagaaataaaaggcatttaaattggaaaggaagaagtaaatctctgcagatggaatggtcttacatgtagaaaatcctaagaaatccattaaagaactattgtagctaatgcataagctgataactctccatggtatgagatcaagatacaaaaatcaaagtatttctatacattaacaatgaacaatcaaaaatgaagttaaccattccaattacaataataataaacataatacaatatttaggataaactttaaaaaagtttaaaacttatactctgaaaatcccaaaacactattgaaagaaaaattttaaagatctaaataaatgtgaaagatctaaataaatgaatggaaaggcttaacaatctttgagatggtaatgcttcccaaaatgttctacaaaataaatgcaatctcagtcaaaatctcagctgatattttgcagaaatgtataagcatgtaatcaaattcatatgcaaattcaaggaatccagtatagccaagacaattttgtttggttttttttttggtttttttattattttattattattatactttaagttttagggtacatgtgcacaatgtgcaggttagttacatatgtatacatgtgccatgctggtgtgctgcacccattaactcgtcatttagcattagatgtatctcctaatgctatccctcccccctacccccaccccacaacagtccccagagtgtgttgttccccttcctgtgtccatgtgttctcattgttcaactcccacctatgagtgagaacatgtggtgtttggttttttgtccttgcgatagtttactgagaatgatggtttccaatttcatccatgtccctacaaaggacatgaactcatccttttttatggctgcatagtattccatggtgtatatgtgccacattttcttaatccagtctatcattgttggacatttgggttggttcaaagtctttgctattgtgaatagtgccgcaataaacatatgtgtgcatgtgtctttatagcagcatgatttatagtcctttgggtatatacccagtaatgggatggctgggtcaaatggtgtttctggttctagctccttgaggaatcgccacactgtcttccacaatggttgaactaattcacactcccaccaacagtgtaaaagtgttcctatttctccacatcctctccagcacctgttgtttcctgactttttaatgatcgccatcctaactgctgtgagatggtatctcattgcggttttgatttgcatttctctgatggccagtgatgatgagccttttttcatgtgtcttttggctgcatagatgtcttcttttgagaagtgtctgttcatatccttcacccactttttgatggggttgtttgtttttttcttgtaaatttgtttgagttcattgtagattctggatattagccctttgtcagatgagtaggttgcaaaaatttactcccattctgtaggttgtctgttcactctgatggtagtttcttttgctgtgcaggagctctttagtttaattagattccattggtcaatttaggcttttgttgccattgcttttggtgcttttagacatgaagtccttgcccatgcctatgtcctgaatggtattgcctaggttttcttctagggtttttatggttttaggtctaacatttcagtctttatccctcttgaattaatttttgtataaggtgtagggaaaggatccagtttcagctttctacatatgtctagccagtttccccaacaccacttattaagtagaaaatcctttccccatttcttgtttttgtcaggtttgtcaaatatcagatagttgtagatgtgtgatgttatttcggaggcctctattctgtccattggtgtatatatctgttttagtgccagtactatgatgttcggttactgtagtcttgtggtaaagtttgaagtcaggtagtgtgatgcctccagctttgttctttttgcttaggattgtcttggcaacgtgggctcttttttggttccatatgaactttaaagcagttttttccaattctgtgaagaaagtgaatggtagcttgatggggatggcattgaatctataaattaccttgggcagtatggccattttcatgatactaactcttcctgttcaagaccatggaatgttcatccatttctttgtgtcctcttttatgtcattgagcagtggtttgtagttctccttgaagaggtccctcacatcccttgtaagttggattcgtaggtattttattccctttgtagcaattgtaaatgagagattactcatgatttggctgtttgtctgttattggtgtataggaatgcttgtgatttttgcacatcgattttctatcctgagacctttgctgaagttgcttatcagcttaaggagattttgggctgaaacaatggggttttctaaatatacaatcatgtcatctgcaaacagggacaatttgacttcctcttttcctagttgaataccctttatttctttctcttgcctgattgccttggccagaatttccaacactatgttgaataggagtggtgagacagggcatccttgtcctgtgctggctttcaaagggaatgcttccagtttttgcccatacaatatgatattggctgtgggattgtcataaatagctcttattattttgagatacgttccatcaatacttagtttattgagactttttaacatgaagggctgttgaattttgtcaaaggccttttctgcatctattgagatatcgtgtggtttttgtcattggttctgtttatgtggtggttaacatttattgatttgcatatgttgaatcagccttgcatcccagagatgaggccgacttgatcgtggtggataagctttttgatgtgctgctggattcggtttgccagtattttactgaggatttttgcattgatgttcatcagggatattggtctaaaattctctttttttgttgtgtctctgccaggttttggcatcaggatgatgctgacctcataaaatgagttagggaggattccctctttttctattgatcagagtagtttcagaaggaatggtaccagctcctccttgtacctctggtagaattcggctgcgaatccatctggtcctggactttttttggttgttaggctatcaattattgcctcaatttcagagcctgttattgatctactcaaagagtcaacttcttcccggtttggtcttgggagggtgtacgtgtacaggaatttatccatttcttctaggttttctagttcatttgcatagaggtgtttatagtattctctgatggtagtttgtatttctgtgggatcagtggtgatattccctttatcattttttattgtgtctatttgattcttctctcatttcttctttattagtcttgctagtggtctattttgttgatcttttcaaaaaaccagctcctggattcattgattttttgaagggtttttgtgtctctatctcctttcagttctgctctaatcttagttatttcttgccttctgctagcttttgaatgtgtttgctcttgcttctctagttcttttaattgtgatgttggggtgtcgattttagatcttttctgctctctcttgtggacatttagtgctataaatttccctctacacactgctttaaatgtgttccagagattctggtacgttgtgtctttgttctcattggtttcaaagaacatttttatttctagcttcatttcgttatgtacccagtagtcattctggagcaggttgttcagtttccatgtagttgagtggttttgagttgatggtttcttaacgctgagttctaatttgtttgcgctgtcgtctgagagacagtttgttctgatttctgttcttttacgttttctgaggagtgcttcacttccaattatgtggtcaattttagaataagtgtgatgtgctgccgagaagaatgtatattctgttgatttggggtgtagagttctgtagatgtctattaggtccacttggtgcagagctgagttcaagtcctagatatccttgttaaccttctgtcttattgatctgtctaatattgacagtggggcattaaagtctcccattattattgtgtgggtatctaaatctctttgtaagtctctaaggactagctttatgaatctgggtgctccggtattggttgcatatatatttaggatagttagctcttcttgttgaattgatccctttaccattaggtagtggccttctttgtgtcttttgatctttgttggtttaaagtctcttttatcagagactaggattgcaaccactgcttgtttttgcttttcatttgctttgtaggtcttcctccatctctttattttgagcctatatgcatctctgaacatgagatgtgtctcctgaatacagcacactgaggggtcttgactgtttatccaatttgccagtctgtgtcttttaattggggcatttagcccatttacatttaaggttaatattgtgtgaatttgatcctgtcattatgatgttagctggctattttgcacgttaattgatgcagtttcttcatagcatcaatggtcttcaccatttggcatgtttttgcagtggctggtaccaattgttcctttccatgtttagtgcttcctgcaggagctcctgtaaggcaggcctggtggtgacaaaatctctccgcatttcttgtggtgtaaaggattttatttctccttcacttatgaaacttagtttggttagatatgagattctggattgaaaattattttctttatgaaggttgaatactggccccaactctcttctggcttgtagggtttctgccaagagatccgctgtaagtctgatgggcttccctttgtgggtaatctgacctttctctctggctgcccttaacattttttccttcatttcaacctcggtgaatctgacaattatgtgtcttggggttgctcttctcgaggaatatctttgtggtgttctctgtatttcccaaatttgaacattggcctggcttgctatattggggacgttctggataatatcctgatgagtgttttccaacttggttccattctcctcgccactttcaggtacaccaatcaaacgtagatttggtcttttcacatagtcccatatttcttggaggctttgttttttctctttttttttttttttttttgctctttttttctccaatcttgtcttcctgctttatttcattcatttgatcttcactccctgtatcctttcttccacttgatcaaatcagctattgaagcttatgcacacatcacaaagttctcgtgccatggttttcagctccgtcaggtcatttaaggtcttctctacacagtttattctagttagtcattcatctaatcttttttcaaggtttttatcttccttgtgatgggttcaaacatgcttacttagctcagagaagtttgttattaccgaccttctgaaacctacttctgtcagcttgtcaaagtcattctctatccagctttgttccattgctggcgaggagctgcaatcctttggaggagaagaggttctctggcttttagaattttcagcttttctccccatctttgtggttttatctactatctttgatgttggtgacctagagatgaagttttggtgtggatgtcctttttgttgatgttgatgctgttcctttctgtttgttagttttccttctaacagtcaggtccctcaactgcaggtctgttggagtttgctggaggtccactccagaccctgtttgcctgggtatcaccagcagaggctgaagagcagcaaatattgcagaacagcaaatattgctgcctgatccttcctctggaagttttgtcccagagaggcacctgcctgtatgaggtgtctgtcggttactactgggaggtgtctcccagttaggctacatgggggtcaggaacccacttgaggaggcagtctttcctttttcagagctcaaacaccatgctgggagaaccactgctcccttcagagctgtcagacagggatatttaagtctgcagaagtttctgctcccttttgttcagctatgccctgcccccagaggtggagtctatagaagcagcctgccttgctgagctgaggtgggccctgcccagtttaagcttcctggccattttatttacctactgaagcctcagcaatggtgaatgcccctcccccaacaaggctgccacctcacaggctgatctcagactgctccgctagcagtgagcaaggctccttgggcttgagaaccaccaagccaggcatgggagagaatctcctggtctggcagttgctaagactgtgggaaaagtgcagtgtttgggcggaagtgtaccaatttttcaggtacagtctgtcacagattcccttggctaggaaagggaaatctcccaaccccttgtgcttcccaggtgaggcaacaccccaccctacttcagctcaccctctgtgggctgcacccactgtccaaccagtctcagtgagctgaaccaggtacctctgttggaaatgcagaaatcacccatcttctgtatcgatcacactgggagctgcagaccagagctattcctattctgctatcttggaatgggaattctaaaattcagcagcataatttctaatagccaaaatttggaaacaacacaaacatccatgaagtgaccaacgaataaacaaagtgtggtctcttagtaaaatgggatattatttggcgataaaaagcaacaaatatactgacacatgctacaacaccaataaaccttaaagtattatgctaagtaaaagaagcagacacaaaaagccatgtattttattactctgcctgaaatgtccagaataggaaaatttgttggacacaaaaagtagagaagttgtctagggcttgggacaaaatggggtatgactgctaattggtatgggctttgtttgggggcagatcaaagtgtatgaaaatagcttgtggtgatgatttcacaactctgctaacttactaaaaactagtgagttgtacattttaaatgacaattatattgtatgtgaattatatctccataaagctatgagagaaagagctttcaatggcttctgtaccatgtccaaattgttctgccagtctttgaaacccctccataccacagaatcagatttcatactatgcctttcacctttcaaaattatttccaactgtatcagttttctatgactgtgtgacaacccacccccaaattcagcaacttaaaacaataaacatgtattaatattatcacacgaattctgtaggtcagaaatctgagagtggcttagctgggtcctctggctcaggcacttgatgtctgccaaggctgtagtcatctcaaggcttgactcaggcaataagttccaacttccaagctcactcatgtggctgtttcaacccctcagattctagctgcttgttggctgaagacatcagttccttgatacacggcagctcatcacacatcagctagcttccctcagagaaagcaatggtgaaacagagtgagtaagatggaagtcagagtcttttgtaagctaatcttggaaatggcatttcatggcatttgctgtgttcttttccttagaagctagtcattagatctggaccacacacaggaagaggccattgcacgacagcataaataccatgaggagggagacagatgggggccatcacagaaactgcctatcaccacactgaacaccatctgatccaaccagtctggttccctctccatctcacaagcacatttcttgaccctgtcgtaactcttaactcactttattcctgggcctctctcctttaccgatctgaagtcagactctcattcaagatgagctcaaattccatgtattccatgaaacttgcccctgattatactttattcctctgaactccttatgtttcataattatactacataatttaccccttaattatctcctgccttgccttattacgcaaagtatgtctcccttaactgcatatgcttcaactgagagtttgtaagaaacacagaatctccagctcctaaaccagaatctgtgttttaacaagacatgcaggtgattttcatgcccatgaaggcatgggaagcaccactgaattgtaccattctctattgtttcacctgtgtaagtcccatatccttgactaggcttgcgtgtatttggagaactggtaccattttctatatttgttctgctcttaaacacacacacccaagcttagaatataggttagtgctgaccacacagtagatcacaaattcaagctactagagaagaaacacctctatgaactggccaaattcagtaacaggaatataaagataaatcaaatcccagctctgctactcaacagatagtgtgactttgggcaagtcatttaattactctacatttccaattctttatctgaatttaaaggggactatcatggaagctccaattttcattttatttctgaatatctactaatccacaataacattagaatcaatatagtttgatattgcttaataaataagaaaacattctggaaccttttagtgcctaaatgtcatcaacatgaacatcagtaataactgcattgtagatgtgagaaaatgttgcttagtccaggattgtttagcaaaaaaacatagaattctatatcctgcatttgatttcccttataatttaggccatgttgaatgtaagtaaattgttaaagaagtttaaaacaaatttttgctggataacaacaagagtatggctagaacaggtgaaatctcccctttgccacactttgggagtcacccctatgctcctcagctctgccttggaaaaggagcttctggctctgtaagagtcagattagaaacaaagactttgccttcaaaagaaaactatgaatttccacctgaggttcaccaccttctcctgtaaaaggaggagctggatttgttatacagagcagcagaactaggctgagtgaataaagaaatggacatatacacaggtggtgctttttgttgttgtttttaaggtactcagtttaatttcttcttctgcctgtaaggtccaatcccacccacagagtttaaattggcacttcccctgaggacctggactccaattacatcttatattttgatctctaaatctcttcaagttcaaggacttcacctcctctccaggaataaagggctgtacttcccagtcacctccctccccgtcatgttgtccttgcttcagccttcagtcctctcttctgcagcaaagaatcctaattccctaccagaattcttaaacatcagtttactttacacatttcttttttggcttctttactgttagaacaagccctgagtgtgtaaaactgttctcactgtgatgacaaggctgtactgaacaattgggggcaagagagctgtggttccagctgagggggctaagttatgggaccatgcatgcctccacacttatttctctaaaattcatcatagctctgaacttcatgcagaactcctagaaaaaaaaacacagtccagctacctacttttaaatttatggacgaatgatggtggtgatgatgatgataacaataataacactagtgctgttagccgcctctgaacagaagcatatttttggtttctgtcctcaggctccaaagtggttcacatgatgcaaactggccgtgtagtcagcgttttggcattttcatttgatttctctgcccactctcccctgttggcactggctgcactttgaaatgaatttctgttcctgtgcaaacaggaccgcaagaacagttaccagactttgagatcactcagggcatgcaggtttccagagatcattatcctagaagcacaattcaagaataaatatgggagctcaaaatccacctaaagaagagagaactcttgtacccctctggcagataatttctgcccacagccaaataatactttggtaaagtcatcctgaaactatttcttctatccggccaaactcgggcacttggacagaagcaccagagacatcacagagaaaagagatgcaatgggaataacacaggaccaaggacagggacccgggttctggtcccagttccaccactaactttaacactgagtgagccacttctcccaagtgcttagctttctctgtgaaagataatagttagatactacagtttccaacttgaacttacctaatcatcagtatcttgtaggacatttattttaaatacagattttcagactgctccctgcagatgctggttcagtatgtttagcagttactgaatactaagtttagttagtattcaggagtttgcattttaacaagtgacccaggaaagtcttcacataagcaagtttaggaaacacgggccacatgacatcagaaagctcttccgggtctgcagttctgcagaatcaagttaattaatggccacgattaagcttctactccctacaaagctctgctagatcctaaaaatgataggaatgatagctaacatttatgtagtactagtcactattttaaaggctttgcatatatgaactattttaatcttcacaacaaccttctgaaccagatactattattatccctattataagacacagaaacagatgcacagagattaagtgatttttcaaagatgacttggccactaaggaggtaggattgggattcaagccaaggcaccccagttatagagcctagcctctcgactgctctgccctgccaagcttcagccttcctgtagatgctgaacagctcagaactcctcagcatcaggcacttggtggacaataagaaaagccctctaataactaataacagtaccttaaggttatagtctgaatgtgtcccccaaagttgatgtgttggaaatgtaatgcccaatgtaacagtgtggagaagcgggactctaagaggtgactaagtcatggtgactctgccctcataagtagattaatgccattatcacaagagttggttagttattgctggagtgggttcctgataaagaggatgtgtccacccactctgccaccctttgtgtgctctctcacccaacccttctgccatgagatgatgcagcaagaaggccttcaccagatatgggccccttgactttagacttcccagcctccagaactgtaagaaacaaatctctgttctttataaattatctagtctcaggcattctgttataggaacagaaaggtctaagacacttaacaaccatctacatcaactagatcagtctcaggagccagggtgagggtactagggaagccaaaaggcaaaccctagacttcacccttgtttcaatcaaagaaaactgattttgtctgtttggtacatgtcacatttcatttgagaaaaaaaaatcttgtgattaaaaatgagatgctagaaatcattgatctaggccccttttattatgtagatgagaaaactgatcccaaaagataaagaaacttttccagattccaggaacaagaagtccttggcaatggcttctaaagccccaggtttccagactcatttctacagaatcaaaatgcacttgtcaaccagatggacttgataaaaacccattaaaattaacatagattaataaagtaaccactggagaactgcagaaactggggcagcagcatttgagaggggacatgcacagtgacaccaaatggaacagaagatgactcaaatcaatggctgtgtcagtattcacaattctctcctccagtaactgtagcatcaccaaaaatcagggggaaaagcccacccagcttgtgttgaagtggaaacatgactacaagtggagtaggaggacttacttatgaactctagctgtgccatttactacctgggtcatgacgctagtgattagacaagccatcaatatgaggctttcatgaaatcatatatgtcagtaagtccacgactgtgaagatgtagtaaacgtaataatcttttagaccagaagctcccactcacagtctggtattactggctttacctgtcattacaattcatgatgttgagaggaaggctgatcatataaatggggatccaaacccctggaaaatttccctgagctccccctttttttgaacttctgtaggcatgatgataatcataaagaaataatctgcaatggttgggcagcagcaagcccagaagatgagctgccagatagatgctgtgaccgttatcagtgattccataaatgactcagttttgccacccaggcaccactcagagatcaccagaggtttgaggttaacgagtaccttggcacagaacctagtgttactgatactacacactattttttgctctcttctgccactgattagtagatgaggagagagttatgtcagcctcaagtgcagattgcaagagggaagacacaggaatatcaaaatagtgccatcacccaaggaagaggcttcattgcaaatcatgctaatatgtgtattccccctgctcgagactgagagctcctagaaggaacatctgataaaatatgtaagtccttcctcatccatcctgatttctcatcattcgttttattgacaaatggatcaggtgttaaaatgtttaagggtaatcgacagaaaaaaagttaaagttaaaaaacttctatgactaacctgacctgtttctgccaactgacaaaagcccacttttattaggcttcagcctgattaagctcaggatgaataattaccatgtgctgagggcttactgtgggccagagaggtgcactatctctgatcccaacgactaccttgtaagttagatattatcacattaattttacagacaaggcaacagagactgaaagagtttactagtctagtgtcccaaagctagtatgtgtcaaattcaggatttgcctccaaactctgcagactgcaaacctctccagtttgcaggaccagattcagacttcttcacttgacccaaaggggccttcaagacccaatctgtttgtagcctgagggcctgctatattccacatccatcacatgtagccctcactacagaataccttgcggaggcctgcatgtatcacagcgtgactcacctccttgccttctcccatgaaatcccttctgcctggagttttattcttccttctttatgcagttatattccagttgctctcaacactcagctcaaaccacattcttcaagaaggtcatctgtgatgttcctcaagctaatataacttctgctttgttggactccccatagcagcctggacaaatttctatgtaatcatttattaattacatttaactacagtttataatttatttttatctccctcactaggcatacaccaacttgaggggaaaaagactttatttaatttttattctcaggcttagcatagtactatatgagtagatactcatttacacaataaatatttgagcaaatgaatgatcacaaaagtgaataaactgattaattaacttccaggactatcttaaaggctcagttaacttaacctcatgtgtttatccaagtgtaatccttctcctccactgggaactcctcggtacagaagcctctcttgtctcttttataatctctggaacctattcgcccgtgattcaataatggctgaatgaccctctgtcaacactatctctgtttgaatcaagaacaatactatatgacaattagataccatgtaaaatggacttctaatgggtttgaaatgttaatgtataaaaaaagtactacaaataaaatgtaaggtaacatatacatgtatacatatatatgcatatatacatgcatatatacatatgagaataggaaaggacttactagccatgacacaaatctcaatagccaaaagagaaaagactcttactacacaaaaatgcaaaaacttctgaagggaaaaaaaaaaaactctatgcaccaagtttacacacagcaaaatgaagaaaatatattcacatatatgacaatggccacaaaaatgttagcatttttgatacatacagggcacgttcaaataagagaaaaaacaaacaaacaaaaattacacaaagaggaaattttcatatgaagaaatgcaaatgaccaatacaacatgaaagtgtcacatcactaattggattaaagtataaatttaaacatcagtgtcatatttctcactaattttatagaaatcacaaagcacatgggaaaatggacctatgaagttgggggcaattttaaagtatatttttgaggccgggcgcggtggcttacacctgtaatcccagcacttcagaagaccaaggcaggtggatcacctgagtttaggggttcgagaccagcctggcccacatgatgatattccgtctctactaaaaatacaaaaattagctgggcgtgatggcaggtgcctgtaattccagctgcttgggaagctgaggcaggagaatagcttgaacctgtgaggtggaggtgggagtgagcagagatcacgccattgtactccagcttgggtgacagaacgagactctgtctccaaaataaataaataaattgataaataaataaattaattaaagtatgtttttggagataagcagtatgttttaaaataagggcatatacttgaaacaactctggaaatttatcataagaaattcagataatacttaagcaactaaacaaggatgtttattgcttattgtttagataatagcaaaaaaaaacaaaatcggattttctatcaagagtgaattcgtgtctttaattacagacaaattatatgtcatataaagttacatattgagagatatcaatctgtctatatctcatatccacacattataataccatgaagtgtttaaaataaagcaatagttaacaagtgatcatggaaagatgtctctgaattaaagaatctcaataaatgactttggtaggcataattccaagacatccttcacagttccctgccacctggtgtacatgttttgcctaatctctaggattgttaatatgatcaattttacccccaggattaggttatatcatgtggcacaatcaactttaagaaagttgacagattgttactggctagaaaacttagagagccacaaggcaaggaatatgggtggcctctaggagttgcaaacatctctggctgacagccagcaaataaatgaagacctcattcctacaaccacaaggaatttaattctgccaacaaactgactgagtttggaagcaaaatttccccagagcctctaaatgaaaactcaacctgtttgacatattgaattcagctttgtaggatcccaagcagagaatcctgtcatgccaccctgccaaacttatagaactgtgaaccaataaagagatgctgtttcaagatgcaagtttgtggtagtttatacacagcaataaaaaactactacaatgccaaccagaataaataaaaagaaaccaatatctagatactgcagaacaagaaagagaaatgaaagatgtttaaagtagctagaaagcaaagaaaaattacctacaaataaataagaactaaattgagagttgacttctcaacagcaataatgaaaagcagaagactgtgaaagaatattttcagtcttctgagagaagataactcagcctggtaatacatagccagccaagccatccttcaagaatgacagcggaataaagatattttcaagaaaaactgaaaattttcaatacaaagctgaatattttcacctaagagtcactgattaaagagtatattccaggaagaagtgaaaatgatccaaaagaaaaatctgaaacacaagaaagattggtgagcagagaatgtgaggaaataatctaagtgaacaaaaatatgtgccaaaatttgaacactgactatataaaataataatagtaatgggagggaatgataaaaatcaggctagtgataaaatactggacagtaattatgtataaaccaggaggctgtgattggtaatttgtgcttaatttttgtaaagtctttgcaatgttatgaaagaatatctgaggtccaattttacttggcaatacatggatatatagaagctttgcttgatagagcagcccctcagatatagtgaagaaatcctacagatcaaaaaatatatataccacaaactgtaatgcttaccaaaacaggaaaaaaagacagattatggcaggggattttaaagtcaggagaaaactaataacttccctttttaaagttcacctaattgaaagatgaaacaacaaaacgaaaacgcttttcttgagttcacccaactcctcaaaagtgaagtagtaaccaccatcccagatcagagaactgacatcataaaccttcaagccaactcaggtttcatctaccctgccctacagaattagacatgtgtgtgtggatggtgatggttccttacatagtttttatctcccaacattcaccaaaactgtgtgaatctctctgaagaggcactagaaacctcttgtgaactagtatttctcaaattgggatgtgagagattgcagtttcctcgggaagtagacatccaaactgagatgctctggctttaccaactatcaaagttgatacaaatgattttttttatacagacccctcgaacgaccaaataaatcatctgctctctcaaccagaaacaaataatctaagtgaacaaaaatacctggttgaaataagtttagtgcagggttatattctgcaccttaaaattgtgtttctgagagatattcctcattgattcataaattcgtcaactttgtactgttcagggtatctggattaagcataagctttggagtcagacagcctgaatttcaaaatcagcctcataacttacgtaactatttcttagccccacttagcctcggtttcatcatctataaagtagcaataataacacctgtcacacacagctattttagaaataagtaagatttcagaaatcgatttcagaaagagctggcaccaaggaagcaatcaatcagtgtcagctatgagtattgataacactaatggaggctgcatctgtgtgactaatctgtaccaggggtttaaatatcactaacattggactttggaatgcatagacacataaaatgttggagtttgcggaggcttaaagttcacccgttccaattctcattttctgtgaggatttgaaacccagacagactcaggaactcgtacaaagtcacacagccagtataaggaactgaaatctgaccctcagccactgagcaccaataacactatttcttctgtgtcacagctcatgtgtcttcttttttaatcttaccagcaaataatttgtgaggttaataaaaattaaaattgaaatacttttcattgagcacaccccagtgaaacggaaggaacaagggaacatggggtctggccaaagagaaaccaagtttgaggaagaacgtgtttggccttcctaaatatagttcaccataatgccaagcaaaggaaaacaaagaggcaaaagattataggctcattctgatatctcgtaggcctatttattaagtacatgatcaatttcatggacaaataatcttatgtttttagcaattgtattttctggccaaaaattctggaaacttgtgctgacaatgagatgaaatattttaaatgcctattatctttaaaaattcaggttacaaaatttaatctaccaaccatttctatcactgtatcttgttttgaccccacccccattccccattggagagatatttctgaaaagtgatttatgggagctggccttatttccattgctttaactaccttggagccctatcttctttgtattctctaaaatgactgccctttctgcacagtcactaaatacctattgagtagcccatggtgtccttctgatgaccatctgatgggaaaacttggacatttgtgctcattatgaaccatctgcccttgagtccaaatttagaatatactaatttttaaattgtatttttcaacccctatagctggtgtaggttgaattcagcaaagaatttttctattctgtctttggggaccctccagcatcttctcattcacatctgaaaggacgtctcccagtccacagtgcctgtgattgccacattctggtgagagctatatggcagattcaaaaatcattgcgcagcctgtcagctgattggggtgaatgatcaatgtaaccggaactaaagttgtatgacatcatctttgccacagcttcacgtgacaaagctcacttcaaaagtgcttctttctcagacaaagccctttagtcagaaaagccacaagtaacagttaaaatcacctggtccaaccatatggaaagactcaccaaggttcataaaaatgaaaccacccaaagtatatgaggtcttctgagtcctaatccagggctttgacatggcatcaccaatgcatcacatcccacatggctctcacttcttccctggcacctctgcaaagttccaggggatttcctgcctctcactcttctctcctcattctctggactcaccctgatctcatttatgtatgcttttatcataattttctctaaagtagccccaagtcataattacattaaaacctgaagtctgataaaatagtgaatataaaaccacagcactgctgtcaaaagtagaagagtgatagtgagcttatctccagggaaatggagaagtggtcaaaccacactgctatttttacctgcctggtattatttgagcctaactgaatgtgtggtcacatggacagtgattcagagatgttaggaaattcaggtcaccatttatttaaagcaactaggttcaaaagatccttattctttcttctgccatgaagttacatatcctgggagtgtaaatgaaccagtctgggcttcattttactcatctgtaaaacgaaggatttggactacttgatatattatatctcatttaatctaggactaaattctattccaattgaagggtgatgtagagggaaattggaaggctgatgaaagagtctgaggattaggtagtgtagtgtggaggaacaatggagacagaaagtcttccttcaaatttagtcttcagctgcattacaagccaagaggctttggtcctgacaccctcagacctgctctcttatctcttctgtccctctacttattagaatgtgtaagctgaagtcatgcttttgaatataaccatattgataaggaggttagaactgtcaatccttcacacatatatgaatccatttgagcaaatgcttaaggtatttcttcttaacatttggtgactcttttgattatgaatttagttatgaaaactagataattctggatatcacagaatgaacaggtctttatagtaggtcacataggctttggcccacaagagtgctatctgtgtagtgagacaactctctaacacggagaagcagcagaaacagagtttgggatatgcctcacatgacctggccatagaaaaacatgctgtttttctaggaacatggataggaaccacagcccttttcacaggactccttcttgttgggctttgggcaccaattctgcctggcctgggccaagtccctgagatgcagtggtggcaagtgagggtatttagtaatacctgcagcccccttggttccatcttcttctgaaaggcaggagcagaagagcctttttcctagcccaattcatggaccacgaaagagttccagagtccaagcaactgacagtatcagagcatctgaaaagcatcatgtgtttttatgattacaagaaggagaaattcattggggctagctaatgtaacaaaataaaaataaaaaggactctaatgtgtgagtgaactctgtcacttgcctacccagttattcattttcttctagcattatgcaatggacaataattggccttagccagggaatgctttaccctcccaacctctctttctgctagaggtggccatgcaatccagtgttcaactggggaagcttctgagaaagtttttctttcttggtaaaaggaacaaaagaggctggttccatcagtttcctcttgaactctgactattgtgactatctagatctgccttatatataaacaccgcatttattttctgtaactaatttctcatttattcataaagaaatcagacataggacataatgagtgtttatggtcccacttaagctgagaaagctttcaaatggcataggagatatctgttcttatactattacaaggtcctccataaccttcaccttaatttcatcataaaaatagctaacattcattactaacttcaatgcatttgacagtcatcattccatctaagcttcacaaaagtcccatgagatatatattattgatattcccattttaccagtgaagaaactgtgtcaaggaaacaggtagaaagcctagacttagattgaaacccaaagcatttggttccagagtcgggaatctaaaccactataacatgaagccacctgcatatatattaaacatttcagttactatccctaagcagactacttctgagtgttgtcctgggagatgatcttctattcctaacctcctggcccagtcttcagatgcagagttgattgttcttcagcttgcccatttgtgcagatgaaagcaggagctgaagtggggctcttgcaagggctactaaaggctttgccttcactctgagtgcttcacatgcccagacatttgtacacattaagcacttgaacctagattaggacaggctgaactcatgggaactcacaggtagaagatgagctgcccctagttgcttctttgcaaagagacaccaacaagatactggattgttcagcatcttttccttctgaagggaataattactttatgggtgatgagaagctcaaaaaaaaacctccccatctgtatttatatgttgcaaaatgctacatgtctctatgttacaggttctcattcctcagcttaaaatgtaactgtcccttataagataacttggaaatttacatctgaaatgtgcagcttaatgtgaatgggtttatcaggtcctttgataaaagctggatttggttagggagaaaaagaaaagaaaaaagatgtacctttaggtagaaacaggaaagcagacaaattttgacttggttacaattccatctaagatcagcccgagtcacatgtgaacaacaggactgagaagttacctagaacacagaattctcagtactaaaaccagtatagtcccagtcaaactgggatggttggtcatcctgtcacagattcacacttttccatctgcctagttctcaggaaaacttttcaattatccgttctctgaagccaattcaacagtcccttactaggttagtagataaacaaaccgtggtagatccaaacaatggaatactggtagcaataaaaaggaacaaattgttgatacatgcaacaactcagatgaatcttaaaggcattatgcttatgaaataagtcagtctcaaaaaattgcatgctatataagaatacatttaaaagacatcctaaaaaaagaaaaaaaaaactatagtgacataggtcaatggttgccaggggttgggaaaagggtttgaatttatattcctaaataaattacaaatatatatttgtatgaatttacattatgttttagattatgttttacaaatatgtatttttatgaatttacattcataaagaatgacatgagaaattttttcaaagtgatagaactgtgctcaatcctggtagggattacacaaatctgtacatgaaataaaatccataggattttataccaaaaggaaaaaagtcactttctgtgataaattttaaaataaaattcatttatgaagaatattcctttaaagaaaatgtttattcacacatgcatctatggcgtaggacatagatgagcattttctgtacaaggaataagaaattctgagttcttgacccaattccttttaactttatgcctaaactttggcaagttgctgcatttcagtcttcagtgttctaatcaataaaatgagagtcacgagctaatttaaagaatgtctgacccagctttaaaattctaagaggctgtaatcttgcaccatatggaattccccaaattgtttgtcttaactttagcgaagactaaaattagctaacggtggcagttggattgggcagacatcaaatgagggtatttcctaattaaagatactctcaaaggagaagtaagggcaaaagtctttcctaccagagaaattttttttaaaggcaaacctcaagcttaataaactttcctaggcattaaaccgcacataggacccagtagaaaacttattcaaatttttaaatcaccttttccccttcttgctcaaaataagaattacaaattaaagggaaagggaaacaaaatcacaactctttctgcctgaaactaagatttatcattttggttttgctactgcattttagaaagggtgataagaaagacgggacttttttttatgctgcataaagagatttgaagttactcaaatgagtgtgtattcaacctgaaagcagtctcatagcttcatattgaaattccagaacagaaaacattgctgtgtcagttacactggaatgtcccttaaaatttttattttcacttctaattgagaaattcctgtggaatggatggaggacaaaaagttcaccttacaaaggactttagaaaaccctaacatacaagtatcttgctggcaccaggaaagaatttcaaagatgattaaataatgggaagtgtggaaaatacaatctcttgaaaatattgttttattatttaaaaattttgatgtcatagatttactccaaattgtaagtaaacactgaatagagtattaaattttatatatggtgctctgattctctcaacacttaatatttaaaagcatttgagtatttaaaaagacctcaactaaatgtcacatgatcttaatttaatttcacattaagtcactgaattgtagtgtcataaaagcctcttcatgtgctgtattgggttaattcagtatactgaatttttctgcaataaaagtatcttgtaatagacaatttgaattttacagcagaaatgaaccttagaagccattgcttcttaaagctattgtgttttaaagttgtaatcgaaatgcaaagcatgttttgaactaagcaaaatatgattccctaggccattatatcttagaatctcagagtttaaagacctcagccatcatcaggtacaacctctgagctgatacagaaatcccactgtattcctttttctggaacacttgctctaccaggctgctcagtgctacctactctgcacaagatcaatgtgacataactccagtcacttgacactccttcagatcacttagactagatctgtttcccaatgcttgcatcccaggacttgtctctgtcctcaaggataccaaagatgttatctttcttgttcacgtttgccctatggctataaaaatagcaatcatgtctcctccttggttttctctttttcacattagacatcaacttccccctcaactagccctcatgtgacttgatttttcagcctctttgcatcctgcctgccctcctataaagaaatcactttaggttcccaatggcctctaaatactgtgtacctagaccagaatacagttactccagtattttcaattggcccagagtccctagctcaaaatgaacaatatactactattgctgtggtattgatggtcctgctgttatttaccacaatatttcttaacatgagatttctgtcaattaggaaatgtccttctctgtaaaatgtaactgatacatgagatcagcagaggagctatttcatcagctgaaagcccatatgatgaccagtattctaaaaagccaagcagggagttgtctagttaaaagcaggctggggccaggcacggtggctctcacctgtaatcccagaactttgggaggccaagatgggtggaccacttgaggccaggagttcaagaccagcctggccaacatggcaaaacccccatctctactaaaaatacaaaaattagctgggtgtggtggtgcacacctgtagtcccagctactcaggaggctgaggcatgataatcgcttgaacccaggaggcagagtttgcagtgagctgagatcacaccactgtactccagaaaaaaaaaaaaaaaaacagggtggaaagctcccctcagcacaatctccaccttcaggcttctcaaatgttcaacggagcctaagattcttgttctgaatgtccagccccagcctcatggtcctctggagaataaaaaaaactttctaactctgagtgttacttgcctctaaaaagctacggctggtaaaataatttatattcccttcttctaggagtggacaattaggtgttgggtattatactaacacctaaatgtcgattcctgtcttcttctatgtgaagagagaggtttttttgttgttgtttctttgtttgctttttggttttggctgctttcttgttgactttggaaaccactttcccaccggaaaattttgaaacaggggccatgtaaaatcaatttaattctgtctctggtttagaaagaacatgaccctgttcactaataattaagaagaaagtttggggcgggggggggggaactaaaactcaaatgctaaaaatggttgagtaaaataagatctacaatatgtatatgtttggtctaaagaacaagggacaaacagaaagggggaaacatgagagaaaaggatgaaacaaagaaggaaaattaaaaggcagagatagaaaaacttcccacacaggacacacagaaaggaaagggaaagagggagagaaagaaaggagagagagtgatatggtttggctgtgtccccacccaaatctcatctcgaattgtaagccccataatcccaggtgtcaaaggagggacttagcattaagctattggatcatgggggtggtttctcctatactgttctcgtgatactgagtgagttctcataaggtcttttttttaatgacataaatttatttttccagttctggagggtggaagtccaagatcaaggtgctggcaaggtagacttcttctgaggtctctatcctcaactttcacgcagctaccttcttgctgtgtcttcatgtagccctttctttgggtgtgtgcatacatgatttctcttccttttctttttttgaatttttattttttattccaataggtttttggggaacgagtggtgtttgattacatggatgggttctttagtggtgatttatgagatttgggtgcaccactcacccaagtagtgtacactgtaccaaatatataggcttttatccctcaccctcctcccaccctttcccccgagtccccaaagtccattgtattattcttatgcctttgcatcgtcatagcttagctcctacttatgagtgagaaaatatgatatttggttttccatttcagtgtggagattctaaaagaactaaaagtagatctaccatttgatccagcaatcccactactgggtatctacccagagggaaaaaaggtcattatacaaaagacacctgcacacacatgtttatagcagcacaatttgtaattgcaaaattatggaatcatcccaaatgcccatcaatcaacaagtagataaagaaaatgtgatatagcatgttctcacttataagtgggagctgaacaatgagaacacatggacacagtgaggggaacaacacacactggagcctgtcagagtggagtagggagagcgtcaggataaatagctaatgcatgcagggcttaatacctaggtggtggattgataggtgcagcaaaccaccatggcacatgtttacctatgaaacaaacctacacatcctgcacatgtatcccagtacttaaaagtaaattagattaaatttaaaaaatacaaattaagaaaaaaaataaataaaataaataaaatgtggtatatattaggttggtgcacaactaatttcagtttttgctattttttttaaaaagtaattgcagaaactgcaattcattttgcaccaacctaatatataccatgggatactactcaggcataataaggaacaaataatggcatttccaccatgccattctatccatgctggatggaattggagaccattattctaagtgaagtaactcaggaatgaagtaactgatgattttataaatcgtagtttcccctgggtttttctctctttcctaccaccttgcaaagaaggtacttgcttcttcttcaccttccgccatgactgtaagtttcctgaggcctccccagccatgcagaactgtgagtcagttaaaccacttttctttctaaattacccagtctcaggtagtatctttatagcagtatgaaaatagagagcgagaaagagagaaagagaaggaaagcggtgagaagaggctgggagggacagcagaaggatatgggattctagcagagaagcattaatgtgatgtactttaggttctctccacacccatccaagagcaaagctctgatctacattccaaaggttcatgttagtgagaatcagacctcccagccaagagccctgcccctccatccctgccaggagagaagtgttcatcccaatgctcagcattcctctcatttgctcacatacagcatgagaaatgctgacattttagcttatactttaagaaattctgggtttcatgagattttcactttgttaatataatataccaaaaaagcctccagtttttaaaagataataattacagtcattataaatgaaagtcacatctaggatcgcatctccaattcctcattttacaatggagaaattgaggctcaaagctgaggtgacttccccagggtttcacaacccagccagccataaactagaacaaggaaggagcaaagtgtcagtaccaacaacgtgagagcaaagtcattttctgaaaatgtacaccattgaaacagcccaagtcgttgtcaccttgagttttctccaccatacatctgcaggcttggctctgtcacatcaccacttcctgatgtggggattgttttcctttcaagaataaataaacagtaagagccaataaactctagaatgatgaatgccctcttttcttaatattttcttcccagaagatggaatatgacacacaaggaaaattcttttcaccagaataataaatatttatcattaagatgcaaacaagacatgaatgaaactcttggggtgattttactttctgtcagtgcaacattttctgcaagtctgtgaggcagtcagcccagagtggacaaacaaagctgactgattgattctgaacatgagtttggccactaagtagcccacgagcgatgggactgctgagacatggtgctgtgttatttgttccactcaagcaaattccaggcattagcttgcctgccccaaacccacctacacagtgacaaattcaattcccacatcaaccagaaataccaggactcactcgctcctgcatctcaaaagagactgtccccaatttctggagggccccagtggagaaagagtcccaccgcaatagaagtcattcctgtgagatgctataagctgctcagcatcacccagccatggctcaagatctcactctactccccaatgcaagtatgatcatttttccttttttttttttgtatgcttaagctcaacgacaagaaatgtatggtctttgtatcatttctaatccaaacgaactcttttcatcccttacaaacacaccgaaaatcctgtgattctaatcacagaactggaaaaatccaagtcaaatgattctcatctaactgaatggagatgaatgggttataagcacttattgaacacccaccacatacaggacaccctatgaggggcctgcactggcctgacggagagagtgaatgatcattacatcctaacataaaactgctgcaaccaaattttgcagatgttcattcaggttagttagctgggctcttggcctttgtcttgactgcattcttgcccttttgtgcccattgttcctattaacacctctttgagaaggtaatatgaaagaatttaaattaagcaagacagtttaaagaaggtctttgttaatatgaaagttgaagattatatctgaatatcattaacccttgctatttgaatcctttattactgccgttggtttgatttggtggtagcataggggttacagaacattgcctgtggcagtgtaacagcactgcgcaggtaaagagagcattactcgtgcagcttctaatccatactttgggtctcagtttttctacttaaagaacaagcattcatgtttctttctcccaaagattattttattattcctgaggactgtgtctgctaagcatgtcaaaatttcccaacattagttagaaaacctgctgcctctttttgccttaacaatccttcacaaatcagtagcaaacacctagagaaattacctttataatctaggatgcaaaagcaagggaacatgggtgcaggtggggaccccccccacagcagaggcacaggtacctctggtcacctcagaagtacaattgcaatagattgacttagatgccatcttgctaaaggcaaggaacatactaggtggcatgtgataaattttctaggcttataaacctaattcttgatttcatgcctccaagagatctgaaattaaagagaagaagttgggtcagcaaaattgaatttcgtggaaagtacagctataggagtgatgcacttaatttcctttactgaccattctattacaaagaaaatggataattgtgattcaagaagctgagggaaataaaatggaaaaaaggtagtcaacatcttctaataaaggtcaatattagaaataacttcagctaaccctttccttgacgtgataattcaatattttacagataaattccctgtagttattttgcccgctaaaatatattaagctcccttccatgcgcatcacacagtgttaaatacggaaagtcgaaaacaaaggacaggggaaagaaattgatccctgacttaaagcaactgcatttttccaatgacaagtttatatcaattggttagtgtttctcaaagtgtcatctatggatcctctgaccagagtcaccataagagctctgcagaaatacaggggctgggccttacctaacacctagtcatttagaatctctgagagaagggcctctttagagggttccctagagactcttaagtacagtaaagtttgagattgctgccataggaacaatcacaagtctacatcattcaggagatttgagtgcaggcctagatgagagaatgactaattaccttggattaaaattcactaagaatctgactttccatttcctgtcttatactgaagctcacattccaactgaattcctttggctcttacggatgaaaagaaattgctgtatattcctgaattacctttactaatagttcagccacactcgaagacctgaatatagtagaaacttgaataatttggacctgctaaaggagaggtcacatagcttggaagcggtcagaatcagcagacatttggcctggcgcagaggctcacaccagtctggccaatatggtgaaaccccatctctactaaaaatacaaaaaaatatttagccgggcatggtagtgcatgcctgtagtcccagatactcaggaggctgaggctggagaatcacttgagcccaggaagcggaggttgcagtaagccaagatcgtgccactgcactccagcctgggtgacagagcaagactctgtctcaaaaaaaaaaaaaaaaaaaaagaatcagtggacatttgagattttttttcctgtggcataatagtaatgtattctggcttacaaaccaatgccgaattttaaatctgagcttttggccttgatgtgattgatatgtactgaaaaatgttcccaccacctcacttatcccctggtccgtcattatcaattgttatgaaatttgttccctctggtttaacaagaattccggttccaaaactgaataacttatggcacagagaaagaaataatttaaagacacaaaatgccagccactgatttccttagccccttttcgtttattgatcatcataactgtgggggtagatgttattagtcctatttatatttgaagataggggatcacatgggtacaaaatgataaagcagatgcaatgtcaggatctccacccccagacgaggaagctgtcttaattgcactaggcagcatcttctttcaatgcaaataggctgaagaaagcaacatgtttgttacactagggagtaaatgacataaagaagaaggagaaggagaataaaagcaaaatatcaggcaagtaaggagtctctggagagtggagggaaaagggtgtcagcaagtggaacaagaataaagaagggaaaaatagatgggcacccaaaccaaacttcttccatttgacaaattttgtgaagacttttctgacagacagaggtccctccatgctggtcttaaagatttttaaaaactcacttagcagcagcagcatattgtaacgaatatgtcaaagaatcccatgtactcatacccaataaaattgttccctgttaaaataacattcttccttgcaattttggtttcactattgctgaaacatggtaaaaactgtttcttgtaataccaaacattttgctctgctctgtagctgacatgacacttgtagcttccacgggttgcttgatgaataagtacaacaccaaaccatcagatggtaaaggctcttctccacatctcatgtctcagctgccacaacacaaacccttcaaaggagacacattcccagtttcatgctccaagtgagacagatgctccccaaagccactacaccacctcagaactagagaaaagaagtcctcagatttccttttgttcctgagaccagcacactgaggctcttttactttccccttggccaccatcctgctgagagctgcaggagagaatgtctgtctagtgtccccatatccccctgtctggctatcaaatgtaaagccaccagacctgacatccctgttgtctattaaccagaaacctgagctccatccctagtactctgtacatttcctactctgacccactttcttaatcatcctgtacctcccaaacccctccagtgtttcctcttgtctgccatcagcaaacactcttgttatgagttgaactgcatccctctctccgttcatatgttgacatcgtaaccctcagtactttagagtgtgaccttattgagaaatagggctgttgcagatgtaattagttaggataaggtcatactgcagtaggatggccctctgatccaatagaactagtgtccttctcaaacaggcaagtttggacaaagacacacacacacatacacacaaaaacaccaaatgaagattggagttatgctgtggcaagccaaggaactaccagaagttaggagaaaggcctggaacagacctcagggaagcacagccctgctggcatcttgactttggacttttggccttcagaactggaagacaataaatttctgttgttttaagccacccagtttgtaatactttgttatacctccccggaaaaagaatacaacccatctttgagcactcccttcacctggctccccactagtggcacagcttctcttgcagtcttactgagggaagatttcttctccttccccttggtacctcaaagcctggagttagatcccattgctccttctaaaccatttcttctccctgtgctcaaaagcccagatcctttgaagtttaagccatcatactctttctacttgctactggcatctacaaaacttggtcactcttcatcacttattgatgactttgtcacctgttctataacttcctttctacccccactcctggcaaaacgtttaatgacatcaactctctggtgtcttggttcctctacattctcatgtccaaaagttgtttcttccattccagttcaatctcctattccttgttgtcatcaccattaactgtactaacttcagaatctcagcctccagcttccccctctctgaacaccacgttctaatctttgagctcatttactcggtacataatctctcaaaattctccactttcagttatggagacttccaatcaactgaaaggtaactgacaccaaacatttcctcttgaacacttttcttatgtcttatcttcttacctagtttgaagtccactcatccttaattagaattacttccttggaaacacctttcactctctctccatttttgtaccagcacatctacttggcaatgctccaacccaggtagtacccaccaatctacctattctgggtttctacctaagaaattaaacttgctggactaaaatatacaactatgccttcagatttccctttaaagtcatgactacgaaagtatatttaccagctgtcatcacttcacacttctagtaataacctctcattccctgaaaagttataacactggattgtctttctttccatcgcttttcctatgaggtattggtgattgtaatattcaaataactaatccatttaatagtatagcatgtcagtttcttgattatctcatttttgttgactttgtcttcatctctacctccagtacccacgtaagtgactatatcctagatcttattattgattgagatctgcaatctcaatttgaagccattctctaatctctaacttccaatcttcccaggtcatgttctcccacatacttactccaaaactctttgggccctactgggatattcagtctattaatcctaccacctacttttctccaatccctccttttcttcttaccaaatgtagattccaggatccatcatttaaccactccttctacagatcctcaactagcttgcttcctttgtacctgtctgacaaaacgccaacctgctaaaacctaaatctctccctatatctgcagctgagcagccaaatgtggctagacaaaaacacaaccatgctgactcttctttcaaatatgacctcaagcttcaaaaacgttaatattgcccagcaattctctatattcttctggcttttccactcttcagacacctcttttacatctttttttttttcctgtccctaaacctctgcattggctctattcttactctcagctgaaatatttgcctcttattttcctaatccaatcgaagtatttaggagataattcccatatcctacctactataagtttatcaaactacctacatctgtaccttgtctcctgttgcaatggatgaactgtccctgctcctatctaaagccaacccctccttttattatctagattccatcccattatcttcactcagactttgctcctacaattatcccctctcttaattgcaacacaagttttcacattgatatacaaattgctcccatatttaaaataccctgcttgatttctttctagcacctggcccatttttctattcttctttgtagcaaaactcctcagagaggatgcctgtgatagctgggttcctattctcaacacttctagtcaggatattgattcaagatttcactaagctcactcacatcatcagctccagtcatcaatggttggtccccacacttttccaaccctcagcagtactgagtagagttaatcatatccttaaaacattttttcccttggttccgagggcgttgcaccttattgttttacagcctacgttgcaggctgctccctttccaatctctttgcaggatgctccttctcttcttaacacaaagttgtgttaagattagggcagctattccccaggtgccctaatctagtccaatagcttaatactctgtctgtatgatgatgactcctgaagtagcaaccctgatcactcacttaaacattagatatgcacgtccagtcacttacaggacacctcttagtaggctccaaacccaactcttgattcctactctgcagcctctaaaatgtctccttcctttgtctctcagtaagtgagcaactaccccatttctaacccattaatccagccagacacctaaggttagtgttcgatgttttccctttctcagactctctacattcaatccatcagcaagactgattcacagtactatctcaacacatacgtatggaatagatgaatacatgaataaataagcaagtgtatacaactgtcctgtgccaggagcagaaactaggaaaaactatccatcccttacagtccaaagattcaaataacagcattcattcttaacatggctttttaataactaatgagaaaatgaaacccgtaataaagtcatatgccaagattcatagcaagagtcctcacttctcaaatttgatcacttccccttaatttactggcacagcctgcccagtgttgctattttgggggatccgatttctcttctgctaaccagtttaaataagtcctaaaattatgcgttttaagttattgctctccaaatataatttctaaacttctataatatattaattgccagccattgataactactcaggtaaaaatctggagaacaaaactaattaaaccaactttctagattaaaggagcgatagttctggtgtactagagatgaaaaacaattaaccatatcaaattatctgtgggaacaacctctgagagttgtgttttttacaacttaatattttaatccaatattttctatttgcttttcaaccccaaattcaatttgaaatgtaaatagttaatgtttttaggaatgggaaacattaattcacttattttctggtacacaaagaacattagtgcctataaaaatgacttgggtcattgctttttcaataaaagagcaaaaatttgagccagggttgactcacatatccattagcatcttaggtttattgactgtcatcccattcctttcaaacgcttttgaattcagagagtactcaccagacaataagtatacaaatcactcactttaataaaattttgttgaatacctagattgtaagtttattcctcacactaagaagtcagtcagtcatgagacagacagtcaatcctgacgaggccccaccacaacacactcagtggttataaagggcaacaggaaagcagaaagcacagcctaacaccactcatggaaaattttatagcacatgcactctttgcaggaagtcaatgtaaatgctatgtaaatactgtattgtttattgtaatactgtaaatactgtaatactgtattgcttttaatttgtattatttttattgttatgttattcatttttatcaacagttttttgactgtggaggtggaacttttggatacaaaatgctgattatatgctgagaaaggataaagaaggaaatgtcttaaatagaagaaaacataagaagatagaaactgcagaggacagaggaccactgtatgccctgaagacatcactaaagtggcacatgaagcacacttttgaacactcaggaagaaattcctgacaatcaatggacacagaaaaacattattaagtaaaacttgtggtgactcttttgctaggcagtggggcatagtggttaaaaatttgggacttgggagccatttccaaaaagaaaaaaaaataatgaacaaaaaagttcgtggagctgcttcctgcggaaatcatgaaaaatgccattgaaagtcaaagccctgcactaataactaattatttgtccttgaacaagttatttgtgaatacagaacccatagatacgaaaggccaactgtacttattgaaaacaatccaggtataactagacccgcacagttcaaacccatgttgttcaagggtcaactgtataattcacttaatccctagagcaactgtaagagctagatacgattgttatccttgttttgtaggagaggaaagtgaggcacagaagtgtggttcaaggaccagaagcatctgcatcacttgggagtttgtgagaaatgcataatcttaaggcccacctcagattgctaaattggaatctgctttttaataaaattagcagattcctacaaatattaaagtatcagaagcacctggctaaaaccatcttcccaaaagtggtggaaggggatttcaagccttacaggtctcactctacactgtactgtataagcctctttgcattagaaaattaatgtaaagtgcctagcatattataagtaataaacattgtccatcattataatattttaggaatagtttgtacatatttctacctggaagtaaaactatttatttatttgtttatttatttatttatttatttatttcaaactaaaatttaaaaagctgaaattatcaccaaacacttagcattgacaggaattcgaattgcagaattatagaacatcaacacttaaagggaacagtaagatctaagaattcaaatctcttgtttcatttctgtggaaatttgtgctaaaagagatatgaagttaatgtgacgaaagtcagccagcaaattaggggcataggaaggacttaaacttgggtcttggtagctaggtctgcaatcatgcaattattactgacttctcttcattccctgccactgcataggtcaaaaaaggcaattggataagtaagcaattcgcagatccaagtggtttcacattactgtttaaagcgctctcccgaccacagaggctacttgtgatgtctatgaatataaaatatcttcataatgtgttttttcagaaatcagctgtttactggttctgtagtagattctctcctgggtatttccttagcatttggctagaacattggtgcaaaattgatgtggtataatattgtaaactttgggcaatagtgtaacctctcccaacaatccttttttctatccaacattaattcttatcattctatccatcttcataggaaattttttaagtaacaatgttttagtcttctctaaattacagcatatttttccctgccctcaagagtccccatcttataaatgtttgcctttcggttagtccagccaaattttatttttttgcctgccacctgcattctaatagtccttttgcctcttatgcacctactcttgaccattcatttctcctcttatctctacagtgttccaagataattatgcaaagtatattcagatcactccttcagcaggctactgcattctttcttcaaattgctcaaaatacatttctcccaaaaagtattttctttacttgttgagaataaccaggaattgattcatattaggcattttattcatctttactatgtaatcagcaatagaaatctgataggcatgatccctgacttcatcttctacgcaatattctatgtatatataccagcttgcctttagccttattactgagttttaattttgatactgtgatagttaactacatgtgtcaacttaactgggccacaagatgccctgatattttaaacagtattctggttgtgtctgtgagcatgtttccagatgagattaacatttgaatcagtagattcccctccccaatgtggtcgggtgttatccaagcctttgaaaggctgaatagaccaaaaaaaaagaataaatgagaattcactgtctctgactgttttagagctgaacattgaccctctcctatactcagactgggacttatgccatcaattcttctggttctcaggatttggactggaactacactaccagctctcctgggtctccaatttgcagatggcagatatcatgggacttcttagcctatgccttataataaattgtgtgtgtgtgtgtgcgtgtgtgtgtgatacatatatatatatatatatatatatatatatatatatatatatatatatatatatgaggtatgtgtgtttgtgtgtgtgtccgtcctattggttctgttcctctggaacagatcagactattagagatagattatgtgccttcacctactcactgttcctgctttccttccatgtgctcactgttccctgtgaatggctcaggatttgctatgggaagtaattttgccttcctatgaaaagtagataaagattcagatccaccctttctaagttgtaagtatttttactttacgtgaacttcctttaatgatttaaacaagaagtggaaagcttaatgtatgctcaactttttaaaaacttaacagaaaaaaattggaacactattgaccaaaacttttagcactttggttatctaacggtgtttccattatgggcgatgttaatttatttatctttgcttttctatatatttcaagattaatcaaacagtatattaatactaatttcgaaaagaccaaaatcattaaaaaaacagatcgttttgattcatactgtcctctagttggtaatccccaacaaatcagcacaaatatcacaccaaaattagaacaaaaatttgttaacaataaaaaagataaaaacccagcaaatctacaccaattaaaaatagatgatcacaaggaagaataaactagtttaagataaacctacataggctactcacagcacattccaccttatatcatgaaacagaaatcaccaaaaaccaaaagataagatctttgccgtttctctccatatgtcctcagattcaaagatccaagaagttacaactgaagaacaaggtagggtttcctttctaccttctgtattctactctgtgaagaaagtagtaactatgcccatttaccatttccaccaccagatggtataatcatctacaaaaaagaaaaaaaaactcatttaattcaaagagtgagaattatataaggtacatttttctcaccacaaagcagaattacaatactttttttttaagtactgtttggaaattagaagaggaaaattacctttaaataaacttggttagaataagaacccgaaactataattacagatcatttagaaaacaccaaaaataaggatacaacatgtaaaaacttagtaaatatggccaagctgtataatcaaagaaatttactttaagtgattttattattaatttctagaaattaaaataaattaacaaaacaatttgctctataaattagagtaaaacagaacaaacttcaggaaaacaagcagaaattgttaataaagtagaaatcaataaattaggaaatactgaaatagaggtaaattcgagtttataaacaaattcaaaagctgtttatttaaaaagaacaatagaataaacaatttagacaaatataacttaagaaagacataagagaaaaaacacagacccctctgaaattagaaaaaaagatcaaactacaacaatgaagataatttttttaatttgaaaaaaagtagatctataatcatggaataaattggggaaaattgtcaaagaaatttttcaaagttagatagacaaataatttctggtctttcaagtaacagataattactttgctatttacattcttaaaaagtatagaaaaaagagccaggcgcagtggctcatgcctgtaatcctagcactttggaaggctgaggcaggcggatcacgaggtcaggaaatcgagaacatcctggctaacacagtgaaaccccgtctctactaaaaacacaaaaaaaattagctgggtatggtggcgggcgcctgtagtcccagctactcaggaggctgaggctggagaatggtgtgaacccgggaggcggagcttgcagtgagccaagatcacgccactgcactccaaccactgcactcaagcctgggcgacagagctagactacgtctcgaacaaaaaaacaaaaaaaaagtatacaaaaaagatgaaatgccacttatgccatttaatcttaccaatttacaataatctttgaatagaaatctgataaagcagatagaagaaatctacaaacttatctaactaataagtattaagtcatggtcatggtgtctttttactttcttttttaaagaaagaaaaaagccatgaccaacaattaatttcatacttacaatcaaaaaaccagaagtgtttctattgacaaatgtgcctgaaggcacaattatttatcactgttctgaaagtctgaacaaaaccaataaggtaaaataaattataagtttaaaagagggggagacagaatcattattattttcatttagtatgattctttacctacaaaacgtgagagaatcaaccaaagacattttaaataagctagttatctgaaaatccacaggttaaaatatattgttatctaataatcttttaaccattgatccagtttggctgcctggaagcattttctaagattgtacattctactttggagggtaagtttttctctatcaatgtagtaagtaaaacatatgacctatttttttctacccatggaatgaagttgtacttggtaaggctatagctctaatccttttttcttcttcttcttcattttcatactcagtggaagaactcaggatccattgtcaaagttttctttgagtctgctgagaaaatcccatcttaaattgcattatatattcagcagttgtcatatattcatttattcattcaacagaacatattctaagtcctaaaacatagaaatacgcactaaagacacacagattaattttatctcactcggcttattatctagtagcagaaaaagacataggcataaaaatttaaggggacaatctcatataataaaagcatgtgctatgaggccataaaaaaaccagtctttacacttatggccctttggaatatgcactcaagggtcctcatatataacaacagaccactactatgtatggactaaggatagttttgaaaattactgagttaatatatcaactactccagtgaaaccaagtgatattttagtgttcaaatttattatcaaattatagtatgtgggcagctatttgctttaaaaataaagtttgctagactatcctttgaaaaaaactttggcctgatttgatttgctatgtgctgttgttgaagtatagggaccaactggattgaagctgctgcctaatcagcaagcatactgctggggcataatacacaaactccttgaattccaatggcattacctactaacccactgaaatgttgatcatggccacactgtgaggccagccttgagcaccctgggccaccgtgtgagatttgtgacaggatgcttgagcagtgaccatctgcaggaagaaatggggcaaccagaccaaggacaagtcaaagaaatgctttcagactctacagaggcccagtgaacaacagggctatgaactgagtcccagtaaccaataaaccaagattcagatattaaaaatgcagctgctcgccataaaaaaggatgagttcatgtcctttgtagggacatggatgaagctggaagccatcattctgagaaaactatcacaagaacagaaaaccaaacatcacatgttctcactcataggtgggaattgaacaatgagatcacttggacacaggaaggggaacatcacacaccaggacctgtcgtggggtggagggagcggggaggaatagcattaggagatatacctaatgtaaatggcgagttaatgggtgcagcacaccaacatggcacatgtatacatatgtaacaaacctgcacgttgtgcacatgtacgctagaactcaaagtataataataaaaaagagaaaaaaaaagaaaaaataagtaaaataaagatacacactgaaaaaaaaattctgctgctcttatttaaacatagtctctgtccagagcacaagtgtagggaaagggccacaacttcagtgagctttgcaaacgaagacttcaggcacaaagtacagattaaaattacagtgcagtcttgacatttgctcagtgtagaagaaaataaaaatacttgatttgacatcaatcatttcagcctcaaatataaaatcaatcaaatatttattgagcatcttatactctgtattccgatctatgttaggagctacagagatatccaaaagaagtgtaagatgtggtctttgacttcaaggtaattacagtctagatgataagtcatgtgctcccatagtacctaaagttatttttcaaagttctaaatataattataagtaattattcaactattttttaatctattgctccatggtgctctaaattccatgagctcaatcaaatctaatccatacctcatccaccactttacttccaatgccaacttcatagtggctgcataagaaatatgtcttggatagatgaataaatggatatgtgagtgacatgtaagaagcatttttaaaagttcggtttttttctgaattgggcttagtgtgaatgagcacacagaatggagagagctctaaggactggagtagtgggaaaacactaattactaccattaatatcatctataaatattaagaccaaatcaaatacacccaaatgtaaaagcttaaaatgtctactgaatgtgttcattacaagctaccactgtgctctggagccaaccatttggaaactgctattcaccaattgtctcctcttggctgttgtttcttccccctaccaatttgctgacaatagaaatacagttctattctttcatttccccacagtatagaagacaaggctgcaaagagtactaaatcttcagtgaatttaggggtatcatgagtacaacctcaacggccatctatgctgttagtagagaggagcacacatggcttctttgaaagtctctggaggccagcatcacctttcccagtggtccccaggaatctattgagaaagagttcacaaagtattctcaccaagcttaggctttaagttgctaacacatgtgtgtcttctaaattcacaggaaggttaaacaacctgctgtggtgatgggaatgctgttgccacacacataactgattggttttccttttttcttttggttaatacattttaattgtccctttttcgatatttctgagagaatccaaacaacagttcttacatttgtttgatttgcccttctttcagaatcactcctacattgcacagagacataaattttactttctcattttaaaaaaaatagctctaccttgacttccattttaatttctaatgtaaatgcatacccctatgtacaagctttggctctactgggtgatgagaaaattaattatatttgtgaaatatggcaaataattagtggccttccaattgaagtgctagtattcttggattatataaaaatgttttaagtataaaagcaaacatgggtaattttaaagcaaggaaatttccagctcctcaggttcatgctgagaatacaattgtagtagagacaccttgctggtccttcaccatcacaacgatacacatgaaaatccatagggatttttacttaagtgccaagttttctgacagctgtcactcatcaatcatttctctgcttccttattcattcttatcataaacttggcagatttggattaaaaataatatgctaggaattaatatattttttgtatactttctactctgaattattttggctattgaattccattgaaccctaattcattccaataatcagtaatatttaaatgcagataatgagctaaagttcctcattatgagcctttttatatagtttatttttatgtttataaatataactgtgtaattatataactataaaaatgtgtaaaataattgcttagtgtcatgataataaagttaaatctattcagaataaatttttattacttagaaacatcacagaaaaaaattaatgtaaaatttcaattcatatacataatttttatagtaaaatatatctgatcaatataagatattcaggaacaaaaatattatattcaattaaaaatctttacagagaagttgaatggaaatggactcaggaagaaaaatatagatggtagaaaatttctgactattaaaatgaccttgtccacgtatttttcaattaaatattgtggccattgaattgttattgtattaagattccattgaaaaagtattaaagtgattcaatagtttgttttaatgttaatatttacaattagactgatattgcatcattttcaacaaattaaacttagagtaaaaaattttagctgttaacttaatgtgaaagaactacatagatttttctacattattttaagtagtatgaagcaaaaaattgaaagtcttctatggtagactatgaaagactaatatggcatactcagccactcatactggggcatgcttagctgatgtgcttttgctctgaggctctgcttcatgacgaaattcatcagttggggcatttctgtagctgacattagatttctcaggaatctagtccaggaatctctgcatcaagttcagatgaatttctttggagacgtgttctccaaacatgagtggagaaagcagacctacaggtccctcctggcagtttttctatcttaaggtctcctgaaaagaacatattgagaaaaaataagacatgcagatacacacagaaccagtttagtctaattcctgatttctctaacttcacaatgatacagttttaataaaggtacattcaatttacattaacagaatatttaatattctagttttcaacttatgaattcagatttaattggctttatattttttctaaaaacaatgaatgggtgtatatagtatattcgtgttgaaggactgtattagataatactcataaaccatttcagatctcttcagcttcacctgtctttcattccagccactgctatgatgaacagtgctgcgcagactctgacccacttcgtgcaggtgcaatctgactgtcctttgtgccagttctgtgccatgcacaacacatcttccacccagcagcttctctgttgatgccatggagtaggatgctacagaaatctgtttcatactaaaccatacacaacccagaagcacaaatctctatttgtcccttgatgtaaacctttgacaaatagatgttggaagccaatgggtaagtatttctccctttcttccccaagagagatggtcctgcaaggcattttatagggtttctaggacagccctttgaagtgatagttgacttgataagattctcttttattgccctccttgctttgtcaccccacaagtctgctttctggaaaaaacattcccccaatgaaatacttgcatatgaatgtttgccagatgttctgctttctggagaaccaagtctatgactgtagataccagaaagcatcctaaaaagcatacactcagcagcagattttaaaactggatcatttaagtataagatggcaataaggacccagtacttgtagtaatggggtagtaataatccatggcattagatagtatcataattactcattttttaaacttttgatttattgggataagatataggctgaagtcaggtagtgggttttgctatggctgtattgcttaaatggtacagaggcagtgatatttacaaagatcagtagagtagcctggcttttgtaaaccctctaccccagtggtagccaatttaataatacttatttgtattggtttttctttaaaaaagaaaatggtaagcttattcaactgtcaacttaaagtagctgtgaaaaccagaaggccaacatggaaattttagagcctaaaggagactcatctcttgcaacatcaggacaaactattgaatatcaggcatgaaatttaatggcaagagtggtaagatggccgagaaaagtgaatatagacttgatagtcgcccatgccaaactgaggccttcataacaaactgaggccttcataagaaacaaagggaactctgagacctgtgatggagatgtttggtggatgagcttgagaatcttgaactctccaattcccttaaattctccataaatgcacaagtagaggagagcaacctgtatacccttcaaggtctccccaggtgtgggtgacttgcaagctgacagatgtcctccttcagagctatccccaccacccctcattgtgcccaggcctataacagggtcaggtgttggcacagctgacagaagaatgtaaagcccatactcagggagggagtagcttacacttgaaggaatcgtaataactggctgacacatactacagggactgagagaacacacgtaagagaggcttttgagggttagtggtccaggaggagaaatataaagccagatagaggagaatttagtagaaaggagaactagccctgctacactatgggaatgctttcttgaaattagacatgatgggtccatggtaaattaggaggacatgccagcacctccttggctaagtagtaagaaagtaccaagaaaggagccagaagagtcagagagattgaaatgttaaagtggatttctcagtaagaacagaaaaccctgataggtgcagcaaatcaccatggcacacgtttacctatgtaacaaacctacacatcctgcacatgtaccctggaactttaaattaagttaaattaaaatgttttttaaaagaccggaaaacccaatacacgtttctgtttcctgggagggcccagaggatacttccttcactaaagccacaagagtgcatgggagaagcatctcaggatgggaggggcagggggatctttaagaaacagctttaagaaacttggtgagggaaagtaatctgaaatatattgttgacagtaaaagagagtgtaatgaagctctgctccgtggtgccaaacgaggtgataggattccagaaaaggagaaatttggggttggcatttaattgttaaaagcaaggtgtatgtagttaccaagtcataaggtcaggagggcatagcaagaatttattaaacaatagaaataatacattccgtccaccttgaattagttgaaaagtggggaaagggggaaataatcattcaggatcaggtccagaaggcacgagtaaattataatgaagaggcagcccagactaccaggtcacatatctttgtcatgctgtcaccaatctctcggcttgtatgtaaggtctcatggggagtggaggggtagttcttcctaccaactgatggaggaggaaacacagtcttggtgcgcagatgagtcagagcaatgtctgtgctgtcaaaaaatggacttctgttgtattatattcccattcaggagtggtcctaaagggcagtcatgaaaggaaatcattccaataggcatatctgcaagtcatttatttgattatttacctagtgtggacagagaagtggtccaagatatggatatgaattcctgagctatgttgaatgacttgggtggttattagaggtctgaaaggaattagattgaagaccaaaacatgactggtgatgaggtatgtgggtgaccagtggaagatggtccaaggcatgcagaatttttttatgctaatacccatctgagaccatcctggaagcaatcaaattctaggtgcacagaatgatttgttcagtgaatgtcaataagtctcactccttggccagtccagtgctaaccagctaaacccatgaacatagtatgcatgctggcaaagatgattaagcacagaaccaacttttacaaactctgatctagctactctcactgctgagtgcccaatctgccagcagcagaaatcaatgttgaatattcaataaggaacaatccaatatagaacaatgcctcaggagaacagctagccatttagtggcaagttgattacattggactctttcaccctgaagagtaccacaatgtgtcctttccagaatttacatctactctcataagaaattgtcttccttgtcccaagtgcttcttgcagcactgccatctaagggcttacagaatgcctgaactatcacatagtatttcccacaacattgtcttagatcaaggagcccattttattgcaaagaagctgtgacaatgagcacataaccattggcctaatcatgtaaccaattgctcagaagtagctgacccaacagaacagtggaagtcttgtcaaaggctcagctacagcaccagcttggggaagacatggtcatagcaaggactgggttgctgtaatccatgacgtggtatatatgttaagccaacagctaaaaactcatgctgtgtccccagtagccataatgcatgcatctcaggatcaaagagtaaaaatcatccctgatgttccatttccagaattggtgcctctacactgggggcttttctgccttccacactggggtttctctgttgatgataaagcaaggggcaccaaaggaacccatttgttgtctatgtatgcactcagaagtgcaggagtcattccttgtggggcaaaccttggaccaatgggagatgagactaatgggcaaacatttcccctaaaatgcatttcatgctgtttctcagaaggctccctgggaccaaacaacctgtctttcccatagtggttccaactttataacactgccttgtattggctctctctctttctctgctttgcttcccctttcccttattcctgccccctgcaaaaaaaagaaaaagaaaaagaaattccagtaaagtactgcccataagtgctctcacattttgcattctgggggaaatctgaacaagccaggtcctgtgctagtggttttcacaaaaaaaaagaaaacttgctccaaaacgtaattagcatctcttttaccagaaccaaggcctctaacaatttttcttctttttaaaatatatatgtgtatatatatatatatatatacacacacatatatatataaaggtataacttacaataaagtgtgtgtatcttaagtgtacagcttaatgaatttttatatttgtatatccttcttgacagcaaattttaaagatgaattatatccccacaaaacaacttaaactcaaatctgtaacaggtgttttattgcatgtcgatttactctcctctttctataaataaaatacgactgctctagctcaggccttcattctctctcactgtaacaactttctaacaagctccctttagtgttttgacttccttaaatcagtccttcactgtcaccagagaaagctatctaaatgaaaactctgactttgtcactcttctgcttacaattcttcagtggctgctgattacctaaaggaactagggttccttatcctggtactcaaggccctcctccttttatattgtccctcaaccctgcctatctacctagataattttactcatcttttacaccttagattatagctcttttactccaggaagcctccttagcctccgttttccaagccatgccccatagtaccctgtgcctatctccctggttgtaccaaacacttgatattttaaatatcagttcgcctgtctgtttgcttctaaagacaagcatcataatcatttgcattccaataaccccagtgcaaagcttattacctgagtcataatatgcaattaataatttttttagtaaatagattaaaggagaaaagaatatttaagacactgtactcaacaaatgtgtaccaaaaaatttaaaaccaagagatggccaggcatggtggctcatgcctgtaatcccagcactttgggaggctgaggtgggtggagagcttgagcctaggggttcaagaccaaccttggcaagatggtgaaaccgcacctctaccaaaaatacaaaagttggccagtctcataacagggtctcaaaataaataaatagataaaaattaaaaataataaataaaaataaaaagaagagcttttgataaaaccaaatcccacatatttgaatttctaccttagagtagacagatcacccctgtaatcatatatctgacttcctacctgcatggaactcaaacatctgctatcaagagattaaactgctctgacgtaggcatcaccctgccctgaggcctggtgctctaaggtgtcctggttacccacagcgctctgagtcagtgttgagccattctcactgatggctcatagtaccatgggtctcaagctattgagttacagttttccatatccccggtattcagtcttagttgttgcctagtttttcatctccctgctttacattctagttcccccaatgtctaagtcttttgccctttatccccatcttctggagactaaatggctgccttccaacaggttgattgcctgatctctgaaactttatgagtcacttaccctagatctgagacttgatttgaaccaaaggtacacgtttgtgatctgtccttcctgcctcttggcctggcacccagttttagatctcccttcaggctcatttcatgcagatatgcttggacttacgattctgatcatcctttgacctgtctcatcatccttctcatatcctaagttagcacttcccccaattattcaacttttgtgtaaatccgtattttgttatattgagtcttcttaacgtgagttatactcatgatacaaagcagagccaagtgtaggagtgagtttggaggtgataactctttctgagtggacccaaacaattcagtactttgtataagatgatttagagtctggtacaaagcacctcttaggggcctattcagtgtgaacaagttctctaacattaagaacctgacagtttggttgaggaagaacaacaggctcctatgaagtcatccagaagtgtatgtttacatggcagctacttagtttatcagcctttctctggctacctgccttaaattatttccttagcaaaatcgactttgcttatccaaatgccctaaaatgcaaaccagttttgcttccccataaatctctggaattaataggaaaaagaatttgaaactttgtcccaaaataagacattgataacatttcctttatgttcaggatcaatttgttggcatttccctaactcatctgcatccttccctaaaggaataaatgacttcccagtttaacgagatctctttattgtgcacagaagaacacatggcacttcatcatgtatgactgccctctgcctttatgaatatcatgaaatttggctatctgctccaagatctccttctttgcttctcctctttttaattccatgaaacctaaccctggagataatatgtatgtacaacctattttctcatgatcagtatttttaaatcacaacaggaaaggagccaacaaagtcatctttggtctttggctatcttcttataggaacactttttccttcatttccttgttattatttggttgtaaagtcctccaagacactgaattgttagacctgcaactttttgtagaagagaattctgttttgctgagtaaagtgcagcatgctgcattctctatgttggagatgaggtattgattttttttaataatatttggttcaggggtatatattgctgctgaattattatgtttcttagaattgaacatttcacaaatggatccaagatggccttttatggattgaaatagttctaaaccgtacattatcaatagcaacaaaataaatcaccaggatgtgaataaaatattgattatatattcctttcctccattaatttgaactttacatctagtatttcaatttttctgtaatgagataccattatatctacattgattatagcctagaaatgttttcagttaaaatttcgaagtaaaaagttttttgtttcaggaaaagtgggctgaaaacaaaatataaggtaacatttcaaaataatttgtatgatgtcaatggcttaacagtggttatttcatgaactcagaaaaatcttattggaaaagtattcatcttatgaacacatatagctgagaaaatagagtttaggctgacttgcagcattaggtcttgatacaatttaaataaagaacttgaaaacattcataacacttatgtcagcctacagagaaaggtacttacttaactcatgttctcccacttaggccatgtgaacagactcttttacataatgtgattgtagtcaaaggtcctcaaacatcattattatcatcctcatggttatcatcacccttaccgccattgtgtacccaactgcaaggtagcatagggacatcaaatcttagcattttcaaggggttaattttaaagaaagagagtgtaaatgtcaaacacagttttaaaactagaaggaaggaactgtcagtggatcaaagctgaggttagttcccagagtccagttagatatattaagagttaaatgagagacgcactgacactaggtcatggtcctgtttcttgacttttgaaccactgctttttcatggcttttatcaacaataacaggatacaaagtgctaaaagtgtgggaagatatttacatttaaggagaacaaaaaaacatgacactgatggtttaaaaaaagtcacatggaagaaaaatgagagagaaagtcataacaaagggctggacttgaagttaaatccctattaggtcctgtaaaatgctgaacacattaggttaaatatttactcttcaaaccaagcttttgtggtgtagataccatttattctattttacagataggaggtgttgggctcggaaagtttaagcaacatgcccaagatcacccagctgggagtagtgaggcagaattaaacaccaagctcaactgatgcaaagattgttctactacagatgattctcaaacctagctcatgttactatcacctagggatctctgaagaagtacctctgcctgggcaccactcaggatcaattgaactagaatctgtaagggagtcaaacatccgtacttttagaaagccctttatacaattatgatgtgtagccaaggttgaaaacactgaactaacagaaggctaccgaaatggtgtgaattagggagcagtgaaaaggccacctgtccaggacacaaagtaaccaatatttccatagactctattgacagagttcctgtaacagccaaaacaaatatttttggcagaagcatatcaacagatgattcttttcagcatgtttacagctaattcatagcccagaaaggaggttgaaaaacaatgataagtggatataacttcctaataaaaacttagaattgcatagatatttcctgtaggaaatatcaaagttaattgaggcataagaattcacttaaaaggccactgcaaaaaaaaaaataaacaaagcttaaaatttgcactttcactttgcaataagtcctcgttgcttttcagaatagtttggtatctaaaaccttgagaaatgtaaggaataagttcaagagagaggtcaagctggagtcttataacaagcatatagagatcatttcaagattggaaattgagggcatcacttagaaaggataataataaaagagaagtgtgcctaagattgaaacttaagtacttaagagactcttaagaaggagtggcaggaaggtacaaggaaaacaggatgacataaggacttgaagttaacagagtacaag

SEQ ID NO: 2 - DNA, 93480 bp, Homo sapiens
acaagggtggcagcctggctctgaagtggaattatgtgcttcaaacaggttgaaaggtaaataggcagcgatcatccaaatacttaaagacagctatatttccttttggaaatacatgggccaagttgttttctcttgcaagttctcatgatattccatgcagtggttactgatgaaaagggaatctataatattatatggcatcacattactttaaataataattattgcatatttccatcagttaacttctaactaggaacatttttagggcttctgttgcttcttatgaagagaagaattactattgctaacaaataatgaaagggatccctatatttctgagccaaggggaccccagtttttttaaatgttactagatattttatgcgatatgatgagccatgcattgcgttctcttgtagttttatagaaaaagctcaggttttattaaacagataaaataactgaataatgttgatcattgagtaaatgagttaattcatgtaactgattaatcaaatagtgtaactctagttgtaaactaacaacggtattattaggaatttctataacgtgttcctttcgcgttctctacagatgaaaggctttttcctcttttttagggggtgcgggggacaaggtctcactctgtcgcccaggctggagtgcaatggtgtgatcatggttcactgcaaccttcacctcccgggtagctgggatgaccgtcacatgccaccacactgggctaatttttgtatttttagtagggacagggtttcaccctgttgcccagcctgatctcgaactcctgggctcaagcaatccaccggcctctgcctcccaaagtgctgggattacaggcatgagccaccacgcctccccagatgaaacgctttttagagtctcactggtgagagcacatattgcttaatctcacttcacttttcttaattttgaatccattttctctgggagttctgaaatttttggctcttcccaagtttagctcatcaaagactagtatttctggagtgtgataagtatgtaactttggcagggagaagaattcatcaccagaagttagctcagcccaggcaccatggttcatgcctgtaatcctagcactttgggaggttgagttgggaggaccgcttgagcccaggagtttgaggttacagtgagctatgataatgccgctgcactccagcctgggctacagagcctggcccttgtctctaaaaaaatttaaaacataaaataaaaaaagaagttaactcagaggaggaggtttctgatgagaaaggctagattaggaggcactgtaatggatcaaatgccctactgagttaattcatagtgacttttgaagagtataaaattccatttatttgctttacttctccatctcttgttatatctcctaaatataccagtaacatttctttgctctgtttccttccttcctttcttccttccttctttccttcttcctccctaatcaaaggttcccatcaaatacaataattcttagggaaaaatgttatgctttttattattttactaaaatactacaatttaaacatttttcatattttttttccagagggaaacagtcttttcctgcttccagacatgaatcaggtcactattcaatgggatgcagtaatagccctttacatactcttcagctggtgtcatggaggtatggtgttttatgtatctattgctatctttcattcaacaaatggaatattgagtgattatcattttcatggttttatgttagaatctctgaagaatacaaatattattagactagaaaaaatgtgtgcataaaaattacatagaatgaaataaaaatggcccaatctccttccaaatgcaggatttctttctcaaatttcccttctagacagccctcttactcacatgtggatatttccacaaaggagcttattagtttgagttggcatgttctattgtcaaactattataactgttatataattacctgtcaaacatttggatgcaattctatttcacaaaccacttgttgatagttactttgtgccgggcacatagtaactatgtgggccattggcaagagtatgattttattcattatattcaaactttactgaaaaaagtaggctttatctaaaaaagaacaaaagattggttgagtagctagagaatggggttcagattctacagtgcctagtgttatttctaatcttacctttgaaaaatactgtagctttataatctctcggttctctactttaaagtaggagagctaattatatttgctctttagacacttagagaagcttccaggctgactctagtcttgtgtcatggtgctgaacatcaataaaagcataacagaggccagacgaggtggctaatgcctgtaatcccagcactttgggaggccgaggcgggtggatcacctgaggtcaggagttcaagatcagcctggccaacatggtgaaacgccatattagccgggcgtggtggtgctcacctgtaatcccagctactcaggaggctaaggcaggagaatcgcttgaacctggaaggcggatgttgcagtgagccgagatcgcaacactgcactgcagcctgggtgacagaatgagactgtctcaaaaaaaaaaaaaaaaaaaaaagcctaacagaaacacactgtaataggctagtttatgaaaagatacatctagtcctaaaactgttttcaatggaatcatttagttaatagaaatgataaatttaaaatgcaatagcatattcttctgaatctcttgatttaatgttttacgtgtaatttattatttttccatttatttctaggaattacaaatataaactgctctggccacatctgggtagaaccagccacaatttttaagatgggtatgaatatctctatatattgccaagcagcaattaagaactgccaaccaaggaaacttcatttttataaaaatggcatcaaagaaagatttcaaatcacaaggattaataaaacaacagctcggctttggtataaaaactttctggaaccacatgcttctatgtactgcactgctgaatgtcccaaacattttcaagagacactgatatgtggaaaagacatttcttctggatgtaagtgttggggcacatttgaaatgcaaacaaaagctagtttaacaattaactggtcattaccacactagtctaaaaatagggacaaaataatatagttcagtttttagattagtttcatacaggagctgcaaactcaaatgtctccagggccaggttggtgaactatatgaactgtagccactgcccagatccagtaatcacaggtgcccggtattcctacatcttttggcttttcaaaggaagacagaaacataaaattttaatgtgtactctctaaattttaaaatgttgccaatacattcaaaaaacaatttttcacctctaccctgaacgaacactgcatatgtgagggccagccccgttagatttaaaatgttgctcttttaatcttcttgtttatttttctaaactaatataatataattaaccttgaagcagatcccattgagaaattttgaggccaaggtttagggagagttgcaaaactagttttgtgctcccactgtactgctaggtttgtgaccttggacaagttgcttaagcctcaattcctcgcctgtaaaaggaggctaattatacaggctgtgttatgcacttaatttcaataatttatttggaagcatatgataaagtttaaagtacatcagtacccaaatccttaaaaatgcatccttttgataaaacatagggagagtctcctacagtcctctgttcctaatctcactattccaatttagaatcactgtgtttgttattgtaactattaatttttttctttttcttaaaacccaccttgaagggaataattattaatttatttaaaagcttagtcaagtcaatcttaaaaatatacgctaaattcaatgtgtgctctgagtgtcctggaggtgtttaaatcactatagtgatatgggctggctcgtataattatatacctagtttgggttctactattatgaagggtgtaaatcaataagttcagcactgttttaactcagatattcattctctatagcacttgaacccattttcagtaaaaaaataaaggaatttgtgtctttgccatgttcccgtcctccacatttttgagaatcaagcctcagttcttgcttcttcttcacctttacaacttagctggggggcagacccatatcaccactaccacctgaccccattgtggctgcttggaaagtaagaggcttccttgctctataatgaggccattgtcctacttaactctgagcagctgatcttgcaagcttttcaagagaaggaactatagaaagttgtagaggtgagttgaggaaggaaagtcataggttttggagtcaaagaataaatctaccttaaactgatgccttaaagctgctaaacttgccctaaattgtggaaattaaaccgcaggcagacatcttatctgatgtaagaaatcttcttagcttgtccacatcctcaggaaacataaccaaacctcaatgcccaaatactttactatttatcctttgggggtgggagttgggtagagagactatacaatttctttggagggcaaataagcaactttcacagttacagaaaaagcttagttgttaatttaagccagaggaaaaaaaaggaagcaaaataaacattgtctctgttaaaaaacaaaatttcagcaaatttagtttttagatctgataggtttttatgtgtgattcatgaatcggcagcatctctttctaaaaaactgatatgtgctctgagtgtcctggaggtgtttaagttactatagtgatatgggctggcttgtataattatatacttagttttggctctactattctaacagaacagttggtttttgtaaggtaggaacaagaaatagaaatttttataaaaactaccagattggttaacattgttacttcaggttattttccttgtaatggttaaagcagaggggatttccttatcatccctgctcaggttgactgggctctttctacgtggttgctgtgaattttctgttttcaggaaaaactagtctgtgggagttttacctgcttccttaaagtttcagcttggttatatgtcacttaacaggagtgacttcattttggtttggtatgtgggggcctagtgcaggagctcagtccaaaacaatggtctcctatacattttatttaacaccaccttccgtgtgttgcagtttgtataactgattactcccaaaagccctcccaagtgttggctggggggcctctgtccccaaacccaacaccaggcaatgtagaaggtaagagaatggactttgttcctcttccaggacagcctaatagaaatacattctctgccgatggctgcttagaaagaacatcactctttccacttatttaacttttgagtttgtgaattgtaactccttacataggcctctggttatgagtgtaaaaatgtaactgccaggtgagaatttactcaaacccctggattcaagctgaatgtcttcttgagctctacattcttagcaaacaaaacttcctatttaagtaggtcaaaaaagggtttttgagggttttgtggcattctagaaatcacattgctttcactggaccactttgatcacaggatatttgtcatgtgagatttattatgtcaacttatcatcactgctttgaacaaaatgattatatgtatttttttagactagagacaaattgtaaaagacaagataaatgagtcattgctttccttgaaaagctatccaaatatattttaaagtgctgtgctttaatgtataatttgtgaaatataaaccatattatcacaatttccatttttagccacaatcctagacacttcttagattcttatctaagactaaatgcacacatattatgggaaatttgttctaatcaggccacaccaatcatgggataaaatacaagatgtctttattcatttaaaaataccaaattaaaccatgcacagtcatgtttccacttaatcatttgtttaaaattttaaatatctttgactttttaaattttaacttttggactttatttacaatgatgaaagcacatttgttattcaataaaagcctctgaatatttgtttctaacattgctattaagtatgattacggaacaattaggctggcctgtgccctgcttgagtaagaaattatagagtgatctttgccagaagcattgcatctctctccattttaatccttcatgttattatcttaatcaagccaatcaattcatggaataaactttattgagtggttactatgcaccaaaaatgtgttaaatgctagaagtacaaagaagaggaaatcacggactgttttcaagaagttctcagtagaaagggatccttggctagactctgaattaacaagaaacctggcctgtgattcctgagtgtcccaataataggggaggggtgggattggcaagaaagagcaaaaacataagcccccttttgtatacctcagagtattctctagtatctagttgtatagccaggttttgttcttggatattgaataacctggcaatgaggcctaacaacaacttgatgttattatcataagcatactcacaggcccatctttacattgcttcatgttaacaaacacttatttgcatgtgtgtgtatagtcatctttttgatcttttgcaaagttaattgaccaccagacatttgtaaccccaagaattcacttcatgtagatgccgtggagtaacacagcccaaacaatctgatagccaaatcaatggcactctgtatctgaaaccagaggaaaaaataacttcagcacaaatcattgtaatcaatgagggaccacaactcaaactccaaaatgatttaaatttatgtttccacacctacttcatttaaggctcaattaaaacaaagatttattttaccctacttggagcccagtttctctccattgtaactcaatgctacaagtcgccaaaatgttgtaaagtactttttagtgtgttcttactgtttgaatataggtttttttaaaaggatattagaccttcgatatatgaaaatagttttttaattataaaatcaaattgcattgaatatagagagtttttaaaatgtagaccagcaaaaaagaaatacctatcattcccataaaaccataactttactagtaaccataatccctctttaataatttggaatatgtctttgaaatttcagaccctctctttctcagtctctcacttaaatgaaaccactaattgaaccattttgcaacctgcttttttcactttgccatatgatcatgacttcactacatatacttttaattccttccaaaggccaagtacttaaactgtcttcttcaacattgcttttggatgtgtttaggactgtattatagtttatttaaccttattttggaaaattaagtttgtttacaattttttcattgttatgaacaataagggaacatcctttaagccaaatattttatatatctatgaacatttcaataaggatgcattcttagatgtataattgctgagttgcaaaatattacacctgcttttacagattgtgatatgtaaagtgctctccataaaggctgtaccaatttgtacttccaccagaagtgtgttagaggatttccatgagtccactccaacactgtgtactatcatttttcaaaatccttgctaatttgataggcttgaatgacatctagtattgccttgatttgcagtgagaataaaatttttaatatatttgtttattgttctacaagcaatatttttggtaagcaatttgcaggtgaagtgaaagcatatatgtacttaagataaatctttggatgtaacttcagacgcataaatgtctgtattttcattctttatgagcccctatccttccatttgtcttgtcattctctgagcagtcaagtgtcccagcagtatttcacacgtagggatgaactaagagagccacacctgaaaagtgaatatttaacaaacactcaatatttctatacttaaaaaaatcttaatcttattcactattgaatgatataactgaaaattctaggtgtcattctatagttgttggggtttcataggctttagaggcccccaaaactcaatgtcatcagaactctctcatcttttccagtctgctaggtattgttttcccctgtgttggcttcattcttaaatagactaaagactgtgaaaaagcctgtttcacaatgacagcaagatggccaccagcaacttacagctcacatgctactcagagatagcagtttcaggaggaaagagtgtgcttcttttacagtggtttagaaaaagtgccttggagaattctgattggttcatcttgagtcatatgttcattcccaaaggcacactcctattagtcggcctacctcagaggcacacttctgtggagagcctcttgattgacaggtcttggggttggggtgggggtgagattggagtgaggggctggaggggatgtcagttctcaagagggaaagacgctagccaatgaaaaataaaaatgtttgccataacatggccacctggagaaagagctgggcaggaatgggggtgggatgagttgagtggagtgacaggaaaagagccttgaaatagagttgcatgaataccaaactttaaaaaaaccttgttgtgttggtggtaaaatacataacataaaatgtaccatttttaccatggttaagtgtgcagttcagtggcactaagtacacttacactgttgtacaaccatcaccaccatttatctccagaaccctcttattgcaaatggaaagtctgcagcctttaaacaaatatccacttccctctcctccaagccccagcaactgccattctactttctacaagtttgactcttccagataccttgtgtaaggggtcatgcaatgtttttcctttagggtctgtcttacttcgcttaatataatgtcttcaagtttcatccacattgtagcatgtgttaaaatttccttcctttttaaggctgagtaatatcttattgcatgtatgaacacccaacttttaaaatgagtagtggaaaatgtgcaagagaagaagatagtctgccagagtagtagaagaaaactacaaagaacccattcgcaagagccaaaggcaaagaggtttcttttcttgtctcgtttttgagacaaggtctcactgtgtcacccaggctggagtgcagtgtcttggtcatagctcactgcagcctcaaactcctaggctccaggaatccttccagcctcagcctcccaagtagctgagactacagatatgagccaccacacccagctttaaaacagagagctttcaaggagggagtgagcaaaatatcaaatgctgctcaatgacatcaaactcaaactcaatgacaaatgaccagagatggggtctgttgaagagaaagtcatcaatgaccttagcaacaatggttttggtggaattgtggaggctgaatcctgattgttttgggtttaaatgcatatgggaaatgaagaataataggctctttaaaagtgtttagctgttaagaaaaaaagaaagatacaggagtagctgggggagactgaatttaggataatatagtcaagtataagaagaagaggaatggatatttgatggtaaagtgggttgcaaggtaaggtagtagggatggaatgaggagcagggaggtgctgagtactgattggcctgacttctcaaataaaattgtcagtttctcaacggactatagagaacttcatatttcttttttttttattcttccattacaaatttattatcttttcagacaagttgagacatactaatttaaaataaattcccttttcagtgaaaagttgataagtttaatcaggtaaagctaatgattaggtaagcaatcaatgtttataattgcatcacaacttttctggccagtgactttttatttatctatttaatttttttaaattatacttgaagttttagggtacatgtgcacaacgtgcaggcttgtttcatacatatacatgtgccatgttggtgtgctgcacccattaactgatcatttaacattaggtatatctcctaatgctatccctcccccctcctcccaccacacaactggccccggtgtgtgatgttccccttcctgtgtccatgtgttctcattgttcagttcccacctatgagtgagaacatgtggtgtgatagtttgctgagaatgatggtttccagcttcatccatgtccctacaaaggacatgaactcatcattttttatggctgcatagtattccatgctatatatgtgccacatttttttaatccactctatcattgttggacatttgggttggttccaagtctttgctattgtgaatagtgccacaataaacatacgtgtgcatgtgtctttacagcagcatgatttataatcctttgggtatatactcagtagtgggatggctcagtcaaatggtatttctagttctagatccctgaggaatcgccacactgacttccacaatggttgaactagtttacagtcccaccaacagtgtaaaagtgttcctatttctccacatcctctccagcacctgttgtttcctgactttttaatgatcaccattctacctggtgtgagatggtatctcattgtggttttgatttgcatttctctgatggccagtgatgatgagcattttttcatgtgtctgttggctgcataaatgtcttcttttgagaagtgtctgttcatatctgttgcccactttttgatggggttgtttgtttttttcttgtaaatttgtttgagttctttgtagattctggatattagccctttgtcagatgagtagattgcaaaaattttctcccattctgtaggttgcctgttcactctgatggtagtttcttttgctgtgcagaagctctttagtttaattagatcccatttgtcaattttggcttttgttgccattgtttttagtgttttcatcatgaagtctttgcccatgcctatgtcctgaatggtattatctaggtattcttctagggtttttatggttttaggtcttatgtttaagtattgaatccatcttgagttaatttttgtataaggtgcaaggaagggatccagtttcagccttatgcatatggctagccagtttcccaacacaatttattaaatagggaatcctttccctattgctcgtttttgtcaggtttgtcaaagatcagatggttgtagatgtgtggtgttatttctgaggtctctgttctgttccattggtctatatatctgttttggtaccagtatcctgctgtttttgttactgtagccttgtagtatagtttgaagtcaggtagcatgatgcctccggctttgtccttttggcttaggattgacttggcgatgcaggcttgtgtttggttccatatgacctttaaactagttttttccaattctgtgaagaaagtcattggtagcttgatggggatggcattgaatctataaattaccttgggcagtatggccattttcacgatattgattcttcctccccatgagcatggaatgttcttccatttgtttgtgtcctcttttatttcattgtacagtggtttgtagttctctttgaagaggtccttcacatcccttgtaagttggattcctaggtattttattctctttgaagcaattgtgaatgggagttcacttttgatttggctctccatttgtctgttattggtgtataagaatgcttgtgatttttgcacattgattttgtatcctgagactttgctaaagttgcctatcagcttaaggagattttgggctgagacgatggggttttctagatatacaatcatgtcatctgcaaacagggacaatttgacttcctcttttcctaattgaataccctttatttccttcacctgcctgattgccctggccagaacttccaacactatgttgaataggagtggtgagagagggcatccccatcttgtgccagttttcaaagggaatgcttccagtttttgcccattcagatgatattggctgtgggtttgtcatagatagctcttattattttgagatacgtcccactaatacctaatttattgagagtttttagcatgaagcttgttgaattttgtcaaaggccttttctgcagctattgagacaatcatgtggtttttgtcattggttctgttgatatgctggattacgtttattgatttgcatatgttgaaccagccttgcctcccagggaggaagcccacttgatcatggtggataagctttttgatgtgctgttggattcggtttgccagtattttattgaggatttttgcatcgatgttcatcagagatattggtctaaaattctcttttttggttgtgtctctgccaggctttggtatcaggaagatgctggcctcataaaatgagttagggaggattccctctttttctattgattggaatagtttcagaaggaatggtaccagcttcttcttgtacctctggtagaatttgtctgtgaatccatctggtcctggactttttttggttggtaggctattaattattgcctcaatttcagagcctattattggcctattcagagattcaacttcttcctggtttagtcttgggagggtgtatgcgtccaggaatttatccatttcttctagactttctagtttatttgcatcaaggtgtttatagtattctctgatggtagtttgtatttctgtgggatcggtggtgatatcccctgtatcattttttattgcatctgtttgatgcttctctcttttcttctttattagtcttgctagtggtctatcaattttgttgattttttcaaaaaaaacagctcctggattcattgattttttgaagggttttttgtgtctctatttccttcagttctgctctgatcttagttatttcttgccttctgctagcttttgaatgtgtttgatcttgcttctccagttcttttaattgtgatgttaggttgtcaattttagatctttcctgctttctcttgtgggcatttagtgctataaatttccctctacacactgctttaaatgtgtcccagagattcgggtatgttgtgtctttgttctcgttggtttcaaagaacatctttatttctgccttcatttcgttatgtacctagtagtcattcaggagcaggttgttcagtttccatgtagttgagcagttttgagtgagtttcttaatcctgagtcctagtttgattgcactgtggtctgagagacagtttgttagaatttctgtttttttacatttgctgaggagtactttacttccgactatgtggtcaatttggaagaggtgtggtgtggtgctgagaagaatatatattctgtggatttagggtggagagttctgtagatgtctattaggtctgcttggtgcagagctgagttcaattcctggatatcattgttaactttctgtctcgttgatctgtctaatgttgacagtggggtgttaaagtctcccattattattgtgtggggagtctaagtctctttgtaggtctctaaggacttgctttatgaatctgggtgctcctgtattgggtgcatatatatttaggatagttagctcttcttgttgaattgatccctttaccattatgtaatggccttttttgtctcttttgatctttgttggtttaaagtctgttttatcagagactaggattgcaacccctgcctttttttgttttccatttgcttggtagatcttcctccatccctttattttgagcctatgtgtgtctctgcacgtgagatgggtttcctgaatacagcacgctgatgggtcttgactctttatccaatttgccagtctgtgtctttcaattggagcatttagcccatttacatttaaggttaatattgttatgtgtgaatttgatcctgtcattatgatgttagctggttattttgctcattagttgatacagtttcttcctagcctcgatggtctttacaatttggcacgtttttgcagtggctggtactggtttttcctttccatgtttattgcttccttcaggagctcttttagggcaggcctggtggtgacaaaatctctcagcatttgcttgtctgtaaagtattttatttctccttcacttatgaagcttagtttggctggatatgaaaatctgagttgaaaattcttttctttaagaatgctgaatattgacctccactctcttctggcttttagagtttctgctgagagatccgctgttagtctgatgggcttccctttgtgggtaacccgacctttctctctggctgtgcttaacattttttccttcatttcaactttggtgaatctgacaatcatgtgtcttggagttggtcttctcgaggagtatctttgtggcgttctctgtatttcctgaatctgaatgttggcctgccttgctagatttgggaagttctcctggatcctactctggggaagtatcctgcagagtgttttccagcttggttccattctccctgtcactttcaggtacaccaatcagacatagatttggtcttttcacatagtcccgtatttcttggaggctttgttcatttctttttattctttttcctctaaacttctcttctcacttcatttcattcatttgatattccatcactgataccctgtcttccagttgatcgaattggctgctgaggcttgtgcattcatcatgtagttctcgtgccatggttttcagctccatcaggtcctttaaggacttctctgcattggttattctagttagccatccgtctaatcttttttcaaggtttttaacttctttgccatgggttcaaacttcctcctttagctcggagaagtttgatcttctgaagccttcttctctcaacttgtcaaagtcattctccatccagctttgttccattgctggtgaggagctgcattcctttggaggaggagaggcgctctgatttttagaattttcagtttttctgctctgttctttccccatctttgtggttttatctatctttggtctttgatgatggtgatgtacagatggggttttggtgtggatgtcctttctgtttgttagttttccttctaacagtcaggaacctcagctgcaggtctgttggagtttgctggaggtccattccagccctgtttgcctgggtatcagcagtggaggctgcagaacagcgaatattggtgaacagcaaatgttgctgcctgattgttcctctggaagttttgtctcagaggagtacctggccatgtgacgtgtcagtctgcccctactggggggtgcctcccagttaggctactcagaggtcagggacccacttgaggaggcagtctgtccattctcagatctcaagctgtgtgctgggagaaccactactctcttcaaagctgtcagacggggacatttaagtctgcagaggtttctgctgccttttgtttagctatgccctgcccccggaggtggagtctacagaggcaggcagtcaggcctccttgagctgcagtgggctccacccagttcgagcttcccagccactttgtttacctactcaagcctcagcaatggcgggcacccctctcccagcctcgctgccaccttgcagtttgatctcagactgctgtgctagcaatgagtgaggcttgatgggcataggaccctctgagccaggcatgggatataagctcctggtgtgccatttgctgagactgtcggaaaagtgcagtattagggtgggagtgaccctattttccaggtgccatctgtcacccctttccttggctaggaaagggaattccctgaccccttgcacttcccgggtgaggcaatgcctcgccctgcttcggctcacactctgtgcactgcacccactgtcccgcacccactgtctgataaaccccagtgagatgaacccggtacctcagttggaaatgaagaaatcgttcgtcttctgcatcattcacgctgggagctgtagactggagctgttcctattcagccatcttggcttgggaccagagaacttcgtatttcttacagcacctcctaagtgttatgttttgttgcagatccgccagatattcctgatgaagtaacctgtgtcatttatgaatattcaggcaacatgacttgcacctggaatgctgggaagctcacctacatagacacaaaatacgtggtacatgtgaagaggtaggtcacttcctcacggcttcatataagcagttccaccccagttcagccagagctctgcctccagcagagatccaagaaatcagcctcaaacattaaatatataccctgatttatcccttatttcctacttatgatcagtgaaactaccaaagcccttttcaagccattaatattttcactctggcaggcaaagtgctctatgatctttctgtcctttttattcattagtaggtaactggtagcaggctcctaatggtggtacagcctagcacagttttaagccattgcttaggacgggcacagtggctcacacctgtaatcccacactttgggaggccaaggtgggtgaataacctgaggtcaggagtttgagaccagcctggccaacatggtgaaacctggtttctactgaaaatacaaaaattagccaggcattgtggcaggcatctgtaattccagctgctcaggaggctgaggcaggagaattgcttgaacccaggaggcagaggttgcagtgagccgagactgtgccactgcactctagcctgggcgacagaacaagactccatctcaaaaaataaaataaaataataaattattgcttaaatccctgctctattcttcttctagggtttcaacaacttaatttccccgaattgctattatttttaatctttaaatgggcctaaaaataaaacataacacacaggtttgttctaattaattatgtggaatttggcttttaaagtgtatataattatgccatattaccactaaattcatggaagctattgttaataatgaccctcagatcttaaatttagtgtctacatagctgatcaactttcctgtttgccttatactaagggggttttcaggacaaaggattttcagttttaaaacctggaaagttgtgggcaaactaggacaagttgcttaccttatccgtgtttcttgttttgtttagtttattaaaaaataaaattccaggccaggcatagtggctcatgcctgtaatcccagcactttgggagactgaggctggtggatcacccgaggtcaggagttcaagatcagcctggccaacatggtgaaaccccatctctactaaaaatacaaaaattagctgggcgtggtggtgggcgcctgtaatcccagctactcaggaggctgaagcagggagaattgcttgaacccaggaggcagaggttgcagtgagctgagatcacgccactgcactccagcctgggcgaaagagcgagactccatctcaaaaataataataaataaaataagatgccggctgggcgcggtggctcccgcctgtaatcccagcactttgggaggctgaggtgggtgcatcacctgagatcaggaatttgaaatgagcctggccaacatggtgaaaccccgtctctactaaaaatgcaaaaattagccaggtgtggtggcacacacttgtaatcccagctactggggaggccgagacaggagaattgcttgaactcaggagacaggagcttgcagtgagccaagatcgtgccattgcactccagcctgggcaacaagagggaaactctgtctcaaaaaataaataaaataaaataaataaaataaaataaaataaaataaaataaaataaaataaaataaaataagccttctgtggtgttgttgaagtatgcttttttttttctgtcactaattcttaaaaatttcacaaaccattttgtaaagcatttattggacccttactgtttgccaagcactgggccaagagctttataaatgctgcctcatttaataccctatacaaccatgtgaagccctgggatcatacaaatgcttatatcctcctaaaattcatatgttgaaacaaacccccaatgcaatagtaacaagagttggggcctttgaagtgagattaggtcatgagggtaaagccctcatggatggaattattgcccttccataagaggcctgaaagagagcttgtttgcccttctaccctgtgaggacacaaccagaaggtgctgtctgtaaagcagagagtgagccctcaccagacactgaatctgctgatgccttgatcttggacttccaagcctccagaactgtaaacaataaatttctgttgtttataaattgcctagtctaaggtatttgtatggcagctcaaagagactaagacagctgcatacaattatttttcccatttgtatagacaagggaactgagacttagtgaggtcaggtaatgaatgtatccaggaccacacagctgcagagaaggagaggtggaatttgaacataggttgcctgactgtcttctcaacttattttctgctgaaggtgtgtggaggaccagatcgcagggcatatttaagatattctactcagttctcatcattatggaagggaataaccttctttttaaaaaaaatctttttatttatttatagggtttttgttttgttttgttttcgtttttgtttttgtttttgagccacggtctctgtcgcccaggctggagtgcagtggcacaatctcagctcactgcaacctctgcctcctgggtttcaagcaattctcccagccaccaagcctgatctctatggaagagaataacctaatcttgctctctctctccactttttggatactcagaataattttgattaagtgaaacctaaaaagaaacacctcatcagtcaagaaagtccacgtagaaactagacagattttatgaacacattgccaggcctagagttaccagctacatgccattggcaagaggcgtacacctttcattctgaacaggagttaaacagaggcatattctggtaccaaagaggtgatgaaaactctgatatcagggatgggtggaattgattgaagagatgcaatatgctaagtactgtgctctgtacttcatgtgcatgacatcttcaattctcaacttaaccttgtgagggaactatcgttattgttcccattttacagatggcgtggggagctcacattacctctccaaagtcacttgactaagaagtaaaggagcagggattctaacagggatttctctggctacaccactggagactaaacctctaccccttgcctatagggtaaagtagaaaagccatgagagcaagacaactagagtctcattgctaagtcaactttaggaatgtgaaggtgagggcagtatggcagcaaactttgcacttcaagcacacagaaaaaccaggcagtgccagattgacagagatagcctagtgtcttgaggatcttcactatctcccagaacctggaaggatctcctgaactgcttatcctggttgtttggttaattcattctttcttttatttttaatataaccctttattaagtataattcatctaccatacaattcaccttatttaaagtatatcattccttgagttttagcacccatcactacaatcaatattgaacattttcatcatgccaaaaaaaaaatcacatccatgagcagtcacttcctatttcccctcaatcttcccagtctatggcagccactatttgctttccgtttctgttgatttgcctacactggacacttcatgtaaaaggaatcatataatatgtggtcttttgtgactggcttctttcatttagtataatgtcttcaaggttatctatggtgtaacatgtatcaggctggagtgcaatggtgtgatctcggctcactacaacctctgcctcctgggttcaagtgattctcctgtctcagcctcctgagtagctgggattacaggtgcacgccaccacgcccagctgattttttttgtattttagtagagactgggtttcaccatgttgcccaggctggtatggaactcctgagctcaggcaaccctcctgccttggcctcccaaagtgctaggattacaggcataagccaacgcgcctggccctttattcctttttttaataatgttccattatttggaacacattttatttatttattccttggttgatgaactttttggttgtttccacttttcagctattatgaattattcttctattaatatttgtgtacacgttttgtgtagacatatttttatttctcttgggcatatatttaggaatagaattcctgggtctcttggtaactctatgtttaatcttttgttcaactgccataatgttttccaaagcagccacaccattttacattcccaacaacagtgtataagggttccagtatctccacattcttgccaacctttgtttttatcttggttattgccatcctagtggatgcaggttaattctttcaagcctgacttacctgccttctgaggttcctgatagtaaaactgcattgcaaattccagacagatgaaccacaatttgaaaaaatatatttgttggatacaattttaaacttgctaacccccacctaatttcctatttagtatcataaaaatattttctgaatattttatatgactatgtattatatatttcttttaaaatcctttaatgtgttcaaaattatgtatgtttacatttatttacacatgacttttgaggaacacattcactttataaagtaaggaatatttgtagtagttatgagtactaagattgttctcagtactcaaaaagtcactctgaaagtattaagaatgagtttactaaagagaatgctttcagaaatattcattgggaagagcatgggctttagagttttagatttaatttccagttctgaaacttaccagtcaggtgtcttttatgatacaaatatcagaaaactcaagtgctggccaggcgcagtggcacacgcttgtaatcccagcactttgggaggctgaggcagatggatcacttgaggtccggagttcaagaccagcctggccaatatggtgaaaccccatctctactaagaatacaaaaattagcagggcatgtaatcccagctacttgggaggctgaggcaggagaatcgcttgaacctgggaggtgaaggttgcagtgagctgagttcatgccattgcactccagcctgggctacagagtgagactccatctcaaaaaacaaaaaaataaaagaaaaataaaaaaactcaagtgcagtcacttaaatagacatggacttatttatatcaggtcataattatatgtagtaacagatggcatgataattcagtaataccccagggatctaggctcttttcctctttctactctgccatcttcagtgtgttgaccttaatcttcagatgctgtatctccaggcttcttatccaagttccaggcaggaaaagtggtaaagagcaaaggagttcctcctgtggacttcgacctacactgagttggccacaacagtgtcacatgtctactctgtctggatgggaacctggcagaagggcattgtaaatggagttggataccgtcatccaacagtatctgccatagttgctagctgtgtaatcttagaagagttatttagccttgctgagcctcagcctcctgtgaaatgaggataattaatgccacaaagcttataaaaaagcaaattataggaagtgacttgcatataggaaatgcttagaattaagtacaacacaaaatgatgtcttagtcccttcaggtgactatgacaaagcacctagactgcgtaatttgtaaaaaacagaaattcgttgctcacagttctggaggctaggaagtcccagatcaagtgtcaacagattttgaaggtctgttcttcatagatggtgacttctatatgttctcacatagtagaaggggccaggcagctcccttcaacctcttttataggggcactaaattgattcatgagggaaaagccctcatgacttaaccactttctaaaggccccacctcttaatactattacattggggtttaagtttcaacatgtaaattttgagggtacactaatattcagactatagcaaaggatattataatattatggcctaagggaaacctgaccgatttgattgagggaaatattaatgtaaagccataaaaattattatttataggatattatagataattttattcatatatttttaagtaatacaataattttggaaaaatctagaaagagatgttgcatgtgctctaatagaaagacttattttttgtatgtcaatagtacatttattcattcaaactagaatgtaggcttaatgtgggcaagaatttttgtttgattttttcattgctatgttgattgaatctagaatggggcctgacacatagtaaatgctcaataaatatttgttgagtaattattctagttgataactagaaatagacatttcctgaatctccccttcactgtctagttaagtaataatataaggtcatatatattttataaattatttttcccatcttctttcttaaaatccaacaataaactataaccaaactctgataaacctaaccaagttctgaattgatggaggccagattaatgggattttattacattctaaacaggacataaaagatataaaaggttagaataaaagtgaatgttctcctctagaaaagtcaaaggactaacttataaatatagacaggtcagaaaaggaaacaaacacctatttatctaatgattagttacaaacaaactaatgagttgaaaaaaattagagaagagggctgggcgcagtggctcatgcctgtaatcccagcactttgggaggctgaggcaggtggattgcttgagctcaggagtttgagaccaacctgggcaacatggcgaaaaatacaaaaaatataaaaatttctacaaaaaatacaaaaactagccaggcgtgttggcgggtgcctggagtccaagctacttgggaagatggcttgaacccaggaggcagaggttgcagtgagccactgtactccagcctgggcgacttagccagaccctgtctcaaaaaaaaagaaagaaaagagcaagtcaatttccctaactaccagaaaaaatctttccctcaaggagaaagctgacttataatgaatgttgtggtcgaggtagtattatgtttaaaggagctcttctaaacgttcagttattagggtattttccacattggtaactaggcagagatcagcatgtgcaaggtttcacttctaactcatgtggttcagttttccagtaaaatcacatgtagagagaagctttttacctgagaccacctactcatttctccagttgcagaaaggcagtgtttcccctatggcaacataggtgccatcacaggcctcctcatgttggggtgaagttgtaaagtagaattttgtctgttttgcttcaagagctttctttcttcccagctcctttgcggtggcctcaccagacagagcctcctggctgttcacataccttcttttacctcctgtattttgctgtttcaaagggtaatgatagggaaaaaaaaaaaccaaaaaaaaaacaaaaccagtgctaataaccatctccctactgtagtcccactatcttcagcaatttcatgtaaatatattttttatttatcttggaaacactggtaatgtctggctcagtgtattctactccataaatatttgttgaataaaaatgcatgaatgatagccctgcattctgttccattcaagtcatcttagcaaatatttatttagcatttgatggtttcctggaattgtattaaactgtagatatttaaaaaaaagagatcatctctgtgcatgacaagctcataaacagtttagaataacatcaatgttgggattttcacgcaccgtagaagaccaataaggcaaatgttttcagatgcagtatcatgttagagcccttaaattcagcggcctactctgctaattcataaggcctgtttgtttttgccacagcttctctacatgcatatttttaataaccagaaagagttcttaggtgtgattccaaatactatttttaaggctacgttaggtttcagtaattcctttaggactcaaaatgtagtttatattttagaatccatttcctcactgattatgagcccagagagctaattgtggctggagacaaacagaaccttgctgactgatactgctttatattcatgaccactaacctcaactgggcccggagagccacacagtaatcctactacatttcactagccaagtcactgtcttgctaagccaggtcattgcttcattactttttctgtcatctctcctctctagtatcttcccttcctcactctctattgaagacctgcttcctactttcctgagaaaacagaagcaatgagaaataccacagcctccctcattgactaatgcattgacatctgtacccatatagttgatctttcctgctattgctatggatgaattaccattgccctaaggtcaactctccacttgggtactagatcttatcctgcctgcctactggaggacactgctccaaaactttcctctcttccttctgcattgtcattgtctctgtctttactggatcattcccattggcttttaagcatgctgtagcctttgcccttttgaaaaatataaccctccctttgttcctgacctctaccacccaatttctctgtttctttttatgaaaaaaaatcaaaagagttgtctgcactgtctgtctctattttctctcttctcttttatctagtttagtcatgttttatgtcctcaccactccactgaaacagctcctgtcaaagtcactgatgatttccccattgccagattgaatggcttattctcagtctttatcttacttatgaaacatttactatccgagcatttaatgtagctcttcacttcctccatttggcttccaggacaggactcccttttgctgctcctcccaccttcctggaaactttatctcagtctcttttgctgattcatcttcatttcagataaattcatagtgacccagagctctatattttgtcagcctaaaaaaagacactagggaagattatcttcaaatttgttgggtttacttgagaatataaataaggagtataatacagaatgcatgacatggcaagccagcagtgcatttggtgagggaaaggataaagggaagcttttattaccaaaaaggaatatatgtgagctatttaggaacagagttcattagttccagaggctcaaagccagagtgattgtcaattaataagtggagatgtattactgggtaagcggtcttccaaaaacatcttatctggattactgcagtcctaaaggatgtctagtgataaaccttatcaaagcaggggatgactgaaaggtttttagaaagtccttgggaacagttcttatctcagacatgtaagcatgggcctcttctccttcaggcctttcttgccctattttgtctgggtctgacaaaagtgatttcatcctggtatgtgcaactttcacacctaggaccttttattatctctatttacagtcattacctaaaggatagccttcaaatttgtggtttaaaattcctcaatgtatatctgtgtctaaatatctccaccaaatacagctgccatgtttctaacctgtcattcaaggtatcttgaagttaacattttcaaaaccaaacacccagtttcctctcccaagacttgcattttccatttcagtaaatggcaactgtatcctttgcattattcaggtcaaaatcaggtagattctcctggattcctctctattatggccttcatctaattgatcagcaaaaccttccggctctatcttttaaaatgacctcaccacctacactgcaatccaagccagatcatctctcacttgggagtataagaacttctaactggtccactcgcttcctccagagtcccttattcatgagttttcacagtaatccttttcacatgcacctcaggccatgtcacccttctgctcatgaacctcccatttcattcagagatcaaagccagagtccctcaaggactctatgtgaccttgcaagtcttctaaactttcccctcttccaaaactctctgattttgtctttaatccctttcctagtaaaacctcactgacctccttgctgctctttgaacaggccaagcaaggtcctgcctcagggcctttgcatgttacctagaatgctcttcctccagagagccacagggtttccttattgccctaatgtcaccctctcagataagactttcctcatctctctaaaaaacaaaaaccttaaaacccacccctactctcagaactgcctaacctttttactccgctttagttttcttctgaacatttctcaccatacatagatcagattctatatttatttgtttccaccaaaatgtgtgcttcatgacagttgggactttatctgttttgttcattgtagccccaatatctaaaaatatacttggaatatagtcagtgttaaataaatatttattgaatgagtgattaaatgaatgaaagacactatttaccattatcaacataaatctgcaatgttttttgaaaatctataaattacaactatatctaggtgctcagggaacatcagaaaaatatgaattatactaactcccttcacaaatcatctgtcatgtattagcccatgaaaaaaagaaatagaaataagatattcaaaaactttgtaatatacagttatgtaaaagatagaggaaaaaccaaagtgttgttctcctttctactctcaacagcactcctgacacgaaatgtgggaagattttccccacacaccaatcaattctcctgcagacatcagctagatgtcctctaatttaattcaattctgactctgtctacctggaggtagcatcagatcccacagctaaaggctcagtcccacaagactcaaccctgttttaaatgtcaatcacaagccccaggtcatgacctgtgcttttgaccaaccggctaaaaattgtggtttccacgatacactcctcaggtttgattaacttgctagagtgtctcatagaactcaggaagacactttacttatatttatccatttattataaagaatataacaaaggctacagatgaacagccagatggaagagatgcatggggcaaagcatgcgggaacaccaccctgcaggcatcctcatatgttcagatatctggaagctccctgaacccagtccttttgggttcttatggaggcttcattacataagcacggttcatcatatcattggccattggggatcattcagcccttctctcttcccaggagatgggggtgggtaaagggcaagggggttggggggactgaaagtttcaatcttctcatcacattgttggttcccctggcaaccagcccctaccctaagcctatccaggagcccccagccatcagccatctcattagcactttggagattccaagggttttaggagctgtgtgctgggaaccagacagaaaccaaatatatatttcttattatgtcacaatatcacaagttctatataaaaataaccctaaggtgtaattagtcattattatcagattcttcaattaaaacagtgctttgtggtctaggtgctaatatggggtagactttaaaagcagaattaaattacttagtaactatggttactgtcagtttgaaaatacatttccattttgacttgtggtctcagtatgtgtggtaaaagaattagccacaaaccaatgaagaacatataatgtggttttctctaactggaaaatattcaaagatgtcctacttaccatttacacatagttgccattttcttgttttcacatttgaattcttacttagcatgaggttaacatactttaacaagttctctgttaaggtatttacgtttcatcttatttcagaaagaatataaggtaactatgtaaacatctcaatttaacggcataatttaacttaagatacagtgatataagagaaattttactggtaagaaatatagcaattttaaaagtttcatttgtattctttagaatgtgtcaaagtcacatatcataaatatcttctttttttatttaaatggacacatagtctcactctgtcatccaggctggagtgcagtggtgcaatgttggctcactgcagcctccatctcctgggctcaggcaatcctcagcttcctgagtagctgggaggacaggtgcctgccaccacacctggctaatttttgtatttttagtagagacagggtttcaccacgttggccaggctagtctcaaactcctgacctcaagtgatctgcctgcctcagcctcccaaagtgctgggattacaggtgtgagccaccatgcctggcctcagccagtatctcttaattcagaaaaattgttgtttaatgagtccttcattaggtgagaagccatctactctgtttcttatttaatttactcatgataacaacaacgttatcattcctatactgaggatgagtaaactgagctgtatagagcttaagtaacttgctccaagtcttacaacttatcaaggggcagagctgggatttgaaggtaggtctgtctctaaaatccaagccctgtgattttagtcaccatgacataccctcctcgtcctctcatggtgttcatagtgatctcacgctggtatgaggaaagaggtaacacagaaaaaatgcactggaaccgaaaaacctgagctgaataggaggagttgctcctcacccctccaatgactatcagccttagagagattccttgaccacttgctgaggttgtcttgttctgatagtagcaataagtttatataaattatataaaatatagaatttctatagtacttagatgcacacatctgcttattgcttggtattaatagcataaggctggttccttttctctgcgtctcagcaagcacgtttcttggattccaaaagatgtaagtggctagcaagtttaatcctggtgggatattttggggctagaaaaatttaggagttgctgaaaagatatatagtagtaacaggacttggtggagccttggtggagggacagtgatttttaagctttatcttctccacaaatccaatcaaggcagtcccctgagagagagggcagttgatggtattagatttaaaaaatcaggttagagtctgcctcaacagcacaaacagtgtaattggaacaagacaaagaagatggcatgacccctgatcaaggacggcatgcaaatttgtgaagtatttccatttgatacaaataagaatgtatatggctgagcacggtggctcatgcttgtaatcctcacgtgggaggatcccttgaggccaagaatttgagaacagcctgggcaacatagcgaggctgcatctcttaaaaaaaaattattttaaataaaaaaattagttaggcctggtggcacatgcctgcgatcccagctactcaggaggctgaggtgggaggatcacttgagcccagaaagtcaaggctgcagtgagttatgatttgtgccactgcactcaaacctgggtgacagagtgagaccctgtctctaaaaaatagaagaaaataagaatgtaggctacttggctttccagcattgttcagatttcaaggttactttccttctaaagtagtatatctatatcaagttaagtttcatgatgtaatgcaattaacttaggagtctgaaagtcataaaccaaacaggtaacaaagacctagaccattgccatagctatcaaaacagggaaaaaagataacataggaaaataatattttaatgcttaaaaaaatgcttaaccttctttagtaattggttggctcttaaaggtgacgaagagaaaattgttaaaaatggcaaactgagaaaggtggaccgcatgcctttgacaacaatgaagacactggcaagaagtgctgatttgcatggaaagataatttagttgtcagttttctatagttctggttccagtcagacatccaaatgaagctcccagtgagggcacaggtatgcgtgagacacacatgtgaaatagcaggtgaagctgagaagggagtgagcccagaaaaaggggtcagggaaaaggcaaacaccagggctgaccttcaatgtgagtgaactcagagagtagggaaagaaacccaggtggaaggctggagggggacatgccactaagtaggtgtgagagaggaaggccctctaacccagaagacaggctttgggtagcaaggaagtgagaattataaccaatcaagagggataaatgcttaaatgaggattcagaggacttcaccttgtaaaggacaccattttaaaagagcagtttcaaggaaaagtaaaggcaaagatcacaataaaaggactaaagaccaaatgggaataagttggatgtagttactaaggaggtagaaaatctgagaagcttaagaacatgaaataatggagtaggtagttcaaggggctcaagaaatgggaactgaggaaattctagtgaaaatacttccatgttacaaatgaatctgattacagattatgttgtgttttggagtctgaggggggccctattcatctctgcagaagaatgagtgaacaaggggtgggacaagaggctaggagatttgaggacactcctgtgttagtccattcttgccttgctataaagaaatatctagaccaggtgtggtggctcacacctgtaatccctgaactttgggaggccaaggcaagaggatcacttgagctcagagctcgaggctggcctgggcaatgtggcaaaaccctgtctctacaaaaaattagctgagagtggtggtgcatgcctgtagtcccagctactcaggaggctgagatgggaggacctcctgagcctgggaggtcaagactgcagtgagctgtggccataccactgcactccagcctgggtgacagagtgagaccctgtcattaaaaaagaaaagaaaagaaaagaaaagaaaagaaatacctgaggctcggtaatttatgaagaaaagagttttatttcagctcacagttctgcaggctgtccaggaagattgtgaacaagatagagggaggggggatatgccacactctttaaaatgaccagatctgcctgaactcagagggagaagtcactcattactgccaggagggcagcaagccattcatgaagggtctgccccacaagtcctcacctccaacactggtgatgactttgcaacatgagatttggagaggatgagggtggaaacaatatcaaatctcttcacagaaatcagaggagcaaagaagcattctccagcactcctttcgccaccgcctccaaccctcgtccactaaaaacatcatgtgattatttaggggtagagatttaaaaacagaggctcaaacattctgtccaaagccagtgaaaacaatttgaaactctggcattaacagatatcattcggaattctaccaagtttattgggctacttggatattctcattcaattataaatacttagataaatatgctttgaaatgatacttaatagccctctctattcccctgttctaaactagatctttcctctttatcttttccatcctcttccacagtgcatgactccctttcccctcatcaaacctgtcattaggtccagtcagttctacactcaaaatccttcatgggttgggcgaggtggctcatgcctataatcccagcactttgggaggccaagacgggcagagtgcttgagttcaagagttggagactagcctgggtatcatgggaaaaccccatgtctacacaaaatacaaaaattaattgggcatggtagtacgtgcctgtggtcccggctactcaggaggctggggtgggaggatcactcgagcccaggaggtcaaggttgcagtgagccaagatggccccactgcacgccagcctgggtgacagagccagaccctgtctcaaaatccttcatatccacccccattggcttccacctatttcagactaataatccaattctggattattgtatagccttttgatttttccactaccaccactttcatttcccacaaatcctccgtaaaggtgctagacagtcccagagaccctcaaaatagtgtgaatctcactatatgcactcactttctatcacttacatgatacagtcctgatccatcagtagctcatggaaggtcttctgacaggcctgctccactacaagtttcttcttgcattttcaggatccaggctattgcacttgttgaaccttctcagaaatgtcatgttatttcacgtatttgtttcagaacatgctggttgtctctgtccaggaggttggccttccccctaccccattgcgatctctcaccccatcctgactcctccccaccactccatcctcaccctatgtcctgatagaaatgtctgacttcacagtccttgcactgaaccaaaatgcagtgctcttttccaaaggcagctattctggctattccaagctatgtccagtgccgacttccctaacaggcacagtaggcacagtggctagggcccacaataattttaggagtccatgaaaatgtttaattttacttaaaatcagaagagaaaaataactgttatgttcatgtatatgcatgtatatggcatgcatatgtatacatctatgtatatgtgtgtatatttttgtgtattttatatatttatataatatatacgctcattgtttttttaatggaggaaagggtccatgaaggcagaagtgttcagggcccattgaaatcatactgtggcccttgtcagatttgaatttattttttaaaattgtgaaaataaaatcaattttgaaatatttttcagtaaaaggttgcatatttgaaagcaggcttaaaaagttagcgtttttgtccaggcacagtggctcatgcctgtaatcctagcactttagaaggctgaaatgcacggattgcttgagtccaggagtttgagaccagcctgagcaacacagcaagaccctgtctctactgtttagaaaattaaattttaattttttgtttttgtttttgtttttgagacggagtctcgctctgtcgcccctgctggagtgcagtggtgtgatctcggctcactgcaagctcctcctcctgggttcacgccattctcctgcctcagcctccccagtagctgggactacaggcacctgccaccacgcccggctaatttctttttgtatttttggtagagacagggtttcaccatgttagccaggatagtctcgatctcctgaccttgtgatccgcccgcctcggcctcccaaagtgctgggattacaagcgtgagccataaattctaattttttttaaaaaggtaacattttaaaaatctggaataattaattcaaactttttttcttttttttttttttgagacagagtctcactctgttgcccaggctggagtgaatggcgtgaccttggctcactgcaggctctgcctcccgggttccagcaattctcctttcgcagcctcccaagtaactgggattacaggcacatgccaccaattattgtatttttttaatagagacagggtttcaccatgttggccaggctggtctcgaactcctgacctcaggtgatccaacccgcttggtctcccaaaatgctaggattacaggtgtgagccaccatgcctggccaattaattcaaactaaatacaatttatgatcatcttttttttttgttttaagtttagagacagaagaagagcaacagtatctcacctcaagctatattaacatctccactgattcattacaaggtggcaagaagtacttggtttgggtccaagcagcaaacgcactaggcatggaagagtcaaaacaactgcaaattcacctggatgatataggtaaagaataagaaattctgtaagtctttaataataaccagtttgtgctgaccttgtcaaatgaggtctagatctgaaagaagttcccctaaagttcctgcagagcatgataaaagaaacagaaattacccataattctacctgcccaagacaacttagagatgagtagtgctgctgccattttgttgtatatccttccagtcttatttctaaattgttgtatgtggctgggccggtggctcacgcctgtaatcccagcattttgggaggtcgaggtgggcgatcacttgaggtcaggatttcgagaccagcctggttaacatggtgaaaccctgtctctactaaaagtacaaaaaaaaaattagccaggcatggtggcgggcacctgtagtcccagctactcaggaggctgaggcaggagaattgcttgaatctgggaggcagaggttgcagtgagccaagattgtgccactgcactccagcctgggtgacacagcaaaactcttgtctaaaaaaaaaaaaaaaaatccatatatgtgtttttacaaatatttattggaaacttcttattttcaagcattgttctaaaggctagagtgaataagatagacaaggtgcctgccctgaaataacttacaatctactagggaaggcaaaaaaaaaaaaaacaaaaaaacactatgatcatgataaagtttgttgagtcttacgttgtgaaaagtacagcatgctaaattcttatactctatacactacttggggacttatgtaaggtttttttgtttgtttgtttttattttgaaaagttttacaaaatacatagaaaattgcagagaattatgtagacctctctatgttcatcagccagatttaacaaacattaatagtttttgtttcagatattttataccctgcctttttatacttatatattagtaataaacatttctattaatttaatctatagttttatttttaatgttgtctgaatatatagcagtttaatccctattgttggagattaacactgttttcagtttttaaccatataaactatattgccatgaacaactttgtggctgaattttagcatagtttttgattgtttccttaagataaattccaaaaagtaattacccttgagccttttacaactacatccaagtcctaaaaccacaattcagccaaatgacaccaagtctgaatcatcctgtctttgcagatcccagtaccaatgattcctgttgttataattaaagatatttcaataagctaacatttattgagcactttcttatgtgctaactactttttctttttttgaggcagagtctcactctgtggcccaggctggagtgcagttgcacaatctcggctcactgcatcctctgcctcctggcttcaagcaattctcatgcctcagcctccctagtaactgggactacaggtgtgtgccaccatgcccagctgatctatattgccacaaagttgttcatggctatatagatttttgtatttttagtagagatgggattttgctatgttggccaggctggtctcaaactcctgagttcaggcaatcctcccacctcagcctcccaaagtgttaggattacaggcgtgagccactgccagctaacagcttttcacatacgttatttaaccctcatatcaaccctattttgtagatgagattataatttatatattagaaaactgaggtttgatggcattaaaaaatttgagatcacagaggtagggaacatttaattcaggggatcctcctgccttagcctctcgagtagctgggacgacaggccacaggccacagtgcctggctaatttttttaaatctcttttagagacagagtctcattttgttgcccaaacttgtctcgaactctgtggctcaagcgatcctcctgccttggcctcacaaaatgctggtatttcaggcgtgagccaccatgaccggcctgaatattagatttgaaaacacactctgttgaatatgatgatggacaattattccattttattgtatatccttggagttacagttctcttgactatgctctatacatttttgtgaatttacattttgaggtagaatgcagtaatatacccttttcttacgatagataaattaatgatgtttaagagctatcatgaccagtatcattttagcagtgcaatttttaggaactaaataataataataatacttgtattattattaccattctgatcttaatatcattgcatcaatttatttagcaccttctgtgttccagacacctctcatgcacatcacgtttcattctggaaacaaccatacatgatactactgtccccattttacagatgaagaaagtgataaaatgatttgcgcaaagtcacttagctagtaatgaggaaagccagatttcatcccagacatgctgacttgctatagtttaccaccatcccacctcccacaaaggaaaaaatcccatctggcctctacttccagactgacttctcacactgggaactaggagtttggagttgtggttccttttccttcttctccttctttctctttccctcctcctcttcctccacctcctcctcctccacctcttcctccccttttgtctctattcctttccttgccttttcattatcccagaatgtactcagaaactcaggttgaaaaatatggattaattgtataggaggccccagccaacaaaaccaccagattattaacagatgagccgtcttcatgggtaacactttaagaatgagacaacgcaaattagataaatgaactgttctatgccactgagaacagaagactgtcctgggaacaatttccacatagcagctacgggctgggctttaatgacattagggaggttatgctgaatggcagcgcagagttagtagcacattaacacttgccaaaaaagcatatgagaggaatgcttttaacttctcttgtagggaaataaattcgggtacagaaattgctgtgctttctgcctgccattttgaatattacttctccccattatataaatatggataaatatgtgttggaaagatgctaaaaggctataatctcgcttttatttcaatacgagtagaatttaaaaatatgacatctataataaaattgatactttttttttctttttttgagacggagtctcgctctgtcgcccaggctggagtgcagtggcgcgatctcggctcactgcaagctccgccttccgggttcacgccattctcctgcctcagcctcccgagtagctgggactacaggcgccagccacctcgccccgctaattttttgtatttttagtagagatggggtttcaccatgttagccaggatggtctcgatctcctgacctcatgatcggcccgcctcgacctcccgatacttttttaaaatgtaagatctggtggaaatatgtgaaacctaatcagtaagaataaaaattcaacatctgagtcttgtgtaacaaactaaatattcaagtatgtattacataataaatgccagtttgcaaaaatatgaactcattcaaacttaaagacttaaatctggattgcactgacctgctttatgctgtgattcttactgtgctatctgcatttctcataagactaaacagatttttactttctcctcagaataattcttttgccaaaagggtcctagaatgcttgtgctgctataacagtggccaagccgaactcagaggtggaggtgggtgacaggaggaggtgagaaatttatggccttgagggtgcttaaaatggtcctaagcaatttcagagccccagggatccatgcccttgactctagaatctcaatactgattttttttttttttccagggttggggttgggcagagtcttgctctgttacccaggctggagtgcagtggcatgatctcagctcgctgcaacctctacttcttggattcaagtgattcttgtgcttcagcctcccaagtagctgggattacaggcgtctgccacaacacctggctaagttttgtatttttagtagagacaggtgtttcgctatgttggccaggctggtcttgaactcctgacctcaaatgatctgcccacctaggcctccccaaagtgttgggattacaggtgtgagccactgcgtccagcctgaatactgattcttattacaagaagcttgcactagcaaaaggaaatcagctatggaattcagatgataaaggttgagtacagtaaacttgttaactaaattaaagctactgctacttctacgccaggtgtagcccccaaaattttattagaggactgtgttactagacagcaaatccttaatttttgtttttctctgtaaatcatctacctttgcacataattttagataataatagtataatagtcattgtagtaaaacaataattttcttaaagaaagttttataaacctagaataaatgaaaataaactaattcatagagaatattaacctgtaatagtggtctctaaaaccacgcctacaggcccttgagttctttgttaaatagtacagctatcaaagtcataggttttcattgttccttaggggagaaatggaaagtgttaatgattctattctgttcagacaaaacaaacttacaagtatttacacagtttagaaacatagcgattccaactatatacctcctactataacaaaacaactaataaaataaccaatcagaatttcagttccttctgtgaaggttattattatagaacatgccaacaatttacaaagccttagagtcttatgagatctgctttcttcaaataaaatttcaaggaaggacagatttattttaaaggagaacaaaacaagttagcaatagatataaatattcatatgtttccctaatacacaagagaaggagaagattattgtgataaaagatctgaattgaacatccatctttctttctttctttctttctttctttctttctttctttctttctttctttttctttctttctttctctttttttttctttctttctttcttgctctttctgtctcttcctttctttctcttttccttcctcctttcccttcctttctttcttttttgacagagtctcgctccgccacccaggctggagtgcagtggcatgatctcagctcactgtaacctctgcctcccgggttcaagcgatcctcctgcctcagcctcctgagtagctgggattggcacgcaccaccaccacgccgggctaattttttctgtttttagtagagacagggttttgccacgacgaccaggctggtctcaaactcctgaccttaagtgatctgcccacctcggcctcccaaatgctgggaatacagacatgagccaccatgcccagcctttctttcttttttttgagacaagctctcattctgtcgtccagggtggagtacagttgcaccatcatggctcactgcaaccttgaactcctgggctcaagtgaccctcccatctcagcctcctgagtagttggaatttcaggcacacatcaccacacctggctcaatttaacatcttttcaaatctgatcatctgaactaaatttgggtgcatatcatctctcctcgaacagatggagggaagaggggatagctcctttatctcatttttatactcttttgttaacaaggctaggctctggaattttattagttaggattgtaagggagtgaaaggtgggggaaagacaggactattagagttcagtttttttttttttaatggggaatgggctgaatagaattttccttaacagcagttttcttctatgtaacaaattagcaaataagaaaatgtccataattgttcattagaccatgaattttatttttttttactttgagctttctcatatctagatattgacgaaaagatgccagtttctccctaggcaagttttaaacagccaggtctttttttttttttttttttttctagtgataccttctgcagccgtcatttccagggctgagactataaatgctacagtgcccaagaccataatttattgggatagtcaaacaacaattgaaaaggtttcctgtgaaatgagatacaaggctacaacaaaccaaacttggaatgtaagctcaactttcattatgctttagcatgtgaatgaatgatttaaaagccaaccatcagtggctacagtggacttattatgtctattttacatgtttttaatctgattgtttgcatgatattcaagccactttagtttttgttttataatttcaacttttattttagattcgggggcacatgtgcaggtttgttacctggatatattgcatggtattgaggtttggggtatgattgatcctgtcacccaggtgctaagcatagttaccaataatttgcttttcaacccttgccttcctaccttcctccacactctagtgtggtccccagtgtctattattgccatctttatgtccatgagtacctgatatgatttggctgcatccccaccaaatcgcaacttgaattgtgtctcccagaattcccaggtgttgtgggagggacccagggggaggtaattgaatcatggaggccagtctttcccacactagtctcgtgatagtgaataagtctcacgagatctgatgcgtttatcagaggtttccgcttttgcttcttcctcattttctcttgccaccactaagtaagaagagccttttgcctcccaccatgattctgaggccttaccagccatgtggaactgtaagtccaattaaacctctttttcttcctagtcttgggtatgtctttatcattagcatgaaaatggattaatacagtaaattggtaccagtagagtggggcattgcttaaaagatacccgaaaatgtggaagcaactttggaactgggtaataggcagaaattggaacagtttgaagggctcagaagaagacaggaaaatgtgggaaatttggaacttcctggagacttgttgaatggctttgcccaaaatgctgatagcaatatggacaataaaatctaggctggggtggtctcagatggagatgaggaacttgttaggaactggaggaaaggtgactcttgttatgttttagcaaagagactggcagcattttgcccctgccctagagatctgtggaactttgaactcgaaagatgatttagggtgtctggtagaagaaatttctacgcagcaaagcattcaagtggtgatttgggtactattaaaggcattcagttttaaaagggaaacagaacataaaagttcagaaaatttgtggcctgactatgcaatagaaaagaaaaacccattctgggggggagaaattcaagccagctgcagaagtttgcacaagtagcaaggagcctaatgttaattccctagaccatggggaaaatgtctccaggccacatcagagacctttacagcagcccctcccatcacgggcttggaggcccaggagaaaaaagtggtttcatgggctgggaccagggtcactgtgctgtgtgcagcttagggacttggtgccctgtgtcccagctgctccaaccatggctcaaagggccaacatccagcttgggctgtggcttcagaaggtggaagccccaagctttgacagcttccacatggtgttgagcctgcgggtacacagaagtcagaattgaggtttgggaacctccacctagatttcagaagatgtatggaaatgcctggatggccaggtgaaagtttgctgcagggacggggccctcatggagaacctctggtagggcagtgtggaagcaaaatgtggggtctgagcctccacacagagtccctagtggggcattgcctagtggagctgtgagaagagggccaccatcctccagattccagaatggtagatccaccaacagcttgaaccgtgcacctggaaaagttgcagacactcaataccagcccatgaaagcagctgggagggagactgtaccctgcaaagtcacagggttggagctgcccaagaccatgggaacccacctcttttatcagcataacctggatgagagacatggaataaaagaagatcatttgggagctttaaagttgccctgctggattttggacttgcatgggcgctgtaacccctttgttttggccaatttctcccatttggaatggctgtattacctaatacctgtaccctcattgtatctaggaagtaactaacttgcttttgattttataggctcataggtggaagggacttgccttgtctcagatgagactttggactgtggacttttgggttaatgctgaaatgagttatgactttgggggacaattgggaaggcatagttggttttgaaatgtgaggacacgaatttggaggggccagggatggaatgacatggttttgctgtgcccccatcaaatctcaacttgaattgtatctcccagaattcccacatgttgtgggagggagccaggggaggtaattgaatcctgggagcccgtctttcccatactattctcatgatagtgaataagtctcatgagatctgatgcatttatcaggggtttccgcttttgctttttcctcattttctcttgctgctgccatgttaagtagtgcctttcaccacccatcatcattctgaggcctctccagccatatggagctataagtccaattaaacctctttttcttcccagtctccagtgtgtctttatcaacagcatgaaaacagactaatgcagtacccagtgtttagctcccactcataagtgagaacatgtggtatttgattttctgttcctgtgttaattcacttaggaaaaccactctagctttaataatgattattgtatgcttgcaaacagagaactgtttcctcaaacgatccacttgccttttattagttgctaaatagacaaagctccgactaagaggaatctaattagctatttgtaattcagtgtctcctagggtgaaatttatatcagtgatcatggataaaaaatttaagtattctgtgctgaaatttgacaggcatccagggaaacaaaaattgttcggaaaggagaactaagatgtatgaatgtttctttttaagtgaaaagtgtatagttcagagtgtaatatttattaccagtataggcctgagtcttaggtgagcttaaagatgatgataatccgagtaaaaatgagtttaaaaaagtgaacgtctagagaaacaaactaaccatccttaaatgccaaggttaaattatctagtgtcttaagaaaggcatagctgcaaggttcattaataaagtcacatcagataacagcacctgggaacaacgtaataaaactcagtaatttcagctgtggaggaacactagcctgatttaggaagaagttccaattttgatatattttaaaagaaatgctatttgattatttttaagcgtaacaaactgccatttagtccaaataattaagaaatagtttctggcctttttttttcccttccgatacttttttaaggttttttttttagagatagactctcacttcatcacccaggctgaagttcagtggtgcaatcatagctcactgcagccacaacctcttgggctcaagtgatcctcctgccttagcctcccaagtagctgggactacagtcactcatcactatgcctggctaatttatttttatttttatttttttgtagattcttgccatgttgcccaggccggtcttgaactagcctcaaataatcctccccctggctttctcgtcagcctcccaaagtgctaggattacaagcatgagccacagcacctggcctggcttttcttaattaacagttatgtatcagctgtgaaattaccagttttcagcgcgttaaataaagtgatatgtatttaacactaaaatacattaaacataacttaatttttctttggtgctaagcatgattccaaatccttttgctgtttaagactgattctgagtatcggttttgctatagagtaatatatcttaaaagtatcaagaagatgggggcaaaactatactaattttattatataccctaaaaattactcattaaaagtaaattccttacgtcaacttgtttacctttgttcactcaaatcataaatgtgaactttatggttgtttgcatacacttaaatgggatccacgttctgcatcatttgattgataatcaagtgaagatcctgctgaattccttttgcatatgcagaatttagattaaatttcaaaacaacacaaatacaattctcaagtcctagattctgaattaatggggttttatcctaataagacacctggggtccttgtatagtatcacagtcatagaatgatattaaagaatactgagtttcttaggctgggtgcagtggctcatgcctgtaatcccagcactttgggaggccaaggcaggcggatcacctgagctcagggattgaagaccagactggccatcatggcaaaaccccgtctctactgaaaatacaaaaaatttagccaagcctggtggtgtgtgcctgtaatcccagctactcagaaggctgaggcaagagaatcgcttgaatctgggaggtggaggttgcaatgagccaagatggagccactgcactccagcctgggtgacagagtgactctgtctccagaggaaaaaaaaaaaaaggataccaaatcctcttacttcatgcaaataggagtatgtaatagactagaaaaagtgtttagaaaatagaaaggaattatattatcagtgtctctgaataagttttcagaagccaactgttttctggttgaaactcttattctctgctccccctggtggtgctacataggccatcttggtaacaggtacatttgagctcacttttcaaaaccttctctttatcagaagtggcaaatagagtagaaagaatatttgttatctcatatgctcttcataacaatcctttgtgataggtagtattagctccattatataaatagggaaatagagtttgaaagaagtcaagccagatttttttgaacttataccgtcagtaactaagtgcctctcacagactctacatcactttaaagaccaaataaatatttagaaaatgaaaagacaggtttcaatccaaagccacttctgtctcctccaccattatttttttcagaaagtttttttaaactcatgactccctcagtcaatctaccacgtttcctttaaacacaaccactaacacagaaaaagtgaattaccatttctatccagatcactcaagccaagtcactgtagccagaatgaagctttgtttacatttgctactgtcaatttcatctgggtcatgcatgaagtgttgtctctgcatgtctgtaggaagacaggaaagtcagagtcaagaggagtgggaggataccaaagattacaggtctcttctaccactttagctccgtggtggcattgcctccattagatattgctggaggtggggggtttacttcaaagcgacaggaaagccttgtggccaagagcacaggctctagagttcaaagcctggctctgctactttctagtcatgcaatcttttttttttttttccttttgagacagggtcttgctctactgcccaggctggagcgcagtggcgcaatctcagctcactgcagccttgacctcctggactcaagcaatcctcctgcctcagtctcccaagtagctgaccacaggagagtgccaccatgcccagctaaattttaaaaatttttctgtagagacagggttgtgtcatgttgcccaggctagtgtcaaggtctctaactgctgagctcaagcaaccatcccgcctcagcctcccaaagtgctggtattacaggcacgagccactgctcctggctaagtcatgcaatctttctttttctttttttttctttttagacagagtctcactgtgttgcccaggctggagtgcagtggcgtgatcttggctcactgcaacctctgcttcccgggttcaagcgattctcctgactcagcctccctagtagctgggattacaggcatcgggcaccacgtctggcttctttttgtatttttagtagagatggggtttcaccacgttggccaggctgatcccaagtgatctgcccatctcggcctcccaaaatgctgagattacaggtgcgagccactgcgcccggcctagtcatgcaatcttgagcatgtttcttatcttctctgtgcctcgttattcccatacataacatggggataagataagcccttatatcatgtgtttgttgtgggaaaggggatccaatgatgtaagaagttagctacaagcctggaatagtgtaattgttcactaaatgctagctagtaatgttactctctagcctctaggggagccatgcagcagggatagtagagcaaaggaatccaactgaaagtcacagtattgagggtgcttttcagcccctttagctaaggtacatacagactgtgaagtatctaagggaattaggctgacgaggcaaggagaatatgtgccacgcagcagtccatgtttcaggcaatgatggatggcatatatgatggtggtcccataaacttatcatgcagctgaaaaatttctgtcacctagtgatttatattacaattgcctgcaggattcagtacagtaacatgctttataggtttgtagcctagaagcaacaggctataccatatagcctaggtgtgtagtaggctataccacccaggtttgtgtaattacaatctatgatgttcgtacaatgatgaaatcgcttaatgatgcatttctcagaatgtatccctgtcgttaagtgacacatgactctagttatctcttgaaagatggttccaggtcaaagcttcagattttgtctctggagacacagctgcctctgggagaacatttacaaacatcttgctcccacctcatttacctctctcccttctgaattccctagaagtctagcttagaccaggttggaatatatacagattttagtaaaggctcagagatagcaagaagaaaataaatctcagctgggaggggtgatacaagcctatattcagctacctgagagactgaggcaggaagatcacttgagccagggttttgaggctgtaatgcactatgatcccaccagggatagccactgcactgcagccggggcaatacagtgagacccccatctctataaaaagaaagaaaataaacatctcagagaaaaaccacctactatgtaatcttgattacaaaatataactagcttttcaaagttggtttgagacctgtcttttcattttctaaatatttatgtggtctttaaagtgatgtagagtctgtgagaggcactcagttactgatggtataagttcaaaaaaatctggcttgacctctttcaaactctgtttccctatttatataatggagctaatactacctgtcacaaaggattgttatgaagaccatatgagataacaaatattctttcagttccatttgccacttctgacaaagagaaggttttgaaaagtgagctcaaatgtacctgttaccaagatggcctatgtagcaccaccagggggagcagagaatgagagtttcaaccagaaaacagttggtttctgaaaacttattcagagacactggtaatataattcctttctattttctaaacatttttctagtctattacatactcctatttacatgaaataagagggattggtatatcggctatctactgctacgattgagggaagagagagaccctctcatattgttttatatttttttatactcagtacctgttttaagaaaaaacaacaaggaagtaaaagcaaagacaggcaacccagcaccaggcccgaaaccaggactgggcctgcctggccaaaacccagtagttaaaaatcaactcataacttagaaagcgatgttattcatagattccagacattgtatagaagaacattgtgaaactccctgccctgttttgtttctctctgaccactggtgcatgcagcctctgtcacgtaccgcctgcttgctcaaatcaatcacgaccctttcatgtgaaatctgtagtgttgtgagccctttaaaaggacagaaattgtgcattcggggagctcggattttaaggcggtagattgccgatgctcccagctgaataaagcccttccttctataactcggtgtctgagaggttttgtctgcggctcgtcctgctacacaataatgtgtaacaaactgccacaaaagctgaagggacatataacaataaacatttatttctcaatgtcagtggggtccagctggtctaaggtgagctttgctggaattgggcatgtgtttgcagctcagctggctcactcctatgtctttgcatgagctctcctctacatgtctcctggtcctctttctgccatcaacaggctagcctgggcatgtccttatggtgatggcagagggcaagagtacctaaggcccaatctttccagtgtctttcaatcctctgcttatatcaggcttaccaatgtctgttggctcaaagcaagctacatggacaaaccagagccagaatgggagaacgttcaaagctacatggtaaagggccaggatatggggctgggaaattacctgggcattattgcaatccacctcaaaatgtcatcaaggctctccacctgctgctggaaccagttcgtgtgaggagaggaagatggtagcatctttccaaacaataaagaccaagaaccttggcagtcttcatcctactctagcatttgacactgttgaccaccttgtcctgaaaatgtctctcctcttggttttcccatttcaagaatgtgttagtcttctatcaatatttctggatgccctttcctttgtaaattattttttctgccaattccctaaacattgacattcccttcatacctaccatctcactgtctcctctctttcttactcctttcacctattcccaaggcctcagctacacctgtatgtaggctagaaactggagccctcccaactctcatgaaagtcagacctatatttctaactatttgtttgtatctttatgggctgtctcctaggcccctcaaattcatgtcaaaaattgagctcccagttgcgaacggcaatccacacctacttccgtatactctgtttcagggcatttgccacattgtatatacttgcctgtttacacttctatttctctactctttaactgtgaactcatggaaggcagagatgtgccatgatcattttttgcatctccattgatttttataatgtcaagttcatgtggataatattttgaaacattagctattactattattattattatttgtctaatggtgtccaactttagtgacagaggcagggaaatctcagctatgtgttcaagtaagtggattattggtgactcatttctctaattaagctacaccagggtctcagcaaactttttattcaagggccaaatggtaaacattttaggctttgcagaacataagatttctgtctcaactactcaactctgtgtttgtagcgtgaaagcagtcacagacaattatgtaaatgaatgggcatagtctgttccaattaaaatttcatttttggacactgaaatttgaatttcacataatttccatacatcatgaaatagtctttttttaaaaaaaatttctttcaaccatttaaaagtgagaaaaccattcttaacttaggctgcacaaaaccaagtatttattcttttcttgaaagggctgatagaacagcatacagatagcaaagacaaatccttggcttcggcatttccaagtctgaaggttggtataagacagaaaatgtggaaaaatgtatgattagtcacaaataagtttccccatcaccaccccatttccctcctctaccaaacactagataaaaattacttgggcagaaaggggaggaggttgtagggtaaagggagaaaccaagggtttagacactggtgggttaccaaggaccctctcatccagtgccatgccccatactgatagttggggaaggtaggaattatagaactctaactaggttggagattttttcttccctccacccaaggatgttcccaggaacttatatttaacataagaaaagagagatctcaaactgaagaatattacgtttcaatgacccctaaccttcagagttgaggctctaagtagaaatggacttacttcaaaatttcttgcactcttgaaattatactatttacagaggcactcaattaagtccagctctttcaatgttcattttagcctccaaagatatcatcctgaggtatcatgttgtacttgcagggccctaagcagccacctggtttgcctcatggtttaaacaactgataacagttccataacgttcttcttactattttggaattctggctgtactttacggtatttctttcctttaagaggacttatctataaatgtaatggcagcaaacatattggagactaacaacttccttacaagagacactaatatgaagcagagtgcatttgagagtcaaatctttggcacagtaagtaaaatttctctagatcccaggagacctcagacactctccagttatattaacattgcagaaaatttccagggccatgaatagagacatggcttatggaaactctgaaagatatctttcatgcagagtttgaaaaatatcattcagagtttccataagccatgtctcaatagacaaaactattctagggcagatgacatcccccaaacaaatattttcatgaattttacattttcagtgcattgcaagttttaaggattaatgttaaggttgggttttaaatggaagcatccctgaatgaggttgatacctcattcttttttttttttaatctcaatcgttgtaactttctctttcatgagtatctgataactgctttcacctagttgttgcataaatgcatttctagacatattgacatattttatgtctgcctttcaatcatgcttcactttttccagtaaatcagttactagaacatactgaagatgctgtacaaatgccttggtttctttctgccaactgaaacagcttgtttggggctcatttttcattacgttttatagcagggtgtagtgctccagggaaaaaaggatttttgtgtttatccctcagctattttctttatcctccattggatttctcagttcaactttcttaatattttaatttttctctgacacatacacatatatatatatatacatacatatacatatatacatatatacacatatatgcatatgtgtgtgtgtgtgtgtgtgtgtgtgtgtgtgtatatatatatatatgtatgtattattgggccttgtagaatctgcaataacctgcaatgaggaccaacttacagcttagcttataggtatttttttctaaaagtcaccattagcgagtaaaattatttcatcattcaggaagaattattgatctaaatatccagctgcaggggagaggggaattcagacaattgctatcagctaccatttctccaccccattaaaagagtatattccaaaattaagaatatattccaaaattaagaatatattccaaaattaaggctgggtatggtggctcactcctgtaatctcaacactttgggaggccaaggcagagagatgacttgtgcccaggagaccagcctgggcaatataatgagaacttatctctacagaaaaatttaaaaattatccaatcatggtagtgcatgcctgtagtcccagctacttgggaggctgaggcaggaggatcacttcagcccaggaggaggtggaggttgcagtgagctgtgatcgagccactgcactccacagtccagcctgggcaacagagtgggaccctatctagaaaaaaaataaaataaaaaatatatatatacacacacacacatataaataaataaatatatatacacacataaataaatatatatacacatatatataatatcacatttggactttctggagatttgagacagttgtcaaacataaagcagtatgggctgggcacggtggctcacacctgtaatcccagcactttgggaggccaaggtgggcggatcacttgaggtcaaaaattcaagaccagcctggccaacatgatgataccccatctttactaaaaatacaaaaaagtagccaggtgttgtagtgcatgactgtaatcccagttacttgggaggctgaggcagaagaatcgcttgaacccgggaggcggaggttgcagtgaactgagatcgagccaccgcactccagcctgggcaatagagcgagactccatctcaaaaaaagcagtgtgtgtttcagttttaatgtatttcagagacagtatttgattatgtacggccacgttttatataaagaacactttgttttcctagagtctagaagacagcttggaacataataggtgttccatacatttctgctaaataaaatagttgttttaaaagcacaccacattttattattgttacccatccattttaggttaaagaatttgacaccaattttacatatgtgcaacagtcagaattctacttggagccaaacattaagtacgtatttcaagtgagatgtcaagaaacaggcaaaaggtactggcagccttggagttcactgttttttcataaaacacctgaaacaggtgagtgtacttatatattttattctgttgggcttttctttatatatcttttctgctgagcacagtggctcacacctataattccagcactttgagaggccaaggcaggaagattgcttgagcctaggagtttgagactggcctgggcaacatagtgagaccctagtctgtacagaaaaataataattattattagcctgggtggtagaatgcatttgtagtcgcagctacttgggaagctgaggtagtaggattgcgtgagcccgggagtttgatgctgcagtgagctatgatcatcccactgctctctagcctggaggaaagaccaagaccctgtttcctaaaaagtttaaaacagccaggtgcagtggcttatgtctgtaatcccagcactttgggaggccaaggtgggtggattaccttaggtcaggacttcaagacctcctcggccgacatggtgaaaccctgtctctactaaaaatacgaaaattagctgggcatggtggcaggtgcctgtaatctcagctactcggaaggctgaggcaggaaaattgcttgaacccaagaagtggaggttgcagtgaactgagattgtaccaccgcactccagcctggccaagagagagagacttggtctcaaaaaaaaataaaaataaaaataataataataaataagttaaaaacaaaataaagctacaagatattttttttctctttacctttgaccaaaattgacaaaactattctagggcagatgataacatttaaattgtcattttgctttgattttcttaaggcttactacaatttatataatggttctcaaacattcctgggtataaaaaccatctgggaggctgggtacgctggtttacacctgtaatcccagcactttgggagaccaaggcagaaagattgcttgagcccaggagttcaagaccagactggacaagatagggagaccccatctctaccaaaaatacaaataaaattacccaggcatggtggcccacgcccatagtcccagctacttggggactacaggaagaattattgatctaaacaggcttcaatcatagctcactgaagcctctgcctcctgggctcaagcaatcctcctgcctcagcctcccaagtggctcagactacagatgcggtccaccatgctagctgaggcaggaggatcacttgaacctgggaggtcgagaatgcagtgagccacgttcatgccactgaactctagcctgggtgacatcctgggcaattgtgagccatacaataagctaagaacttgttttgaaaaatattatctgggtgtggtggctcaagcctgtaatcccagcactttcagaggcggaggcagttggatcacttgagcccaggagcttgagaccagcctggccaacgtggcaaaatcccatctctactgaaaatacaaaaattagctgggcatggcggtacatgcctgtaatcccagctattcaggagtctgaggcaggaaaatggcttgaacccaggagacagaggttggagtgagccgagatcgcgccactgcactccagcctgggtgacagagtgagactccatctcagaaaattttattaagaacatattctcctaaaagagacacaaaacaatcatatcaataaacgttatctactatatgtaaagcaatcagaaagttaaaactcctaaacacagaattctcttctcctcaacaggatttagctgatttattgtttatctttagatattttcctgcttctaatattattagtttaattttatattcatttattcatgccattcaataacatcttagccaaatagacttcagttgaagcacataataaaatgaatctattgtaattgtaataatactcctaatggccccattaacctaaagatgtctccctactgtaggtaccttatacactagatttaacattttcttttcagaactgaaggactttattataatcccagtatgaatagcatcattattataacattctggaattgtgttttatgttttcaaggcactttgacatacattataaatattgatggctgccaaattagtagagggagcaaagagcatgacttgctgcaaagctcttctggtatctcctactgcccaaatcttagaaatactttggccaggtgcagtggctcatgcctgtaatcccagcacattgggaaatggaggcaggtggattgcttgagcctaggagttcaaacccagcctgggcaatatagtgagactctgcttctatttaaaaaaaataaaaataaatagaaattaaatttaaaaatgttaaaatgaagaaacaacttattagggaatccttttctctttctttctttctttcttttttttttttttttttttttttttttgacagtcttgctctcttgctcttttgcccagcctgaggtacagtggtgtgatctcggctcactgcaacctccacctcctgggttcaagagattcttgtgcctcagcctaccgagtagctgggattacaggcacccaccactatgcctggctaatttttgtatttttaatagaggcggggtttcaccatggtgggcaggctggtctcgaactcacgacctcaggtgatccgcccgctttggcctcccaaagtgctgggattacaggcgtgagccatggcacccagccagggaattattttcagtatcagttctcttttttaaaagttagtttttaattgaaggtattctgtcaagttgatactgttaacaattcgtggactttagatgccattttataatagtcagaatttagttgataacccttttttttaatgaactctataactgcctagcaagattatgcaaattgataactaccatttatcatttacgaagtactcctgtgtataagcttgtttgattatgatgtcagccatatttggtagtgtaattagcgctactttacaaaagcggaaactgggcatgacttactaaatagtacattgctggtgggtaatgacacctaaactataacaaaacttttcttattcaaaatattgaactgcttggctaggtcagttggtagagtatgagacttttaatcccagtgtgaaaatactgtgcattttccccaccatccctcagcaatttcattctttaatttcagggaagcagaggagcaacttacttaagtattctaagtataggactacaaatgttcttctttaaacataaaagtcttggcgaggtgtggtggctcatgcctgtaaccccagcactttaagaggccaaggcgagtggatcacctaaggtcaggagtttaagaccaccctggccaacatggtgaaaccccgtctctactaaaaatacaaaaattaactgggtgtggtggcaggtgcctgtaatcccagctactagggaggctgaggcaggagaatctcttgaacttgagaggcggaggttgcagtgagccaagatcctgccactgcactccagcctgggtgacagagcgagactctgtctctaaataaataaataaataaagtaaaataaagataaaagtcttaagcttcaggtagaaggaaataggaacaccacagtttaaatttaaggtctgtttcctgaggagaaaaatcacttaagagacaaaaataccaattaaaattaagtatccctgaaaacttggatttattaaagtttaacatgttagctaagagaaaccatagactgttctcttggtacaaattcccttctaagacacattacatgagaaacagtaaaagtgtgttagggaaagtgctcatgttaaatctctttgaaaatgtacctttttttctgtgtgtaaaagcaatgtaagtttactgtagtatgcaacctaaaaacatccactatttacatttatttaatttaatactagtttttcctatgcatttttttaatgtttggggctatgatgtatatactattttatatcctgattttcttacttaatctaccctgttaagtttttaaaaactgatttcatggctgcacggcattctcgtttatgtatgtactataatttatttaggcatttccctacctaaataacatctaggtcatttccattttatccattatcaataaacttctttgcatagctttgtatataaatggtctttattcctttagttctaaagaagaattattgcatcaagagttaagcaccttttaagatgctgatgtatgttgtcgaactgctttttaccgaatctttaatattgattgctttttaaaaagggacctatgaaaagacagtcattcacaaaatactcatttagcctctaccatgtgccaggcatttttagatccttgcagaacctcagtgagcaaaggagacaaaattccctgccttggacaagtttccttctggagaagacacacagtaaaaatcaagcataataattacataaattacattgcatcatacataaagatgtaaataaaagcagtaagaatcagacttcacagctgaagtatgaattgcctcacaaatgaaaaaataaaatgatatcatttctgtaattatttatttaaccaacttgtttgataatgaggttttttggccagccaaacttttccagtgcttaaaggtttatttatgaagaaacacagcccaggcagtaatgcctcttccaagcaggacttttctgaattagtctctaagattctgacatgtgtattactgaattgattcatctcaatacaatgtgtttgcaaaattctgcccagctaagaccagaggacaaatcctgaagtgggtatggcttgtcctgcagcctcggcattcttattcttttctgttgctttccctctgaatagtgcctggcttggccattaaaaacctggtctcaaaaatgaaccaaaccaactcacttataatcaactttaatgacttattctctttttcttacttagagtatttagactttaataggacaaacaaagccttatactgaaaaagaaaatcaatcctccagaatacccatttattctatctgatataatagcaagaggccaattaacagactgaatcccagggtgacttcaggataataaataataatagctaacatttattggatgcttactaagtgttgggcattttgataagcattttacatatgaattgttctttatttatcactgcagctctataaaaggagtgtaattattatcccagttgcacagatgaaaccatatggcccaagcagttttgtcaaacattacaaggctggtaaacagtagagccagggtctaactcaggctatgggattccagcacccatgttatttatagctctgctagtctccttcctaagagacctaggagggtcctgcatccgcctgtgaaacaaaatcattctctacacattagctagtaataaaactatgccaacagagcttcatttctaatgcagtgtttgttatggaaatgcaaacgcatgagaaaagaaccagaagatactactgactactgctactttggcttgaatacaagatgtggaagcagatttgcagtagatgatgagaatgaaaccatagtactataggaggtttctgataggataggtaaggaaggggcagtctgagatgaggataaaggataggtaaaaaaggcagccttagccaagacaggaccatcatgggcaattcatcatgtgctttaccagctaattatgggctatctagaaagcactgtttctcatagtagggtctgtggacaatctacattaagaatgcttgggcactttttttttttttgatacagtctcgctctgtcacccaggctagagtgtagtgcaccatctcagatcactgcaacctccgcctcctggttcaaacaattctcctgcctcagcctctcaagtagctgggactacagacatccaccacacctggctatttttttttgcatttttagtagagacagggtttcgtcatgttggccaggctggtctcgaactcctgacctcaaatgatccccctgccttggcctcccagagtgctgggattacaagcacgagccactgtgcctggcctgaatgcttgggcacttcttaaagtacagattcctgagccctttgccaagcgatacttctgcagtgaagcataaaatccattgctgtagaggtcagacacactctttaagagaaggaagtgtcatcataaaagacaacatagggaatggacagaaaatgtggacagaaaggcagagtggatatgattgcccaagccattgaaacgggagagttccctgactcctgtcgcatatcatgtggctcatctattctgccaaggcacatgctcaaacccgttacaggagggggagcacacagatggacaggtgcgcaggagctggggtgagcaccttggggctccagccccacagtagcatctaggggtggatgcctgtgactcccaaagcccaagtgggaatgtgatacagttcactattttagctttgctgtctgcagacagcttaactgttaaccagctcagtgccctcttggtacccaggtccttgtccagtgtccaggaagaatcaggtcacacacagatttgaaggatgaatgtgggggttttattgagtggtggaggtggctcttagcgggatagatagggagctggaagggggatagagtgggaggatgatcttcccctggagttggctgtccagcggccgatctcctctctgatcgtccccaggggaacttctctcagcattcagatgctccttttcttctgtccttctctgccgagccattctgccattcttctgctcttctatttatctctctgtctgcttctggaacctggggtctggagtttatgagggtacaggatagcggggcatagcaggccaaaaggcaacttttgagcacgaaaacaagaatgcctgcttctatttagggctatgggtttccaagcttgagggtgggacctctgccagggaaccaccctcatttattcagtattttcctgtttgctgtttgtatcaccatcgccttagtagtagtggtttgggtgacagaaaagggtgggcatgtgagaatcttaggccattatctctagtactatctggtcacctctctgtttcctatttctcctgagtcactgaccaccccagcccacctgggcattacaggaaccccccaaagcacaggatataggactgttgtcgtaattaaccatcacatcacacatgaccctcttctgcctagaactcaccctgtctctggatgtgaggtttcagctgagattatgagaaccgctgagagctgctgaagttagagcttctgatccaccaaccttgataggcactgttatcctggggtctttcagcagtgcggaaagagccccaaaggggaaaagacaatttgtcattgtagaaaaccaacactgaaaatgtataagtcagtagtctaaatttgctactactgacttaaaaaagctgaaacaacaatggctagccaggaatctttttcaattctgcattgtaatatagatccctttttaacgaagaaaagacacaggttctataattcctttaataacaggttaaaggcttaagggaagggcagtcacatttcatgatgactaattcacttgcttgaattgctgcttgccatagaaaagtttcccagagaactcatattactctttttttttttttgacagttttccagaggaaggcaagtatgagcaagatattaaacaacattttgctttcctctcacaaaacagtattggttgttgcactaccagtgactccttgaagatgctcattaatttttcaggtttttaagtggctaaaagggaaaaagcaaatattatgttaacggaactaaatatcagtgctattaaatataatgcaaattagatgatggcacacagaatgtatttttcaaacattttattatgtgtaattagagatttgtgaattgtccacatagagatgggttctgaacttgtagactctattggttactaacccatgttaaacagacacggtgtttcttgaaattctaatcactttagacttttcatttgggtttaactttcttcaggtcctgcctctgataaaccaccaagtttctgcagaataggaaacaattcagaagagttccataatctccctgaagtttgcaacctattactacagaacaaagatctttctttctttctttctttctttctttctttctttctttctttctttctttctttctttctttctttctttctttcttcctttctttcttttctttctttctctttctttctttctttctctgtctctctctttctttcttttctttctttctttctttctttctctctctttctttctttctttccttctttctttctgtctttctttttccttccttccttccttccttccttccttccttccttccttccttcctcccttcttttttagacagggtctctctctgtcacccaggctggagtgcagtggtgtgatctcagctcataagatctgggctcaaacaatcctctcaccttggcctttcaagtacctggaactacaggcatgcaccaccatacccagctaattttttttcttcctttttttttttttttgtagagacagggtttcaccatgtttgccaagttggtcttgaactcctggactcaagaactctgcccaccttggcctcccaaagtgctgggcttacaggcaggagccaccatgcctggcctatgattatgctttttcttgaagtcatcatcttctatattagtttcctattactactgtcacaaatcatcacaaacttgaaagcttaaaacaacatgaatttattatcttatagttctggaggtgagaaatccaaaatcacaaccactgggctgaaatcagggtgtcaatagggttatgttccttctggaggctacaggacagaatcagtttcattgctttttctatattctagaggctgcctacattcctcagctcatggctccatccttcattttcaaagccagaagcatagcatcttccaatctctttctctgactctcaaccttctcccttctgcttataagaactcttgtgattttaccagatccacccatgtaatctaggatagccacttcctctcaattcccttttgccttataaggtaactgagtagaaagaacatactcaatttctcttgctatatagtactttcacaacacaggtcacttctgtgacctctgattaccaacatgtgtatggggatttctccccctcagcaacaaattatctggcagattctttgagggacaccagctgtgtgtcctccaagtcaattcattctggcactatctgcctagacatagtgtcagatcctacaactcccacctcagatgccagtctcaagtaataggtggttacctatacttccatctgattggctataaattagagtttgtttcctgatctttttttcagatttgatcaatttgctaggatgccccacagaacttgggaaaacacttatgattactggtttatgataaacagcattacaaaggatacagtcgaacagccagatggaagagatgcagtgggcaaggcatgtggggaggggcatggagcttccataagctttccaggtgcatcacctgccaggaacctccatgtgttcagctatcctgaagctcctagaacttaggcctttggggttttttgtaggcttcattacataagcatgattgattaaaccattggccattggtgatcaacttaatcttcatcccctctcccctctgggaggttggggatgggttgaaaaatcccaaccttctaatcgtgccttgatctttcctctgaacaacccccatcctgaaactacataggggcggccagccactagtcagctcgttagcatacaaaatgaccgttaccactttgggcattctaaggattttaggacttgtatgccagaaaaccaggatgaagacacaatatgtacttcgtaatattatagtaacctgttcacagattccagggattaggacacggacatctttatgatgccattatttgtcctactacatctccctacctccccaaccactaccaccaattgcagtttttactagtcaggatgattaggtgtaagcaaaataaatcaaggctggttatcttaagcagaaagagaattattagaaaggtagctgaaaatcagtagaaagcctggcaaaatgggcatgaacaggatccgagtgagagcaggacccagaaccacagccaaaatcacagcctaggaaaagtctggcgaggaccctgcagctgacgctgatcactgccactgcctgccctggtgccactgtcactgtaccctggatacagctatggtgcccacattggattctaagtggtttctggaagccctatgtcaacccttcaacattcacagcctgggtgttaggggagggggcatctggttggtgaggtttaggccatttgcttgtagccctactgcctctcagcttttttaggatgcccacaaagaagtatatcctagtgctgggcaaataaaacatggcaaatgtctgctagactatacagattggaagatgctatgcctggagagcaagggtgtacttctgaggaatctgttggccactgtgtagacttggaagctgtccattctttcatatagagttttgctacactttgacatcagaccttctctgtatggtccttgtcatttaggaaaatttcctgggtggctactgctttttgccttttcctcctcttattctttctttttaacaatgtaactgccatatgtagatttcttaccttctccctacataccaagtacctaaagcccaggaatataagaaggaagataaacccttgaatatgtctgtgtgtaccctgctacccttatatacaccgctgatactataatccactaaatgattcattgcttgacttgtttttcaaggtgtggtaatgagtttgttctgacacaccgttttccctgccctgggcttctggtgcctatttgatatgttggcattagttttgccttgctaattgacatactcaactctttacccttggcccgataatgttgaattctgaccacagagctaaactggcctggcttggagaaatggcacttaagtgctgtgaaacagctgaattattaaaaggagatgcgaaagagagaaaccaaaaggaggtatttgtgttttgagaaaaataggagggagggtgggggcgaatctccaatgatttaaaaaaaaacacatcctttgtctcctttaatatgactcctctttcttgaatatctagaacccctgaaaggtccagaaaagtccatgtgtctgctctaatcaaagcctttatgatgcaggccttggttactgtggctttgacatcaagattggcaagcgcaaaagtaaataaagaaaccttagagatgtctataccctttggctcagtaattcctcttgtagagatctggccttaagaaataatcagtggaaggaaatttatgtacaatgatatacatccaaaacctaattataatagtaagaatttggaaccagtctaaaagttcaacaatagcaataattagtgcttattaaggttttatttgtgtaaaacatggtactaagtgcttgatatgaactgtctcatcaaacctcatgacctcactgcttttatctaattttacaaatgaagaaactgagtcactgaggggttaagcaatttgcccagggccatggaactccatctagtagagccaggacttggactgagtttcgtttgagtctgcagttggtgtttgaaaccacctagctatacagtttctcaataggggtaagattgaataaatacagtaaaacatgatgaaacattaactagccatagcatgatgttttgaacaatttttaataaaatgaggaaatttttattaagtaatattgaataaggctgggtgtggtggctcacacctgtaattctagcactttgggaggctgaggcaggcagatcacctgaggtcaggagtttcagaccagcctagccaacacagcaagacctcatctctactaaaagtacaaaaattagctgggcgtggtagtgtgcacctgtaattccagctactcaggaggctgaggctggaggatagcttgagcccagggggcagaggttacagtgagccaagatcacaccactgcactctagcttgggtgatggtgtgagactctgtctcaaaagaaaaaaattgaataaaaaaggcaagacaaaacatttataaagagtatgatctccactaggctaccaacatacacaaaagactgttaacattttttttaaataatagtttctagtttgcaaacaaatataaaattgtattatcttctccatacccttctatacttttcaagcatcctgtaattagcatgtattacttgtgtaataaaggaaagtagtcggttttaagaagaaaataactctacgctagaagatttaaagcatcagtgctaaatgggttagcacagtagggcttgtggggtccagagccccacaaggccaggcagccccaacacaccatggcgagtcctcagtggagcaatgctaagtctcatcctgggagaggctgcttgcagggctgactctgcctcccaccctgagccatcctgctgtggaagtgaggttattaatttaatgagatcagtgaccagcacaaactacatgtcagaactggatgggaatttccactcttcaccctctacctaggtgactttgagcaagttatttatcctttctactcttttgtttccttatccgtataatgaggataataatattgtctgtgtgattgggttgttgtgaggattaaatgagagaaaacatgtaaagtttagcatgttacctgtgacataattaatactcattaaatggtcactttaaaaggatgacaaaacacactttgccccatggttgatgatatggtttggctgtgtctccacccaaatctcatcttgaattgtagttcccataatccccatgagtcgcgggaggtaattgaatggtgggggcagttgcagtcatgctgttcttgtgatagtaagttctcatgagatatgatggttttataaggggcttttccccctttgcttggcacttatctggcctgttgtcatgtaagacatgcctgtttccccgatcaccatgattataagtttcctgaggcctccccagacatgtggaactgtgagtcaattaaaattctcttctttataaatttcccagttttgtccaggtgcggtgcctcacacctgtaatcccagcattttgggaggctgaagcaggtggatcacctgaggtcaggagttcgagaccagtctgaccaatatggtgaaatcctgtctctactaaaaattccaaaaaaaaaaaaaaaaaaaaaaaagccacgcgtggtggcatgctcctgtaatcccagctacttgggaggttgagacaggagaattgctagaacccaggaggcagaagttgcagtgagccaggatcatgccactgcactccagcctgggcaacagagggagattctgtcttaaaaaaaaaaatccggttttgattatgtcttcatagcagtgtgaaaacagactagtacggttgatgtagaaagaagagctgaggtgatgatttggcatcatccttaaaatacagatggaatacgttattgctaaaaccaggtccttttgagtggatttgattaaactagcctggtgttttggtaggccaaaaaatatagttgttatgctttaaattttgtccaacaataagaaaccatatttctcgtttgagatcactctaaattcccacaggcacattgtcttcttgtaagactaaagtttggtgccagtgtgtacaagttatataaaaattcttcccaaattaaagataatttggattttttttagtatattcaagtatgtcctgtgagattaataggcataagttaatattctgttgcaaggatggaactgtctcattatggacgtaacaccacaaacaccaaatacagtaagatatcatgaggttttttttttttttagcaaattaggcaatgcactgatgacagaactgatataaaaaacagtttccagtcataaaggctgtgtgtgtgtgtgtgtgtgtgtgtgtgtgtgtgtgtgtgtgtgagattctgtgtttgctaatgcttttgtatgttaaaatacttgcaaataccataaagcactaatagcaggtggattaaaaggatgggattagggatggttctttttttctccttttaatttctcctgttgaaaatttttctgtactttaattatcgcttttacagtcaaagatgttaagtataatttaaaaagaaatgacgtagccacgtggtggcagtattccatgaagaatgtaatctacatagctttgtttcagtcctatgtctaacattggcttgatttcttctctgacaattttgtttataaattgtcttattttagataactgattccttgtgaaatttccctatgaggattaagcacctcttataagtgactttaatttaatgcacctgtttgatttggaactatgataacaaatagactctaaaggaagattgtatttattccctgttagggctaataaaaccctagtcagatatgaaaattgtttttatatgatgcaaattaataactgaatgaagtttcattgccaataactacctgtagtttatacaacagaattacattaacctgtattaaaagccaacaacaccatatattacttttaaattaaaaatgctaattttaaaataataaaagtgaaaacaaaaatactacagcagcatatgctataagcccatgattttgctaaattgaaaattgtgttaccttttaatgtagtatttttactcttttttttttttttttttgagatggagtcttgttctgttgcccaggctggagtgcagtggtgtgatctcagctcactgcagcctctgcctcctgggttcaagtgattctcctgcctcagcctcccgagtagctgggactataggcacctgccaccatgcctggctaatttttgtatttttagtagagatggggtttcaccatattggccaggctgatcttgaactcctgacaccctcctcagcatcccaaagtgctgggattacaggcatgagccactgcaactggccagtatttttattcttaaaatttcaaaataatggagattcagactcctgcctgagctccctgcaaatgaacattggcctgtaaagatagcactttagatgaataaacctgactgttggccatcagggcataaccttgttccctcagagtttggtggaaccatatacccatggagcaaaagagacttagcgaggcattgagaagccaaaaacaatttctgatttgtcacaattccgggtgaaacattttcctcttgctgtagagaagccaggacaaaaaacacgggaaataacttttggcttttctgatgttttcatcttctaaaatccctcacccactgttctcagaaaactaccctcctcacgcttatgcctgcacccagagcagccttttttcttttttctttttttttttttgtgaaatggagtctccctctgtcacccaggccggagtgctatggcacgatctagactcactgcagcctccacctcccgggttcaagtgattctcctgtctcagcctcccaagtagctggtattacaggcacgcaccaccatacctggctaatttttgtatttttagtagaaacagggtttcaccatgttgcccaggctggtctcaactcctggcctcaagtgatccacctgccttggcttcccaaagtgctgagattacaggcatgagccactgtgtctggccagaccagcctttttcaaaaatttctgaagccccgtagaaaccttttcattggaaatgtaggacacttttctcaaagacagtcacatgggccacactactccttatacaagcctgtgatatctcagcctcaagttgtgatttctccctgcccccaaaagtaatttttttaaaagaaaaatttgagcactgaaatcagagtgacggaatacacagggcttgaagcaatctcagggatccaaggcttagagccatcttcagaaatgtcttctgccagttgggagaattgtgtccttgaagtcacttctgtcagcttttaattatcaggaaggaggagactggcaaggctgcaccaggacccctttgagttcagactgaaagttaggtaccagggttgctcaccccaccctggtcagaatcattcattagcagtttcctgacagcctttataactagaccaggctgccaggaaaagaaaagagcagagagaagtcattggtgacatcccacccaggtgttacaacaccaaagtgcctgctaaccaaaattaggtttcttatgaggagccaggagaaggtgaacagcatcccaaagattgtgcaaaggcaaaagtgacacatcttggggaccctcaaggaaatctgaaccttattgcaccatcaattgcaaggaatcaaatagagattagttatcatgcgttttttctggctcagtaaaataaagctctttggtatttgctgcctagaagcctcatgaaattagtggacacaccttgaagttttacaacctaggtagattatagttgcccttgcagtgtttcctcctttgagatttacccaattaaataaaaagaagaagaaaagacacatactcgaagactattgcttttattccacccttcaagcccattaaagttattggtttactttagtcatctctaaacaagggaagaaactccgttgggaaatttggcaagcagagtgcaaaatgtaagaaactgacactctttccttttggtttaagttccccaggtcacatcaaaagcattccaacatgacacatggaattctgggctaacagttgcttccatctctacagggcaccttacttctggtaagaaaatacaacttaggctttttgagtagtcttttagtaattgcccattttaacccatcatactgaaaaaatcacatcaggtgttaagtttctggacaataagatatgccttatgtcttccataggaaaataatagacaaagtacaaagatctgcttaaaactgaatgtaagaagtggcttaggtggattttgccggcttttgcaatagattgtatacattttttaaaatttttatttattttattttattttttgagacgaagccttgttctgtcacccaggctggagtgcaatggtgcaatctcggctcactgcaacctccgcctcccaggttcaagcgattctgctgcctcagccttctgagtagctgggattacaggcatccgccatcacgcccagctaatttttgcatttttgtagagacgggtttcaccatgttagccagactggtcttgaactcctgacctcaggtgatccacccacctcagcctcccaaagtgctgggattacaggtgtgagccaccgcacctggccacatttttattttaatagctgctagggctcaagacccaaaaacttctttcaaaacaaattacttacctatcttgtgctaggacttgtctagacatcttcttcaatctttaaaacaacccatgagataagtgttacgcatctattttataatgaggaaactgaaacttagagtagttgaggaaacttttcaaggtcatagagctgctaagtgacagactaaaattcaaatccttttctttcaatgtcctggagtctattgtctttcttttatacaaaccagctcccatttcagttgttagcatgactattatcgtattgatgagcttgcctaaacgttttaggcgtaaaaaaattgagatctggttgtacaatgggtgaacataactctaaatagaaatttcgagttgaaaacttttaggcatgactatttcaccattgaccactgacagaccatttatctccttgtgtagaaatcaggcaggaatagtataagaacttttgcaggttgtattgctattctttgcaccttcatttatttaatattcattaagcaactcctgtgtacgaggcactgttctgggtactggggaatcagcagtgaaaacaatgagcaaagcccctgtcttcatggagcttctattctagccagacagggcagaaaaacagcaaacaaaacaagaagaaaagtcaggtggtggtgaagtgtcgtaaagaaacatgaagtgggtaggcatggtggctcacattttgtaatcccagcactttgggaggccaaggcaggcagattgcttgagtccaggagtttgagaccagcctgcacaacatggcaaaaccccatttttacaaaaaatacaaaaattagatgggtgtggtgtcatgtacctgtagtcccagctacttgggaggctgaggtgggaggatcaactgagcaccagagatagaggctgcagtgagctccactgcattcccgcctgggcaacagagcaagaccctgtctcaggaaaaaaaaaaaaaagaggaaaaagaagaaaaagaaaaagaaacatgaagaaaggtaagggcactctgaattatcaatcaattgcaagccaagtgcttaggttcagtacagttccctaattatagatgcctacacagacctacctacaccttgatatttctgtgggatcagtggaggttaggaactcatggcagctctgttagataagtattattatctctattttccaaaagagaaaacctgagactcagcaagttcataattatgccccaaggtcacagagctgataagaggcagagtttaattcaaacccaggtatatcaggccacgctcttggtcattctgctctactgcttagacccctttgccgagcactgtgttgacctgagggctgtctatcctcttccaggcactaattaatgcccaagggtgtgtggcagccagtacccccaattagttgcttcaaatagtcagaaatagctctttagccctgggaaaacattaatttcatatcccatcaagattttgcagtgagctagagttactcagactcctgcctgctgattggctttagaagagggctgggcagcctggagaccccggtatgtgaaataactagggtggggcaaacacagtgtctgccacatggtaagtacccaataaacatttgctgaaggaagggagggagtgaggaaggaggatggaagggaagccaatacccaacatggctctagaacaaattccaatgtgataagtaatgcctcaactatcttctatatttgaaaatagggctttttcatgtaccagggagaaagcatgatgagcctggtgggtaatatgtgttgaataaattatattaattatttaaatattttaggagattaactcaactttgacatgcaagaaaagcattggttttgtttgtttgtttgtttgtttttgagacacagtctggctctgtcgcccaggctggagtgcagcggcacgacctcagctcactgcaacctccacctcccaggctcaagccatcttcctaccttggcctcccaagtagcggggactacaggcacatgccaccacacccggctaatttttgtaatttttgtagagatggagttctcactttgttgcccaggttggtctcaaacttctgagctcaagtgatcctcccacctcggcctctcaaagtgctgggattataggcgtgagtcactgtgcccagccagcattgttttttaactaagtgtgagtgcagcataaaggcaattttgatttcttttttattttttatttttatcttaacttgccagtgtatgatttctttcttatcacgtatattgctctaagtaaagtggatgtcctttctcagcaggaaaaatttcatgagcaatagattttgaagaatgatgacaaattgcacacattctatctcacacaaaaagttaaaaaaaatcagtatgattgtaaccagctttagacattgttacagcaattgggaattctcacctgtgtcagacaagccaaatgaagctcaccactaagaatttatacgaaatttgcatgcacaagccgaccacatttgccagagatgcacttctaaaaacccactgacatcagatacatgtagcccaactttctcaaacaaaaagttgtttcctggggtagttgtgcactctggaaaaacagtcactctgtggcctaaagtaaaggttaattttgcttccccccaccctttctcctttgagacctttgctttgagcagagtaaagagaatagtaattctggtatcaaatgaagactaatgcttggttaaaattatttttctttcctttcattagacaacagaggagacattggacttttattgggaatgatcgtctttgctgttatgttgtcaattctttctttgattgggatatttaacagatcattccgaactgggtaggtttttgcagaatttctgttttctgatttagactacatgtatatgtatcaccaaaatttagtcatttcagttgtttactagaaaaatctgttaacatttttattcagataaaggaaaataaaaagaacaatgtttaataagtacttacccatgccaaactctctacaaatgtctttcctttaatcctcaaaatgaccctgccagaaaagcttcctggcctattttacaggtgacttaaatgaggcttaaagaggctaagtcctcagcccagaatcactgaacagtaagccctgagtccaaacacagctgatatcaaagcccatttctctcctttactacacggcgttttccattgtgcctcaaatttacctacagtgcctagattcttgagagtggtgaagtcacaaattgccttgtgttaaaagaaaaacttcagccaaattaaatttaaaggagtttaattgagcaatgaatgtgcgaattgggcagcccccagaattacagcagattcagaaagactccagggttgccttgtggtcagagcaaatgtatagacaaaaaaaaaagtgacacatagaaattggcagtgaggtacagaaacagctggataggttatggattggcatttgccttatttgaacacagtttgaacacttagcagtctatgagtggttggagtatggctgctgggattggccaagactcagctattgttacgggcacatactactaagttaggttttcaatcttgtctgcctattaggctaggttacagttcatccacaaggacttgaatatagaagtatggagtccttctcagatcatatttagtttgctttaataattccccccttttggtgattttctcaattttgagagattgaccaaaactttagtcattgatgtcactatcaccattgtaaatgtacttatatggtctttaaacccactgggaaacagtagaacagtgggttctgtaaggtggcaacaaggactgagtagaaggtacctccttatgctggaacatcctgtttataggagaaaaacaaaacctggtctgttctaggatctatgtgtttccttaaagtcttagtttgactttgtcacatttagcatgaacaactccattttggtttggtttggtctgttggagtctagtgcatgagctcagtccaaaacaatggcctcccataatttcgttttaaaaaaaaaatccccctttttggtcaggttctcacttaggtgacagtgtgaccaaaacctagggcctcagtgccactctcagttaccatcattttgggtttctgttctcagcatgtaattcataggttatggtgtcctgatggtcacacatttcttttagctcttgttattccagttgaagagagaccatttgacattctggagatggctgcttgtaaacatttaaaacctttgagagaatacagcataccagggagattactattaggactattgggagaataatactaagagtttgggttatgttccttacccagggtcctcataaaccaaaccaaaccatctaaaaatcaaatagatcaaagaatgagctacatgaagagtctactcacttaagtgatttttttcattaatcccctacaactgaatcactataatacccgatgttttctccatacatcataagtgccagcagctgtttagccaatacttttcggtttggccaattctattatttcgcataactttcacaagagaatttaaagttttttgtgtaaccataggctttacagtagaatctgctatagaacctatcatgagggatacatttctaatcagtgcctcttttacttcaaactatggaaaaaagacctaacaaatgatgccctgctagaagagtgaaggcctcctggcaatgttctctttagcccatgatgtggcttaagaggaatgaatcaatgttctgtttctgactgactatgaggcaatatatgaacccttaaaatttctcacctgcattgggccttcatcttttatccatcaaagtataacattatccatgtataaggctggctgcaaaatccttcacaaataaacatataccccataatacacacataacagacccccttttcacttctattgttcatagaggcataagcaagaaaaaaaatattcaaagttaagagtctcatgatagtagagaagtcttaatccatgattttaggataagctgttcacattaaggacgctgtcttcttgggagaagctgtcctggttagctttaccttaagggttccgatgggtgtggagggaccctcctcagttgtgagattatgaaagttgtgggatctctgaaaccaaaagttcaaggtcccaaagttttgctgtagtgtggatggcaaggacagtctttctctaatgttctcagaagattcattctttgggttctagattgtgaaggggttgattgtcctcagtgaaccataaacagctttttttaactatgtaaaaatgtactgcagcataataatctactattataacatcagtcttcttgcatgggaaagctttcatacaaccagaaaacatgcattgaaagtgacaattgaatgaaatcccttataaatatttaaatgtccaatcaggtagccaaatgtacctgaagctttgattgttttcccaggaatatgggtttgacaagccaaatattgtttataactattttagtagtttataagtcaccacacaaacatatttaatttggatcattttatcttttccattacaagtcgtaaaatgcagaacttttaataatgaaagccttaaggactctggaaggataaggtggctgtcttggttctccacgagtccatgcttaaaaatggacttatgtcctcttgaataccagttgtttctccaatttaggtgcatagcactgataactgatgggttatcataggtaatttggcttagatcatggagtttattcaaattgtatatctaaacaattttagtattggctgatttagcatgcggatttctctttttttttcttatactgtaagttctgggatacatgtgcagaacatgcaggttcgttacataggtatacatgtgccatggtggtttgctgcacccatcaacccgtcatctacattaggaatttctcctaatgctatccctcccccagcctcctaccccttgacaggccccaatgtgtgatgttcccctccttgtgtccgtgtgttctcattgttcagctcccacttatgagtgagaacatgtggtgtttggttttctgttcttgtgttagtttgctgagaatgacggtttccagattcatccatgtccctgcaaaggacatgaactcatccttttttatggctgcatagtattcaatgatgtatgtgtgccacattttctttatccaatctatctttgatgtgcatttgggttggttccaaatctttgctattgtgaacagtgctgcaataaacatacatgtgcatgtgtctttatagtagaatgatttataatcctttgggtatatacccagcaatgggattgctgggtcaaatggtatttctgatcctagatccttgaggaattgccacactgtcttccacaatggttgaactcatttacactcccaccaacagtgtaaaagcattcctatttctccacagccttgccagcgtctcttgtttcctgactttttaatgatctccattctaactggcatgagatggtatctcattgtggttttgatttgcatttctctaatgactagtgatgacgagctttttttcatatgtttgttggccgcataaatgtcttcttttgagaagtgtctgttcatatccttcacccactttttgatgggttggtttgtttgttttcttgtaaatttgtttaagttccttgtagattctggatattagccctttgtcagatgggtagattgcaaaaattttctcccatactctaggttgcctgttcactttgatgatagtttcttttgctgtgcagaagctctttagtttaattagatcccatttgtcaattttggcttttgttgccattgtttttagtgttttcatcatgaactctttgcccatgcctatgtcctgaatggtattatctaggtattcttctaggttttttatggttattaggtcttatgtttaagtattgaatccatcttgagttaatttttgtataaggtgcaaggaagggatccagtttcagccttatgcatatggctagccagtttcccaacacaatttattaaatagggaatcctttccctattgctcgtttttgtcaggtttgtcaaagatcagatggttgtagatgtgtggtgttatttctgaggtctctgttctgttccattggtctatatatctgttttggtaccagtatcctgctgtttttgttactgtaaccttgtagtatagtttgaagtcagatagcgtgatgcctccaggtttgttctttttgcttagaattgtcttggctatgtgggctcttttttgattccatatgaaatttaaagtagttttttccaattctgtgaagaaagtcaatggtagcttgatggggatagcattgaatctataaattactttgggcaatatggccattttcatgatattgattcttcctacccatgagcatggaatgtttttccatttgtttgtgtcctctcttatttccttgagcagtggtttgtagttctttttgaagaggtctttcacatcccttgtaagttgtattcctagttattttattctctttgtagcaattgtgaatgggagttcactcatgatttggctcactgtttgcctgttattggcgtataggaatgcttctgatttttgcacattaattttgtatcctgagagtttgctgaagttgcttatcagtgtgaggagtttttgggctgagatgatggggttttctaaatatacagtcatgtcatctgcagagacaatttgacatcctcttttcctaattgaataccctttatttctttctcttgcctgattgccctggccagaacttccaatactatgttgaataggagtgcggagagacagcatccttgtcttgtgctggttttcaaagggaatgcttccagtttttgtccattcagtatgatattgtctgtgggtttgtcataaatagttcttattattttgagatacattccatcaatacctagttcattgagagtttttagcatgaagggctgttgaattttgtcaaaggccttttctgcatctattcaggttatcatgtggttttcatcattagttctgtttatgtgatggattacgtttattcatttgcctatgttgaaccagccttgcatcccaggaataaagccagcttgattgtggtggataagctttttgatgtgctgctggattccgtttgccagtattttattgaggattttcacatcgatgtttatcagggatattggcctaaaattttcttttttttgttgtgtctctgccaggttttggtatcagaatgatgctggcctcataaaatgagttagggagtattccctctttttctactgtttggaacagtttcagaaggaatggtaccagctcctctttgtacctctggtagaatgtggctgtgaatccgtctggtcctggactttttttgattggtaggctattaattactgcctcaatttcagaactggtttttggtctattcagggatttcacttcttcctggtttagtgttgggaaggtgtatgtgtccaggaatttatccatttcttctagattttctagtttatttgcacagaggtatttatagtattctctgatgatagtttatatttctgtgggattggtggtgatatctcctttatcattttttattgcatctatttgattcttctctcttttcttctttattagtctggctagcagtctatctattttgttgatcttttcaaaaaaccagctcctggattcattgatttttttgaagagtttttcatgtctctgtctccttcagtgcttctctgatcttagttacttcttgtcttctgctagcttttgaatttgtttgctcttgcttctctaattcttttaattgtgatgttagggtgtcaattttagatctttccagctttgtgatgtgggcatttagtgctataaatttccttctacacactgctataaatgtgtcccagagattctggtacattgtgtgttttttctcattggtttcaaagaacatctttatttctgccttcattttgttatttacccagtagtcactcaggagcagattgttcagtttccatgtagttgtgcagttttgagtgagtttcttaatcctgagttctaatttgatttcactgtggtctgagagacagtttgttatgatttccattcttttgcatttgctgaggagtgttttacttacaattatgtgatcaattttagagtatgtgtgatgtagtcctgagaagaatgtatattctgttgatttggggtggacagttctgtagatgtctataggtcccacttggtccagagctgagttcaagtcctggatatccttgttaattttctgtctcgctgatctgtctaatattgacagtggggtgttaaagtctcccactattattgtgtgggagtctaagtctctttgtaggtctctaagaactgactttataaatctggatgcttctgtattgggtgcatatatatttaggatagttagctcttcttgttgcatttatccttttatcattatgtaatgcccttctttgtctcttttgatctttgttggtttaaagtctgttttatcagagactaggattgcaacccctgcttttttttttctttccattttcttggtaaatcttccttcatccctttattttgtgtgtgtttttgcacatgagatgggtctcctgaatacagcactactgatgggtcttattctttatccaatttgccagtctgtgtcttttaattggggcatttagcccatttactttttttttttttttgagatggagtttcacttttgttgcccagactggagtgcaatggcacgatcttggctccctgcaacctctgcctcccaggtgcaagggattctcctgcctcagcctccctagtagctgggattacaggcatgtgccaccatgcctggctaattttgtatttttagtagagatggagtttctccatgttggtcaggtaggtctcaaactcctgacctcaggtgatccgcccaccttggcctcccaaagtgctgggattacaggcatgagccaccgtgcccagcctagtccatttacatttaaggttaatattgttatctgtgagtttgatcttgtcattatgatgttagctggttattttgcccattagttgatgcggtttcttcatagtgttaatggtctttacaatttggtatgtttttgcagtggctggtactggttcctttccatgtttagtgcttccttcaggagctcttgtaaggcaggcctggtggtgacagaatctctcagcatttgcttgtctggaaaggattttatttctccttcgcttatgaagcttagtttggctggatatgaaattctaggttgaaaattctttcctttaagaatgttgaacattgacccccactgtcttctggcttgtggggtttctgctgagacatctgcttttagtctgatgggcttccctttgtaggtaaccagacctttttctctggctgcccttaacattttttccttcatttcaacgtcggtgtatctgatgagttgtgtcttagggttcctgttctccaggaatatctttgtggtgttctctatatttcctgaatttaaagtttggcctgttttgctaggttggggaagttctcctggataatatcctgaaatgtgttttacaacttggttccattcttcttgtcacttttaggtacaccgttcaaacatagatttggtcttttcacatattcccatatttcttggaggctttgtttgattcttttcattcttttttctctgatcttgtcttcttgctttatttcattgagttaatcttcaatctctggtattctttcttccacttgattgactcagctattgatacttgtgtatgcttcacgaagttcttgtgctgtgtttttcagcaccatcaggtcatttatgttcttctctaccctggttattctagttagcaattcgtctttttttcaaggttcttagcttccttgcattgggttagaacatgctgctttaactcagaggagtttgttattacccaccttctaaagtctacttctgtcaattcatcaaactcattctccatccagttttgttcccttgctggcgaggagttgtgatcctttggaggggaagaggcattctggtttttggaaatttcagcttttttgtgctggtttctcccaatcaagccagtggatcttagcttgctgggctctgtgggggtgggacccactgagccaggcaccggagggaatctcctgatctgctggttgtgaagaccgtggggaaagcacagtatctgggccagagtatactgttcctccccgtacagtctgtcatggcttcttttgactaggaaagggaaatcccccagccccttgtgcttcctgggtgagacgatgccccaccctgcttcagccccccctccatgagctgtacccactatccaaccagtcccaatgggatgaacttggtacctcagatggaaatgcagaaataacccgccttctgcgttgatctctctgggagctgcagaccagacctgttcctattcagccaccttgccagctctccagcatgctgatttttcacgctgatttagcatgaaaatctggcatagtattttcttggtatttaattaatttttgttctactttggttagcagtcttataaaccagttagtctttttattaaagttttagggattcttaccgagtctaaatgatacgaatttaaagttattagaaacctgtattcaagagtgctttttagcatcctttttatcctttcatgaactttctaaaagataccatattctaggattttccgtgtttgtgaagttttcagaaattgcattagcattaagtaattaacttaatgtaatgactttaaacagtgatatttaaaaacacaattcacaaggaaatgtggttatctctgtggtctgtgataaattaacataataaccttaattatgattgaaaggatatactcagacattagaattttagaaatcctatacaattttggaacatatattaatattatttaacctgaagaagattaaacattgtttttattttgacaatcccatgtaactaaacatgtcagataatcctatttacctctcctttggatgctccaagggccctctgtagcatccaaaatttgggggttagaaagacaattttgaagctgaaatttcattttgggaagcctataaaatatgttaaaggtttaaaatacttgatataatgcttaaccagtttgaccatgaggtgaaattcttgtaaacattttgtaaccctttacaaatttttgttaaaaagcagatcagtgctctaagaaaaacatgttgtgcttttatttcaattttaatttatagaaaaacttagcaatacccctttatctttagccaatgtccacacagaatttctttttataagattaacttttcataaaccttccacaatgtattcaagcctttagctttattttaatctaatttaaaacaatattttaaccctctaacctagacaaaaatttacattcctgtgccttcttataaccttttagtaaaaacgcattttagtttcctgacacaccttgcatgtaaatctattttcagtagtctggattacatgttgtaatggtaactgttagcaacttttaattttggtgcctaaatttcctttcatgtatcctttcatgacttatgcagaccatctatgacatgcttagactttctgacttgtcctaatcatccctctttttaagcaaccagttattttactttaggacaagaatttaccatacaactttctttcttatataaaatctattttctttataatcttttttgcatagctaggggagcatggctaattccacatgtccccaggccttattgagaatctaatgcctccaagataggtaaattgaacaattttcaaaagtcaaagcagtttatgacctgaaagcattagtaaacctaatatctgacctgtctaatttagacaaaatgtctttattttaccaataatcgttaaagccatttttatttcccaaagattactaaagttacttgaactaagacattacagtttttattttttcttccaaaagtgttttatctaagcacttatttttctttaagccaattacttagagttcttttatatgaacatcacacacacaacacatatataactacacagagaacaagatccagtagttgtaggatttttcatttgccagtttcttcattggattactgaccttggggtggagcccatcaagaaatggctaggaaaacatgcagtttctggggtctaataagcaggcacagctggaaggcaaaatggatttcgagagagatctatttgcttttaattcttgaggttccatgaggaaaacagagggttttttccaaaacaggatcagtggcaccttctttatttttcccaaggagtcctaggctatcagaagttatcttagggcctctcatgtgtgccttaagagtggcaagacaaaatggagaaaaataattcagtcgactgagaagaaaaaacctttttcagcaaaacaagatccacaaaaaggaaagacataaatgtggccaggcgcagtggctcatgcctgtaattccagcactttgggaggccgagctaggcagatcacgaggtcagcagatcgagaccatcctggctaacacggtgaaaccccgtctctactaaaaatacaacaaattagccaggtgtggtggcaggcacctgtagtcccagctacttgggaggctgaggcaggagaatggtgtgaacccaggaggtggagcttgcagtgagctgagatcatgccactgcactccagcctgggtgacagagcgagactctgtctcaaaaaaaaaaaaaaaggaaaagacataaaggccttttaaatatacctatagcttggatatccacttttaattaagctgactcttaactatagtgctctttaaaaaatccttttaaatctcttgttacccaactttagccatgccaagtggcctgtatttctggcttttgaactttacctcccaggtgctcagagaaaggaaaattcaagacaattcatggaagggaagagaatcagcaaatgataaagttcccacaggtatcaaaccagaaaggactcattccttaacccaggaattgaacccaggccaccactgtgaaagtaaaaaactttagctactgagctacagtactgggtagtctccattgtgcttcccagaagggctctaaagtacttaattttgagcttgcaaaagcttttaactactcaacttaatttttagagctaactgtgacatgaaccctaaaattcctgttcccttgaaggcagagaccaagaaaaagtaccaccacgtggttacaaagtcaagctcccaagaatgtaaaacaagatggagacctcatccagtttttttttaatgcacttcagtgtgttgttgttcatttggaatgttccattgtaagttatctttagtaaaattttgctatttctgtaagactttgctgcctcccaagcctaatgtataaactggaaggaactctgcttttcagaaattaaggatctcatttttacctaaaatattggctttgctttcaggttcccttgatgaacttagccaatgatttttcctacctaagtgtgcaagaaaaatgaaacaaaggtgtagaaaaccaaaaatccctgtgaatttccaaatgccaaattttacaacccctgcaatactaccatttactaccagtttctttctgacctagtcaaatctaagaggcctctaattggatcctagcctgttaattactgatcaaatccaatcctggacccagtccagtttctgttgtgacttctgaacccagtttggaacaggaatttgctcaaagaaacttggagagttcaaaacacaaatctgtggagctctgaaatccaaaagagaacttgccatgatccccagccactctgagagatcaaaggacacaagtaggtccaacaggttccttgcttgttcactcagtgctcctgggggtcgttagaaggtctaattcatatcccgcttctgacaccatctagtaaaagaaaaacttcagccaatttaaaggagtttaattgagcaatgaacaatttgggaatttggcagcagaatcacagcagattcagagaaactctagggatgccttgtggtcagaacaaatttatagacaaaaaaaaaaaaaaagagagaagtgacatacagaaattggcagtgatgtacagaaacagctggttggttacaggttggcatttgtcttatttgaacacagtttgaacatttagcagtctatgagtggttgaagtatggtccactgggattggccaagactcagttactgttacaggcacatactcctaagtcaggttttcactcttgtctgcctgttaagttaggttacagttcatccacagggattcaaatatagaggtatgaagtccttctcaggccatatttagtttgctttaacacttgaattccacccaaacaaatcagcttgaatcagtgtgaagggaagtgtgacaatttttaaatttgtcaattccttaccaattgttttagtgttttagcattcagccccaaagtcccttactcttggcccccttcagttttttccctcctgtgatggtattagtaaagatctatggttagttatttaaaatgagactttgagaaaagcaagacccttggatttctaaattttactgatgcattgagtatttctaagctgctcgatagattagagttgtttggtgtggcagttccccagtgtgtccagttgctcacaaattttgacttgaatgttctttgccgaattggcactgagtttctccttcttgccatcatttgcttcatgaaataatctttctttcgtttacatttataatcaagtgcagtagaaagattttaaatgagctattataaagtctactaatgatttcttatctacataggttttttgctcagaacttaatatttcaaaatttaaattacacattaataaacatattcctaatacccttgtaaggaaggcaaactaatacggaattttatttgaggctgttttaaaatatacttgattatgaagtcccttgaaatattttaatgttcaaattaataccccataaaaaaatcaatatttgtgttattatttaaacatgccctagtgtaaaagtttaagtaacactaagttcataactaagaatagcctaaaaactaattttcagttcataacaaggaattagggcaattctttgctcatttaattatttaaataagtactctttttaaaaagataaaacaagacataattccaatatatttattttacatttgaacagatacttttatttaatacaactattcattatctataaaagccttagttctgaaacagaagtgagcaacagaaagcaaaatattttatttatttatttatttattattattttttgagacagagtctcgctctatcatccatactggagtgcagtggtgcaatctcggctcactgcaacctccatctcctgggttcaagtgattctcatgcctcagcctcccaagtagctaggaatacaggcacacaccaccatttccaactaatttttatatttttggtggagacgggatttcaccatgttggccaggctgctcttgagctcttggcctcaagtgatctgcctgtctttgcctcccaaagtgttgggattacaggtgtgagccaacatgcccggcccaaaacattttaaatataataaaaccaaaagtgggtaaatatatatacatatatatatatatattctcactgtatcagtgtactgtataaaaactttactccaatagcctgaatttactaatttggtctaattttaaatacagaagagagtgtgaattagtaaacaaaacaacaacaaaaaaaatggaatcataaaacattgtataaagatgagtaaagaaataatatttaccctaaagtccaaggtcaaattgggctagcaaagttcactcagccattttggttgttatttagtacaggtcctttgagaaccaatttaaacaaataagtctgatccgttgctaacaggatccattgttaatatgtaaaaaggtgtctgagatcagtttagtctcttctaagttcttggtacaacctttctgcccaaaccaatttgtactgtcaatgcagagagtgtttttaaacacaatggcagtgctaatagatagcttgggggaaaaagtgggatgtttatgacttctaccttttctttccaaagccccaccccactgatccttaaaataagcacatggtctaggtagaaaccagttctcactatgccacacaactgcaagacatgttccccagcctccttctccatccgcatcaagaaagtttggagctgtcaccatcattacaaccctatttgaaataattacaaaatctagactcagaacagcctaaattttagggctttattatataacattctctttttaaatatgcggtagttacggtcaccttggaaagttctacaaaatatcccttaagttttttgaactttcccacatgggaatcttctggttatgagagtttgctctatttaatatgtgtacggtttcactgctagggtggttctcccacttatcttgaatctagtgtgagtgttttgaaatgtttctgctcattgaaaagaagcagagcaatagagatgagaggaaaatctgaaaagataatgacacaatttcccacttaattttcattaagtaagagatgaaaactttagcctcggcatcaggaagtttgatttctttaattaattttttttttgagtcagggtctcactctgttgcccagagtgagtgcagtggcatggtcacagctcactacagccttgacctcccaggctcaagcgatctttccacctcagcctcccaagtagctgggaccacaggcatgcaccaccacacccagctaattttttaatattctgtagagacagggtcttgctatgttgcccaggctggtctcgaactcctggactcaagcaatcctcccacctcagcctcccaaagtgctgggattacaggcatgagccactgtgcccgacctaggaaatttgatttttaatatacattttattctagttgacttcctaatctcctatatgattgcctgcttcttctaacgtgtcatttttgttttttaggattaaaagaaggatcttattgttaataccaaagtggctttatgaagatattcctaatatgaaaaacagcaatgttgtgaaaatgctacaggtaacctaacatcatccaacaaaaactgagtggcaattgagaccaagagtgcagtctaataattagaatagaatttccattcaaataaataaatgtgcacacatattaacataattatatagacatgtgtaaagacaaaactgtacctgtttcaaatcatacttaaaagagatgagacaatagagttgggggaaagaaaacagagaagaaataaagaaacaaacctgttcaaaagagaaaagtgaagtggaagattgtgggccttgtgcacgagtcacctggtgaccaaacaatggtagatgtcttcaggaacaaagggagtagggaagaaaagctcaatgaaatgagcctcaaagattcaaagggcttctgtttcacatgagatggagagccaggtggagcccaagaagggcatcattttactctttaaacccagtggaatttgagaaaagcaatgtgagagaactgtgaacaatagtgtcacaggggtgggctaggtttccattctggcaaagagaaggccacacaccaggaagcccctgagggtacagggacattactgattataaaggagggaaggaacaagctatgtgtgttcctgataacccctggccctcgggattggctgtcaaggggctcaaaacccagtccaagggacaaacacatcatccaagccttgcaatgcagtgatgtaagtgcaatgatagaaatgagcccaaacatgtgatgggagtggaaaggaaacactgaaaggagaggagctgctcaggagaagcagggagaattcaaagtcagggggacccctgggggttagaactgcatgttgggtaagaagatgaggtgtttgtaaaacaaatagaagtggctctgtttcaagctctggtaagcctattagctaactctttccccaacctcatgtcatctgaacaaagggtttctaggctaaaaataaaatactttttaaaagttcaaaaacaactggtcaacagaatagagtctgagttctgtaacacaagacttctgtgatctgatccactcaccattccagctttactccagccactcctcacgtcaagatttttgatgaagcaatacccaatgtgctgttcttacccaaacctggcaagctccctaaggaggctggggctgaagaaaattggaatgacacttcatgtctgtttattcaagatctccttctttgctaaactcctttcctattctgggctcctggaattagaggcaaaccttagggcttccctcagataaggttttgttttctgcccgagagactaaggaatgcttagtcggcttgggtggcttaaacaacaaacatttatttgtcacagtgctggaggctgggaagtccatgatcaaggagccagtcaattcacttgctagtgagggctgtcttgtggacagctgccttctcacatggcagaggaagagatcatctctttcctgtctcttcttcactaatcccattcatgaaggctccgccctcatgacctaattaccacccaaagtctccactttcaaataacatcacattggcttcaatgtgaggggggctggggagagggcacacaaacgtttagtccgtaacagggggctataatattataattgtgttgggtgacagtttgcccaggaaggccagccatctgttggaaggcctccagtccatttattgcccagaaaagtcatcaggctaaaaccttcacccaccctccttatcatcacaaacatgtctgaattgtgagtagctttatttatttatttgttgttttttgttttgagacagagtcttgctctgtcacccaggctagagtgcagtggcatgatctcagctcactgcaacctccgcctcccgggctcaagcaattctcctgtctcagcctcctgagtagctgggattacaggcttgcgctactacgcctggctgatttttgtatttttagtagagacagggtttcaccatgttggccaggctggtcttgaactcctgatctcaagtgatctgcctgccttggcctcaaagtgctgggattacaggccttatccacctttagaacctcggcatcaagcatttgtcacacactcattctcggtatgtgagcaaaataggattctccccaacctttttttatttaagcaacaaagtattttcttgtaacaagaaatattaatattaatattcttgcctcagaagaccaacttttaaaaggccctaaaattaagtaataagtaataagttaatatataagactcaacttttctctttaaaacttttaaaccaagaaatgtgcttaagaagtggattatttttactattccttttcttcagtcttctcatgcaaatttaataaagatttggaagcaagcaggataacaaaagacaaaaggaggaaatcatatgggaagataaacaatatgagaaaatggagggagaaaggaaagttgaaagaaagcaaaattcctatcctcattcaattatccagttggttcttttaaatgtcttttgcaaaataaaatgatgtctttttttcctaggaaaatagtgaacttatgaataataattccagtgagcaggtcctatatgttgatcccatgattacagagataaaagaaatcttcatcccagaacacaagcctacagactacaagaaggagaatacaggacccctggagacaagagactacccgcaaaactcgctattcgacaatactacagttgtatatattcctgatctcaacactggatataaaccccaaatttcaaattttctgcctgagggaagccatctcagcaataataatgaaattacttccttaacacttaaaccaccagttgattccttagactcaggaaataatcccaggttacaaaagcatcctaattttgctttttctgtttcaagtgtgaattcactaagcaacacaatatttcttggagaattaagcctcatattaaatcaaggagaatgcagttctcctgacatacaaaactcagtagaggaggaaaccaccatgcttttggaaaatgattcacccagtgaaactattccagaacagaccctgcttcctgatgaatttgtctcctgtttggggatcgtgaatgaggagttgccatctattaatacttattttccacaaaatattttggaaagccacttcaataggatttcactcttggaaaagtagagctgtgtggtcaaaatcaatatgagaaagctgccttgcaatctgaacttgggttttccctgcaatagaaattgaattctgcctctttttgaaaaaaatgtattcacatacaaatcttcacatggacacatgttttcatttcccttggataaatacctaggtaggggattgctgggccatatgataagcatatgtttcagttctaccaatcttgtttccagagtagtgacatttctgtgctcctaccatcaccatgtaagaattcccgggagctccatgcctttttaattttagccattcttctgcctcatttcttaaaattagagaattaaggtcccgaaggtggaacatgcttcatggtcacacatacaggcacaaaaacagcattatgtggacgcctcatgtattttttatagagtcaactatttcctctttattttccctcattgaaagatgcaaaacagctctctattgtgtacagaaagggtaaataatgcaaaatacctggtagtaaaataaatgctgaaaattttcctttaaaatagaatcattaggccaggcgtggtggctcatgcttgtaatcccagcactttggtaggctgaggtaggtggatcacctgaggtcaggagttcgagtccagcctggccaatatgctgaaaccctgtctctactaaaattacaaaaattagccggccatggtggcaggtgcttgtaatcccagctacttgggaggctgaggcaggagaatcacttgaaccaggaaggcagaggttgcactgagctgagattgtgccactgcactccagcctgggcaacaagagcaaaactctgtctgg

(19) United States
(12) Patent Application Publication
(10) Pub. No.: US 2010/0136543 A1
(43) Pub. Date: Jun. 3, 2010

(54) METHOD FOR DETERMINING THE GENOTYPE AT THE CROHN'S DISEASE LOCUS

(75) Inventors: Michel Georges, Villers-aux-tours (BE); Edouard Louis, Liege (BE); Cecile Libioulle, Angleur (BE); Mark Lathrop, Paris (FR)

Correspondence Address: KNOBBE MARTENS OLSON & BEAR LLP, 2040 MAIN STREET, FOURTEENTH FLOOR, IRVINE, CA, 92614, US

(73) Assignees: UNIVERSITE DE LIEGE, Angleur (BE); CENTRE HOSPITALIER UNIVERSITAIRE DE LIEGE, Liege (BE); COMMISSARIAT A L'ENERGIE ATOMIQUE, Paris (FR)

(21) Appl. No.: 12/529,690

(22) PCT Filed: Feb. 29, 2008
(86) PCT No.: PCT/EP08/52497

§ 371 Date: Dec. 30, 2009

(30) Foreign Application Priority Data
Mar. 2, 2007 (EP) 07103460.7

(51) Int. Cl. C12Q 1/68; C07H 21/00

(52) U.S. Cl. 435/6; 536/23.1

(57) ABSTRACT

The present invention refers to a method for determining the genotype of an individual at the 5p13.1 Crohn's disease risk locus, the method comprising: providing a sample from the individual; determining whether a DNA sequence corresponding to a DNA sequence polymorphism located between coordinated 40,300,000 and 40,600,000 of human chromosome (coordinates corresponding to the march 2006 assembly of the human genome) is present in the sample; and determining the nature of the DNA sequence polymorphism genotype located between coordinated 40,300,000 and 40,600,000 of human chromosome as it relates to the genetic risk to develop Crohn's disease.

This invention refers to a method for determining the genotype of an individual at the 5p13.1 Crohn's disease risk locus, by determining DNA sequence polymorphism located between coordinated 40,300,000 and 40,600,000 of human chromosome, allowing the estimation of its genetic risk to develop Crohn's disease and allowing to tailor drug treatment according to the patients genotype.

### BACKGROUND OF THE INVENTION

Crohn's disease (CD) is a chronic relapsing inflammatory disorder of the intestinal tract, described for the first time in the 1920ies. Lifetime prevalence has increased to current estimates of ˜0.15% in Caucasians. The precise environmental causes underlying this rise remain essentially unknown, but familial clustering and twin-studies clearly identify an inherited component to predisposition. More than ten susceptibility loci have been identified by linkage and/or association studies and convincing causative mutations have been reported, particularly in CARD15 (Schreiber S. et al. Nat Rev Genet. 6:376-388 (2005); Hugot J P et al. Nature 411:599-603 (2001)). As known loci don't fully account for the genetic risk for CD in the present studies a genome-wide association scan (WGA) was performed to contribute to the identification of additional susceptibility loci.

### FIELD OF THE INVENTION

Many of the common human diseases including cancer, hypertension, diabetes, asthma and CD are multifactorial diseases. This means that what determines the fact that some individuals will be afflicted by the disease and others not are a series of environmental that act in concert with a series of genetic risk factors. Risk variants at susceptibility loci (=genetic risk factors) cause the mis-regulation of specific genes which ultimately cause an increased propensity to suffer from the disease.

Identifying the corresponding genetic risk variants for common diseases is presently one of the most important objectives of medical genetics. Indeed these findings pave the way towards individualized, predictive medicine and towards the identification of novel drug targets. Individuals that are genetically predisposed to the disease may alter their behaviour undergo preventive treatment to decrease the risk of becoming sick. Knowing the genetic risk variants of specific individuals may orient the choice of treatment on the basis of their genetically altered molecular biology. Moreover, the products of genetically misregulated genes are prime targets for drug development.

Therefore the object of the present invention was to provide a method for allowing an improved estimation of the genetic risk of an human individual to develop CD.

### SUMMARY OF THE INVENTION

In this invention, the identification of a novel susceptibility locus for Crohn's disease (CD) located on human chromosome 5p13.1 is described. The 5p13.1 CD risk locus corresponds to a region located between positions ˜40,300,000 and ˜40,600,000 (defined according to the march 2006 assembly of the human genome) on human chromosome 5. The region corresponds to a “gene desert”, i.e. it doesn't contain any protein-encoding gene known at the time or writing. However, the invention demonstrates that genetic variants of the 5p13.1 CD risk locus modulate the expression levels of the closest gene coding for the prostaglandin receptor EP4 or PTGER4. PTGER4 is a very strong candidate gene for CD as its inactivation by genetic (PTGER4 knock-out mouse) or by pharmacological means increases susceptibility to colitis in the mouse, while its activation on the other hand protects mice from developing colitis (Kabashima et al., J Clin Invest. 109:883-893 (2002)).

The object of the present invention was solved by a method for determining the genotype of a human individual at the 5p13.1 Crohn's disease (CD) risk locus, the method comprising:

a) providing a sample from the individual;

b) determining whether a DNA sequence corresponding to a DNA sequence polymorphism located between coordinated 40,300,000 and 40,600,000 of human chromosome (coordinates corresponding to the march 2006 assembly of the human genome) is present in the sample;

c) determining the nature of the DNA sequence polymorphism genotype located between coordinated 40,300,000 and 40,600,000 of human chromosome as it relates to the genetic risk to develop Crohn's disease.

The present invention provides a method for determining the genotype of an individual at the 5p13.1 CD risk locus, the method comprising:

a) obtaining a sample of material containing genomic DNA from the individual, wherein the sample can be any material containing nucleated cells from said individual including blood, buccal swaps, urine as well as any other tissues, and

b) ascertaining:
i. whether a DNA sequence corresponding to a DSP located between coordinated 40,300,000 and 40,600,000 of human chromosome (coordinates corresponding to the march 2006 assembly of the human genome) is present in the sample, and
ii. the nature of the DSP genotype as it relates to the genetic risk to develop CD.

Further, the present invention provides a method for determining the genotype of an individual at the 5p13.1 CD risk locus, the method comprising:

a) obtaining a sample of material containing RNA from the individual, wherein the sample can be any material containing nucleated cells from said individual including blood, buccal swaps, urine as well as any other tissues, and

b) converting the RNA in cDNA by means of a reverse transcriptase, and

c) ascertaining:
i) whether a DNA sequence corresponding to a DSP located between coordinated 40,300,000 and 40,600,000 of human chromosome (coordinates corresponding to the march 2006 assembly of the human genome) is present in the sample, and
i) the nature of the DSP genotype as it relates to the genetic risk to develop CD.

In addition, the present invention provides a method for determining the genotype of an individual at the 5p13.1 CD risk locus, the method comprising:

a) obtaining a sample of material containing genomic DNA from the individual, wherein the sample can be any material containing nucleated cells from said individual including blood, buccal swaps, urine as well as any other tissues, and

b) ascertaining:
i) whether a DNA sequence corresponding to a DSP located between coordinated 40,300,000 and 40,600,000 of human chromosome (coordinates corresponding to the march 2006 assembly of the human genome) is present in the sample, and
i) the nature of the DSP genotype as it relates to optimizing treatment for CD.

Still further, the present invention provides a method for determining the genotype of an individual at the 5p13.1 CD risk locus, the method comprising:

a) obtaining a sample of material containing RNA from the individual, wherein the sample can be any material containing nucleated cells from said individual including blood, buccal swaps, urine as well as any other

b) converting the RNA in cDNA by means of a reverse transcriptase, and

c) ascertaining:
i) whether a DNA sequence corresponding to a DSP located between coordinated 40,300,000 and 40,600,000 of human chromosome (coordinates corresponding to the march 2006 assembly of the human genome) is present in the sample, and
i) the nature of the DSP genotype as it relates to optimizing treatment for CD.

In a preferred method the DNA sequence polymorphism is any of the SNPs (single nucleotide polymorphisms) listed in Table 2. Table 2 gives the identification number of the marker, the position of the SNP on the chromosome according to the march 2006 assembly of the human genome, the frequency of the indicated nucleotide in patients having Crohn's disease and in normal individuals (control group [Ctl]), respectively.

It is further preferred that the method includes

i) the determination if or if not an allele associated with increased risk for Crohn's disease as indicated in Table 2 is present;

ii) the judgment if or if not said individual is having a genetic risk to develop Crohn's disease, based on the information of step i).

In another embodiment of the present invention the method includes

i) the determination if an allele associated with increased risk for Crohn's disease as indicated in Table 2 is present;

ii) the judgment that said individual is having a genetic risk to develop Crohn's disease, if an allele associated with increased risk for Crohn's disease was determined.

In another preferred embodiment the sample is any material containing nucleated cells from said individual including blood, buccal swaps, urine as well as any other tissue.

Further preferred RNA is obtained from said sample and the RNA is converted into cDNA by means of a reverse transcriptase.

According to one embodiment of the present invention the allele associated with increased risk for Crohn's disease is selected from haplotypes consisting of IIIA, IIIC, IIA, IIB, IIC, IVB as indicated in FIG. 2C. It should be noted that haplotypes (e.g. IIIA, IIIC, IIA, IIB, IIC, IVB) each represent groups of similar haplotypes. With other words, in a preferred embodiment of the present invention the allele associated with increased risk for Crohn's disease is selected from haplotypes comprised in the haplotype groups IIIA, IIIC, IIA, IIB, IIC, IVB as indicated in FIG. 2C.

A further preferred method includes

iii) the determination if a further allele selected from the group consisting of

CARD15, IL23R, OCTN, DLG5, TNFSF15 and ATG16L1 associated with increased risk for Crohn's disease is present in said individual; and

iv) the judgment that said individual is having a further increased genetic risk to develop Crohn's disease, if in addition to the presence of risk alleles at the 5p13.1 Crohn's disease risk locus any one or more of the allele associated with increased risk for Crohn's disease indicated in iii) was determined.

The 5p13.1 CD risk locus encompasses a large number of DNA sequence polymorphisms (DSP) of different types including single nucleotide polymorphisms (SNPs), insertion-deletions (indels), and microsatelles. Many of these are known and compiled in public databases including dbSNP. These DSP are in linkage disequilibrium with each other and define five so-called haplotype blocks. Each block contains a limited number of common haplotypes. Some of these haplotypes increase the risk to develop CD, while others are protective. The present inventors have defined which haplotypes are associated with increased risk (e.g. haplotypes IIIA, IIIC, IIA, IIB, IIC, IVB; see FIG. 2C) and which are associated with (a relative) decreased risk in the Caucasian population (e.g. haplotypes IVA, IIIB). Knowing the boundaries of the CD 5p13.1 risk locus, the person skilled in the art will be able to identify other disease-associated haplotypes that may be prevalent in the same or other populations.

The genetic composition of an individual at the 5p13.1 CD risk locus can be determined by genotyping the individual using one or preferably several DSP. This can be accomplished using a variety of genotyping methods known by those skilled in the art. Ideally the DSP are chosen to allow unambiguous discrimination of the haplotypes present in the DNA of tested individual.

Knowing the haplotype composition of a given individual at the 5p13.1 CD risk locus will allow an estimation of its risk to develop CD. The risk haplotypes at the 5p13.1 risk locus increase the relative risk by a factor of approximately 1.5. The best prediction will be based on the genotype at the 5p13.1 locus in combination with other known CD genetic risk loci including CARD15, the IL23R, OCTN, DLG5, TNFSF15 and ATG16L1. This is useful as it allows the physician to prescribe preventive behaviour and treatment. Moreover, as the 5p13.1 modulates the expression level of the prostaglandin EP4 receptor, knowledge of the genotype may help the physician to choose for or against medication that acts on this receptor or the corresponding pathway, or to adjust the dose.

The present invention also provides a method for judging a possibility of the onset of Crohn's disease, wherein a sample from a human individual is tested, wherein a human individual in which the DNA sequence located between coordinated 40,300,000 and 40,600,000 of human chromosome (coordinates corresponding to the march 2006 assembly of the human genome) contains an allele associated with increased risk for Crohn's disease as indicated in Table 2 is judged to have a risk of the onset of Crohn's disease. In a preferred embodiment of the method the allele associated with increased risk for Crohn's disease is selected from the CD risk haplotypes consisting of IIIA, IIIC, IIA, IIB, IIC, IVB as indicated in FIG. 2C.

The present invention also provides the use of a genetic marker located on the human 5p13.1 locus for the judgement whether a human individual has increased risk of the onset of Crohn's disease, wherein said marker is represented by DNA sequence polymorphisms.

In a preferred use the DNA sequence polymorphism is any of the single nucleotide polymorphisms listed in Table 2. Further preferred, said marker is represented by single nucleotide polymorphisms associated with increased risk for Crohn's disease as indicated in Table 2. Still further preferred, said marker is represented by alleles associated with increased risk for Crohn's disease selected from the Crohn's disease risk haplotypes consisting of IIIA, IIIC, IIA, IIB, IIC, IVB as indicated in FIG. 2C.

The present invention also provides an oligonucleotide for determining the genotype of a human individual at the 5p13.1 Crohn's disease risk locus, selected from the group consisting of:

a) an oligonucleotide comprising from 12 to 30 contiguous nucleotides of the sequence located between coordinated 40,300,000 and 40,600,000 of human chromosome (coordinates corresponding to the march 2006 assembly of the human genome), wherein said oligonucleotide include one position of the SNPs listed in Table 2, and wherein said position is occupied by a nucleotide corresponding to the respective SNPs correlated with the risk of Crohn's disease as listed in Table 2.

b) an oligonucleotide which is entirely complementary to the oligonucleotide of (a).

### Definitions

Throughout the description of the present invention, several terms are used that are specific to the science of this field. For the sake of clarity and to avoid any misunderstanding, these definitions are provided to aid in the understanding of the specification and claims:

Allele: One of a pair, or series, of forms of a gene or non-genic region that occur at a given locus in a chromosome. Alleles are symbolized with the same basic symbol (e.g., B for dominant and b for recessive; B1, B2, Bn for n additive alleles at a locus). In a normal diploid cell there are two alleles of any one gene (one from each parent), which occupy the same relative position (locus) on homologous chromosomes. Within a population there may be more than two alleles of a gene. See multiple alleles. SNPs also have alleles, i.e., the two (or more) nucleotides that characterize the SNP.

Amplification of nucleic acids: refers to methods such as polymerase chain reaction (PCR), ligation amplification (or ligase chain reaction, LCR) and amplification methods based on the use of Q-beta replicase. These methods are well known in the art. Reagents and hardware for conducting PCR are commercially available. Primers useful for amplifying sequences from the disorder region are preferably complementary to, and preferably hybridize specifically to, sequences in the disorder region or in regions that flank a target region therein.

cDNA: refers to complementary or copy DNA produced from an RNA template by the action of RNA-dependent DNA polymerase (reverse transcriptase). Thus, a cDNA clone means a duplex DNA sequence complementary to an RNA molecule of interest, included in a cloning vector or PCR amplified. This term includes genes from which the intervening sequences have been removed.

cDNA library: refers to a collection of recombinant DNA molecules containing cDNA inserts that together comprise essentially all of the expressed genes of an organism or tissue. A cDNA library can be prepared by methods known to one skilled in the art. Generally, RNA is first isolated from the cells of the desired organism, and the RNA is used to prepare cDNA molecules.

Complement of a nucleic acid sequence (complementary sequence): refers to the antisense sequence that participates in Watson-Crick base-pairing with the original sequence.

Gene: Refers to a DNA sequence that encodes through its template or messenger RNA a sequence of amino acids characteristic of a specific peptide, polypeptide, or protein. The term “gene” also refers to a DNA sequence that encodes an RNA product. The term gene as used herein with reference to genomic DNA includes intervening, non-coding regions, as well as regulatory regions, and can include 5′ and 3′ ends. A gene sequence is wild-type if such sequence is usually found in individuals unaffected by the disorder or condition of interest. However, environmental factors and other genes can also play an important role in the ultimate determination of the disorder. In the context of complex disorders involving multiple genes (oligogenic disorder), the wild type, or normal sequence can also be associated with a measurable risk or susceptibility, receiving its reference status based on its frequency in the general population.

GeneMaps: are defined as groups of gene(s) that are directly or indirectly involved in at least one phenotype of a disorder (some non-limiting example of GeneMaps comprises varius combinations of genes from tables 8-10). As such, GeneMaps enable the development of synergistic diagnostic products, creating “theranostics”.

Genotype: Set of alleles at a specified locus or loci.

Haplotype: The allelic pattern of a group of (usually contiguous) DNA markers or other polymorphic loci along an individual chromosome or double helical DNA segment. Haplotypes identify individual chromosomes or chromosome segments.

The presence of shared haplotype patterns among a group of individuals implies that the locus defined by the haplotype has been inherited, identical by descent (IBD), from a common ancestor. Detection of identical by descent haplotypes is the basis of linkage disequilibrium (LD) mapping. Haplotypes are broken down through the generations by recombination and mutation. In some instances, a specific allele or haplotype may be associated with susceptibility to a disorder or condition of interest, e.g., Crohn's disease. In other instances, an allele or haplotype may be associated with a decrease in susceptibility to a disorder or condition of interest, i.e., a protective sequence.

Host: includes prokaryotes and eukaryotes. The term includes an organism or cell that is the recipient of an expression vector (e.g., autonomously replicating or integrating vector).

Hybridizable: nucleic acids are hybridizable to each other when at least one strand of the nucleic acid can anneal to another nucleic acid strand under defined stringency conditions. In some embodiments, hybridization requires that the two nucleic acids contain at least 10 substantially complementary nucleotides; depending on the stringency of hybridization, however, mismatches may be tolerated. The appropriate stringency for hybridizing nucleic acids depends on the length of the nucleic acids and the degree of complementarity, and can be determined in accordance with the methods described herein.

Identity by descent (IBD): Identity among DNA sequences for different individuals that is due to the fact that they have all been inherited from a common ancestor. LD mapping identifies IBD haplotypes as the likely location of disorder genes shared by a group of patients.

Identity: as known in the art, is a relationship between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. Identity and similarity can be readily calculated by known methods.

Isolated nucleic acids: are nucleic acids separated away from other components (e.g., DNA, RNA, and protein) with which they are associated (e.g., as obtained from cells, chemical synthesis systems, or phage or nucleic acid libraries). Isolated nucleic acids are at least 60% free, preferably 75% free, and most preferably 90% free from other associated components. In accordance with the present invention, isolated nucleic acids can be obtained by methods described herein, or other established methods, including isolation from natural sources (e.g., cells, tissues, or organs), chemical synthesis, recombinant methods, combinations of recombinant and chemical methods, and library screening methods.

Linkage disequilibrium (LD): the situation in which the alleles for two or more loci do not occur together in individuals sampled from a population at frequencies predicted by the product of their individual allele frequencies. In other words, markers that are in LD do not follow Mendel's second law of independent random segregation. LD can be caused by any of several demographic or population artifacts as well as by the presence of genetic linkage between markers. However, when these artifacts are controlled and eliminated as sources of LD, then LD results directly from the fact that the loci involved are located close to each other on the same chromosome so that specific combinations of alleles for different markers (haplotypes) are inherited together. Markers that are in high LD can be assumed to be located near each other and a marker or haplotype that is in high LD with a genetic trait can be assumed to be located near the gene that affects that trait. The physical proximity of markers can be measured in family studies where it is called linkage or in population studies where it is called linkage disequilibrium.

LD mapping: population based gene mapping, which locates disorder genes by identifying regions of the genome where haplotypes or marker variation patterns are shared statistically more frequently among disorder patients compared to healthy controls. This method is based upon the assumption that many of the patients will have inherited an allele associated with the disorder from a common ancestor (IBD), and that this allele will be in LD with the disorder gene.

Locus: a specific position along a chromosome or DNA sequence. Depending upon context, a locus could be a gene, a marker, a chromosomal band or a specific sequence of one or more nucleotides.

Markers: an identifiable DNA sequence that is variable (polymorphic) for different individuals within a population. These sequences facilitate the study of inheritance of a trait or a gene. Such markers are used in mapping the order of genes along chromosomes and in following the inheritance of particular genes; genes closely linked to the marker or in LD with the marker will generally be inherited with it. Two types of markers are commonly used in genetic analysis, microsatellites and SNPs.

Microsatellite: DNA of eukaryotic cells comprising a repetitive, short sequence of DNA that is present as tandem repeats and in highly variable copy number, flanked by sequences unique to that locus.

Mutant sequence: if it differs from one or more wild-type sequences. In some cases, the individual carrying this allele has increased susceptibility toward the disorder or condition of interest. In other cases, the mutant sequence might also refer to an allele that decreases the susceptibility toward a disorder or condition of interest and thus acts in a protective manner. The term mutation may also be used to describe a specific allele of a polymorphic locus.

Non-conservative variants: are those in which a change in one or more nucleotides in a given codon position results in a polypeptide sequence in which a given amino acid residue in a polypeptide has been replaced by a non-conservative amino acid substitution. Non-conservative variants also include polypeptides comprising non-conservative amino acid substitutions.

Nucleic acid or polynucleotide: purine- and pyrimidine-containing polymers of any length, either polyribonucleotides or polydeoxyribonucleotide or mixed polyribo polydeoxyribonucleotides. This includes single-and double-stranded molecules, i.e., DNA-DNA, DNA-RNA and RNA-RNA hybrids, as well as protein nucleic acids (PNA) formed by conjugating bases to an amino acid backbone. This also includes nucleic acids containing modified bases.

Nucleotide: a nucleotide, the unit of a DNA molecule, is composed of a base, a 2′-deoxyribose and phosphate ester(s) attached at the 5′ carbon of the deoxyribose. For its incorporation in DNA, the nucleotide needs to possess three phosphate esters but it is converted into a monoester in the process. Operably linked: means that the promoter controls the initiation of expression of the gene. A promoter is operably linked to a sequence of proximal DNA if upon introduction into a host cell the promoter determines the transcription of the proximal DNA sequence(s) into one or more species of RNA. A promoter is operably linked to a DNA sequence if the promoter is capable of initiating transcription of that DNA sequence.

Phenotype: any visible, detectable or otherwise measurable property of an organism such as symptoms of, or susceptibility to, a disorder.

Polymorphism: occurrence of two or more alternative genomic sequences or alleles between or among different genomes or individuals at a single locus. A polymorphic site thus refers specifically to the locus at which the variation occurs. In some cases, an individual carrying a particular allele of a polymorphism has an increased or decreased susceptibility toward a disorder or condition of interest.

Probe or primer: refers to a nucleic acid or oligonucleotide that forms a hybrid structure with a sequence in a target region of a nucleic acid due to complementarity of the probe or primer sequence to at least one portion of the target region sequence. Protein and polypeptide: are synonymous. Peptides are defined as fragments or portions of polypeptides, preferably fragments or portions having at least one functional activity (e.g., proteolysis, adhesion, fusion, antigenic, or intracellular activity) as the complete polypeptide sequence.

Recombinant nucleic acids: nucleic acids which have been produced by recombinant DNA methodology, including those nucleic acids that are generated by procedures which rely upon a method of artificial replication, such as the polymerase chain reaction (PCR) and/or cloning into a vector using restriction enzymes.

Sample: as used herein refers to a biological sample, such as, for example, tissue or fluid isolated from an individual or animal (including, without limitation, plasma, serum, cerebrospinal fluid, lymph, tears, nails, hair, saliva, milk, pus, and tissue exudates and secretions) or from in vitro cell culture-constituents, as well as samples obtained from, for example, a laboratory procedure.

Single nucleotide polymorphism (SNP): variation of a single nucleotide. This includes the replacement of one nucleotide by another and deletion or insertion of a single nucleotide. Typically, SNPs are biallelic markers. For example, SNP A\C may comprise allele C or allele A. Thus, a nucleic acid molecule comprising SNP A\C may include a C or A at the polymorphic position. For a combination of SNPs, the term “haplotype” is used, e.g. the genotype of the SNPs in a single DNA strand that are linked to one another. In certain embodiments, the term “haplotype” is used to describe a combination of SNP alleles, e.g., the alleles of the SNPs found together on a single DNA molecule. In specific embodiments, the SNPs in a haplotype are in linkage disequilibrium with one another.

Sequence-conservative: variants are those in which a change of one or more nucleotides in a given codon position results in no alteration in the amino acid encoded at that position (i.e., silent mutation).

Substantially homologous: a nucleic acid or fragment thereof is substantially homologous to another if, when optimally aligned (with appropriate nucleotide insertions and/or deletions) with the other nucleic acid (or its complementary strand), there is nucleotide sequence identity in at least 60% of the nucleotide bases, usually at least 70%, more usually at least 80%, preferably at least 90%, and more preferably at least 95-98% of the nucleotide bases. Alternatively, substantial homology exists when a nucleic acid or fragment thereof will hybridize, under selective hybridization conditions, to another nucleic acid (or a complementary strand thereof). Selectivity of hybridization exists when hybridization which is substantially more selective than total lack of specificity occurs. Typically, selective hybridization will occur when there is at least about 55% sequence identity over a stretch of at least about nine or more nucleotides, preferably at least about 65%, more preferably at least about 75%, and most preferably at least about 90%. The length of homology comparison, as described, may be over longer stretches, and in certain embodiments will often be over a stretch of at least 14 nucleotides, usually at least 20 nucleotides, more usually at least 24 nucleotides, typically at least 28 nucleotides, more typically at least 32 nucleotides, and preferably at least 36 or more nucleotides.

### DETAILED DESCRIPTION OF THE PREFERRED EMBODIMENTS

In one aspect, the present invention provides a method to determine the genotype of an individual at the 5p13.1 CD risk locus by analyzing its genomic DNA. The method includes obtaining a sample of material containing genomic DNA from the individual and genotyping it for DSP/markers mapping between coordinates 40,300,000 and 40,600,000 of human chromosome 5 (coordinates corresponding to the march 2006 assembly of the human genome). The markers can be any single or combination of microsatellite markers, single nucleotide polymorphisms (SNPs) or insertion-deletions (indels). Many of these are listed in public databases including dbSNP, but additional ones can easily be generated by the person skilled in the art by re-sequencing the corresponding region from one or more individuals. Based on the genotype of these markers and given the information presented in later sections of the present patent the person skilled in the art can determine whether the individuals has a genotype that increases or decreases the risk to have CD, or whether a CD patient should be administered drugs that affect the function of the PTGER4 receptor or not. The sample can be any material containing nucleated cells from said individual.

There are several methods known by those skilled in the art for determining the genotype of an individual at a DSP. These include the amplification of a DNA segment encompassing the polymorphism by means of the polymerase chain reaction and interrogate the variant nucleotide position by means of allele specific hybridization, or the 3′exonuclease assay (Taqman assay), or the use of allele-specific restriction enzymes, or direct sequencing, or the oligonucleotide ligation assay, or pyrosequencing, or the invader assay, or minisequencing, or DHPLC, or SSCP, or combinations of these methods. Alternatively the gene sequence and mutation can be ascertained by means of allele specific PCRs using primers that are specific for either the allele. This list of methods is not meant to be exclusive, but just to illustrate the diversity of available methods. Some of these methods can be performed in microarray format.

In another aspect, the present invention provides a method for determining the genotype of individual at the 5p13.1 CD susceptibility locus by analyzing its RNA. The method includes obtaining a sample of material containing RNA from the individual and genotyping it for polymorphic markers mapping between coordinates 40,300,000 and 40,600,000 of human chromosome 5 (coordinates corresponding to the march 2006 assembly of the human genome). The sample can be any material containing nucleated cells from said individual. There are several methods known by those skilled in the art for determining whether a particular nucleotide sequence is present in a RNA sample. These include the conversion of the RNA in cDNA by means of a reverse transcriptase, and the application of the methods mentioned above or variants thereof that are known by those skilled in the art to genotype a given polymorphism.

### DESCRIPTION OF THE FIGURES

FIG. 1 shows the results of the whole genome association for CD. P-values (-log(p)) for the 10,000 best SNPs out of 311,882 are shown (light-gray circles). The position of previously described susceptibility loci are marked by arrows. The p-values obtained in our cohorts with the reportedly associated SNPs/mutations are shown by the filled black dots, and the corresponding odds ratios (OR) indicated. The p-values obtained with SNPs included in the Illumina panel at ≦50 Kb from these SNPs/mutations are marked by black circles. SNPs genotyped in the confirmation cohort are shown as dark-gray dots. Two singleton SNPs, located respectively on chromosome 3 (rs11128423) and 6 (rs10485060), yielding p-values <10⁻¹⁰ in the WGA experiment were genotyped in the replication samples but did not provide confirmatory evidence of association (data not shown).

FIG. 2 shows in panel (A) pair-wise LD analysis between the 111 SNPs in the 250 Kb window. r² (lower left) and D′ (upper right) values were computed using standard procedures from the genotypes phased with PHASE (Stephens M. et al., Am J Hum Genet. 68:978-989 (2001)). Values>0.93 are marked in light-gray, values ≦0.93 in dark-gray. The five LD blocks are easily identified and marked by corresponding boxes I to V. (B) Dots: results of single-marker association analyses for CD using 111 SNPs located in a 250 Kb window spanning the positions of the most significant 5p13.1 markers in the WGA. The results are expressed as log(1/p) where p corresponds to the p-value of the association determined by chi-squared analysis. The positions of the 111 markers are indicated by the small triangles. The limits between the LD blocks (I-V) are indicated by filled triangles. Diamonds: log(1/p) values of the effect of marker genotype on PTGER4 expression levels for the 28 HumanHap300 Genotyping Beadchip SNPs mapping to the 250 Kb window. Values are only shown when exceeding 2. (C) Haplotype analysis of LD blocks II, III and IV. The panel is showing the haplotypes. Note that the haplotypes shown in this panel do not represent contiguous sequences. This means that only the SNP positions are shown, while the nucleotide sequences between these SNP positions are not given. Haplotypes accounting jointly for>93% of studied chromosomes are shown. The ancestral allele is in grey when known. Within each block, similar haplotypes are grouped in “clades” (e.g. IIA, IIB and IIC). For blocks II and III, supposedly recombinant haplotypes are represented under the major clades and marked accordingly. The frequency of the corresponding haplotypes and clades in CD patients (CD) and controls (CTR) are given. p-values (chi-squared test) of the clade-based association tests for CD are given underneath for intervals bounded by recombination events. The approximate positions of within-block recombinations are marked by vertical lines between p-values. The two haplotypes forming the IIIBa sub-clade are indicated.

### EXAMPLES

### Example 1

### Genotyping

Genotyping for the whole genome scan was performed on a Illumina HumanHap300 Genotyping Beadchip (Gunderson K. L. et al, Nat Genet. 37:549-554 (2005)). Genotyping of individual SNPs was performed on an ABI7900HT Sequence Detection System using TaqMan MGB probes from “Pre-designed SNP Genotyping” or “Custom TaqMan SNP Genotyping” Assays (Applied Biosystems, Foster City, Calif.).

### Example 2

### Association Analyses

Association analyses were conducted using Fisher's exact test (whole genome scan) or chi-squared tests of independence (confirmation analysis). The logistic regression method of Setakis et al. (Genome Research 16: 290-296 (2006)) was applied to test for the possible effect of population structure on the most significant association results. The 110 control markers included in the logistic regression had 100% genotype success rate with minor allele frequency >30%, and no two markers were within 20 Mb. To test for an effect of block I conditional on the effect of an adjacent block II, the proportion of I haplotype clades nested within a given II clade (f.i. proportion of IA, IB and IC within IIA) was compared between cases and controls by chi-squared. Chi-squared values (and d.f.) were summed across II clades to yield an overall (I|II) test statistic.

### Example 3

### Expression Database

The database genome-wide expression analysis data was provided by W. Cookson (Imperial College, London). Briefly, expression data were generated from RNA extracted from EBV-transformed cells from 378 genotyped offspring in nuclear families. Annotations for individual transcripts on the Affymetrix arrays were extracted from the Affymetrix NetAffx database (www.affymetrix.com). Data from the gene expression experiment was normalized together using the RMA (Robust Multi-Array Average) package (Irizarry R. A. et al., Biostatistics 4: 249-264 (2003), Bolstad B. M. et al., Bioinformatics 19: 185-193 (2003)) to remove any technical or spurious background variation. An inverse normalization transformation step was also applied to each trait to avoid any outliers. A variance components method was used to estimate heritability of each trait using the Merlin-regress (RandomSample option) (Abecasis G. R. et al., Nat Genet 30: 97-101 (2002); Sham P. C. et al., Am J Hum Genet 71: 238-253 (2002)). For PTGER4, a mean quantitative expression value of −0.017 and a variance of 0.722 was obtained while the heritability estimate for PTGER4 estimated using the sibship data was 0.844. Association analysis was applied with Merlin (FASTASSOC option). An additive effect for SNPs was estimated and its significance tested using a score test that adjusts for familiality and takes into account uncertainty in the inference of missing genotypes.

### Results

Genotype data from the Illumina HumanHap300 Genotyping Beadchip were obtained on 547 Caucasian CD patients from Belgium and compared to genotypes for 928 healthy controls from Belgium and France. Genotype call rates were>93% for all individuals included in the study. Of the total 317,497 SNPs available, 5,615 with genotyping success rate of less than 91% or deviating from Hardy-Weinberg proportions in controls (Fisher's exact test p≦10⁻³) were eliminated from further analysis as it is known that less reliable markers generate spurious associations. For the remaining 311,882 SNPs, we compared allele frequencies between cases and controls as outlined below.

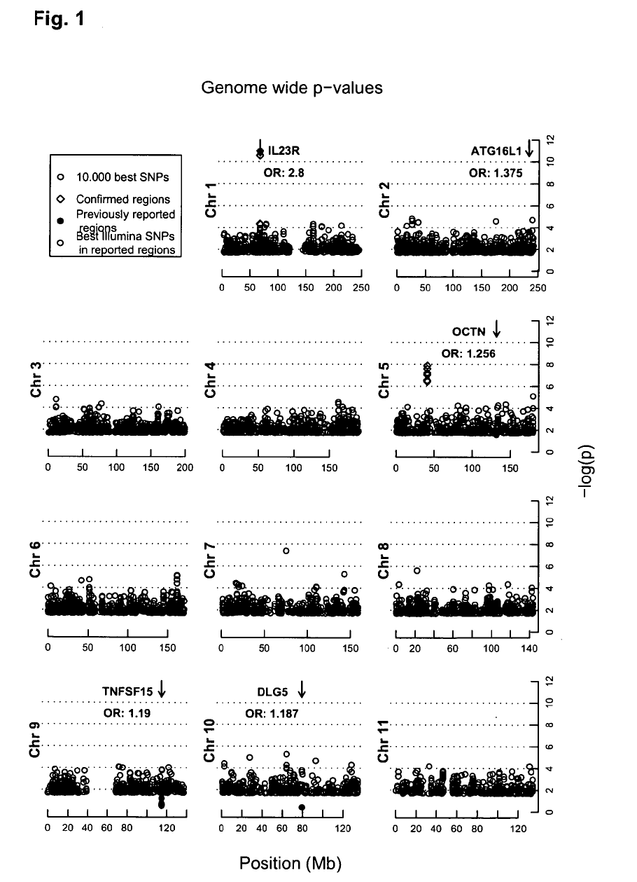

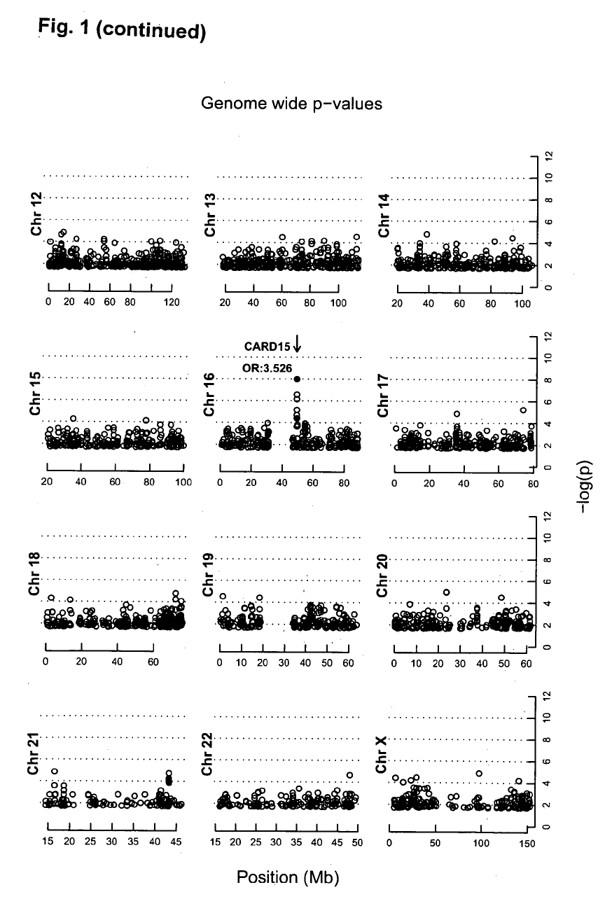

FIG. 1 shows the 10,000 most significant p-values obtained across the human genome. Regions on chromosomes 1, 5 and 16 harboured clusters of markers with suggestive evidence of association at significance levels between 10⁻⁶ and 10⁻¹⁰. The significance of tests of association with these markers remained within this range after controlling for possible effects of population structure using a backwards stepwise regression. The strongest association was found with markers of the IL23R gene on chromosome 1 which has recently been identified as a novel CD susceptibility locus in a case-control and family-based association study of Caucasian and Jewish cohorts. In the present data, two markers of the IL23R gene, rs11209026 and rs11465804, gave the most significant association signals (p<10⁻⁹). Rs11209026 corresponds to an Arg381 Gln substitution in IL23R while rs11465804 is intronic and in strong LD with the former marker. A marker within the CARD15 gene on chromosome 16, which is the first susceptibility gene to have been identified in CD, also showed suggestive evidence of association (rs5743289; p<10⁻⁶). The results of the WGA with respect to other previously reported susceptibility loci, including OCTN, DLG5, TNFSF15 and ATG16L1 were also examined. None of these obtained a similar level of significance for association in the present study. Genotyping our cohorts for other SNPs at these loci that are reported in the literature to be associated with CD did not improve the signals, with the exception of rs224188 corresponding to a Thr to Ala substitution within ATGL16L1 (p<2×10⁻⁴), thus providing confirmation of this novel susceptibility locus for the first time.

On chromosome 5p13.1, a region of approximately 250 Kb was identified that contained six markers with p<10⁻⁶ in the association test. This region has not previously been reported as a CD susceptibility locus. 10 markers from the regions of IL23R and 5p13.1 were selected for confirmation genotyping in up to 1,266 additional Caucasian CD patients and 559 additional controls. The IL23R locus was included in the confirmation genotyping. The associations at these two loci were clearly replicated with p-values as low as 4.2×10⁻⁷ at the IL23R and 3.7×10⁻⁴ at 5p13.1 (Table 1). In the combined data from the WGA and replication studies, p-values as low as 2.2×10⁻¹⁸ at IL23R and 2.1×10⁻¹² at the 5p13.1 locus were obtained. In addition, trios with non-affected parents for the same SNPs were genotyped to perform a transmission disequilibrium test (TDT). The 10 SNPs were typed on 137 trios with affected offspring included in the case-control study, while two of the 5p13.1 SNPs were typed on an additional 291 independent trios originating also from Belgium. Significant over-transmission of the associated alleles were found at both loci, thus providing additional confirmatory evidence in support of the IL23R1 and 5p13.1 susceptibility loci (Table 1).

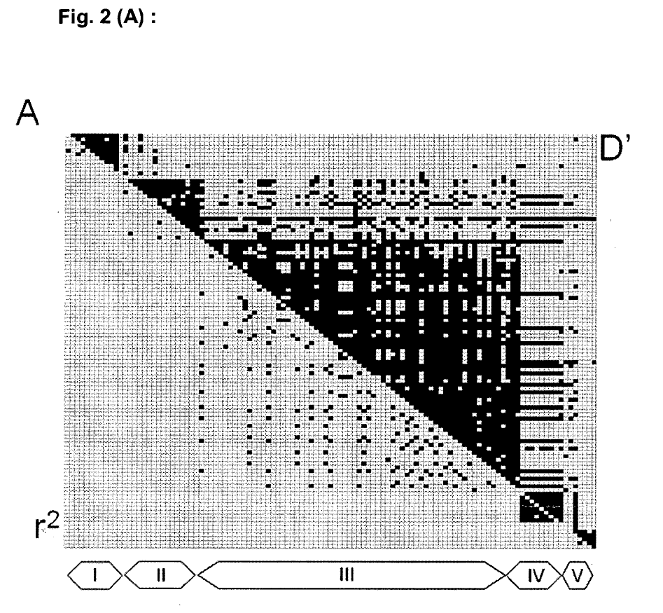

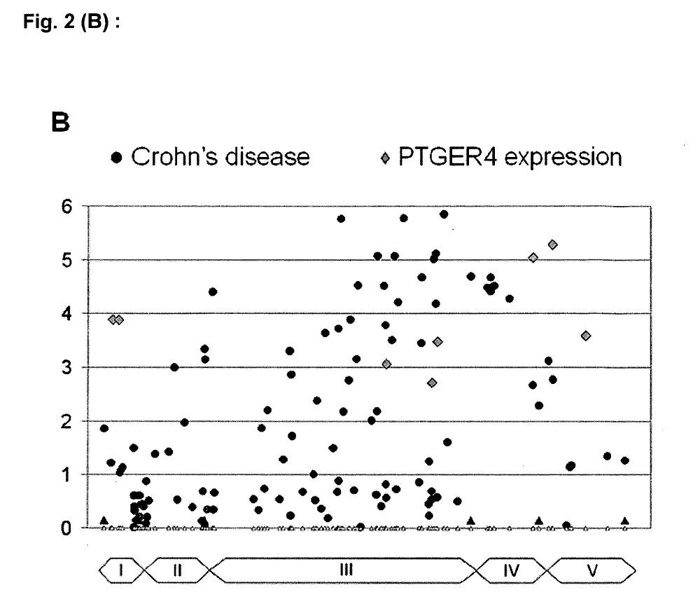

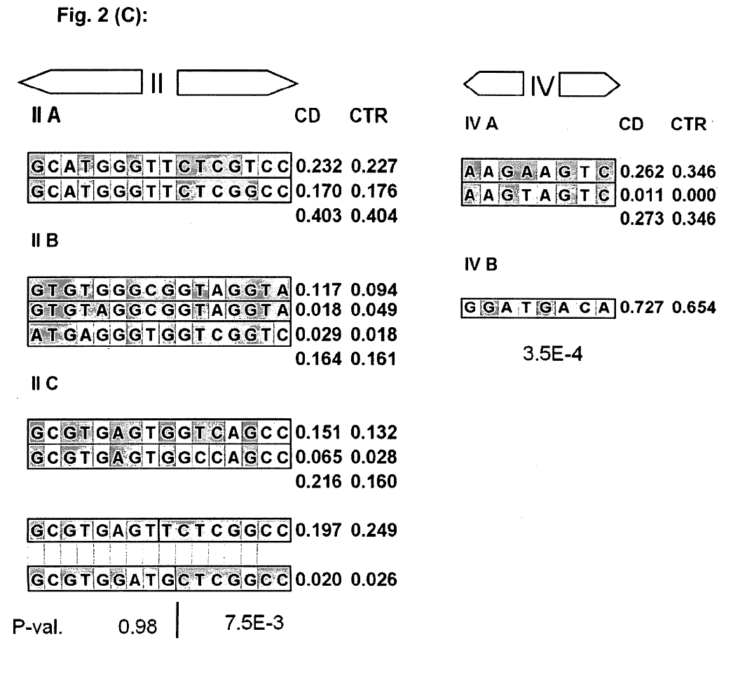

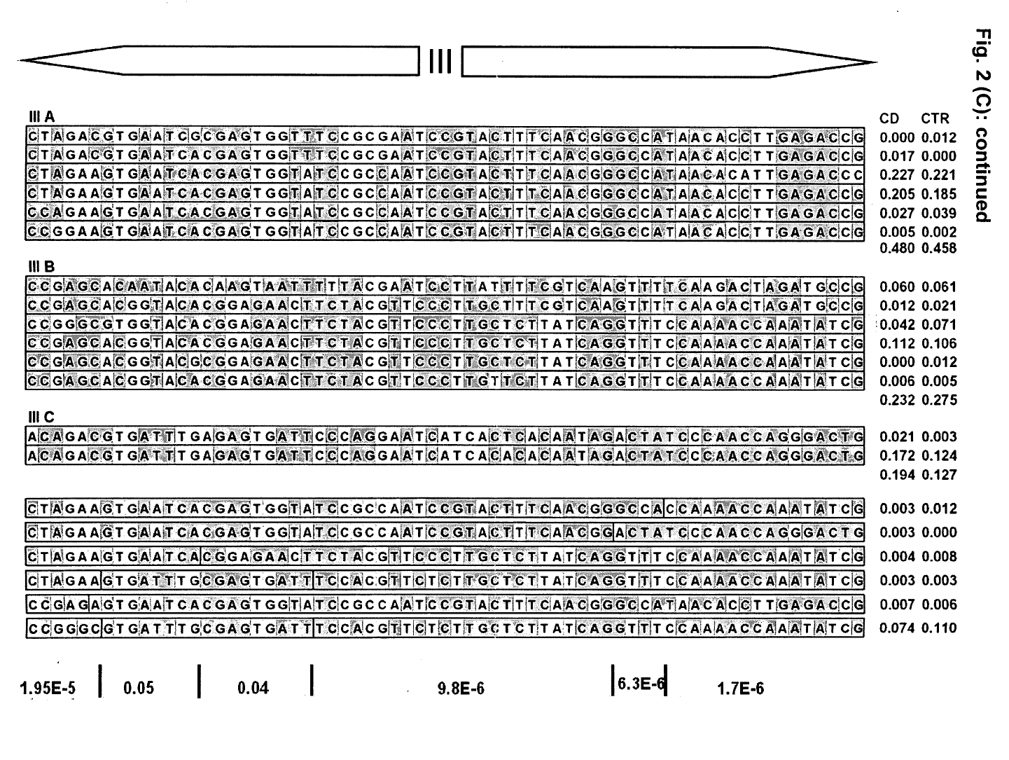

To further characterize the novel 5p13.1 locus, a subset of 1,092 CD patients and 374 Belgian controls were genotyped for 111 markers (Table 2)(average interval: 2.3 Kb) spanning the 250 Kb segment. The most likely linkage phase for each individual was determined using PHASE, and the corresponding haplotype frequencies was used to quantify the level of linkage disequilibrium (LD) between all marker pairs. The 250 Kb encompass five clearly delineated LD blocks, the central one (block III) being the largest and spanning 122 Kb (FIG. 2A). First single-marker association analyses were performed. The strongest effects were observed within the 122 Kb block III with several SNPs yielding p-values <10⁻⁵. P-values<10⁻³ and 10⁻⁴ were observed in flanking blocks II and IV, respectively (FIG. 2B). Then haplotype analysis of the region spanned by blocks II to IV was performed. For block III, 20 haplotypes accounted for 93% of the observed chromosomes. These could be grouped in three clades comprising respectively six (IIIA), six (IIIB) and two (IIIC) haplotypes, plus a group of six haplotypes that apparently originated from various recombination events. Likewise, evaluation of block II revealed three clades (with respectively two (IIA), three (IIB) and two (IIC) haplotypes) and two recombinant haplotypes, while block IV was characterized by two clades with two (IVA) and one (IVB) haplotype respectively. The clade frequencies in cases and controls were compared at intervals bounded by ancestral recombination events (FIG. 2C). In agreement with the results of the single-marker analysis, the most significant associations were found in block III followed by IV and II. To verify whether the entire 5p13.1 effect could be attributed to block III (i.e. the effects observed for blocks II and IV would be mere echos of the block III effect), a multi-variate analysis was performed as described. The clade effects of blocks II and IV conditional on the effect of block III and vice versa, remained significant (p_{(II|III)}=0.023; p(_{III|II)}=0.0004; P_{(IV|III)}=0.003; p_{(III|IV)}0.026), suggesting that multiple variants in the region may jointly account for the observed effect on CD. Commonly occurring recombinant haplotypes in blocks II and III caused local drops in significance thus suggesting that causal variants lie outside the corresponding sub-segments (FIG. 2C).

No known genes or CpG islands were found within the region of association on 5p13.1 after examination with the Ensembl and UCSC genome browsers. The region has an average G+C content of 38%, and an excess of interspersed repeats given GC content (58.36% vs 42.3%), which is mainly due to an excess of LINE1's (33.05% vs 19.6%) and LTR elements (15.36% vs 7.70%). It contains 98 Phastcons conserved elements. It is part of a 1.25 Mb gene desert 30 between DAB2 (850 Kb distally from the block) and PTGER4 (270 Kb proximally from the block). Interestingly several of the genes flanking the region have been implicated in pathogenesis of CD, or are related to genes that have been implicated in the disease. These include a member of the caspase recruitment domain family (CARD6), three complement factors (C6, C7 and C9), and—most notably—the prostaglandin receptor EP4 (PTGER4), which resides closest to the group of disease associated markers.

One hypothesis is that the disease-associated region contains cis-acting regulatory elements that control the expression levels of the causal gene(s) located in the vicinity, and that the causal variants modulate the activity of these elements. As a first step to test this, the effect of SNPs in the disease-associated region on the expression levels of neighbouring genes was studied. To that end a database of genome-wide gene expression (Affymetrix HG-U133 Plus 2.0 chips) measured in EBV-transformed lymphoblastoid cell lines from 378 individuals genotyped with the Illumina HumanHap300 Genotyping Beadchip was exploited. Remarkably, seven of the 26 Illumina markers spanning 264 Kb coinciding precisely with the CD-associated region yielded p-values between 6.7×10⁻⁵ and 1×10⁻³ for PTGER4 (FIG. 2B). Three of the markers influencing PTGER4 expression are located in block III (rs16869977, rs10512739 and rs6880934). The first two are tagging the IIIBa sub-clade (r²=1) (FIG. 2C), while the third one is in complete LD with it (D′=1). The corresponding SNPs and IIIBa haplotypes did not show evidence for association with CD. Two strongly associated SNPs (D′=0.84) located respectively in block IV (rs4495224) and V (rs7720838) were showing the most significant effect on PTGER4 expression and were also associated with CD (Table 1). The rs4495224 A and rs7720838 T risk alleles were associated with increased PTGER4 expression. These results tend to support the hypothesis that the disease-associated polymorphisms may be related to the expression levels of one or more genes in the region.

CD is the most common form of inflammatory bowel disease (IBD), the other being ulcerative colitis (UC). In the studies of the present invention a cohort of 246 Belgian UC patients (Caucasians) was genotyped for IL23R (rs11209026), ATG16L1 (rs2241880) and the novel 5p13.1 locus (rs4613763). A significant association was found for IL23R (p=1.2×10⁻³; OR: 2.51) but not for ATG16L1 (p=0.78). There was no effect of the novel 5p13.1 locus on UC (p=0.54). While additional studies will be needed to exclude completely a role in UC, these results suggests that the principal susceptibility effects of the 5p13.1 locus are for CD. The restriction to CD risk observed for ATG16L1 and the 5p13.1 locus is similar to that found for CARD15.

The present invention describes the localisation of a novel major susceptibility locus for CD on 5p13.1 by WGA. The region of strongest association coincides with a gene desert devoid of known protein-coding genes. The observed effect may be mediated by as of yet unknown transcripts mapping within the region. As a matter of fact limited numbers of spliced and unspliced ESTs originating from the HT1080 fibrosarcoma cell line or medulla (e.g. BG182136, BG184600) map to the region. An alternative explanation, however, is that the disease-associated region contains cis-acting elements controlling the expression of more distant genes. The present invention provides evidence in support of this hypothesis by demonstrating that genetic variants in the CD-associated region differentially regulate the expression levels of PTGER4, the closest known gene located at 270 Kb proximally. PTGER4 is a strong candidate gene for CD as it is known that knock-out (KO) mice develop severe colitis upon dextran sodium sulphate treatment contrary to mice deficient in either of the seven other types of prostanoid receptors. Increased susceptibility to colitis is also observed in wild-type mice administered an EP4-selective antagonist, while EP4-selective agonist are protective. In particular, it was observed that the CD susceptibility allele at marker rs4495224 is associated with increased PTGER4 transcript levels in lymphoblastoid cell lines. This finding establishes a direct link between disease susceptibility and PTGER4 expression, although the direction of the effect apparently contradicts the results in KO mice. Detailed studies of the effect of genetic variants in the disease-associated region on PTGER4 expression in different tissues and of a possible connection between PTGER4 levels and CD susceptibility are certainly needed and work towards that goal is in progress. The hypothesis that the 5p13.1 CD-susceptibility locus operates by modulating PTGER4 expression levels could—at least in theory—be tested by replacing the corresponding murine sequences with the human orthologous variants and quantitatively complement the murine KO allele. The present results suggest that the 5p13.1 effect on CD could result from the combined action of multiple susceptibility variants. Extensive sequencing of the most common haplotypes in the region of association is being conducted towards their identification.

TABLE 1
Results of primary and confirmatory association analysis for the IL23R and 5p13.1
loci, as well as of TDT for 5p13.1 (controls [Ctl], cases [Cas.]).

| | | | | Confirmatory | | | | |
|---|---|---|---|---|---|---|---|---|
| | | Primary data | | data | | Combined | | |
| Locus | SNP | Ctl | Cas. | Ctl | Cas. | Ctl | Cas. | TDT |
| IL23R | rs11465804 | 0.915^{#} | 0.971^{ε} | 0.934 | 0.970 | 0.922 | 0.970 | 16:4° |
| | 67475114^{§} | 923^{&} | 553^{£} | 555 | 928 | 1,478 | 1,481 | 137^{@} |
| | | 0.98^{$} | 3.2E−8^{%} | 0.96 | 1.7E−5 | 0.99 | 3.5E−15 | 0.04^{φ} |
| | | | 3.00* | | 2.30 | | 2.74 | |
| | rs11209026 | 0.918 | 0.972 | 0.934 | 0.972 | 0.924 | 0.972 | 17:5 |
| | (67478546) | 906 | 550 | 550 | 1,255 | 1,456 | 1,807 | 135 |
| | Arg381Gln | 0.93 | 1.5E−8 | 0.64 | 4.2E−7 | 0.99 | 2.2E−18 | 0.045 |
| | | | 3.20 | | 2.48 | | 2.92 | |
| | rs1343151 | 0.641 | 0.712 | 0.655 | 0.722 | 0.646 | 0.719 | 76:39 |
| | (67491717) | 928 | 554 | 556 | 1,266 | 1,484 | 1,820 | 137 |
| | | 0.88 | 3.0E−4 | 0.32 | 2.9E−4 | 0.87 | 2.3E−9 | 0.0003 |
| | | | 1.38 | | 1.36 | | 1.40 | |
| | rs10889677 | 0.291 | 0.354 | 0.31 | 0.36 | 0.30 | 0.36 | 69:44 |
| | (67497708) | 927 | 550 | 559 | 1,263 | 1,486 | 1,813 | 135 |
| | | 0.91 | 0.002 | 0.75 | 0.015 | 0.73 | 2.4E−6 | 0.009 |
| | | | 1.33 | | 1.25 | | 1.31 | |
| 5p13.1 | rs348601 | 0.589 | 0.686 | 0.629 | 0.668 | 0.604 | 0.673 | 72:64 |
| | (40355763) | 928 | 552 | 545 | 1,261 | 1,473 | 1,813 | 138 |
| | | 0.24 | 5.1E−7 | 0.53 | 0.067 | 0.82 | 6.6E−7 | 0.05 |
| | | | 1.54 | | 1.19 | | 1.36 | |
| | rs1002922 | 0.665 | 0.762 | 0.697 | 0.741 | 0.675 | 0.747 | 62:44 |
| | (40422312) | 903 | 550 | 441 | 1,212 | 1,344 | 1,762 | 134 |
| | | 0.46 | 9.1E−8 | 0.45 | 0.04 | 0.95 | 1.7E−9 | 0.040 |
| | | | 1.63 | | 1.25 | | 1.43 | |
| | rs4613763 | 0.120 | 0.191 | 0.139 | 0.183 | 0.127 | 0.185 | 139:113 |
| | (40428485) | 929 | 553 | 545 | 1,247 | 1,474 | 1,800 | 428 |
| | | 0.99 | 6.1E−7 | 0.13 | 6.2E−3 | 0.37 | 1.2E−9 | 0.050 |
| | | | 1.74 | | 1.38 | | 1.56 | |
| | rs10512734 | 0.666 | 0.762 | 0.685 | 0.742 | 0.673 | 0.748 | 61:46 |
| | (40429362) | 929 | 553 | 543 | 1,236 | 1,472 | 1,789 | 136 |
| | | 0.30 | 9.7E−8 | 0.91 | 1.8E−3 | 0.62 | 9.2E−11 | 0.073 |
| | | | 1.63 | | 1.33 | | 1.45 | |
| | rs1373692 | 0.585 | 0.690 | 0.607 | 0.674 | 0.593 | 0.679 | 214:177 |
| | (40466940) | 929 | 554 | 552 | 1,235 | 1,481 | 1,789 | 428 |
| | | 0.13 | 4.1E−8 | 0.89 | 3.7E−4 | 0.43 | 2.1E−12 | 0.030 |
| | | | 1.59 | | 1.35 | | 1.46 | |
| | rs4495224 | 0.651 | 0.746 | 0.675 | 0.708 | 0.659 | 0.720 | 66:43 |
| | (40513272) | 926 | 552 | 544 | 1,237 | 1,470 | 1,789 | 137 |
| | | 0.60 | 2.2E−7 | 0.99 | 0.134 | 0.71 | 6.6E−7 | 0.013 |
| | | | 1.59 | | 1.17 | | 1.33 | |

^{§}Chromosomal position on march 2006 assembly.
Controls:
^{#}allelic frequency of risk allele;
^{&}number of individuals with genotype;
^{$}p-value of Hardy-Weinberg proportions (Fisher's exact test).
Cases:
^{ε}allelic frequency of risk allele;
^{£}number of individuals with genotype;
^{%}p-value of allelic association (chi-squared test);
*Odds Ratio
Results in “Primary data” were obtained after re-genotyping of the initial samples using the Taqman assay conducted to verify the Illumina genotypes.
TDT: °times transmitted:times non-transmitted;
^{@}number of genotyped trios;
^{φ}p-value of segregation distortion (one-sided chi-squared test)

TABLE 2
SNPs in the 5p13.1 CD-associated region.

| | | | | | | | Allele more | |
|---|---|---|---|---|---|---|---|---|
| | | | | | Frequency | Frequency | frequent in | |
| No: | Marker | Position | Variation | All | Crohn | Control | Crohn | Block |
| 1 | rs755989 | 40307432 | T/C | C | 0.24209486 | 0.28825137 | T | Block I |
| 2 | rs17225380 | 40310556 | A/G | A | 0.16666667 | 0.20094937 | G | |
| 3 | rs348615 | 40310881 | A/G | A | 0.7877451 | 0.82044199 | G | |
| | rs971212 | 40311450 | C/T | | | | | |
| | rs2860001 | 40314605 | A/G | | | | | |
| 4 | rs17823954 | 40315168 | A/C | A | 0.76663357 | 0.73569482 | A | |
| 5 | rs12514517 | 40315833 | G/A | A | 0.24111675 | 0.2740113 | G | |
| 6 | rs348617 | 40316826 | A/G | A | 0.21057884 | 0.17945205 | A | |
| 7 | rs6883840 | 40322167 | A/G | A | 0.23451327 | 0.19589041 | A | |
| 8 | rs4957263 | 40322254 | C/T | | 0.50100705 | 0.50140056 | | |
| 9 | rs348619 | 40322436 | A/G | G | 0.90032154 | 0.9375 | A | |
| 10 | rs348620 | 40322578 | A/C | A | 0.11638955 | 0.12953368 | C | |
| 11 | rs7723858 | 40322619 | A/C | A | 0.14960239 | 0.13674033 | A | |
| 12 | rs529750 | 40323642 | A/C | A | 0.8218842 | 0.81621622 | A | |
| 13 | rs348581 | 40324506 | A/G | A | 0.03121951 | 0.02291105 | A | Block II |
| 14 | rs4245975 | 40324635 | C/T | C | 0.83667622 | 0.82786885 | C | |
| 15 | rs13186205 | 40325745 | G/A | A | 0.39444444 | 0.375 | A | |
| 16 | rs348582 | 40326383 | T/A | A | 0.02917505 | 0.02322404 | A | |
| 17 | rs12519421 | 40327723 | A/G | A | 0.03468208 | 0.05882353 | G | |
| 18 | rs10067892 | 40327748 | A/G | G | 0.40755467 | 0.41208791 | A | |
| 19 | rs10068265 | 40328261 | A/G | A | 0.02272727 | 0.02857143 | G | |
| 20 | rs6878901 | 40328934 | C/T | T | 0.14087302 | 0.15633423 | C | |
| 21 | rs9292764 | 40331661 | G/T | G | 0.4029703 | 0.35972222 | G | |
| 22 | rs1564269 | 40338324 | C/G | C | 0.61750246 | 0.66076294 | G | |
| 23 | rs11743023 | 40340893 | C/T | C | 0.06109482 | 0.0296496 | C | |
| 24 | rs348568 | 40342500 | A/C | A | 0.14306641 | 0.15896739 | C | |
| 25 | rs16869807 | 40345756 | A/G | A | 0.20617111 | 0.15830721 | A | |
| 26 | rs11953052 | 40349760 | G/T | G | 0.77810651 | 0.79268293 | T | |
| 27 | rs7716277 | 40354396 | C/T | C | 0.82662083 | 0.82113821 | C | |
| 28 | rs1963925 | 40354887 | A/C | A | 0.13685239 | 0.15582656 | C | |
| 29 | rs1445002 | 40355634 | A/C | A | 0.1894317 | 0.13215259 | A | Block III |
| 30 | rs10512732 | 40355676 | C/T | | 0.52056075 | 0.52380952 | | |
| 31 | rs348601 | 40355763 | C/T | T | 0.67892157 | 0.60906516 | T | |
| 32 | rs348599 | 40356915 | A/G | A | 0.20111732 | 0.22619048 | G | |
| 33 | rs348595 | 40359471 | A/G | A | 0.6839196 | 0.59916201 | A | |
| 34 | rs10043093 | 40359977 | A/C | A | 0.47502498 | 0.45890411 | A | |
| 35 | rs348593 | 40360289 | A/G | A | 0.1888454 | 0.20967742 | G | |
| 36 | rs7726744 | 40379033 | C/T | C | 0.18768473 | 0.20564516 | T | |
| 37 | rs12518245 | 40381478 | A/G | A | 0.06299213 | 0.07083333 | G | |
| 38 | rs16869831 | 40383016 | A/G | A | 0.83041788 | 0.78961749 | A | |
| 39 | rs12697408 | 40384330 | A/T | A | 0.49305556 | 0.46457766 | A | |
| 40 | rs11742533 | 40385962 | A/T | A | 0.23058014 | 0.28091398 | T | |
| 41 | rs7725639 | 40391593 | C/T | C | 0.72696078 | 0.74726776 | T | |
| 42 | rs6451489 | 40393420 | A/G | A | 0.73327138 | 0.80357143 | G | |
| 43 | rs11743463 | 40396215 | A/C | A | 0.18560235 | 0.12967914 | A | |
| 44 | rs11744376 | 40396782 | A/G | A | 0.06375502 | 0.06963788 | G | |
| 45 | rs12523599 | 40397093 | A/G | A | 0.83169291 | 0.77823691 | A | |
| 46 | rs12518585 | 40397433 | A/G | A | 0.16409537 | 0.20700637 | G | |
| 47 | rs12514679 | 40402560 | G/T | G | 0.16966068 | 0.20987654 | T | |
| 48 | rs7725523 | 40407980 | A/G | A | 0.22761194 | 0.28571429 | G | |
| 49 | rs7730693 | 40408862 | A/G | A | 0.52383475 | 0.54647887 | G | |
| 50 | rs12515954 | 40409631 | C/T | C | 0.1722561 | 0.22027027 | T | |
| 51 | rs13186168 | 40411804 | T/A | A | 0.4610951 | 0.44339623 | A | |
| 52 | rs17227583 | 40413623 | C/T | C | 0.19212062 | 0.13207547 | C | |
| 53 | rs4957279 | 40415107 | C/T | C | 0.9320298 | 0.92261905 | C | |
| 54 | rs1031168 | 40417377 | C/T | C | 0.75843254 | 0.7173913 | C | |
| 55 | rs2120855 | 40419377 | A/G | A | 0.51094527 | 0.53794038 | G | |
| 56 | rs895123 | 40419818 | C/G | C | 0.80799605 | 0.86891892 | G | |
| 57 | rs11952844 | 40420052 | C/G | C | 0.47230465 | 0.43989071 | C | |
| 58 | rs12523046 | 40420998 | A/T | A | 0.7530426 | 0.66071429 | A | |
| 59 | rs12523160 | 40421547 | A/T | A | 0.75217391 | 0.61572052 | A | |
| 60 | rs1002922 | 40422312 | T/C | C | 0.24559687 | 0.30253623 | T | |
| 61 | rs1550761 | 40424886 | T/C | C | 0.92134831 | 0.88235294 | C | |
| 62 | rs12187530 | 40425609 | C/A | A | 0.19116187 | 0.12872629 | A | |
| 63 | rs12658567 | 40427689 | G/T | G | 0.48857994 | 0.46091644 | G | |
| 64 | rs4613763 | 40428485 | T/C | C | 0.19129159 | 0.13561644 | C | |
| 65 | rs10512734 | 40429362 | A/G | A | 0.75440313 | 0.6741573 | A | |
| 66 | rs2166194 | 40430693 | T/C | C | 0.93108652 | 0.93051771 | C | |
| 67 | rs11738106 | 40435777 | T/A | A | 0.17026497 | 0.12972973 | A | |
| 68 | rs6883686 | 40438290 | T/C | C | 0.42539526 | 0.45068493 | T | |
| 69 | rs6883975 | 40438434 | A/T | A | 0.20833333 | 0.12937063 | A | |
| 70 | rs1025969 | 40438670 | T/C | C | 0.76565558 | 0.68206522 | C | |
| 71 | rs7723981 | 40440669 | A/G | G | 0.9255 | 0.91553134 | G | |
| 72 | rs6890268 | 40441807 | A/T | T | 0.68452381 | 0.59945504 | T | |
| 73 | rs6896243 | 40442742 | C/T | C | 0.8080402 | 0.87016575 | T | |
| 74 | rs10072596 | 40442790 | A/G | A | 0.50695825 | 0.53773585 | G | |
| 75 | rs16869977 | 40443075 | A/G | A | 0.07635009 | 0.10119048 | G | |
| 76 | rs10512737 | 40445800 | A/G | A | 0.1908284 | 0.13207547 | A | |
| 77 | rs6451494 | 40447048 | T/C | C | 0.68597858 | 0.59504132 | C | |
| 78 | rs12655827 | 40448092 | C/T | C | 0.4891945 | 0.46091644 | C | |
| 79 | rs10473191 | 40448701 | A/T | A | 0.6872428 | 0.56857143 | A | |
| 80 | rs10071761 | 40451368 | T/C | C | 0.2335 | 0.32417582 | T | |
| 81 | rs7730306 | 40459014 | A/C | A | 0.4814257 | 0.44959128 | A | |
| 82 | rs11740512 | 40459805 | A/C | A | 0.81155015 | 0.87016575 | C | |
| 83 | rs6896969 | 40460183 | A/C | A | 0.32844575 | 0.42162162 | C | |
| 84 | rs1899980 | 40463555 | A/G | A | 0.92405063 | 0.91346154 | A | |
| 85 | rs6874500 | 40463640 | A/C | A | 0.5148368 | 0.55585831 | C | |
| 86 | rs13160782 | 40463818 | C/A | A | 0.22326733 | 0.21348315 | A | |
| 87 | rs6880934 | 40464949 | C/T | C | 0.4244403 | 0.38095238 | C | |
| 88 | rs6880809 | 40465007 | A/T | A | 0.5242915 | 0.55163043 | T | |
| 89 | rs1545334 | 40465875 | A/G | A | 0.24679803 | 0.33150685 | G | |
| 90 | rs1373693 | 40466932 | A/G | A | 0.81127451 | 0.8760218 | G | |
| 91 | rs1373692 | 40466940 | A/C | C | 0.68638171 | 0.59444444 | C | |
| 92 | rs10512739 | 40467638 | A/G | A | 0.92387218 | 0.89880952 | A | |
| 93 | rs12514415 | 40470793 | C/T | C | 0.76413255 | 0.67379679 | C | |
| 94 | rs7734434 | 40472455 | C/T | C | 0.82528958 | 0.88129496 | T | |
| 95 | rs13165432 | 40477402 | C/G | C | 0.22749511 | 0.20945946 | C | |
| 96 | rs4957295 | 40483754 | G/A | A | 0.2685743 | 0.35294118 | G | Block IV |
| 97 | rs13358164 | 40491719 | A/G | A | 0.26607319 | 0.34688347 | G | |
| 98 | rs7709690 | 40492628 | A/G | A | 0.72927073 | 0.64824798 | A | |
| 99 | rs6890667 | 40492989 | A/T | A | 0.2546706 | 0.33651226 | T | |
| 100 | rs11955354 | 40493216 | A/G | A | 0.26764706 | 0.34782609 | G | |
| 101 | rs10941508 | 40494824 | A/G | A | 0.73196393 | 0.65027322 | A | |
| 102 | rs4957303 | 40502034 | C/T | C | 0.728739 | 0.65013405 | C | |
| 103 | rs4495224 | 40513272 | C/A | A | 0.73043053 | 0.66997167 | A | |
| 104 | rs6876228 | 40516156 | A/G | A | 0.89344262 | 0.85386819 | A | Block V |
| 105 | rs10077544 | 40520695 | A/G | A | 0.87760159 | 0.82782369 | A | |
| | rs7720838 | 40522652 | G/T | | | | | |
| 106 | rs7725052 | 40523027 | C/T | C | 0.35621242 | 0.42191781 | T | |
| 107 | rs13181935 | 40529403 | C/T | C | 0.92662779 | 0.92527174 | C | |
| 108 | rs4957310 | 40531137 | C/G | C | 0.71752266 | 0.68169014 | C | |
| 109 | rs9687948 | 40532033 | A/G | A | 0.71884498 | 0.68233618 | A | |
| 110 | rs7703539 | 40549071 | C/T | C | 0.28621379 | 0.32627119 | T | |
| | rs1553575 | 40538688 | A/G | | | | | |
| 111 | rs10941516 | 40557969 | A/G | G | 0.47896282 | 0.52016129 | | |

The limits of the LD blocks as shown in FIG. 2 are marked in the right-side column. Numbered SNPs correspond to the ones shown in FIG. 2 thus allowing for the identification of the alleles associated with increased versus decreased risk. The table gives in column “position” the nucleotide position of human chromosome wherein the coordinates are corresponding to the march 2006 assembly of the human genome. The table further gives the variation of the position and indicates the allele which is more frequent in Crohn than in the control.

1. A method for determining the genotype of a human individual at the 5p13.1 Crohn's disease risk locus, the method comprising:
a) providing a sample from the individual;
b) determining whether a DNA sequence corresponding to a DNA sequence polymorphism located between coordinated 40,300,000 and 40,600,000 of human chromosome (coordinates corresponding to the march 2006 assembly of the human genome) is present in the sample;
c) determining the nature of the DNA sequence polymorphism genotype located between coordinated 40,300,000 and 40,600,000 of human chromosome as it relates to the genetic risk to develop Crohn's disease.

2. The method according to claim 1, wherein the DNA sequence polymorphism is any of the SNPs (single nucleotide polymorphisms) associated with increased risk for Crohn's disease.

3. The method according to claim 1, including
i) the determination if or if not an allele associated with increased risk for Crohn's disease as indicated in Table 2 is present;
ii) the judgment if or if not said individual is having a genetic risk to develop Crohn's disease, based on the information of step i).

4. The method according to claim 1, including
i) the determination if an allele associated with increased risk for Crohn's disease is present;
ii) the judgment that said individual is having a genetic risk to develop Crohn's disease, if an allele associated with increased risk for Crohn's disease was determined.

5. The method according to claim 3, wherein the allele associated with increased risk for Crohn's disease is selected from the CD risk haplotypes consisting of IIIA, IIIC, IIA, IIB, and IVB.

6. The method according to claim 5, wherein the judgement considers that the presence of the CD risk haplotypes at the 5p13.1 risk locus increase the relative risk by a factor of approximately 1.5 compared to cases wherein the CD risk alleles are absent.

7. The method according claim 3, wherein the method includes
iii) the determination if a further allele selected from the group consisting of CARD15, IL23R, OCTN, DLG5, TNFSF15 and ATG16L1 associated with increased risk for Crohn's disease is present in said individual; and
iv) the judgment that said individual is having a further increased genetic risk to develop Crohn's disease, if in addition to the presence of risk alleles at the 5p13.1 Crohn's disease risk locus any one or more of the allele associated with increased risk for Crohn's disease indicated in iii) was determined.

8. The method according to claim 1, wherein RNA is obtained from said sample and the RNA is converted into cDNA by means of a reverse transcriptase.

9. A method for judging a possibility of the onset of Crohn's disease, wherein a sample from a human individual is tested, wherein a human individual in which the DNA sequence located between coordinated 40,300,000 and 40,600,000 of human chromosome (coordinates corresponding to the march 2006 assembly of the human genome) contains an allele associated with increased risk for Crohn's disease is judged to have a risk of the onset of Crohn's disease.

10. The method of claim 9, wherein the allele associated with increased risk for Crohn's disease is selected from the CD risk haplotypes consisting of IIIA, IIIC, IIA, IIB, IIC and IVB.

11. (canceled)

12. (canceled)

13. (canceled)

14. (canceled)

15. An oligonucleotide for determining the genotype of a human individual at the 5p13.1 Crohn's disease risk locus, selected from the group consisting of:
a) an oligonucleotide comprising from 12 to 30 contiguous nucleotides of the sequence located between coordinated 40,300,000 and 40,600,000 of human chromosome (coordinates corresponding to the march 2006 assembly of the human genome), wherein said oligonucleotide include one position of the SNPs, and wherein said position is occupied by a nucleotide corresponding to the respective SNPs correlated with the risk of Crohn's disease.
b) an oligonucleotide which is entirely complementary to the oligonucleotide of (a).
